(19) United States
(12) Patent Application Publication
(10) Pub. No.: US 2025/0101439 A1
(43) Pub. Date: Mar. 27, 2025

(54) MATERIALS AND METHODS FOR CONTROLLING GENE EDITING

(71) Applicants: CRISPR THERAPEUTICS AG, Zug (CH); BAYER HEALTHCARE LLC, Whippany, NJ (US)

(72) Inventors: Ryo TAKEUCHI, Cambridge, MA (US); Abraham SCARIA, Cambridge, MA (US)

(21) Appl. No.: 18/971,791

(22) Filed: Dec. 6, 2024

Related U.S. Application Data
(63) Continuation of application No. 16/912,999, filed on Jun. 26, 2020, now Pat. No. 12,173,290.
(60) Provisional application No. 62/868,209, filed on Jun. 28, 2019.

(51) Int. Cl. C12N 15/64; C12N 9/22; C12N 15/86

(52) U.S. Cl. CPC C12N 15/64; C12N 9/22; C12N 15/86; C12N 2310/122; C12N 2310/20; C12N 2750/14143; C12N 2750/14151; C12N 2800/22

(57) ABSTRACT

The present application provides materials and methods for controlling gene editing. The present application also provides materials and methods for controlling transcriptional expression of guide RNAs and/or post-transcriptional expression of Cas nuclease.

### RELATED APPLICATIONS

This application is a continuation of U.S. patent application Ser. No. 16/912,999, filed Jun. 26, 2020, allowed as U.S. Pat. No. 12,173,290, which claims the benefit of U.S. Provisional Application Ser. No. 62/868,209, filed on Jun. 28, 2019, which are hereby incorporated by reference in their entireties.

### INCORPORATION BY REFERENCE OF SEQUENCE LISTING

The text of the computer readable sequence listing filed herewith, titled “CRISP_42650_303_SequenceListing.xml”, created Nov. 27, 2024, having a file size of 292,062 bytes, is hereby incorporated by reference in its entirety.

### FIELD

The present disclosure generally relates to the field of molecular biology, including materials and methods for controlling gene editing by a Cas nuclease.

### BACKGROUND

The use of proteins, polynucleotides, and other technology related to clustered regularly interspaced short palindromic repeats (CRISPR) systems is a possible avenue of treatment for various diseases caused by genetic defects and/or ameliorated by genetic treatments. For example, a vector carrying CRISPR-related genes can be introduced into diseased cells in vitro or in vivo, and the resulting cells can then ameliorate or eliminate the disease, based on genetic modification of the cells by the CRISPR-related genes or gene products. A CRISPR system can perform at least some of the system's intended genetic modification functions, leading to amelioration of a disease. However, the CRISPR-related genes may continue to be expressed after the expression is no longer desired.

Currently, there is a lack of efficient and effective materials and methods for controlling expression of CRISPR-related genes that are delivered to cells.

### SUMMARY

This section provides a general summary of the disclosure, and is not comprehensive of its full scope or all of its features.

Provided herein is a CRISPR/Cas system. The CRISPR/Cas system comprises: a nuclease segment comprising a codon optimized nucleotide sequence that encodes a Cas nuclease or variant thereof; a guide RNA (gRNA) segment comprising a nucleotide sequence that encodes a gRNA or sgRNA; and a promoter segment comprising a nucleotide sequence that encodes a first promoter comprising one or more tetracycline operator sequence, wherein the gRNA segment is operably linked to the promoter segment.

Also provided herein is a pharmaceutical composition. The pharmaceutical composition comprises a CRISPR/Cas system. The CRISPR/Cas system comprises: a nuclease segment comprising a codon optimized nucleotide sequence that encodes a Cas nuclease or variant thereof; a gRNA segment comprising a nucleotide sequence that encodes a gRNA or sgRNA; and a promoter segment comprising a nucleotide sequence that encodes a first promoter comprising one or more tetracycline operator sequence, wherein the gRNA segment is operably linked to the promoter segment.

Also provided herein is a packaging cell. The packaging cell comprises a CRISPR/Cas system. The CRISPR/Cas system comprises: a nuclease segment comprising a codon optimized nucleotide sequence that encodes a Cas nuclease or variant thereof; a gRNA segment comprising a nucleotide sequence that encodes a gRNA or sgRNA; and a promoter segment comprising a nucleotide sequence that encodes a first promoter comprising one or more tetracycline operator sequence, wherein the gRNA segment is operably linked to the promoter segment.

Also provided herein is a method of controlling transcription of gRNAs during AAV packaging. The method comprises contacting a packaging cell with a CRISPR/Cas system. The CRISPR/Cas system comprises: a nuclease segment comprising a codon optimized nucleotide sequence that encodes a Cas nuclease or variant thereof; a gRNA segment comprising a nucleotide sequence that encodes a gRNA or sgRNA; and a promoter segment comprising a nucleotide sequence that encodes a first promoter comprising one or more tetracycline operator sequence, wherein the gRNA segment is operably linked to the promoter segment.

Also provided herein is a method of reducing mutagenesis at one or more SIN site in a recombinant AAV vector. The method comprises contacting a packaging cell with a CRISPR/Cas system. The CRISPR/Cas system comprises: a nuclease segment comprising a codon optimized nucleotide sequence that encodes a Cas nuclease or variant thereof; a gRNA segment comprising a nucleotide sequence that encodes a gRNA or sgRNA; and a promoter segment comprising a nucleotide sequence that encodes a first promoter comprising one or more tetracycline operator sequence, wherein the gRNA segment is operably linked to the promoter segment.

Provided herein is a recombinant Adeno-associated virus (AAV) vector. The recombinant AAV vector comprises: a nuclease segment comprising a codon optimized nucleotide sequence that encodes a Cas nuclease or variant thereof; a gRNA segment comprising a nucleotide sequence that encodes a gRNA or sgRNA; and a promoter segment comprising a nucleotide sequence that encodes a first promoter comprising one or more tetracycline operator sequence, wherein the gRNA segment is operably linked to the promoter segment.

Also provided herein is a pharmaceutical composition. The pharmaceutical composition comprises a recombinant AAV vector. The recombinant AAV vector comprises: a nuclease segment comprising a codon optimized nucleotide sequence that encodes a Cas nuclease or variant thereof; a gRNA segment comprising a nucleotide sequence that encodes a gRNA or sgRNA; and a promoter segment comprising a nucleotide sequence that encodes a first promoter comprising one or more tetracycline operator sequence, wherein the gRNA segment is operably linked to the promoter segment.

Also provided herein is a genetically modified cell. The genetically modified cell comprises a recombinant AAV vector. The recombinant AAV vector comprises: a nuclease segment comprising a codon optimized nucleotide sequence that encodes a Cas nuclease or variant thereof; a gRNA segment comprising a nucleotide sequence that encodes a gRNA or sgRNA; and a promoter segment comprising a nucleotide sequence that encodes a first promoter comprising one or more tetracycline operator sequence, wherein the gRNA segment is operably linked to the promoter segment.

Also provided herein is a method of controlling transcription of gRNAs during AAV packaging. The method comprises: contacting a packaging cell with a nucleic acid encoding a recombinant AAV vector; and contacting the packaging cell with at least one vector comprising nucleic acid sequence encoding a tetracycline repressor. The recombinant AAV vector comprises: a nuclease segment comprising a codon optimized nucleotide sequence that encodes a Cas nuclease or variant thereof; a gRNA segment comprising a nucleotide sequence that encodes a gRNA or sgRNA; and a promoter segment comprising a nucleotide sequence that encodes a first promoter comprising one or more tetracycline operator sequence, wherein the gRNA segment is operably linked to the promoter segment.

Also provided herein is a method of reducing mutagenesis at one or more SIN site in a recombinant AAV vector. The method comprises: contacting a packaging cell with nucleic acid encoding a recombinant AAV vector; and contacting the packaging cell with at least one vector comprising nucleic acid sequence encoding a tetracycline repressor. The recombinant AAV vector comprises: a nuclease segment comprising a codon optimized nucleotide sequence that encodes a Cas nuclease or variant thereof; a gRNA segment comprising a nucleotide sequence that encodes a gRNA or sgRNA; and a promoter segment comprising a nucleotide sequence that encodes a first promoter comprising one or more tetracycline operator sequence, wherein the gRNA segment is operably linked to the promoter segment.

Also provided herein is a method of producing a recombinant AAV vector. The method comprises: introducing into a packaging cell: (i) a first vector comprising a repressor segment, wherein the repressor segment comprises a nucleotide sequence that encodes a tetracycline repressor protein; (ii) a nucleic acid comprising sequence encoding a recombinant AAV vector; and (iii) one or more viral components for producing the recombinant AAV vector; culturing the packaging cell; and isolating the recombinant AAV vector comprising the nucleic acid from the packaging cell. The recombinant AAV vector comprises: a nuclease segment comprising a codon optimized nucleotide sequence that encodes a Cas nuclease or variant thereof; a gRNA segment comprising a nucleotide sequence that encodes a gRNA or sgRNA; and a promoter segment comprising a nucleotide sequence that encodes a first promoter comprising one or more tetracycline operator sequence, wherein the gRNA segment is operably linked to the promoter segment.

Also provided herein is a method of producing a recombinant AAV vector. The method comprises: introducing into a packaging cell a nucleic acid comprising a sequence encoding a recombinant AAV vector; introducing into the packaging cell one or more viral components for producing the AAV; culturing the packaging cell; and isolating the recombinant AAV vector comprising the nucleic acid from the packaging cell. The packaging cell expresses a tetracycline repressor protein. The recombinant AAV vector comprises: a nuclease segment comprising a codon optimized nucleotide sequence that encodes a Cas nuclease or variant thereof; a gRNA segment comprising a nucleotide sequence that encodes a gRNA or sgRNA; and a promoter segment comprising a nucleotide sequence that encodes a first promoter comprising one or more tetracycline operator sequence, wherein the gRNA segment is operably linked to the promoter segment.

Provided herein is a CRISPR/Cas system. The CRISPR/Cas system comprises: a nuclease segment comprising a codon optimized nucleotide sequence that encodes a Cas nuclease or variant thereof; a gRNA segment comprising a nucleotide sequence that encodes a gRNA or sgRNA; and a short-hairpin RNA (shRNA) segment comprising a nucleotide sequence that encodes a shRNA that comprises sequence that is complementary to a transcript from the nuclease segment.

Also provided herein is a pharmaceutical composition comprising a CRISPR/Cas system. The CRISPR/Cas system comprises: a nuclease segment comprising a codon optimized nucleotide sequence that encodes a Cas nuclease or variant thereof; a gRNA segment comprising a nucleotide sequence that encodes a gRNA or sgRNA; and a shRNA segment comprising a nucleotide sequence that encodes a shRNA that comprises sequence that is complementary to a transcript from the nuclease segment.

Also provided herein is a packaging cell comprising a CRISPR/Cas system. The CRISPR/Cas system comprises: a nuclease segment comprising a codon optimized nucleotide sequence that encodes a Cas nuclease or variant thereof; a gRNA segment comprising a nucleotide sequence that encodes a gRNA or sgRNA; and a shRNA segment comprising a nucleotide sequence that encodes a shRNA that comprises sequence that is complementary to a transcript from the nuclease segment.

Also provided herein is a method of controlling post-transcriptional expression of Cas nuclease during AAV packaging. The method comprises: contacting a packaging cell with a CRISPR/Cas system. The CRISPR/Cas system comprises: a nuclease segment comprising a codon optimized nucleotide sequence that encodes a Cas nuclease or variant thereof; a gRNA segment comprising a nucleotide sequence that encodes a gRNA or sgRNA; and a shRNA segment comprising a nucleotide sequence that encodes a shRNA that comprises sequence that is complementary to a transcript from the nuclease segment.

Also provided herein is a method of reducing mutagenesis at one or more SIN site in a recombinant AAV vector. The method comprises: contacting a cell with a CRISPR/Cas system. The CRISPR/Cas system comprises: a nuclease segment comprising a codon optimized nucleotide sequence that encodes a Cas nuclease or variant thereof; a gRNA segment comprising a nucleotide sequence that encodes a gRNA or sgRNA; and a shRNA segment comprising a nucleotide sequence that encodes a shRNA that comprises sequence that is complementary to a transcript from the nuclease segment.

Also provided herein is a method of controlling post-transcriptional expression of Cas nuclease during AAV packaging. The method comprises: contacting a packaging cell with a nucleic acid comprising a sequence encoding a recombinant AAV vector; and contacting the packaging cell with at least one vector comprising nucleic acid sequence encoding a shRNA segment. The recombinant AAV vector comprises: a nuclease segment comprising a codon optimized nucleotide sequence that encodes a Cas nuclease or variant thereof; a gRNA segment comprising a nucleotide sequence that encodes a gRNA or sgRNA; and a promoter segment comprising a nucleotide sequence that encodes a first promoter comprising one or more tetracycline operator sequence, wherein the gRNA segment is operably linked to the promoter segment.

Also provided herein is a method of producing a recombinant AAV vector. The method comprises: introducing into a packaging cell: (i) a first vector comprising a shRNA segment comprising a nucleotide sequence that encodes a shRNA that comprises sequence that is complementary to a transcript from the nuclease segment; (ii) a nucleic acid comprising sequence encoding a recombinant AAV vector; and (iii) one or more viral components for producing the recombinant AAV vector; culturing the packaging cell; and isolating the recombinant AAV vector comprising the nucleic acid from the packaging cell. The recombinant AAV vector comprises: a nuclease segment comprising a codon optimized nucleotide sequence that encodes a Cas nuclease or variant thereof; a gRNA segment comprising a nucleotide sequence that encodes a gRNA or sgRNA; and a promoter segment comprising a nucleotide sequence that encodes a first promoter comprising one or more tetracycline operator sequence, wherein the gRNA segment is operably linked to the promoter segment.

Also provided herein is a method of producing a recombinant AAV vector. The method comprises: introducing into a packaging cell a nucleic acid comprising sequence encoding a recombinant AAV vector; introducing into the packaging cell one or more viral components for producing the AAV; culturing the packaging cell; and isolating the recombinant AAV vector comprising the nucleic acid from the packaging cell. The packaging cell expresses a shRNA. The recombinant AAV vector comprises: a nuclease segment comprising a codon optimized nucleotide sequence that encodes a Cas nuclease or variant thereof; a gRNA segment comprising a nucleotide sequence that encodes a gRNA or sgRNA; and a promoter segment comprising a nucleotide sequence that encodes a first promoter comprising one or more tetracycline operator sequence, wherein the gRNA segment is operably linked to the promoter segment.

Provided herein is a CRISPR/Cas system. The CRISPR/Cas system comprises: a nuclease segment comprising a codon optimized nucleotide sequence that encodes a Cas nuclease or variant thereof; a gRNA segment comprising a nucleotide sequence that encodes a gRNA or sgRNA; a promoter segment comprising a nucleotide sequence that encodes a first promoter comprising one or more tetracycline operator sequences, wherein the gRNA segment is operably linked to the promoter segment; and a shRNA segment comprising a nucleotide sequence that encodes a shRNA that comprises sequence that is complementary to a transcript from the nuclease segment.

Also provided herein is a pharmaceutical composition comprising a CRISPR/Cas system. The CRISPR/Cas system comprises: a nuclease segment comprising a codon optimized nucleotide sequence that encodes a Cas nuclease or variant thereof; a gRNA segment comprising a nucleotide sequence that encodes a gRNA or sgRNA; a promoter segment comprising a nucleotide sequence that encodes a first promoter comprising one or more tetracycline operator sequences, wherein the gRNA segment is operably linked to the promoter segment; and a shRNA segment comprising a nucleotide sequence that encodes a shRNA that comprises sequence that is complementary to a transcript from the nuclease segment.

Also provided herein is a packaging cell comprising a CRISPR/Cas system. The CRISPR/Cas system comprises: a nuclease segment comprising a codon optimized nucleotide sequence that encodes a Cas nuclease or variant thereof; a gRNA segment comprising a nucleotide sequence that encodes a gRNA or sgRNA; a promoter segment comprising a nucleotide sequence that encodes a first promoter comprising one or more tetracycline operator sequences, wherein the gRNA segment is operably linked to the promoter segment; and a shRNA segment comprising a nucleotide sequence that encodes a shRNA that comprises sequence that is complementary to a transcript from the nuclease segment.

Also provided herein is a method of controlling transcription of gRNAs and post-transcriptional expression of Cas nuclease during AAV packaging. The method comprises: contacting a packaging cell with a CRISPR/Cas system. The CRISPR/Cas system comprises: a nuclease segment comprising a codon optimized nucleotide sequence that encodes a Cas nuclease or variant thereof; a gRNA segment comprising a nucleotide sequence that encodes a gRNA or sgRNA; a promoter segment comprising a nucleotide sequence that encodes a first promoter comprising one or more tetracycline operator sequences, wherein the gRNA segment is operably linked to the promoter segment; and a shRNA segment comprising a nucleotide sequence that encodes a shRNA that comprises sequence that is complementary to a transcript from the nuclease segment.

Also provided herein is a method of reducing mutagenesis at one or more SIN site in a recombinant AAV vector. The method comprises: contacting a cell with a CRISPR/Cas system. The CRISPR/Cas system comprises: a nuclease segment comprising a codon optimized nucleotide sequence that encodes a Cas nuclease or variant thereof; a gRNA segment comprising a nucleotide sequence that encodes a gRNA or sgRNA; a promoter segment comprising a nucleotide sequence that encodes a first promoter comprising one or more tetracycline operator sequences, wherein the gRNA segment is operably linked to the promoter segment; and a shRNA segment comprising a nucleotide sequence that encodes a shRNA that comprises sequence that is complementary to a transcript from the nuclease segment.

Also provided herein is a method of controlling transcription of gRNAs and post-transcriptional expression of Cas nuclease during AAV packaging. The method comprises: contacting a packaging cell with a nucleic acid comprising sequence encoding a recombinant AAV vector; and contacting the packaging cell with a nucleic acid sequence encoding a tetracycline repressor segment and a shRNA segment. The recombinant AAV vector comprises: a nuclease segment comprising a codon optimized nucleotide sequence that encodes a Cas nuclease or variant thereof; a gRNA segment comprising a nucleotide sequence that encodes a gRNA or sgRNA; and a promoter segment comprising a nucleotide sequence that encodes a first promoter comprising one or more tetracycline operator sequence, wherein the gRNA segment is operably linked to the promoter segment.

Also provided herein is a method of producing a recombinant AAV vector. The method comprises: introducing into a packaging cell: (i) a first vector comprising a repressor segment, wherein the repressor segment comprises a nucleotide sequence that encodes a tetracycline repressor protein; (ii) a second vector comprising a shRNA segment comprising a nucleotide sequence that encodes a shRNA that comprises sequence that is complementary to a transcript from the nuclease segment; (iii) a nucleic acid comprising sequence encoding a recombinant AAV vector; and (iv) one or more viral components for producing the recombinant AAV vector; culturing the packaging cell; and isolating the recombinant AAV vector comprising the nucleic acid of (iii) from the packaging cell. The recombinant AAV vector comprises: a nuclease segment comprising a codon optimized nucleotide sequence that encodes a Cas nuclease or variant thereof; a gRNA segment comprising a nucleotide sequence that encodes a gRNA or sgRNA; and a promoter segment comprising a nucleotide sequence that encodes a first promoter comprising one or more tetracycline operator sequence, wherein the gRNA segment is operably linked to the promoter segment.

Also provided herein is a method of producing a recombinant AAV vector. The method comprises: introducing into packaging cell a nucleic acid comprising sequence encoding a recombinant AAV vector; introducing into the packaging cell one or more viral components for producing the AAV; culturing the packaging cell; and isolating the recombinant AAV vector comprising the nucleic acid from the packaging cell. The packaging cell expresses a tetracycline repressor protein and a shRNA. The recombinant AAV vector comprises: a nuclease segment comprising a codon optimized nucleotide sequence that encodes a Cas nuclease or variant thereof; a gRNA segment comprising a nucleotide sequence that encodes a gRNA or sgRNA; and a promoter segment comprising a nucleotide sequence that encodes a first promoter comprising one or more tetracycline operator sequence, wherein the gRNA segment is operably linked to the promoter segment.

The foregoing summary is illustrative only and is not intended to be in any way limiting. In addition to the illustrative examples and features described herein, further aspects, examples, objects and features of the disclosure will become fully apparent from the drawings and the detailed description and the claims.

### BRIEF DESCRIPTION OF THE DRAWINGS

Various aspects of materials and methods disclosed and described in this specification can be better understood by reference to the accompanying figures, in which:

FIGS. 1A-1F show a reporter sequence comprising a P23H target site in an endogenous β-tubulin gene locus of human embryonic kidney (HEK) 293 FT cells; a depiction of pSIA113; a depiction of pSIA122; and data (flow cytometry and western blot) generated using HEK 293 FT reporter cells transfected with a RFP-expressing plasmid (NC026) and either pSIA113 or pSIA122.

FIG. 1A is a depiction of a reporter sequence comprising a P23H target site integrated in an endogenous β-tubulin gene locus of HEK 293 FT cells, generating HEK 293 FT reporter cells.

FIG. 1B is a depiction of pSIA113.

FIG. 1C is a depiction of pSIA122.

FIG. 1D shows flow cytometry data for the HEK 293 FT reporter cells transfected with pSIA113 and NC026.

FIG. 1E shows flow cytometry data for HEK 293 FT reporter cells transfected with pSIA122 and NC026.

FIG. 1F is a western blot showing SaCas9 and β-actin expression in HEK 293 FT reporter cells transfected with NC026 and either pSIA113 or pSIA122.

FIGS. 2A-2D show a depiction of pSIA071; a depiction of pSIA087; and data (flow cytometry and western blot) generated using HEK 293 FT reporter cells transfected with NC026 and either pSIA071 or pSIA087.

FIG. 2A is a depiction of pSIA071.

FIG. 2B is a depiction of pSIA087.

FIG. 2C is a bar graph showing the percentage of BFP negative (“BFP−”) cells and RFP positive (RFP+) cells within the HEK 293 FT reporter cells transfected with NC026 and either pSIA071 or pSIA087.

FIG. 2D is a western blot showing SaCas9 and β-actin expression in HEK 293 FT reporter cells transfected with NC026 and either pSIA071 or SIA087.

FIGS. 3A-3E show a depiction of pSIA132; a depiction of pDL122; a generalized depiction of pDL151-pDL153; and data (western blot and flow cytometry) generated using HEK 293 FT reporter cells co-transfected with pSIA132, NC026, and either (1) pDL122, (2) pDL151, (3) pDL152; or (4) pDL153.

FIG. 3A is a depiction of pSIA132.

FIG. 3B is a depiction of pDL122.

FIG. 3C is a generalized depiction of pDL151, pDL152, and pDL153.

FIG. 3D is a western blot showing SaCas9 and β-actin expression in HEK 293 FT reporter cells co-transfected with pSIA132, NC026, and either (1) pDL122, (2) pDL151, (3) pDL152, or (4) pDL153.

FIG. 3E is a bar graph showing the percentage of BFP-cells and RFP+ cells in HEK 293 FT reporter cells co-transfected with pSIA132, NC026, and either (1) pDL122, (2) pDL151, (3) pDL152, or pDL153.

FIGS. 4A-4D show depictions of pSIA111, pSIA121, pSIA142, and pSIA119.

FIG. 4A is a depiction of pSIA111.

FIG. 4B is a depiction of pSIA121.

FIG. 4C is a depiction of pSIA142.

FIG. 4D is a depiction of pSIA119.

FIG. 5 shows an alkaline electrophoresis gel comparing AAV2 vectors packaged with either (1) pSIA113, (2) pSIA111, or (3) pSIA121 as the template for recombinant AAV vector production. FIG. 5 also compares AAV5 vectors packaged with either pSIA142 or pSIA119.

FIG. 6 shows images of a PCR analysis comparing AAV2 vector packaged with pSIA113 and AAV5 vectors packaged with either pSIA142 or pSIA119 as the template for recombinant AAV vector production.

FIG. 7 shows T7E1 assay results comparing AAV2 vectors packaged with either (1) pSIA111, (2) pSIA113, or (3) pSIA121 as the template for recombinant AAV vector production and AAV5 vector packaged with pSIA142 as the template for recombinant AAV vector production.

FIGS. 8A-B show a depiction of pSIA187 and PCR results comparing an AAV5 vectors packaged with either (1) pSIA185, (2) pSIA186, (3) pSIA187, or (4) pSIA036 as the template for recombinant AAV vector production.

FIG. 8A is a depiction of pSIA187.

FIG. 8B shows PCR results comparing AAV5 vectors packaged with either (1) pSIA185, (2) pSIA186, (3) pSIA187, or (4) pSIA036 as the template for recombinant AAV vector production.

FIGS. 9A-9C show a depiction of pSIA190; an alkaline electrophoresis gel comparing AAV6 vectors packaged with either (1) pSIA069, (2) pSIA071, (3) pSIA188, (4) pSIA189, or (5) pSIA190 as the template for recombinant AAV vector production; and a bar graph showing the percentage of BFP-HEK 293 FT reporter cells transduced with a recombinant AAV vector prepared with pSIA190.

FIG. 9A is a depiction of pSIA190.

FIG. 9B is an alkaline electrophoresis gel comparing AAV6 vectors packaged with either (1) pSIA069, (2) pSIA071, (3) pSIA188, (4) pSIA189, or (5) pSIA190 as the template for recombinant AAV vector production.

FIG. 9C is a bar graph showing the percentage of BFP-HEK 293 FT reporter cells transduced with a recombinant AAV vector prepared with pSIA190 as the template for recombinant AAV vector production.

FIGS. 10A-10F show a depiction of a BFP splicing reporter sequence integrated in Jump-In™ Grip Tite™ HEK 293 cells; a depiction of pD105; a generalized depiction of pDL258, pDL259, pDL260, pDL261, and pDL262; a depiction of a transfection control, NC026; and flow cytometry results (ratio of GFP to RFP and % BFP+) for BFP splicing reporter cells co-transfected with pD105, NC025, and one of either (1) pDL122, (2) pDL258, (3) pDL259, (4) pDL260, (5) pDL261, or (6) pDL262.

FIG. 10A is a depiction of a BFP splicing reporter sequence integrated in Jump-In™ Grip Tite™ HEK 293 cells, generating BFP splicing reporter cells.

FIG. 10B is a depiction of pD105.

FIG. 10C is a representative depiction of pDL258, pDL259, pDL260, pDL261, and pDL262.

FIG. 10D is a depiction of a transfection control, NC026.

FIG. 10E shows flow cytometry results (ratio of GFP to RFP) for BFP splicing reporter cells co-transfected with pD105, NC025, and one of either (1) pDL122, (2) pDL258, (3) pDL259, (4) pDL260, (5) pDL261, or (6) pDL262.

FIG. 10F shows flow cytometry results (% BFP+) for BFP splicing reporter cells co-transfected with pD105, NC025, and one of either (1) pDL122, (2) pDL258, (3) pDL259, (4) pDL260, (5) pDL261, or (6) pDL262.

### BRIEF DESCRIPTION OF THE SEQUENCE LISTING

SEQ ID NOs: 1 and 2 are sgRNA sequences that target the P23H allele of the human RHO gene.

SEQ ID NO: 3 is an H1 promoter sequence comprising 2 tetracycline operator (TetO) sites.

SEQ ID NO: 4 is a TetO site.

SEQ ID NOs: 5-8 are plasmid sequences comprising recombinant AAV vector sequences.

SEQ ID NOs: 9-11 are shRNA sequences which target SaCas9.

SEQ ID NO: 12 is a plasmid sequence comprising a recombinant AAV vector sequence.

SEQ ID NO: 13 is a plasmid sequence comprising a sequence encoding a tetracycline repressor (TetR) construct.

SEQ ID NOs: 14-16 are plasmid sequences comprising the shRNA sequences of SEQ ID NOs: 9-11.

SEQ ID NOs: 17-20 are plasmid sequences comprising recombinant AAV vector sequences.

SEQ ID NOs: 21-27 are sequences of oligonucleotide primers used during PCR.

SEQ ID NO: 28 is a sequence comprising a blue fluorescent protein (BFP) reporter construct.

SEQ ID NO: 29 is the sequence of the TetR construct of SEQ ID NO: 13.

SEQ ID NO: 30 is the spacer sequence of SEQ ID NO: 1.

SEQ ID NO: 31 is the DNA sequence of the DNA targeted by the spacer sequence of SEQ ID NO: 30.

SEQ ID NO: 32 is the DNA sequence of the DNA on the strand opposite the sequence of SEQ ID NO: 31.

SEQ ID NO: 33 is the spacer sequence of SEQ ID NO: 2.

SEQ ID NO: 34 is the DNA sequence of the DNA targeted by the spacer sequence of SEQ ID NO: 33.

SEQ ID NO: 35 is the DNA sequence of the DNA on the strand opposite the sequence of SEQ ID NO: 34.

SEQ ID NOs: 36-43 are plasmid sequences comprising recombinant AAV vector sequences.

SEQ ID NO: 44 is a sequence comprising a BFP reporter construct.

SEQ ID NO: 45 is a plasmid sequence comprising a recombinant AAV vector sequence.

SEQ ID NO: 46 is a sgRNA sequence that targets an exon 2 splice donor site in the BFP reporter construct of SEQ ID NO: 44.

SEQ ID NO: 47 is the spacer sequence of SEQ ID NO: 46.

SEQ ID NO: 48 is the DNA sequence of the DNA targeted by the spacer sequence of SEQ ID NO: 47.

SEQ ID NO: 49 is the DNA sequence of the DNA on the strand opposite the sequence of SEQ ID NO: 48.

SEQ ID NOs: 50-54 are plasmid sequences comprising sequence encoding shRNAs that target a sRGN gene transcript. One shRNA is encoded on each plasmid.

SEQ ID NOs: 55-59 are shRNAs sequences encoded by the plasmids of SEQ ID NOs: 50-54.

SEQ ID NO: 60 is a sRGN protein sequence.

SEQ ID NO: 61 is a sRGN nucleotide sequence.

SEQ ID NO: 62 is a TetR nucleotide sequence.

SEQ ID NO: 63 is a TetR protein sequence.

SEQ ID NO: 64 is a S. aureus Cas nuclease nucleotide sequence.

SEQ ID NO: 65 is a S. aureus Cas nuclease protein sequence.

SEQ ID NO: 66 is an AAV vector sequence from pSIA111 or pSIA121.

SEQ ID NO: 67 is an AAV vector sequence from pSIA142 or pSIA119.

SEQ ID NO: 68 is an AAV vector sequence from pSIA185-187 or pSIA036.

SEQ ID NO: 69 is an AAV vector sequence from pSIA188-190 or pSIA069.

The exemplifications set out in the accompanying sequence listing and description thereof illustrate certain examples, in one form, and such exemplifications are not to be construed as limiting the scope of the examples in any manner.

### DETAILED DESCRIPTION

Self-inactivating viral vectors can be used to deliver and express CRISPR-related genes and proteins in order to effect desired changes in cells (e.g., gene editing or modulation of gene expression). After the desired changes in cells have been achieved, or at least partly achieved, further expression of CRISPR-related genes (e.g., a Cas nuclease gene and/or a gRNA gene) can be detrimental. Therefore, self-inactivating viral vectors can be advantageous because the viral vectors can inactivate the further expression of CRISPR-related genes after a period of time, thereby reducing the detrimental effects while still achieving desired changes in cells.

A self-inactivating viral vector system can be a 2-vector system or a 1-vector system. The 1-vector system can also be termed an all-in-one self-inactivating vector system. All-in-one self-inactivating vector systems can be advantageous because they comprise the Cas nuclease sequence, gRNA sequence, self-inactivation (SIN) sites and appropriate promoters to drive expression of the Cas nuclease and gRNA in the same vector instead of 2 vectors. However, for all-in-one self-inactivating viral vector systems, production of the viral vector systems can be challenging due to premature editing, DNA shuffling, and/or mutagenesis at SIN sites in the all-in-one self-inactivating viral vector DNA which is packaged in AAV capsids.

Applicants have discovered novel materials and methods for controlling gene editing. The materials and methods can control transcriptional expression of gRNAs and/or post-transcriptional expression of a Cas nuclease. The materials and methods can control transcriptional expression of gRNAs during AAV packaging, control post-transcriptional expression of a Cas nuclease during AAV packaging, and reduce mutagenesis (e.g., indels and/or deletions) at one or more SIN site in recombinant AAV vectors during packaging.

### Novel Materials and Methods for Controlling Gene Editing

The novel materials and methods for controlling gene editing provided herein include the following:
(1) materials and methods that control transcriptional expression of gRNAs using a tetracycline operator/repressor system.
(2) materials and methods that control post-transcriptional expression of a Cas nuclease using a shRNA that is complementary to a Cas nuclease transcript.
(3) materials and methods that control both transcriptional expression of gRNAs using a tetracycline operator/repressor system and control post-transcriptional expression of Cas nuclease using a shRNA that is complementary to a Cas nuclease transcript.
   CRISPR/Cas Systems that Control Transcriptional Expression of gRNAs Using a Tetracycline Operator/Repressor System

Provided herein are CRISPR/Cas systems for controlling transcriptional expression of gRNAs using a tetracycline operator/repressor system. More specifically, provided herein are CRISPR/Cas systems for controlling transcriptional expression of gRNAs during AAV packaging using a tetracycline operator/repressor system.

Provided herein are CRISPR/Cas systems for reducing mutagenesis at one or more SIN site in a recombinant AAV vector using a tetracycline operator/repressor system. More specifically, provided herein are CRISPR/Cas systems for reducing mutagenesis at one or more SIN site in a recombinant AAV vector during AAV packaging using a tetracycline operator/repressor system.

The CRISPR/Cas system disclosed herein can comprise (1) a nuclease segment comprising a codon optimized nucleotide sequence that encodes a Cas nuclease or variant thereof; (2) a gRNA segment comprising a nucleotide sequence that encodes a gRNA or sgRNA; and (3) a promoter segment comprising a nucleotide sequence that encodes a first promoter comprising one or more tetracycline operator sequence. The gRNA segment can be operably linked to the promoter segment. The CRISPR/Cas system can further comprise a repressor segment comprising a nucleotide sequence that encodes a tetracycline repressor protein. The CRISPR/Cas system can further comprise one or more self-inactivating segments comprising a SIN site.

CRISPR/Cas Systems that Control Post-Transcriptional Expression of Cas Nuclease Using a shRNA that is Complementary to a Cas Nuclease Transcript

Provided herein are CRISPR/Cas systems for controlling post-transcriptional expression of Cas nuclease using a shRNA that is complementary to a Cas nuclease transcript. More specifically, provided herein are CRISPR/Cas systems for controlling post-transcriptional expression of Cas nuclease during AAV packaging using a shRNA that is complementary to a Cas nuclease transcript.

Provided herein are CRISPR/Cas systems for reducing mutagenesis at one or more SIN site in a recombinant AAV vector using a shRNA that is complementary to a Cas nuclease transcript. More specifically, provided herein are CRISPR/Cas systems for reducing mutagenesis at one or more SIN site in a recombinant AAV vector during AAV packaging using a shRNA that is complementary to a Cas nuclease transcript.

The CRISPR/Cas system disclosed herein can comprise (1) a nuclease segment comprising a codon optimized nucleotide sequence that encodes a Cas nuclease or variant thereof; (2) a gRNA segment comprising a nucleotide sequence that encodes a gRNA or sgRNA; and (3) a shRNA segment comprising a nucleotide sequence that encodes a shRNA that comprises sequence that is complementary to a transcript from the nuclease segment. The CRISPR/Cas system can further comprise one or more self-inactivating segments comprising a SIN site.

CRISPR/Cas Systems that Control Transcriptional Expression of gRNAs and Control Post-Transcriptional Expression of Cas Nuclease Using a Tetracycline Operator/Repressor System and shRNA that is Complementary to a Cas Nuclease Transcript

Provided herein are CRISPR/Cas systems for controlling transcriptional expression of gRNAs using a tetracycline operator/repressor system and controlling post-transcriptional expression of Cas nuclease using a shRNA that is complementary to a Cas nuclease transcript. More specifically, provided herein are CRISPR/Cas systems for controlling transcriptional expression of gRNAs using a tetracycline operator/repressor system and controlling post-transcriptional expression of Cas nuclease using a shRNA that is complementary to a Cas nuclease transcript during AAV packaging.

Provided herein are CRISPR/Cas systems for reducing mutagenesis at one or more SIN site in a recombinant AAV vector using a tetracycline operator/repressor system and a shRNA that is complementary to a Cas nuclease transcript. More specifically, provided herein are CRISPR/Cas systems for reducing mutagenesis at one or more SIN site in a recombinant AAV vector during AAV packaging using a tetracycline operator/repressor system and a shRNA that is complementary to a Cas nuclease transcript.

The CRISPR/Cas system disclosed herein can comprise (1) a nuclease segment comprising a codon optimized nucleotide sequence that encodes a Cas nuclease or variant thereof; (2) a gRNA segment comprising a nucleotide sequence that encodes a gRNA or sgRNA; (3) a promoter segment comprising a nucleotide sequence that encodes a first promoter comprising one or more tetracycline operator sequences; and (4) a shRNA segment comprising a nucleotide sequence that encodes a shRNA that comprises sequence that is complementary to a transcript from the nuclease segment. The gRNA segment is operably linked to the promoter segment. The CRISPR/Cas system can further comprise a repressor segment comprising a nucleotide sequence that encodes a tetracycline repressor protein. The CRISPR/Cas system can further comprise one or more self-inactivating segments comprising a SIN site.

Recombinant AAV Vectors that Control Transcriptional Expression of gRNAs Using a Tetracycline Operator/Repressor System

Provided herein are recombinant AAV vectors for controlling transcriptional expression of gRNAs using a tetracycline operator/repressor system. More specifically, provided herein are recombinant AAV vectors for controlling transcriptional expression of gRNAs during AAV packaging using a tetracycline operator/repressor system.

Provided herein are recombinant AAV vectors for reducing mutagenesis at one or more SIN site in a recombinant AAV vector using a tetracycline operator/repressor system. More specifically, provided herein are recombinant AAV vectors for reducing mutagenesis at one or more SIN site in a recombinant AAV vector during AAV packaging using a tetracycline operator/repressor system.

The recombinant AAV vectors disclosed herein can comprise (1) a nuclease segment comprising a codon optimized nucleotide sequence that encodes a Cas nuclease or variant thereof; (2) a gRNA segment comprising a nucleotide sequence that encodes a gRNA or sgRNA; and (3) a promoter segment comprising a nucleotide sequence that encodes a promoter comprising one or more tetracycline operator sequence. The gRNA segment can be operably linked to the promoter segment. The recombinant AAV vectors can further comprise one or more self-inactivating segments comprising a SIN site. The recombinant AAV vectors can comprises a nucleic acid sequence having at least 85% sequence identity to SEQ ID NOs: 66-69. The recombinant AAV vectors can comprise a nucleic acid sequence having at least 85%, 86%, 87%, 88%, 89%, 90%, 91%, 92%, 93%, 94%, 95%, 96%, 97%, 98% or 99% sequence identity to SEQ ID NOs: 66-69. The recombinant AAV vectors can comprise SEQ ID NOs: 66-69.

### Additional Recombinant AAV Vectors

Provided herein are recombinant AAV vectors prepared by any one of the methods of producing a recombinant AAV vector disclosed herein.

### Nuclease Segment

The nuclease segment can comprise a codon optimized nucleotide sequence that encodes a Cas nuclease or variant thereof.

CRISPR/Cas loci define Class 1 and Class 2 CRISPR/Cas systems, having Types I to V or VI. See, e.g., Makarova et al., Nat Rev Microbiol, 13 (11): 722-36 (2015); Shmakov et al., Molecular Cell, 60:385-397 (2015). Class 2 CRISPR/Cas systems have single protein effectors. Cas proteins of Types II, V, and VI may be single-protein, RNA-guided endonucleases, herein called “Class 2 Cas nucleases”. The Cas nuclease can be a class 2 Cas nuclease.

The Cas nuclease can be selected from a group consisting of: S. pyogenes Cas nuclease, S. aureus Cas nuclease, S. thermolphilus Cas nuclease, C. jejuni Cas nuclease, T. denticola Cas nuclease, N. meningitides Cas nuclease, S. lugdunensis Cas nuclease, S. hyicus Cas nuclease, S. microti Cas nuclease, S. pasteuri Cas nuclease, and Cpf-1.

The Cas nuclease can be any one of the Cas orthologs from other bacterial strains including but not limited to, Cas proteins identified in Acaryochloris marina MBIC11017; Acetohalobium arabaticum DSM 5501; Acidithiobacillus caldus; Acidithiobacillus ferrooxidans ATCC 23270; Alicyclobacillus acidocaldarius LAA1; Alicyclobacillus acidocaldarius subsp. acidocaldarius DSM 446; Allochromatium vinosum DSM 180; Ammonifex degensii KC4; Anabaena variabilis ATCC 29413; Arthrospira maxima CS-328; Arthrospira platensis str. Paraca; Arthrospira sp. PCC 8005; Bacillus pseudomycoides DSM 12442; Bacillus selenitireducens MLS10; Burkholderiales bacterium 1_1_47; Caldicelulosiruptor becscii DSM 6725; Candidatus Desulforudis audaxviator MP104C; Caldicellulosiruptor hydrothermalis_108; Clostridium phage c-st; Clostridium botulinum A3 str. Loch Maree; Clostridium botulinum Ba4 str. 657; Clostridium difficile QCD-63q42; Crocosphaera watsonii WH 8501; Cyanothece sp. ATCC 51142; Cyanothece sp. CCY0110; Cyanothece sp. PCC 7424; Cyanothece sp. PCC 7822; Exiguobacterium sibiricum 255-15; Finegoldia magna ATCC 29328; Ktedonobacter racemifer DSM 44963; Lactobacillus delbrueckii subsp. bulgaricus PB2003/044-T3-4; Lactobacillus salivarius ATCC 11741; Listeria innocua; Lyngbya sp. PCC 8106; Marinobacter sp. ELB17; Methanohalobium evestigatum Z-7303; Microcystis phage Ma-LMM01; Microcystis aeruginosa NIES-843; Microscilla marina ATCC 23134; Microcoleus chthonoplastes PCC 7420; Nitrosococcus halophilus Nc4; Nocardiopsis dassonvillei subsp. dassonvillei DSM 43111; Nodularia spumigena CCY9414; Nostoc sp. PCC 7120; Oscillatoria sp. PCC 6506; Pelotomaculum_thermopropionicum_SI; Petrotoga mobilis SJ95; Polaromonas naphthalenivorans CJ2; Polaromonas sp. JS666; Pseudoalteromonas haloplanktis TAC125; Streptomyces pristinaespiralis ATCC 25486; Streptomyces pristinaespiralis ATCC 25486; Streptomyces viridochromogenes DSM 40736; Streptosporangium roseum DSM 43021; Synechococcus sp. PCC 7335; and Thermosipho africanus TCF52B (Chylinski et al., RNA Biol., 2013; 10 (5): 726-737.

The Cas nuclease can be a a synthetic, RNA-Guided Nuclease (sRGN), such as any one of the sRGN disclosed in International Application No. PCT/US19/23044 (Publication No: WO/2019/183150), which is incorporated by reference in its entirety. The Cas nuclease can comprise a polypeptide sequence having at least 85%, 86%, 87%, 88%, 89%, 90%, 91%, 92%, 93%, 94%, 95%, 96%, 97%, 98%, or 99% sequence identity to SEQ ID NO: 60. The Cas nuclease can comprise SEQ ID NO: 60. A polynucleotide or polypeptide has a certain percent “sequence identity” to another polynucleotide or polypeptide, meaning that, when aligned, that percentage of bases or amino acids are the same, and in the same relative position, when comparing the two sequences. Sequence identity can be determined in a number of different manners. To determine sequence identity, sequences can be aligned using various methods and computer programs (e.g., BLAST, TCOFFEE, MUSCLE, MAFFT, etc.), available over the world wide web at sites including ncbi.nlm.nili.gov/BLAST, ebi.ac.uk/Tools/msa/tcoffee/, ebi.ac.uk/Tools/msa/muscle/, or mafft.cbrc.jp/alignment/software/. See, e.g., Altschul et al. (1990), J. Mol. Bioi. 215:403-10. Sequence alignments standard in the art are used according to the invention to determine amino acid residues in a Cas ortholog that “correspond to” amino acid residues in another Cas ortholog. The amino acid residues of Cas orthologs that correspond to amino acid residues of other Cas orthologs appear at the same position in alignments of the sequences.

A Cas nuclease can be codon-optimized for expression in the cell containing the target sequence. For example, if the intended target sequence is in a human cell, a human codon-optimized polynucleotide encoding Cas nuclease is contemplated for use for producing the Cas polypeptide.

The codon optimized nucleotide sequence that encodes a Cas nuclease or variant thereof can comprise one or more intron.

A nucleic acid sequence encoding a promoter can be operably linked to the codon optimized nucleotide sequence that encodes a Cas nuclease or variant thereof. The promoter can be a spatially-restricted promoter, bidirectional promoter, or an inducible promoter. The spatially-restricted promoter can be selected from a group consisting of: any tissue or cell type specific promoter, a hepatocyte-specific promoter, a neuron-specific promoter, an adipocyte-specific promoter, a cardiomyocyte-specific promoter, a skeletal muscle-specific promoter, lung progenitor cell specific promoter, a photoreceptor cell-specific promoter, and a retinal pigment epithelial (RPE) selective promoter.

A tissue specific promoter can be used to control transcription of Cas nuclease by directing the expression of Cas nuclease only in certain host cells. For example, the photoreptor-specific promoter, GRK1, can direct transcription of a coding sequence in photoreceptor cells, but may not activate transcription of the same coding sequence at a similar level in non-photoreceptor cells. As a result, GRK1 promoters can be used in packaging cells, such as HEK 293T cells, to control transcription of Cas nuclease. This same approach can be used with other tissue-specific promoters.

A ubiquitously expressed promoter can also be used. A viral promoter can be used.

Although nomenclature is used herein to indicate the species of origin for a given Cas nuclease, it is understood that the Cas nuclease and/or the nucleic acid encoding the Cas nuclease can be modified compared to the sequence occuring in the species of origin. For example, “SpCas” indicates that the Cas gene/protein in question originated in Streptococcus pyogenes and was modified, such as by addition of a nuclear localization signal (NLS) (s) and/or the performance of codon optimization. For example, “SaCas” indicates that the Cas gene/protein in question originated in Staphylococcus aureus and was modified, such as by addition of NLS(s) and/or the performance of codon optimization.

gRNA Segment

The gRNA segment can comprise a nucleotide sequence that encodes a gRNA or sgRNA.

The gRNA segment can comprise a nucleotide sequence that encodes any gRNA or sgRNA that is complementary to a target sequence. A “target DNA” as used herein is a DNA polynucleotide that comprises a target sequence. “Target sequence” as used herein refers to a nucleic acid sequence present in a target DNA which can be edited by a Cas nuclease-gRNA complex directed thereto, provided sufficient conditions for binding exist. For example, the target sequence 5′-GAGCATATC-3′ within a target DNA is targeted by the gRNA sequence 5′-GAGCAUAUC-3′ (e.g., via binding of the opposite strand of the target DNA sequence 5′-GATATGCTC-3′). Suitable DNA/RNA binding conditions include physiological conditions normally present in a cell. Other suitable DNA/RNA binding conditions (e.g., conditions in a cell-free system) are known in the art; see, e.g., Sambrook, J., Fritsch, E. F. and Maniatis, T. Molecular Cloning: A Laboratory Manual, Second Edition, Cold Spring Harbor Laboratory Press, Cold Spring Harbor (1989), particularly Chapter 11 and Table 11.1 therein; and Sambrook, J. and Russell, W., Molecular Cloning: A Laboratory Manual, Third Edition, Cold Spring Harbor Laboratory Press, Cold Spring Harbor (2001). The target DNA can be a double-stranded DNA. The strand of the target DNA that is complementary to and hybridizes with the gRNA can be referred to as the “complementary strand” and the strand of the target DNA that is complementary to the “complementary strand” (and is therefore not complementary to the gRNA) can be referred to as the “noncomplementary strand” or “non-complementary strand.”

The gRNA segment can comprise a nucleotide sequence that encodes a gRNA or sgRNA comprising a spacer sequence can be substantially complementary to a target sequence within the genomic DNA of a cell of a patient. By “complementary,” it is meant that a nucleic acid (e.g. DNA) comprises a sequence of nucleotides that enables it to non-covalently bind, e.g.: form Watson-Crick base pairs, “anneal”, or “hybridize,” to another nucleic acid in a sequence-specific, antiparallel, manner (i.e., a nucleic acid specifically binds to a complementary nucleic acid) under the appropriate in vitro and/or in vivo conditions of temperature and solution ionic strength. As is known in the art, standard Watson-Crick base-pairing includes: adenine (A) pairing with thymidine (T), adenine (A) pairing with uracil (U), and guanine (G) pairing with cytosine (C) [DNA, RNA]. By “substantially complementary,” it is meant that a nucleic acid comprises a sequence where 1-10% or 1-20% of its sequence of nucleotides non-covalently bind to another nucleic acid in a sequence-specific, antiparallel, manner under the appropriate in vitro and/or in vivo conditions of temperature and solution ionic strength.

The gRNA segment can comprise a nucleotide sequence that encodes a gRNA or sgRNA that can be substantially complementary to a SIN site. The gRNA or sgRNA can be fully complementary to the nucleotide sequence of the SIN site except for at one base pair. The gRNA or sgRNA can be fully complementary to the nucleotide sequence of the SIN site except for at two base pairs.

The gRNA segment can comprise a nucleotide sequence that encodes a gRNA or sgRNA that can be substantially complementary to a SIN site and to a target sequence within the genomic DNA of a cell of a patient.

### Promoter Segment

The promoter segment can comprise a nucleotide sequence that encodes a promoter comprising one or more tetracycline operator sequence. The gRNA segment can be operably linked to the promoter segment. The term “operably linked” means that the nucleotide sequence of interest (i.e., the gRNA segment comprising a nucleotide sequence that encodes a gRNA or sgRNA) is linked to regulatory sequence(s) (i.e., the promoter segment comprising a nucleotide sequence that encodes a first promoter comprising one or more tetracycline operator sequences) in a manner that allows for expression of the nucleotide sequence. The term “regulatory sequence” is intended to include, for example, promoters, enhancers and other expression control elements (e.g., polyadenylation signals). Such regulatory sequences are well known in the art and are described, for example, in Goeddel; Gene Expression Technology: Methods in Enzymology 185, Academic Press, San Diego, CA (1990).

For expressing gRNAs used in connection with Cas nucleases, various promoters such as RNA polymerase III promoters, including for example U6 and H1, can be advantageous. Descriptions of and parameters for enhancing the use of such promoters are known in art, and additional information and approaches are regularly being described; see, e.g., Ma, H. et al., Molecular Therapy—Nucleic Acids 3, e161 (2014) doi: 10.1038/mtna.2014.12.

The promoter comprising one or more tetracycline operator sequences can be any RNA polymerase III promotor. The promoter can be selected from a group consisting of: H1 promoter, U6 promoter, 7SK promoter, and portions of any thereof.

The one or more tetracycline operator sequence can comprise a nucleic acid sequence having at least 85%, 86%, 87%, 88%, 89%, 90%, 91%, 92%, 93%, 94%, 95%, 96%, 97%, 98%, 99%, or 100% sequence identity to SEQ ID NO: 4. The one or more tetracycline operator sequence can comprise SEQ ID NO: 4. The one or more tetracycline operator sequences can be bound by a tetracycline repressor protein.

### Repressor Segment

The repressor segment can comprise a nucleotide sequence that encodes a tetracycline repressor protein. The nucleotide sequence that encodes a tetracycline repressor protein can comprise a nucleic acid sequence having at least 85%, 86%, 87%, 88%, 89%, 90%, 91%, 92%, 93%, 94%, 95%, 96%, 97%, 98%, 99%, or 100% sequence identity to SEQ ID NO: 62. The nucleotide sequence that encodes a tetracycline repressor protein can comprise SEQ ID NO: 62. The tetracycline repressor protein can comprise an amino acid sequence having at least 85%, 86%, 87%, 88%, 89%, 90%, 91%, 92%, 93%, 94%, 95%, 96%, 97%, 98%, 99%, or 100% sequence identity to SEQ ID NO: 63. The tetracycline repressor protein can comprise SEQ ID NO: 63.

Binding of the tetracycline repressor protein to the one or more tetracycline operator sequences within the H1promoter can prevent RNA polymerase from binding to the H1 promoter and thereby control transcriptional expression of gRNAs or sgRNAs.

Short-Hairpin RNA (shRNA) Segment

shRNA is an artificial RNA molecule with a hairpin turn that can be used to silence (e.g., reduce) target gene expression via RNA interference (RNAi). Expression of shRNA in cells is typically accomplished by delivery of plasmids or through viral or bacterial vectors. shRNA is an advantageous mediator of RNAi in that it has a relatively low rate of degradation and turnover.

The shRNA segment can comprise a nucleotide sequence that encodes a shRNA that comprises sequence that is complementary to a transcript from the nuclease segment. The shRNA can comprise a sequence having at least 85% sequence identity to any one of SEQ ID NOs: 9-11 or 55-59. The shRNA can comprise a sequence having at least 85%, 86%, 87%, 88%, 89%, 90%, 91%, 92%, 93%, 94%, 95%, 96%, 97%, 98% or 99% sequence identity to any one of SEQ ID NOs: 9-11 or 55-59. The shRNA can comprise any one of SEQ ID NOs: 9-11 or 55-59.

### Self-Inactivating (SIN) Segments

The SIN segments can comprise a SIN site. A “SIN site” as used herein is a site that comprises a target sequence and neighboring protospacer adjacent motif (PAM). For example, a SIN site can comprise 5′-N₁₇₋₂₁NRG-3′ or 5′-N₁₉₋₂₄NNGRRT-3′ wherein N₁₇₋₂₁ or N₁₉₋₂₄ represent a target sequence and NRG or NNGRRT represent PAMs for S. pyogenes Cas nuclease or S. aureus Cas nuclease, respectively. The spacer sequence of a gRNA or sgRNA can hybridize to the complementary strand of the target sequence of the SIN site.

One or more SIN segments can be located in at least one of: (i) at the 5′ end of the codon optimized nucleotide sequence that encodes a Cas nuclease or variant thereof; (ii) at the 3′ end of the codon optimized nucleotide sequence that encodes a Cas nuclease or variant thereof; and (iii) in an intron within the codon optimized nucleotide sequence that encodes a Cas nuclease or variant thereof.

One or more SIN segments can be located in: (i) at the 5′ end of the codon optimized nucleotide sequence that encodes a Cas nuclease or variant thereof; and (ii) in an intron within the codon optimized nucleotide sequence that encodes a Cas nuclease or variant thereof.

The one or more SIN segments can be located within 1-2000 nucleotides, 1-1000 nucleotides, 1-500 nucleotides, 1-250 nucleotides, 1-200 nucleotides, 1-150 nucleotides, 1-100 nucleotides, 1-50 nucleotides, 1-25 nucleotides, 1-10 nucleotides, 1-5 nucleotides, or 1 nucleotide of the 5′ end of the codon optimized nucleotide sequence that encodes a Cas nuclease or variant thereof.

The one or more SIN segments can be located within 1-2000 nucleotides, 1-1000 nucleotides, 1-500 nucleotides, 1-250 nucleotides, 1-200 nucleotides, 1-150 nucleotides, 1-100 nucleotides, 1-50 nucleotides, 1-25 nucleotides, 1-10 nucleotides, 1-5 nucleotides, or 1 nucleotide of the 3′ end of the codon optimized nucleotide sequence that encodes a Cas nuclease or variant thereof.

One of the one or more SIN segments can be located upstream of the codon optimized nucleotide sequence that encodes a Cas nuclease or variant thereof and downstream of a NLS.

The PAM sequence in the SIN site can be selected from a group consisting of: NNGRRT, NRG, NAAAAN, NAAAAC, NNNNGHTT, YTN, NNNNACAC, NNVRYAC, NNNNVRYM, NNAAAAW, NNAGAAW, and NNGG. The PAM sequence in the SIN site can be any known PAM sequence located downstream from a target sequence and recognized by a Cas nuclease-sgRNA complex.

### 2-Vector System

The nuclease segment, the gRNA segment, and the promoter segment can be provided together in a first vector and the repressor segment can be provided in a second vector. The term “vector” refers to a nucleic acid molecule capable of transporting another nucleic acid to which it has been linked. One type of vector is a “plasmid”, which refers to a circular double-stranded DNA loop into which additional nucleic acid segments can be ligated. Another type of vector is a viral vector; wherein additional nucleic acid segments can be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) are integrated into the genome of a host cell upon introduction into the host cell, and thereby are replicated along with the host genome.

The nuclease segment, the gRNA segment, the promoter segment, and the one or more SIN segments can be provided together in a first vector and the repressor segment can be provided in a second vector.

The nuclease segment and the gRNA segment can be provided together in a first vector and the shRNA segment can be provided in a second vector.

The nuclease segment, the gRNA segment, and the one or more SIN segments can be provided together in a first vector and the shRNA segment can be provided in a second vector.

The nuclease segment, the gRNA segment, and the promoter segment can be provided together in a first vector and the repressor segment and/or the shRNA segment can be provided in a second vector.

The nuclease segment, the gRNA segment, the promoter segment, and the one or more SIN segments can be provided together in a first vector and the repressor segment and/or the shRNA segment can be provided in a second vector.

The first vector and the second vector can be AAV vectors or plasmids. The AAV vectors can be AAV1 serotype vectors, AAV2 serotype vectors, AAV3 serotype vectors, AAV4 serotype vectors, AAV5 serotype vectors, AAV6 serotype vectors, AAV7 serotype vectors, AAV8 serotype vectors, AAV9 serotype vectors, or AAVrh8R serotype vectors.

### 1-Vector System (All-In-One Vector System)

The nuclease segment, the gRNA segment, the promoter segment, the one or more SIN segments, and the repressor segment can be provided in the same vector.

The nuclease segment, the gRNA segment, the one or more SIN segments, and the shRNA segment can be provided in the same vector.

The nuclease segment, the gRNA segment, the promoter segment, the one or more SIN segments, the repressor segment, and the shRNA segment can be provided in the same vector.

### Pharmaceutical Compositions

Disclosed herein are pharmaceutical compositions comprising any one of the CRISPR/Cas systems disclosed herein.

Disclosed herein are pharmaceutical compositions comprising any one of the recombinant AAV vectors disclosed herein.

### Packaging Cells

Disclosed herein are packaging cells comprising any one of the CRISPR/Cas systems disclosed herein.

“Packaging cells” as used herein are cells used to form virus particles or viral vectors capable of infecting a host cell. An example of a packaging cell can be a HEK 293T cells. The packaging cells can package one or more viral vector (e.g., based on one or more plasmid templates) comprising any one of the CRISPR/Cas systems disclosed herein into a viral vector particle. The viral vector can contain viral sequences required for packaging. Any missing viral functions can be supplied in trans by the packaging cell. For example, AAV vectors used in gene therapy typically only possess inverted terminal repeat (ITR) sequences from the AAV genome and the balance of the vector can comprise therapeutic sequences. The ITR sequences can be required for packaging into AAV capsids. The packaging cell can also contain a plasmid that encodes other AAV genes (e.g., rep and cap), but lacks ITR sequences. The plasmid that encodes rep and cap genes can not be packaged in significant amounts due to a lack of ITR sequences. The packaging cell can also be infected with adenovirus as a helper virus, which promotes replication of the AAV vector and expression of AAV genes from the plasmid that encodes rep and cap genes. The packaging cell can also be transfected with a helper plasmid encoding gene products of helper viruses, such as adenovirus, which promotes replication of the AAV vector and expression of AAV genes from the plasmid that encodes rep and cap genes.

Purification of AAV particles from a packaging cell involves growth of the packaging cells which produce the viral vectors, followed by collection of the viral vector particles from the cell supernatant and/or from the crude lysate. AAV can then be purified by methods known in the art including ion exchange chromatography (e.g. see U.S. Pat. Nos. 7,419,817 and 6,989,264), ion exchange chromatography and CsCl or iodixanol density centrifugation (e.g. PCT publication WO2011094198A10), immunoaffinity chromatography (e.g. WO2016128408) or purification using AVB Sepharose (e.g. GE Healthcare Life Sciences).

### Genetically Modified Cells

“Genetically modified cell” refers to a cell that comprises at least one genetic modification introduced by genome editing (e.g., using a CRISPR/Cas nuclease system).

Disclosed herein are genetically modified cells comprising any one of the recombinant AAV vectors disclosed herein.

The genetically modified cells can be selected from a group consisting of: a eukaryotic cell, a somatic cell, a germ cell, a stem cell, an animal cell, a mammalian cell, a mouse cell, a non-human primate cell, and a human cell.

The term “genetic modification” refers to any change in the DNA genome (or RNA genome in some cases) of a cell, organism, virus, viral vector, or other biological agent.

Non-limiting examples of genetic modifications include an “insertion,” a “deletion,” a “substitution,” “indels,” a procedure such as a transfection or transformation where exogenous nucleic acid is added to a cell and/or organism, and cloning techniques.

“Insertion” refers to an addition of one or more nucleotides in a nucleic acid sequence. Insertions can range from small insertions of a few nucleotides to insertions of large segments such as a cDNA or a gene.

The term “deletion” refers to a loss or removal of one or more nucleotides in a nucleic acid sequence or a loss or removal of the function of a gene. In some cases, a deletion can include, for example, a loss of a few nucleotides, an exon, an intron, a gene segment, or the entire sequence of a gene. In some cases, deletion of a gene refers to the elimination or reduction of the function or expression of a gene or its gene product. This can result from not only a deletion of sequences within or near the gene, but also other events (e.g., insertion, nonsense mutation) that disrupt the expression of the gene.

“Substitution” refers to a replacement of one or more nucleotides in a nucleic acid sequence with an equal number of nucleotides.

“Indel” or “indels” refers to a series of insertions and/or deletions of nucleotides in a nucleic acid sequence. This can be caused by, for example, a double-stand break in a DNA molecule (e.g., one caused by gene editing nucleases) which is then repaired via a cell's endogenous DNA repair machinery. The double-strand break repair can cause the formation of indels.

Genetic modification of a nucleic acid sequence can result in a “recombinant” sequence. For example, the present disclosure provides “recombinant AAV vectors,” which have been genetically modified to comprise elements disclosed herein.

Methods of Controlling Transcription of gRNAs During AAV Packaging

Disclosed herein are methods of controlling transcription of gRNAs during AAV packaging. The methods can comprise: contacting a packaging cell with any one of the disclosed CRISPR/Cas systems that control transcriptional expression of gRNAs using a tetracycline operator/repressor system. The methods can comprise: contacting a packaging cell with a nucleic acid encoding any one of the disclosed recombinant AAV vectors that control transcriptional expression of gRNAs using a tetracycline operator/repressor system; and contacting the packaging cell with at least one vector comprising a nucleic acid sequence encoding a tetracycline repressor.

### Methods of Controlling Post-Transcriptional Expression of Cas Nuclease During AAV Packaging

Disclosed herein are methods of controlling post-transcriptional expression of Cas nuclease during AAV packaging. The methods can comprise: contacting a packaging cell with any one of the disclosed CRISPR/Cas systems that control post-transcriptional expression of Cas nuclease during AAV packaging. The methods can comprise: contacting a packaging cell with a nucleic acid comprising a sequence encoding any one of the disclosed recombinant AAV vectors that control transcriptional expression of gRNAs using a tetracycline operator/repressor system; and contacting the packaging cell with at least one vector comprising nucleic acid sequence encoding a shRNA segment.

Methods of Controlling Transcription of gRNAs and Post-Transcriptional Expression of Cas Nuclease During AAV Packaging

Disclosed herein are methods of controlling transcription of gRNAs and post-transcriptional expression of Cas nuclease during AAV packaging. The methods can comprise: contacting a packaging cell with any one of the disclosed CRISPR/Cas systems that control transcription of gRNAs and post-transcriptional expression of Cas nuclease during AAV packaging. The methods can comprise: contacting a packaging cell with a nucleic acid comprising a sequence encoding any one of the disclosed recombinant AAV vectors that control transcriptional expression of gRNAs using a tetracycline operator/repressor system; and contacting the packaging cell with a nucleic acid sequence encoding a tetracycline repressor segment and a shRNA segment.

### Methods of Reducing Mutagenesis at One or More SIN Site in a Recombinant AAV Vector

Disclosed herein are methods of reducing mutagenesis at one or more SIN site in a recombinant AAV vector.

The methods can comprise: contacting a packaging cell with any one of the disclosed CRISPR/Cas systems that control transcriptional expression of gRNAs using a tetracycline operator/repressor system during AAV packaging.

The methods can comprise: contacting a packaging cell with a nucleic acid comprising a sequence comprising any one of the disclosed recombinant AAV vectors that control transcriptional expression of gRNAs using a tetracycline operator/repressor system; and contacting the packaging cell with at least one vector comprising nucleic acid sequence encoding a tetracycline repressor.

The methods can comprise: contacting a packaging cell with any one of the disclosed CRISPR/Cas systems that control post-transcriptional expression of Cas nuclease during AAV packaging.

The methods can comprise: contacting a packaging cell with a nucleic acid comprising a sequence encoding any one of the disclosed recombinant AAV vectors that control transcriptional expression of gRNAs using a tetracycline operator/repressor system; and contacting the packaging cell with at least one vector comprising nucleic acid sequence encoding a shRNA segment.

The methods can comprise: contacting a packaging cell with any one of the disclosed CRISPR/Cas systems that control transcription of gRNAs and post-transcriptional expression of Cas nuclease during AAV packaging.

The methods can comprise: contacting a packaging cell with a nucleic acid comprising a sequence encoding any one of the disclosed recombinant AAV vectors that control transcriptional expression of gRNAs using a tetracycline operator/repressor system; and contacting the packaging cell with a nucleic acid sequence encoding a tetracycline repressor segment and a shRNA segment.

### Methods of Producing a Recombinant AAV Vector

Disclosed herein are methods of producing a recombinant AAV vector.

The methods can comprise: introducing into a packaging cell: (i) a first vector comprising a repressor segment, wherein the repressor segment comprises a nucleotide sequence that encodes a tetracycline repressor protein; (ii) a nucleic acid comprising sequence encoding any one of the disclosed recombinant AAV vectors that control transcriptional expression of gRNAs using a tetracycline operator/repressor system; and (iii) one or more viral components for producing the recombinant AAV vector; culturing the packaging cell; and isolating the recombinant AAV vector comprising the nucleic acid from the packaging cell.

The methods can comprise: introducing into a packaging cell a nucleic acid comprising a sequence encoding any one of the disclosed recombinant AAV vectors that control transcriptional expression of gRNAs using a tetracycline operator/repressor system; introducing into the cell one or more viral components for producing the AAV; culturing the packaging cell; isolating the recombinant AAV vector comprising the nucleic acid from the packaging cell. In these methods, the packaging cell can express a tetracycline repressor protein.

The methods can comprise: introducing into a packaging cell: (i) a first vector comprising a shRNA segment comprising a nucleotide sequence that encodes a shRNA that comprises sequence that is complementary to a transcript from the nuclease segment; (ii) a nucleic acid comprising sequence encoding any one of the disclosed recombinant AAV vectors that control transcriptional expression of gRNAs using a tetracycline operator/repressor system; and (iii) one or more viral components for producing the recombinant AAV vector; culturing the packaging cell; isolating the recombinant AAV vector comprising the nucleic acid from the packaging cell.

The methods can comprise: introducing into a packaging cell a nucleic acid comprising a sequence encoding any one of the disclosed recombinant AAV vectors that control transcriptional expression of gRNAs using a tetracycline operator/repressor system; introducing into the cell one or more viral components for producing the AAV; culturing the packaging cell; isolating the recombinant AAV vector comprising the nucleic acid from the packaging cell. In these methods, the packaging cell can express a shRNA.

The methods can comprise: introducing into a packaging cell: (i) a first vector comprising a repressor segment, wherein the repressor segment comprises a nucleotide sequence that encodes a tetracycline repressor protein, (ii) a second vector comprising a shRNA segment comprising a nucleotide sequence that encodes a shRNA that comprises sequence that is complementary to a transcript from the nuclease segment, (iii) a nucleic acid comprising sequence encoding any one of the disclosed recombinant AAV vectors that control transcriptional expression of gRNAs using a tetracycline operator/repressor system, and (iv) one or more viral components for producing the recombinant AAV vector; culturing the packaging cell; and isolating the recombinant AAV vector comprising the nucleic acid of (iii) from the packaging cell.

The methods can comprise: introducing into a packaging cell a nucleic acid comprising a sequence encoding any one of the disclosed recombinant AAV vectors that control transcriptional expression of gRNAs using a tetracycline operator/repressor system; introducing into the packaging cell one or more viral components for producing the AAV; culturing the packaging cell; isolating the recombinant AAV vector comprising the nucleic acid from the packaging cell. In these methods, the packaging cells can express a a tetracycline repressor protein and shRNA.

The nucleic acid can further comprise one or more viral components. The one or more viral components can be introduced via separate vector other than the nucleic acid. The one or more viral components can be encoded in a cellular genome.

Guide RNA (gRNA) or Single-Molecule gRNA (sgRNA)

A gRNA can comprise a spacer sequence and a CRISPR repeat sequence. In Type II systems, the gRNA can also comprise a second RNA called a tracrRNA sequence. In the Type II gRNA, the CRISPR repeat sequence and the tracrRNA sequence hybridize to each other to form a duplex. In the Type V gRNA, the crRNA forms a duplex. In both systems, the duplex can bind a Cas nuclease, such that the gRNA and Cas nuclease form a complex.

A sgRNA in a Type II system can comprise, in the 5′ to 3′ direction, an optional spacer extension sequence, a spacer sequence, a minimum CRISPR repeat sequence, a single-molecule guide linker, a minimum tracrRNA sequence, a 3′ tracrRNA sequence and an optional tracrRNA extension sequence. The optional tracrRNA extension can comprise elements that contribute additional functionality (e.g., stability) to the gRNA. The single-molecule guide linker can link the minimum CRISPR repeat and the minimum tracrRNA sequence to form a hairpin structure. The optional tracrRNA extension can comprise one or more hairpins.

As used herein, “gRNA” means a two-molecule gRNA as described above, a single-molecule gRNA (sgRNA) as described above, or means that either form of gRNA is suitable for the purpose described. As used herein, “sgRNA” means a single-molecule gRNA (sgRNA).

### Spacer Sequence

The spacer sequence hybridizes to a sequence in a target sequence. The spacer sequence of a gRNA or sgRNA can interact with a target sequence in a sequence-specific manner via hybridization (i.e., base pairing). The nucleotide sequence of the spacer sequence can vary depending on the sequence of the target sequence. The spacer sequence can be designed to hybridize to a target sequence that is located 5′ of a PAM of the Cas nuclease used in the system. The spacer sequence can perfectly match the target sequence or can have mismatches. Each Cas nuclease has a particular PAM sequence that it recognizes in a target sequence. For example, S. pyogenes recognizes in a target sequence a PAM that comprises the sequence 5′-NRG-3′, where R comprises either A or G, where N is any nucleotide and N is immediately 3′ of the target sequence targeted by the spacer sequence. For example, S. aureus Cas recognizes in a target sequence a PAM that comprises the sequence 5′-NNGRRT-3′, where R comprises either A or G, where N is any nucleotide and N is immediately 3′ of the target sequence targeted by the spacer sequence. S. aureus Cas can recognize in a target sequence a PAM that comprises the sequence 5′-NNGRRN-3′, where R comprises either A or G, where N is any nucleotide and the N is immediately 3′ of the target sequence targeted by the spacer sequence. For example, C. jejuni recognizes in a target sequence a PAM that comprises the sequence 5′-NNNNACA-3′ or 5′-NNNNACAC-3′, where N is any nucleotide and N is immediately 3′ of the target sequence targeted by the spacer sequence. C. jejuni Cas can recognize in a target sequence a PAM that comprises the sequence 5′-NNNVRYM-3′ or 5′-NNVRYAC-3′, where V comprises either A, G or C, where R comprises either A or G, where Y comprises either C or T, where M comprises A or C, where N is any nucleotide and the N is immediately 3′ of the target sequence targeted by the spacer sequence.

The target sequence can comprise 20 bases immediately 5′ of the first nucleotide of the PAM. For example, in a sequence comprising 5′-NNNNNNNNNNNNNNNNNNNNNRG-3′, the target sequence can comprise the sequence that corresponds to the Ns, wherein N is any nucleotide, and the underlined NRG sequence is the S. pyogenes PAM. The target sequence can comprise less than 20 nucleotides. The target sequence can comprise more than 20 nucleotides. The target sequence can comprise at least: 5, 10, 15, 16, 17, 18, 19, 20, 21, 22, 23, 24, 25, 30, 35 or more nucleotides. The target sequence can comprise at most: 5, 10, 15, 16, 17, 18, 19, 20, 21, 22, 23, 24, 25, 30, 35 or more nucleotides.

The spacer sequence that hybridizes to the target sequence can have a length of at least 15 nucleotides (nt), 16 nt, 17 nt, 18 nt, 19 nt, 20 nt, 21 nt, 22 nt, 23 nt, 24 nt, 25 nt, 26 nt, 27 nt, 28 nt, 29 nt, 30 nt, 31 nt, 32 nt, 33 nt, 34 nt, 35 nt. The gRNA or sgRNA can comprise a spacer sequence comprising 17 to 24 nucleotides. The gRNA or sgRNA can comprise a spacer sequence comprising 17 to 25 nt, 17 to 26 nt, 17 to 27 nt, 17 to 28 nt, 17 to 29 nt, 17 to 30 nt, 17 to 31 nt, 17 to 32 nt, 17 to 33 nt, 17 to 34 nt, or 17 to 35 nt.

The percent complementarity between the spacer sequence and the target sequence is at least about 30%, at least about 40%, at least about 50%, at least about 60%, at least about 65%, at least about 70%, at least about 75%, at least about 80%, at least about 85%, at least about 90%, at least about 95%, at least about 97%, at least about 98%, at least about 99%, or 100%. The percent complementarity between the spacer sequence and the target sequence is at most about 30%, at most about 40%, at most about 50%, at most about 60%, at most about 65%, at most about 70%, at most about 75%, at most about 80%, at most about 85%, at most about 90%, at most about 95%, at most about 97%, at most about 98%, at most about 99%, or 100%.

The spacer sequence can be designed or chosen using a computer program. The computer program can use variables, such as predicted melting temperature, secondary structure formation, predicted annealing temperature, sequence identity, genomic context, chromatin accessibility, % GC, frequency of genomic occurrence (e.g., of sequences that are identical or are similar but vary in one or more spots as a result of mismatch, insertion or deletion), methylation status, presence of SNPs, and the like.

### Minimum CRISPR Repeat Sequence

A minimum CRISPR repeat sequence can be a sequence with at least about 30%, about 40%, about 50%, about 60%, about 65%, about 70%, about 75%, about 80%, about 85%, about 90%, about 95%, or 100% sequence identity to a reference CRISPR repeat sequence (e.g., crRNA from S. pyogenes).

A minimum CRISPR repeat sequence can comprise nucleotides that can hybridize to a minimum tracrRNA sequence in a cell. The minimum CRISPR repeat sequence and a minimum tracrRNA sequence can form a duplex, i.e. a base-paired double-stranded structure. Together, the minimum CRISPR repeat sequence and the minimum tracrRNA sequence can bind to Cas nuclease. At least a part of the minimum CRISPR repeat sequence can hybridize to the minimum tracrRNA sequence. At least a part of the minimum CRISPR repeat sequence can comprise at least about 30%, about 40%, about 50%, about 60%, about 65%, about 70%, about 75%, about 80%, about 85%, about 90%, about 95%, or 100% complementary to the minimum tracrRNA sequence. At least a part of the minimum CRISPR repeat sequence can comprise at most about 30%, about 40%, about 50%, about 60%, about 65%, about 70%, about 75%, about 80%, about 85%, about 90%, about 95%, or 100% complementary to the minimum tracrRNA sequence.

The minimum CRISPR repeat sequence can have a length from about 7 nucleotides to about 100 nucleotides. For example, the length of the minimum CRISPR repeat sequence is from about 7 nt to about 50 nt, from about 7 nt to about 40 nt, from about 7 nt to about 30 nt, from about 7 nt to about 25 nt, from about 7 nt to about 20 nt, from about 7 nt to about 15 nt, from about 8 nt to about 40 nt, from about 8 nt to about 30 nt, from about 8 nt to about 25 nt, from about 8 nt to about 20 nt, from about 8 nt to about 15 nt, from about 15 nt to about 100 nt, from about 15 nt to about 80 nt, from about 15 nt to about 50 nt, from about 15 nt to about 40 nt, from about 15 nt to about 30 nt, or from about 15 nt to about 25 nt. The minimum CRISPR repeat sequence can be approximately 9 nucleotides in length. The minimum CRISPR repeat sequence can be approximately 12 nucleotides in length.

The minimum CRISPR repeat sequence can be at least about 60% identical to a reference minimum CRISPR repeat sequence (e.g., wild-type crRNA from S. pyogenes) over a stretch of at least 6, 7, or 8 contiguous nucleotides. For example, the minimum CRISPR repeat sequence can be at least about 65% identical, at least about 70% identical, at least about 75% identical, at least about 80% identical, at least about 85% identical, at least about 90% identical, at least about 95% identical, at least about 98% identical, at least about 99% identical or 100% identical to a reference minimum CRISPR repeat sequence over a stretch of at least 6, 7, or 8 contiguous nucleotides.

Minimum tracrRNA Sequence

A minimum tracrRNA sequence can be a sequence with at least about 30%, about 40%, about 50%, about 60%, about 65%, about 70%, about 75%, about 80%, about 85%, about 90%, about 95%, or 100% sequence identity to a reference tracrRNA sequence (e.g., wild type tracrRNA from S. pyogenes).

A minimum tracrRNA sequence can comprise nucleotides that hybridize to a minimum CRISPR repeat sequence in a cell. A minimum tracrRNA sequence and a minimum CRISPR repeat sequence form a duplex, i.e. a base-paired double-stranded structure. Together, the minimum tracrRNA sequence and the minimum CRISPR repeat can bind to a Cas nuclease. At least a part of the minimum tracrRNA sequence can hybridize to the minimum CRISPR repeat sequence. The minimum tracrRNA sequence can be at least about 30%, about 40%, about 50%, about 60%, about 65%, about 70%, about 75%, about 80%, about 85%, about 90%, about 95%, or 100% complementary to the minimum CRISPR repeat sequence.

The minimum tracrRNA sequence can have a length from about 7 nucleotides to about 100 nucleotides. For example, the minimum tracrRNA sequence can be from about 7 nt to about 50 nt, from about 7 nt to about 40 nt, from about 7 nt to about 30 nt, from about 7 nt to about 25 nt, from about 7 nt to about 20 nt, from about 7 nt to about 15 nt, from about 8 nt to about 40 nt, from about 8 nt to about 30 nt, from about 8 nt to about 25 nt, from about 8 nt to about 20 nt, from about 8 nt to about 15 nt, from about 15 nt to about 100 nt, from about 15 nt to about 80 nt, from about 15 nt to about 50 nt, from about 15 nt to about 40 nt, from about 15 nt to about 30 nt or from about 15 nt to about 25 nt long. The minimum tracrRNA sequence can be approximately 9 nucleotides in length. The minimum tracrRNA sequence can be approximately 12 nucleotides. The minimum tracrRNA can consist of tracrRNA nt 23-48 described in Jinek et al., Science, 337 (6096): 816-821 (2012).

The minimum tracrRNA sequence can be at least about 60% identical to a reference minimum tracrRNA (e.g., wild type, tracrRNA from S. pyogenes) sequence over a stretch of at least 6, 7, or 8 contiguous nucleotides. For example, the minimum tracrRNA sequence can be at least about 65% identical, about 70% identical, about 75% identical, about 80% identical, about 85% identical, about 90% identical, about 95% identical, about 98% identical, about 99% identical or 100% identical to a reference minimum tracrRNA sequence over a stretch of at least 6, 7, or 8 contiguous nucleotides.

The duplex between the minimum CRISPR RNA and the minimum tracrRNA can comprise a double helix. The duplex between the minimum CRISPR RNA and the minimum tracrRNA can comprise at least about 1, 2, 3, 4, 5, 6, 7, 8, 9, or 10 or more nucleotides. The duplex between the minimum CRISPR RNA and the minimum tracrRNA can comprise at most about 1, 2, 3, 4, 5, 6, 7, 8, 9, or 10 or more nucleotides.

The duplex can comprise a mismatch (i.e., the two strands of the duplex are not 100% complementary). The duplex can comprise at least about 1, 2, 3, 4, or 5 or mismatches. The duplex can comprise at most about 1, 2, 3, 4, or 5 or mismatches. The duplex can comprise no more than 2 mismatches.

3′ tracrRNA Sequence

A 3′ tracrRNA sequence can comprise a sequence with at least about 30%, about 40%, about 50%, about 60%, about 65%, about 70%, about 75%, about 80%, about 85%, about 90%, about 95%, or 100% sequence identity to a reference tracrRNA sequence (e.g., a tracrRNA from S. pyogenes).

The 3′ tracrRNA sequence can have a length from about 6 nucleotides to about 100 nucleotides. For example, the 3′ tracrRNA sequence can have a length from about 6 nt to about 50 nt, from about 6 nt to about 40 nt, from about 6 nt to about 30 nt, from about 6 nt to about 25 nt, from about 6 nt to about 20 nt, from about 6 nt to about 15 nt, from about 8 nt to about 40 nt, from about 8 nt to about 30 nt, from about 8 nt to about 25 nt, from about 8 nt to about 20 nt, from about 8 nt to about 15 nt, from about 15 nt to about 100 nt, from about 15 nt to about 80 nt, from about 15 nt to about 50 nt, from about 15 nt to about 40 nt, from about 15 nt to about 30 nt, or from about 15 nt to about 25 nt. The 3′ tracrRNA sequence can have a length of approximately 14 nucleotides.

The 3′ tracrRNA sequence can be at least about 60% identical to a reference 3′ tracrRNA sequence (e.g., wild type 3′ tracrRNA sequence from S. pyogenes) over a stretch of at least 6, 7, or 8 contiguous nucleotides. For example, the 3′ tracrRNA sequence can be at least about 60% identical, about 65% identical, about 70% identical, about 75% identical, about 80% identical, about 85% identical, about 90% identical, about 95% identical, about 98% identical, about 99% identical, or 100% identical, to a reference 3′ tracrRNA sequence (e.g., wild type 3′ tracrRNA sequence from S. pyogenes) over a stretch of at least 6, 7, or 8 contiguous nucleotides.

The 3′ tracrRNA sequence can comprise more than one duplexed region (e.g., hairpin, hybridized region). The 3′ tracrRNA sequence can comprise two duplexed regions.

The 3′ tracrRNA sequence can comprise a stem loop structure. The stem loop structure in the 3′ tracrRNA can comprise at least 1, 2, 3, 4, 5, 6, 7, 8, 9, 10, 15 or 20 or more nucleotides. The stem loop structure in the 3′ tracrRNA can comprise at most 1, 2, 3, 4, 5, 6, 7, 8, 9 or 10 or more nucleotides. The stem loop structure can comprise a functional moiety. For example, the stem loop structure can comprise an aptamer, a ribozyme, a protein-interacting hairpin, a CRISPR array, an intron, or an exon. The stem loop structure can comprise at least about 1, 2, 3, 4, or 5 or more functional moieties. The stem loop structure can comprise at most about 1, 2, 3, 4, or 5 or more functional moieties.

The hairpin in the 3′ tracrRNA sequence can comprise a P-domain. The P-domain can comprise a double-stranded region in the hairpin.

tracrRNA Extension Sequence

A tracrRNA extension sequence can be provided whether the tracrRNA is in the context of single-molecule guides or double-molecule guides. The tracrRNA extension sequence can have a length from about 1 nucleotide to about 400 nucleotides. The tracrRNA extension sequence can have a length of more than 1, 5, 10, 15, 20, 25, 30, 35, 40, 45, 50, 60, 70, 80, 90, 100, 120, 140, 160, 180, 200, 220, 240, 260, 280, 300, 320, 340, 360, 380, or 400 nucleotides. The tracrRNA extension sequence can have a length from about 20 to about 5000 or more nucleotides. The tracrRNA extension sequence can have a length of more than 1000 nucleotides. The tracrRNA extension sequence can have a length of less than 1, 5, 10, 15, 20, 25, 30, 35, 40, 45, 50, 60, 70, 80, 90, 100, 120, 140, 160, 180, 200, 220, 240, 260, 280, 300, 320, 340, 360, 380, 400 or more nucleotides. The tracrRNA extension sequence can have a length of less than 1000 nucleotides. The tracrRNA extension sequence can comprise less than 10 nucleotides in length. The tracrRNA extension sequence can be 10-30 nucleotides in length. The tracrRNA extension sequence can be 30-70 nucleotides in length.

The tracrRNA extension sequence can comprise a functional moiety (e.g., a stability control sequence, ribozyme, endoribonuclease binding sequence). The functional moiety can comprise a transcriptional terminator segment (i.e., a transcription termination sequence). The functional moiety can have a total length from about 10 nt to about 100 nucleotides, from about 10 nt to about 20 nt, from about 20 nt to about 30 nt, from about 30 nt to about 40 nt, from about 40 nt to about 50 nt, from about 50 nt to about 60 nt, from about 60 nt to about 70 nt, from about 70 nt to about 80 nt, from about 80 nt to about 90 nt, or from about 90 nt to about 100 nt, from about 15 nt to about 80 nt, from about 15 nt to about 50 nt, from about 15 nt to about 40 nt, from about 15 nt to about 30 nt, or from about 15 nt to about 25 nt. The functional moiety can function in a eukaryotic cell. The functional moiety can function in a prokaryotic cell. The functional moiety can function in both eukaryotic and prokaryotic cells.

Non-limiting examples of suitable tracrRNA extension functional moieties include a 3′ poly-adenylated tail, a riboswitch sequence (e.g., to allow for regulated stability and/or regulated accessibility by proteins and protein complexes), a sequence that forms a dsRNA duplex (i.e., a hairpin), a sequence that targets the RNA to a subcellular location (e.g., nucleus, mitochondria, chloroplasts, and the like), a modification or sequence that provides for tracking (e.g., direct conjugation to a fluorescent molecule, conjugation to a moiety that facilitates fluorescent detection, a sequence that allows for fluorescent detection, etc.), and/or a modification or sequence that provides a binding site for proteins (e.g., proteins that act on DNA, including transcriptional activators, transcriptional repressors, DNA methyltransferases, DNA demethylases, histone acetyltransferases, histone deacetylases, and the like). The tracrRNA extension sequence can comprise a primer binding site or a molecular index (e.g., barcode sequence). The tracrRNA extension sequence can comprise one or more affinity tags.

### Single-Molecule Guide Linker Sequence

The linker sequence of a single-molecule guide nucleic acid can have a length from about 3 nucleotides to about 100 nucleotides. In Jinek et al., supra, for example, a simple 4 nucleotide “tetraloop” (-GAAA-) was used, Science, 337 (6096): 816-821 (2012). An illustrative linker has a length from about 3 nt to about 90 nt, from about 3 nt to about 80 nt, from about 3 nt to about 70 nt, from about 3 nt to about 60 nt, from about 3 nt to about 50 nt, from about 3 nt to about 40 nt, from about 3 nt to about 30 nt, from about 3 nt to about 20 nt, from about 3 nt to about 10 nt. For example, the linker can have a length from about 3 nt to about 5 nt, from about 5 nt to about 10 nt, from about 10 nt to about 15 nt, from about 15 nt to about 20 nt, from about 20 nt to about 25 nt, from about 25 nt to about 30 nt, from about 30 nt to about 35 nt, from about 35 nt to about 40 nt, from about 40 nt to about 50 nt, from about 50 nt to about 60 nt, from about 60 nt to about 70 nt, from about 70 nt to about 80 nt, from about 80 nt to about 90 nt, or from about 90 nt to about 100 nt. The linker of a single-molecule guide nucleic acid can be between 4 and 40 nucleotides. The linker can be at least about 100, 500, 1000, 1500, 2000, 2500, 3000, 3500, 4000, 4500, 5000, 5500, 6000, 6500, or 7000 or more nucleotides. The linker can be at most about 100, 500, 1000, 1500, 2000, 2500, 3000, 3500, 4000, 4500, 5000, 5500, 6000, 6500, or 7000 or more nucleotides.

Linkers can comprise any of a variety of sequences, although in some examples the linker will not comprise sequences that have extensive regions of homology with other portions of the guide RNA, which might cause intramolecular binding that could interfere with other functional regions of the guide. In Jinek et al., supra, a simple 4 nucleotide sequence -GAAA- was used, Science, 337 (6096): 816-821 (2012), but numerous other sequences, including longer sequences can likewise be used.

The linker sequence can comprise a functional moiety. For example, the linker sequence can comprise one or more features, including an aptamer, a ribozyme, a protein-interacting hairpin, a protein binding site, a CRISPR array, an intron, or an exon. The linker sequence can comprise at least about 1, 2, 3, 4, or 5 or more functional moieties. The linker sequence can comprise at most about 1, 2, 3, 4, or 5 or more functional moieties.

### Ribonucleoprotein Complex (RNP)

The gRNA or sgRNA can interact with at least one domain of the Cas nuclease to form a RNP complex and direct the RNP complex to the target sequence. The gRNA can interact with the Cas nuclease and the target sequence such that, once directed to the target sequence, the Cas nuclease is capable of cleaving the target sequence. By “cleavage” it is meant the breakage of the covalent backbone of a DNA molecule. Cleavage can be initiated by a variety of methods including, but not limited to, enzymatic or chemical hydrolysis of a phosphodiester bond. Both single-stranded cleavage and double-stranded cleavage are possible, and double-stranded cleavage can occur as a result of two distinct single-stranded cleavage events. DNA cleavage can result in the production of either blunt ends or staggered ends. A ribonucleoprotein complex comprising a gRNA and a Cas nuclease can be used for targeted double-stranded DNA cleavage.

### AAV (Adeno Associated Virus)

A recombinant AAV vector can be used for delivering polynucleotides. Production of rAAV typically requires that the following components are present within a single packaging cell: a rAAV genome, AAV rep and cap genes separate from (i.e., not in) the rAAV genome, and helper virus functions. The AAV rep and cap genes can be from any AAV serotype for which recombinant virus can be derived, and can be from a different AAV serotype than the rAAV genome ITRs, including, but not limited to, AAV serotypes described herein. Production of pseudotyped rAAV is disclosed in, for example, international patent application publication number WO 01/83692.

### AAV Serotypes

AAV particles packaging polynucleotides encoding compositions of the present disclosure, e.g., CRISPR/Cas systems or recombinant AAV vectors) can comprise or be derived from any natural or recombinant AAV serotype. According to the present disclosure, the AAV particles can utilize or be based on a serotype selected from any of the following serotypes, and variants thereof including but not limited to AAV1, AAV10, AAV106.1/hu.37, AAV11, AAV114.3/hu.40, AAV12, AAV127.2/hu.41, AAV127.5/hu.42, AAV128.1/hu.43, AAV128.3/hu.44, AAV130.4/hu.48, AAV145.1/hu.53, AAV145.5/hu.54, AAV145.6/hu.55, AAV16.12/hu.11, AAV16.3, AAV16.8/hu.10, AAV161.10/hu.60, AAV161.6/hu.61, AAV1-7/rh.48, AAV1-8/rh.49, AAV2, AAV2.5T, AAV2-15/rh.62, AAV223.1, AAV223.2, AAV223.4, AAV223.5, AAV223.6, AAV223.7, AAV2-3/rh.61, AAV24.1, AAV2-4/rh.50, AAV2-5/rh.51, AAV27.3, AAV29.3/bb.1, AAV29.5/bb.2, AAV2G9, AAV-2-pre-miRNA-101, AAV3, AAV3.1/hu.6, AAV3.1/hu.9, AAV3-11/rh.53, AAV3-3, AAV33.12/hu.17, AAV33.4/hu.15, AAV33.8/hu.16, AAV3-9/rh.52, AAV3a, AAV3b, AAV4, AAV4-19/rh.55, AAV42.12, AAV42-10, AAV42-11, AAV42-12, AAV42-13, AAV42-15, AAV42-1b, AAV42-2, AAV42-3a, AAV42-3b, AAV42-4, AAV42-5a, AAV42-5b, AAV42-6b, AAV42-8, AAV42-aa, AAV43-1, AAV43-12, AAV43-20, AAV43-21, AAV43-23, AAV43-25, AAV43-5, AAV4-4, AAV44.1, AAV44.2, AAV44.5, AAV46.2/hu.28, AAV46.6/hu.29, AAV4-8/r11.64, AAV4-8/rh.64, AAV4-9/rh.54, AAV5, AAV52.1/hu.20, AAV52/hu.19, AAV5-22/rh.58, AAV5-3/rh.57, AAV54.1/hu.21, AAV54.2/hu.22, AAV54.4R/hu.27, AAV54.5/hu.23, AAV54.7/hu.24, AAV58.2/hu.25, AAV6, AAV6.1, AAV6.1.2, AAV6.2, AAV7, AAV7.2, AAV7.3/hu.7, AAV8, AAV-8b, AAV-8h, AAV9, AAV9.11, AAV9.13, AAV9.16, AAV9.24, AAV9.45, AAV9.47, AAV9.61, AAV9.68, AAV9.84, AAV9.9, AAVA3.3, AAVA3.4, AAVA3.5, AAVA3.7, AAV-b, AAVC1, AAVC2, AAVC5, AAVCh.5, AAVCh.5R1, AAVcy.2, AAVcy.3, AAVcy.4, AAVcy.5, AAVCy.5R1, AAVCy.5R2, AAVCy.5R3, AAVCy.5R4, AAVcy.6, AAV-DJ, AAV-DJ8, AAVF3, AAVF5, AAV-h, AAVH-1/hu.1, AAVH2, AAVH-5/hu.3, AAVH6, AAVhE1.1, AAVhER1.14, AAVhEr1.16, AAVhEr1.18, AAVhER1.23, AAVhEr1.35, AAVhEr1.36, AAVhEr1.5, AAVhEr1.7, AAVhEr1.8, AAVhEr2.16, AAVhEr2.29, AAVhEr2.30, AAVhEr2.31, AAVhEr2.36, AAVhEr2.4, AAVhEr3.1, AAVhu.1, AAVhu. 10, AAVhu.11, AAVhu.11, AAVhu.12, AAVhu.13, AAVhu.14/9, AAVhu.15, AAVhu. 16, AAVhu.17, AAVhu.18, AAVhu.19, AAVhu.2, AAVhu.20, AAVhu.21, AAVhu.22, AAVhu.23.2, AAVhu.24, AAVhu.25, AAVhu.27, AAVhu.28, AAVhu.29, AAVhu.29R, AAVhu.3, AAVhu.31, AAVhu.32, AAVhu.34, AAVhu.35, AAVhu.37, AAVhu.39, AAVhu.4, AAVhu.40, AAVhu.41, AAVhu.42, AAVhu.43, AAVhu.44, AAVhu.44R1, AAVhu.44R2, AAVhu.44R3, AAVhu.45, AAVhu.46, AAVhu.47, AAVhu.48, AAVhu.48R1, AAVhu.48R2, AAVhu.48R3, AAVhu.49, AAVhu.5, AAVhu.51, AAVhu.52, AAVhu.53, AAVhu.54, AAVhu.55, AAVhu.56, AAVhu.57, AAVhu.58, AAVhu.6, AAVhu.60, AAVhu.61, AAVhu.63, AAVhu.64, AAVhu.66, AAVhu.67, AAVhu.7, AAVhu.8, AAVhu.9, AAVhu.t 19, AAVLG-10/rh.40, AAVLG-4/rh.38, AAVLG-9/hu.39, AAVLG-9/hu.39, AAV-LK01, AAV-LK02, AAVLK03, AAV-LK03, AAV-LK04, AAV-LK05, AAV-LK06, AAV-LK07, AAV-LK08, AAV-LK09, AAV-LK10, AAV-LK11, AAV-LK12, AAV-LK13, AAV-LK14, AAV-LK15, AAV-LK17, AAV-LK18, AAV-LK19, AAVN721-8/rh.43, AAV-PAEC, AAV-PAEC11, AAV-PAEC12, AAV-PAEC2, AAV-PAEC4, AAV-PAEC6, AAV-PAEC7, AAV-PAEC8, AAVpi.1, AAVpi.2, AAVpi.3, AAVrh.10, AAVrh.12, AAVrh.13, AAVrh.13R, AAVrh.14, AAVrh.17, AAVrh.18, AAVrh.19, AAVrh.2, AAVrh.20, AAVrh.21, AAVrh.22, AAVrh.23, AAVrh.24, AAVrh.25, AAVrh.2R, AAVrh.31, AAVrh.32, AAVrh.33, AAVrh.34, AAVrh.35, AAVrh.36, AAVrh.37, AAVrh.37R2, AAVrh.38, AAVrh.39, AAVrh.40, AAVrh.43, AAVrh.44, AAVrh.45, AAVrh.46, AAVrh.47, AAVrh.48, AAVrh.48, AAVrh.48.1, AAVrh.48.1.2, AAVrh.48.2, AAVrh.49, AAVrh.50, AAVrh.51, AAVrh.52, AAVrh.53, AAVrh.54, AAVrh.55, AAVrh.56, AAVrh.57, AAVrh.58, AAVrh.59, AAVrh.60, AAVrh.61, AAVrh.62, AAVrh.64, AAVrh.64R1, AAVrh.64R2, AAVrh.65, AAVrh.67, AAVrh.68, AAVrh.69, AAVrh.70, AAVrh.72, AAVrh.73, AAVrh.74, AAVrh.8, AAVrh.8R, AAVrh8R, AAVrh8R A586R mutant, AAVrh8R R533A mutant, BAAV, BNP61 AAV, BNP62 AAV, BNP63 AAV, bovine AAV, caprine AAV, Japanese AAV 10, true type AAV (ttAAV), UPENN AAV 10, AAV-LK16, AAAV, AAV Shuffle 100-1, AAV Shuffle 100-2, AAV Shuffle 100-3, AAV Shuffle 100-7, AAV Shuffle 10-2, AAV Shuffle 10-6, AAV Shuffle 10-8, AAV SM 100-10, AAV SM 100-3, AAV SM 10-1, AAV SM 10-2, and/or AAV SM 10-8.

The AAV serotype can be, or have, a mutation in the AAV9 sequence as described by N Pulicherla et al. (Molecular Therapy 19 (6): 1070-1078 (2011), such as but not limited to, AAV9.9, AAV9.11, AAV9.13, AAV9.16, AAV9.24, AAV9.45, AAV9.47, AAV9.61, AAV9.68, AAV9.84.

The AAV serotype can be, or have, a sequence as described in United States Patent No. U.S. Pat. No. 6,156,303, such as, but not limited to, AAV3B (SEQ ID NO: 1 and 10 of U.S. Pat. No. 6,156,303), AAV6 (SEQ ID NO: 2, 7 and 11 of U.S. Pat. No. 6,156,303), AAV2 (SEQ ID NO: 3 and 8 of U.S. Pat. No. 6,156,303), AAV3A (SEQ ID NO: 4 and 9, of U.S. Pat. No. 6,156,303), or derivatives thereof.

The serotype can be AAVDJ or a variant thereof, such as AAVDJ8 (or AAV-DJ8), as described by Grimm et al. (Journal of Virology 82 (12): 5887-5911 (2008)). The amino acid sequence of AAVDJ8 can comprise two or more mutations in order to remove the heparin binding domain (HBD). As a non-limiting example, the AAV-DJ sequence described as SEQ ID NO: 1 in U.S. Pat. No. 7,588,772, can comprise two mutations: (1) R587Q where arginine (R; Arg) at amino acid 587 is changed to glutamine (Q; Gln) and (2) R590T where arginine (R; Arg) at amino acid 590 is changed to threonine (T; Thr). As another non-limiting example, can comprise three mutations: (1) K406R where lysine (K; Lys) at amino acid 406 is changed to arginine (R; Arg), (2) R587Q where arginine (R; Arg) at amino acid 587 is changed to glutamine (Q; Gln) and (3) R590T where arginine (R; Arg) at amino acid 590 is changed to threonine (T; Thr).

The AAV serotype can be, or have, a sequence as described in International Publication No. WO2015121501, such as, but not limited to, true type AAV (ttAAV) (SEQ ID NO: 2 of WO2015121501), “UPenn AAV10” (SEQ ID NO: 8 of WO2015121501), “Japanese AAV10” (SEQ ID NO: 9 of WO2015121501), or variants thereof.

According to the present disclosure, AAV capsid serotype selection or use can be from a variety of species. In one example, the AAV can be an avian AAV (AAAV). The AAAV serotype can be, or have, a sequence as described in United States Patent No. U.S. Pat. No. 9,238,800, such as, but not limited to, AAAV (SEQ ID NO: 1, 2, 4, 6, 8, 10, 12, and 14 of U.S. Pat. No. 9,238,800), or variants thereof.

In one example, the AAV can be a bovine AAV (BAAV). The BAAV serotype can be, or have, a sequence as described in United States Patent No. U.S. Pat. No. 9,193,769, such as, but not limited to, BAAV (SEQ ID NO: 1 and 6 of U.S. Pat. No. 9,193,769), or variants thereof. The BAAV serotype can be or have a sequence as described in United States Patent No. U.S. Pat. No. 7,427,396, such as, but not limited to, BAAV (SEQ ID NO: 5 and 6 of U.S. Pat. No. 7,427,396), or variants thereof.

In one example, the AAV can be a caprine AAV. The caprine AAV serotype can be, or have, a sequence as described in United States Patent No. U.S. Pat. No. 7,427,396, such as, but not limited to, caprine AAV (SEQ ID NO: 3 of U.S. Pat. No. 7,427,396), or variants thereof.

The AAV can be engineered as a hybrid AAV from two or more parental serotypes. In one example, the AAV can be AAV2G9 which comprises sequences from AAV2 and AAV9. The AAV2G9 AAV serotype can be, or have, a sequence as described in United States Patent Publication No. US20160017005.

In one example, the AAV can be a serotype generated by the AAV9 capsid library with mutations in amino acids 390-627 (VP1 numbering) as described by Pulicherla et al. (Molecular Therapy 19 (6): 1070-1078 (2011). The serotype and corresponding nucleotide and amino acid substitutions can be, but is not limited to, AAV9.1 (G1594C; D532H), AAV6.2 (T1418A and T1436X; V473D and I479K), AAV9.3 (T1238A; F413Y), AAV9.4 (T1250C and A1617T; F417S), AAV9.5 (A1235G, A1314T, A1642G, C1760T; Q412R, T548A, A587V), AAV9.6 (T1231A; F411I), AAV9.9 (G1203A, G1785T; W595C), AAV9.10 (A1500G, T1676C; M559T), AAV9.11 (A1425T, A1702C, A1769T; T568P, Q590L), AAV9.13 (A1369C, A1720T; N457H, T574S), AAV9.14 (T1340A, T1362C, T1560C, G1713A; L447H), AAV9.16 (A1775T; Q592L), AAV9.24 (T1507C, T1521G; W503R), AAV9.26 (A1337G, A1769C; Y446C, Q590P), AAV9.33 (A1667C; D556A), AAV9.34 (A1534G, C1794T; N512D), AAV9.35 (A1289T, T1450A, C1494T, A1515T, C1794A, G1816A; Q430L, Y484N, N98K, V606I), AAV9.40 (A1694T, E565V), AAV9.41 (A1348T, T1362C; T450S), AAV9.44 (A1684C, A1701T, A1737G; N562H, K567N), AAV9.45 (A1492T, C1804T; N498Y, L602F), AAV9.46 (G1441C, T1525C, T1549G; G481R, W509R, L517V), 9.47 (G1241A, G1358A, A1669G, C1745T; S414N, G453D, K557E, T582I), AAV9.48 (C1445T, A1736T; P482L, Q579L), AAV9.50 (A1638T, C1683T, T1805A; Q546H, L602H), AAV9.53 (G1301A, A1405C, C1664T, G1811T; R134Q, S469R, A555V, G604V), AAV9.54 (C1531A, T1609A; L511I, L537M), AAV9.55 (T1605A; F535L), AAV9.58 (C1475T, C1579A; T492I, H527N), AAV.59 (T1336C; Y446H), AAV9.61 (A1493T; N4981), AAV9.64 (C1531A, A1617T; L511I), AAV9.65 (C1335T, T1530C, C1568A; A523D), AAV9.68 (C1510A; P504T), AAV9.80 (G1441A; G481R), AAV9.83 (C1402A, A1500T; P468T, E500D), AAV9.87 (T1464C, T1468C; S490P), AAV9.90 (A1196T; Y399F), AAV9.91 (T1316G, A1583T, C1782G, T1806C; L439R, K528I), AAV9.93 (A1273G, A1421G, A1638C, C1712T, G1732A, A1744T, A1832T; S425G, Q474R, Q546H, P571L, G578R, T582S, D611V), AAV9.94 (A1675T; M559L) and AAV9.95 (T1605A; F535L).

In one example, the AAV can be a serotype comprising at least one AAV capsid CD8+ T-cell epitope. As a non-limiting example, the serotype can be AAV1, AAV2 or AAV8.

In one example, the AAV can be a variant, such as PHP.A or PHP.B as described in Deverman. 2016. Nature Biotechnology. 34 (2): 204-209.

In one example, the AAV can be a serotype selected from any of those found in SEQ ID NOs: 4697-5265 and Table 5.

In one example, the AAV can be encoded by a sequence, fragment or variant as described in SEQ ID NOs: 4697-5265 and Table 5.

A method of generating a packaging cell involves creating a cell line that stably expresses all of the necessary components for AAV particle production. For example, a plasmid (or multiple plasmids) comprising a rAAV genome lacking AAV rep and cap genes, AAV rep and cap genes separate from the rAAV genome, and a selectable marker, such as a neomycin resistance gene, are integrated into the genome of a cell. AAV genomes have been introduced into bacterial plasmids by procedures such as GC tailing (Samulski et al., 1982, Proc. Natl. Acad. S6. USA, 79:2077-2081), addition of synthetic linkers containing restriction endonuclease cleavage sites (Laughlin et al., 1983, Gene, 23:65-73) or by direct, blunt-end ligation (Senapathy & Carter, 1984, J. Biol. Chem., 259:4661-4666). The packaging cell line can then be infected with a helper virus, such as adenovirus. The advantages of this method are that the cells are selectable and are suitable for large-scale production of rAAV. Other examples of suitable methods employ adenovirus or baculovirus, rather than plasmids, to introduce rAAV genomes and/or rep and cap genes into packaging cells.

General principles of rAAV production are reviewed in, for example, Carter, 1992, Current Opinions in Biotechnology, 1533-539; and Muzyczka, 1992, Curr. Topics in Microbial. and Immunol., 158:97-129). Various approaches are described in Ratschin et al., Mol. Cell. Biol. 4:2072 (1984); Hermonat et al., Proc. Natl. Acad. Sci. USA, 81:6466 (1984); Tratschin et al., Mol. Cell. Biol. 5:3251 (1985); Mclaughlin et al., J. Virol., 62:1963 (1988); and Lebkowski et al., 1988 Mol. Cell. Biol., 7:349 (1988). Samulski et al. (1989, J. Virol., 63:3822-3828); U.S. Pat. No. 5,173,414; WO 95/13365 and corresponding U.S. Pat. No. 5,658,776; WO 95/13392; WO 96/17947; PCT/US98/18600; WO 97/09441 (PCT/US96/14423); WO 97/08298 (PCT/US96/13872); WO 97/21825 (PCT/US96/20777); WO 97/06243 (PCT/FR96/01064); WO 99/11764; Perrin et al. (1995) Vaccine 13:1244-1250; Paul et al. (1993) Human Gene Therapy 4:609-615; Clark et al. (1996) Gene Therapy 3:1124-1132; U.S. Pat. Nos. 5,786,211; 5,871,982; and 6,258,595.

AAV vector serotypes can be matched to target cell types. For example, the following exemplary cell types can be transduced by the indicated AAV serotypes among others. See Table 1.

TABLE 1
Tissue/Cell Types and Serotypes

| Tissue/Cell Type | Serotype |
|---|---|
| Liver | AAV3, AA5, AAV8, AAV9 |
| Skeletal muscle | AAV1, AAV7, AAV6, AAV8, AAV9 |
| Central nervous system | AAV1, AAV4, AAV5, AAV8, AAV9 |
| RPE | AAV5, AAV4, AAV2, AAV8, AAV9, |
| | AAVrh8r |
| Photoreceptor cells | AAV5, AA8, AAV9, AAVrh8R |
| Lung | AAV9, AAV5 |
| Heart | AAV8 |
| Pancreas | AAV8 |
| Kidney | AAV2, AAV8 |

### Materials and Methods of the Invention

Various aspects of the invention according to the present disclosure include, but are not limited to, the aspects listed in the following numbered clauses:

1. A CRISPR/Cas system comprising:
a nuclease segment comprising a codon optimized nucleotide sequence that encodes a Cas nuclease or variant thereof;
a guide RNA (gRNA) segment comprising a nucleotide sequence that encodes a gRNA or sgRNA; and
a promoter segment comprising a nucleotide sequence that encodes a first promoter comprising one or more tetracycline operator sequence, wherein the gRNA segment is operably linked to the promoter segment.
   2. The CRISPR/Cas system of clause 1, wherein the first promoter is selected from a group consisting of: H1 promoter, U6 promoter, 7SK promoter, and portions of any thereof.
   3. The CRISPR/Cas system of clauses 1 or 2, wherein the one or more tetracycline operator sequence comprises a nucleic acid sequence having at least 85% sequence identity to SEQ ID NO: 4.
   4. The CRISPR/Cas system of any one of clauses 1-3, wherein the one or more tetracycline operator sequence comprises SEQ ID NO: 4.
   5. The CRISPR/Cas system of any one of clauses 1-4, further comprising:
a repressor segment comprising a nucleotide sequence that encodes a tetracycline repressor protein.
   6. The CRISPR/Cas system of clause 5, wherein the tetracycline repressor comprises a nucleic acid sequence having at least 85% sequence identity to SEQ ID NO: 62.
   7. The CRISPR/Cas system of clause 5, wherein the tetracycline repressor comprises a nucleic acid sequence comprising SEQ ID NO: 62.
   8. The CRISPR/Cas system of any one of clauses 1-7, wherein the one or more tetracycline operator sequence is capable of being bound by the tetracycline repressor protein.
   9. The CRISPR/Cas system of any one of clauses 1-8, further comprising:
one or more self-inactivating segments comprising a SIN site;
wherein the gRNA or sgRNA is substantially complementary to the SIN site;
wherein the gRNA or sgRNA is substantially complementary to a genomic target sequence within a cell of a patient.
   10. The CRISPR/Cas system of clause 9, wherein the one or more self-inactivating segments are located in at least one of:
(i) at the 5′ end of the codon optimized nucleotide sequence that encodes a Cas nuclease or variant thereof;
(ii) at the 3′ end of the codon optimized nucleotide sequence that encodes a Cas nuclease or variant thereof; and
(iii) in an intron within the codon optimized nucleotide sequence that encodes a Cas nuclease or variant thereof.
   11. The CRISPR/Cas system of clause 9, wherein the one or more self-inactivating segments are located in:
(i) at the 5′ end of the codon optimized nucleotide sequence that encodes a Cas nuclease or variant thereof; and
(ii) in an intron within the codon optimized nucleotide sequence that encodes a Cas nuclease or variant thereof.
   12. The CRISPR/Cas system of any one of clauses 9-11, wherein one of the one or more self-inactivating segments are located upstream of the codon optimized nucleotide sequence that encodes a Cas nuclease or variant thereof and downstream of a NLS.
   13. The CRISPR/Cas system of any one of clauses 9-11, wherein the SIN site comprises a PAM sequence.
   14. The CRISPR/Cas system of clause 13, wherein the PAM sequence in the SIN site is selected from a group consisting of: NNGRRT, NRG, NAAAAN, NAAAAC, NNNNGHTT, YTN, NNNNACAC, NNVRYAC, NNNNVRYM, NNAAAAW, NNAGAAW, and NNGG. 15. The CRISPR/Cas system of any of clauses 1-14, wherein the gRNA or sgRNA is fully complementary to the nucleotide sequence of the SIN site except for at one base pair.
   16. The CRISPR/Cas system of any of clauses 1-14, wherein the gRNA or sgRNA is fully complementary to the nucleotide sequence of the SIN site except for at two base pairs.
   17. The CRISPR/Cas system of any one of clauses 1-16, wherein the Cas nuclease is a Class 2 Cas nuclease.
   18. The CRISPR/Cas system of any one of clauses 1-17, wherein the Cas nuclease is selected from a group consisting of: S. pyogenes Cas, S. aureus Cas, S. thermolphilus Cas, C. jejuni Cas, T. denticola Cas, N. meningitides Cas, S. lugdunensis Cas, S. hyicus Cas, S. microti Cas, and S. pasteuri Cas.
   19. The CRISPR/Cas system of any one of clauses 1-16, wherein the Cas nuclease is a sRGN.
   20. The CRISPR/Cas system of clause 19, wherein the Cas nuclease comprises an amino acid sequence having at least 85% sequence identity to SEQ ID NO: 60.
   21. The CRISPR/Cas system of any one of clauses 1-20, wherein a nucleic acid sequence encoding a second promoter is operably linked to the codon optimized nucleotide sequence that encodes a Cas nuclease or variant thereof.
   22. The CRISPR/Cas system of clause 21, wherein the second promoter is a spatially-restricted promoter, bidirectional promoter, or an inducible promoter.
   23. The CRISPR/Cas system of clause 22, wherein the spatially-restricted promoter is selected from a group consisting of: any tissue or cell type specific promoter, a hepatocyte-specific promoter, a neuron-specific promoter, an adipocyte-specific promoter, a cardiomyocyte-specific promoter, a skeletal muscle-specific promoter, lung progenitor cell specific promoter, a photoreceptor-specific promoter, and a RPE selective promoter.
   24. The CRISPR/Cas system of any one of clauses 1-23, wherein the gRNA or sgRNA comprising a spacer sequence comprising 17 to 24 nucleotides.
   25. The CRISPR/Cas system of any of clauses 1-25, wherein the nuclease segment, the gRNA segment, and the promoter segment are provided together in a first vector and the repressor segment is provided in a second vector.
   26. The CRISPR/Cas system of any one of clauses 9-25, wherein the nuclease segment, the gRNA segment, the promoter segment, and the one or more self-inactivating segments are provided together in a first vector and the repressor segment is provided in a second vector.
   27. The CRISPR/Cas system of any one of clauses 9-25, wherein the nuclease segment, the gRNA segment, the promoter segment, the one or more self-inactivating segments, and the repressor segment are provided in a vector.
   28. The CRISPR/Cas system of any one of clauses 25-26, wherein the first vector and the second vector are AAV vectors or plasmids.
   29. The CRISPR/Cas system of clause 28, wherein the AAV vectors are AAV2 serotype vectors, AAV5 serotype vectors, or AAV6 serotype vectors.
   30. A pharmaceutical composition comprising the CRISPR/Cas system of any one of clauses 1-29.
   31. A packaging cell comprising the CRISPR/Cas system of any one of clauses 1-29.
   32. A method of controlling transcription of gRNAs during AAV packaging, the method comprising:
contacting a packaging cell with the CRISPR/Cas system of any one of clauses 1-29.
   33. A method of reducing mutagenesis at one or more SIN site in a recombinant AAV vector, the method comprising:
contacting a packaging cell with the CRISPR/Cas system of any one of clauses 1-29.
   34. A recombinant AAV vector comprising:
a nuclease segment comprising a codon optimized nucleotide sequence that encodes a Cas nuclease or variant thereof;
a gRNA segment comprising a nucleotide sequence that encodes a gRNA or sgRNA; and
a promoter segment comprising a nucleotide sequence that encodes a first promoter comprising one or more tetracycline operator sequence, wherein the gRNA segment is operably linked to the promoter segment.
   35. The recombinant AAV vector of clause 34, wherein the first promoter is selected from a group consisting of: H1 promoter, U6 promoter, 7SK promoter, and portions of any thereof.
   36. The recombinant AAV vector of any one of clauses 34 or 35, wherein the one or more tetracycline operator sequence comprises a nucleic acid sequence having at least 85% sequence identity to SEQ ID NO: 4.
   37. The recombinant AAV vector of any one of clauses 34-36, wherein the one or more tetracycline operator sequence comprises SEQ ID NO: 4.
   38. The recombinant AAV vector of any one of clauses 34-37, wherein the one or more tetracycline operator sequences is capable of being bound by a tetracycline repressor.
   39. The recombinant AAV vector of any one of clauses 34-38, further comprising:
one or more self-inactivating segments comprising a SIN site;
wherein the gRNA or sgRNA is substantially complementary to the SIN site;
wherein the gRNA or sgRNA is substantially complementary to a genomic target sequence within a cell of a patient.
   40. The recombinant AAV vector of clause 39, wherein the one or more self-inactivating segments are located in at least one of:
(i) at the 5′ end of the codon optimized nucleotide sequence that encodes a Cas nuclease or variant thereof;
(ii) at the 3′ end of the codon optimized nucleotide sequence that encodes a Cas nuclease or variant thereof; and
(iii) in an intron within the codon optimized nucleotide sequence that encodes a Cas nuclease or variant thereof.
   41. The recombinant AAV vector of clause 39, wherein the one or more self-inactivating segments are located in:
(i) at the 5′ end of the codon optimized nucleotide sequence that encodes a Cas nuclease or variant thereof; and
(ii) in an intron within the codon optimized nucleotide sequence that encodes a Cas nuclease or variant thereof.
   42. The recombinant AAV vector of any one of clauses 39-41, wherein one of the one or more self-inactivating segments are located upstream of the codon optimized nucleotide sequence that encodes a Cas nuclease or variant thereof and downstream of a NLS.
   43. The recombinant AAV vector of any one of clauses 39-41, wherein the SIN site comprises a PAM sequence.
   44. The recombinant AAV vector of clause 43, wherein the PAM sequence in the SIN site is selected from a group consisting of: NNGRRT, NRG, NAAAAN, NAAAAC, NNNNGHTT, YTN, NNNNACAC, NNVRYAC, NNNNVRYM, NNAAAAW, NNAGAAW, and NNGG.
   45. The recombinant AAV vector of any one of clauses 34-44, wherein the gRNA or sgRNA is fully complementary to the nucleotide sequence of the SIN site except for at one base pair.
   46. The recombinant AAV vector of any one of clauses 34-44, wherein the gRNA or sgRNA is fully complementary to the nucleotide sequence of the SIN site except for at two base pairs.
   47. The recombinant AAV vector of any one of clauses 34-46, wherein the Cas nuclease is a Class 2 Cas nuclease.
   48. The recombinant AAV vector of any one of clauses 34-47, wherein the Cas nuclease is selected from a group consisting of: S. pyogenes Cas, S. aureus Cas, S. thermolphilus Cas, C. jejuni Cas, T. denticola Cas, N. meningitides Cas, S. lugdunensis Cas, S. hyicus Cas, S. microti Cas, and S. pasteuri Cas.
   49. The recombinant AAV vector of any one of clauses 34-46, wherein the Cas nuclease is a SRGN.
   50. The recombinant AAV vector of clause 49, wherein the Cas nuclease comprises an amino acid sequence having at least 85% sequence identity to SEQ ID NO: 60.
   51. The recombinant AAV vector of any one of clauses 34-50, wherein a nucleic acid sequence encoding a second promoter is operably linked to the codon optimized nucleotide sequence that encodes a Cas nuclease or variant thereof.
   52. The recombinant AAV vector of clause 51, wherein the second promoter is a spatially-restricted promoter, bidirectional promoter, or an inducible promoter.
   53. The recombinant AAV vector of clause 52, wherein the spatially-restricted promoter is selected from a group consisting of: any tissue or cell type specific promoter, a hepatocyte-specific promoter, a neuron-specific promoter, an adipocyte-specific promoter, a cardiomyocyte-specific promoter, a skeletal muscle-specific promoter, lung progenitor cell specific promoter, a photoreceptor-specific promoter, and a RPE selective promoter.
   54. The recombinant AAV vector of any one of clauses 34-53, wherein the gRNA is a sgRNA.
   55. The recombinant AAV vector of any one of clauses 34-54, wherein the gRNA or sgRNA comprising a spacer sequence comprising 17 to 24 nucleotides.
   56. The recombinant AAV vector of any of clauses 34-55, wherein the nuclease segment, the gRNA segment, and the promoter segment are provided together in a vector.
   57. The recombinant AAV vector of any of clauses 39-56, wherein the nuclease segment, the gRNA segment, the promoter segment, and the one or more self-inactivating segments are provided together in a vector.
   58. The recombinant AAV vector of any one of clauses 34-57, wherein the recombinant AAV vector is a AAV2 serotype vector, AAV5 serotype vector, or AAV6 serotype vector.
   59. The recombinant AAV vector of any one of clauses 34-58, wherein the recombinant AAV vector comprises a nucleic acid sequence having at least 85% sequence identity to any one of SEQ ID NOs: 66-69.
   60. The recombinant AAV vector of any one of clauses 34-58, wherein the recombinant AAV vector comprises any one of SEQ ID NOs: 66-69.
   61. A pharmaceutical composition comprising the recombinant AAV vector of any of clauses 34-60.
   62. A genetically modified cell comprising the recombinant AAV vector of any of clauses 34-60.
   63. The genetically modified cell of clause 62, wherein the genetically modified cell is selected from a group consisting of: a eukaryotic cell, a somatic cell, a germ cell, a stem cell, an animal cell, a mammalian cell, a mouse cell, a non-human primate cell, and a human cell.
   64. A method of controlling transcription of gRNAs during AAV packaging, the method comprising:
contacting a packaging cell with a nucleic acid encoding the recombinant AAV vector of any one of clauses 34-60; and
contacting the packaging cell with at least one vector comprising nucleic acid sequence encoding a tetracycline repressor.
   65. A method of reducing mutagenesis at one or more SIN site in a recombinant AAV vector, the method comprising:
contacting a packaging cell with nucleic acid encoding the recombinant AAV vector of any one of clauses 34-60; and
contacting the packaging cell with at least one vector comprising nucleic acid sequence encoding a tetracycline repressor.
   66. The method of clause 65, wherein the packaging cell is a human cell.
   67. A method of producing a recombinant AAV vector, the method comprising:
introducing into a packaging cell:
   (i) a first vector comprising a repressor segment, wherein the repressor segment comprises a nucleotide sequence that encodes a tetracycline repressor protein;
   (ii) a nucleic acid comprising sequence encoding the recombinant AAV vector of any one of clauses 34-60; and
   (iii) one or more viral components for producing the recombinant AAV vector;
culturing the packaging cell; and
isolating the recombinant AAV vector comprising the nucleic acid from the packaging cell.
   68. A method of producing a recombinant AAV vector, the method comprising:
introducing into a packaging cell a nucleic acid comprising a sequence encoding the recombinant AAV vector of any one of clauses 34-60;
introducing into the packaging cell one or more viral components for producing the AAV;
culturing the packaging cell; and
isolating the recombinant AAV vector comprising the nucleic acid from the packaging cell.
wherein the packaging cell expresses a tetracycline repressor protein.
   69. The method of any one of clauses 67-68, wherein the one or more viral components are encoded by the nucleic acid.
   70. The method of any one of clauses 67-68, wherein the one or more viral components are introduced via separate vector other than the nucleic acid.
   71. The method of any one of clauses 67-78, wherein the one or more viral components are encoded in a cellular genome.
   72. A CRISPR/Cas system comprising:
a nuclease segment comprising a codon optimized nucleotide sequence that encodes a Cas nuclease or variant thereof;
a gRNA segment comprising a nucleotide sequence that encodes a gRNA or sgRNA; and
a shRNA segment comprising a nucleotide sequence that encodes a shRNA that comprises sequence that is complementary to a transcript from the nuclease segment.
   73. The CRISPR/Cas system of clause 72, wherein the shRNA comprises a sequence having at least 85% sequence identity to any one of SEQ ID NOs: 9-11 or 55-59.
   74. The CRISPR/Cas system of clause 72, wherein the shRNA comprises any one of SEQ ID NOs: 9-11 or 55-59.
   75. The CRISPR/Cas system of any one of clauses 72-74, further comprising:
one or more self-inactivating segments comprising a SIN site;
wherein the gRNA or sgRNA is substantially complementary to the SIN site;
wherein the gRNA or sgRNA is substantially complementary to a genomic target sequence within a cell of a patient.
   76. The CRISPR/Cas system of clause 75, wherein the one or more self-inactivating segments are located in at least one of:
(i) at the 5′ end of the codon optimized nucleotide sequence that encodes a Cas nuclease or variant thereof;
(ii) at the 3′ end of the codon optimized nucleotide sequence that encodes a Cas nuclease or variant thereof; and
(iii) in an intron within the codon optimized nucleotide sequence that encodes a Cas nuclease or variant thereof.
   77. The CRISPR/Cas system of clause 75, wherein the one or more self-inactivating segments are located in:
(i) at the 5′ end of the codon optimized nucleotide sequence that encodes a Cas nuclease or variant thereof; and
(ii) in an intron within the codon optimized nucleotide sequence that encodes a Cas nuclease or variant thereof.
   78. The CRISPR/Cas system of any one of clauses 75-77, wherein one of the one or more self-inactivating segments are located upstream of the codon optimized nucleotide sequence that encodes a Cas nuclease or variant thereof and downstream of a NLS.
   79. The CRISPR/Cas system of any one of clauses 75-77, wherein the SIN site comprises a PAM sequence.
   80. The CRISPR/Cas system of clause 79, wherein the PAM sequence in the SIN site is selected from a group consisting of: NNGRRT, NRG, NAAAAN, NAAAAC, NNNNGHTT, YTN, NNNNACAC, NNVRYAC, NNNNVRYM, NNAAAAW, NNAGAAW, and NNGG.
   81. The CRISPR/Cas system of any of clauses 72-80, wherein the gRNA or sgRNA is fully complementary to the nucleotide sequence of the SIN site except for at one base pair.
   82. The CRISPR/Cas system of any of clauses 72-80, wherein the gRNA or sgRNA is fully complementary to the nucleotide sequence of the SIN site except for at two base pairs.
   83. The CRISPR/Cas system of any one of clauses 72-82, wherein the Cas nuclease is a Class 2 Cas nuclease.
   84. The CRISPR/Cas system of any one of clauses 72-83, wherein the Cas nuclease is selected from a group consisting of: S. pyogenes Cas, S. aureus Cas, S. thermolphilus Cas, C. jejuni Cas, T. denticola Cas, N. meningitides Cas, S. lugdunensis Cas, S. hyicus Cas, S. microti Cas, and S. pasteuri Cas.
   85. The CRISPR/Cas system of any one of clauses 72-82, wherein the Cas nuclease is a sRGN.
   86. The CRISPR/Cas system of clause 85, wherein the Cas nuclease comprises an amino acid sequence having at least 85% sequence identity to SEQ ID NO: 60.
   87. The CRISPR/Cas system of any one of clauses 72-86, wherein a nucleic acid sequence encoding a promoter is operably linked to the codon optimized nucleotide sequence that encodes a Cas nuclease or variant thereof.
   88. The CRISPR/Cas system of clause 87, wherein the promoter is a spatially-restricted promoter, bidirectional promoter, or an inducible promoter.
   89. The CRISPR/Cas system of clause 88, wherein the spatially-restricted promoter is selected from a group consisting of: any tissue or cell type specific promoter, a hepatocyte-specific promoter, a neuron-specific promoter, an adipocyte-specific promoter, a cardiomyocyte-specific promoter, a skeletal muscle-specific promoter, lung progenitor cell specific promoter, a photoreceptor-specific promoter, and a RPE selective promoter.
   90. The CRISPR/Cas system of any one of clauses 72-89, wherein the gRNA is a sgRNA.
   91. The CRISPR/Cas system of any one of clauses 72-90, wherein the gRNA or sgRNA comprising a spacer sequence comprising 17 to 24 nucleotides.
   92. The CRISPR/Cas system of any of clauses 72-91, wherein the nuclease segment and the gRNA segment are provided together in a first vector and the shRNA segment is provided in a second vector.
   93. The CRISPR/Cas system of any of clauses 75-91, wherein the nuclease segment, the gRNA segment, and the one or more self-inactivating segments are provided together in a first vector and the shRNA segment is provided in a second vector.
   94. The CRISPR/Cas system of any of clauses 75-91, wherein the nuclease segment, the gRNA segment, the one or more self-inactivating segments, and the shRNA segment are provided in a vector.
   95. The CRISPR/Cas system of any of clauses 92-93, wherein the first vector and the second vector are AAV vectors or plasmids.
   96. The CRISPR/Cas system of clause 95, wherein the AAV vectors are AAV2 serotype vectors, AAV5 serotype vectors, or AAV6 serotype vectors.
   97. A pharmaceutical composition comprising the CRISPR/Cas system of any of clauses 72-96.
   98. A packaging cell comprising the CRISPR/Cas system of any of clauses 72-96.
   99. A method of controlling post-transcriptional expression of Cas nuclease during AAV packaging, the method comprising:
contacting a packaging cell with the CRISPR/Cas system of any one of clauses 72-96.
   100. A method of reducing mutagenesis at one or more SIN site in a recombinant AAV vector, the method comprising:
contacting a cell with the CRISPR/Cas system of any one of clauses 72-96.
   101. The method of clauses 99 or 100, wherein the packaging cell is a human cell.
   102. A method of controlling post-transcriptional expression of Cas nuclease during AAV packaging, the method comprising:
contacting a packaging cell with a nucleic acid comprising a sequence encoding the recombinant AAV vector of any one of clauses 34-60; and
contacting the packaging cell with at least one vector comprising nucleic acid sequence encoding a shRNA segment.
   103. A method of producing a recombinant AAV vector, the method comprising:
introducing into a packaging cell:
   (i) a first vector comprising a shRNA segment comprising a nucleotide sequence that encodes a shRNA that comprises sequence that is complementary to a transcript from the nuclease segment;
   (ii) a nucleic acid comprising sequence encoding the recombinant AAV vector of any one of clauses 34-60; and
   (iii) one or more viral components for producing the recombinant AAV vector;
culturing the packaging cell; and
isolating the recombinant AAV vector comprising the nucleic acid from the packaging cell.
   104. A method of producing a recombinant AAV vector, the method comprising:
introducing into a packaging cell a nucleic acid comprising sequence encoding the recombinant AAV vector of any one of clauses 34-60;
introducing into the packaging cell one or more viral components for producing the AAV;
culturing the packaging cell;
isolating the recombinant AAV vector comprising the nucleic acid from the packaging cell;
wherein the packaging cell expresses a shRNA.
   105. The method of any one of clauses 103-104, wherein the one or more viral components are encoded by the nucleic acid.
   106. The method of any one of clauses 103-104, wherein the one or more viral components are introduced via separate vector other than the nucleic acid.
   107. The method of any one of clauses 103-104, wherein the one or more viral components are encoded in a cellular genome.
   108. A CRISPR/Cas system comprising:
a nuclease segment comprising a codon optimized nucleotide sequence that encodes a Cas nuclease or variant thereof;
a gRNA segment comprising a nucleotide sequence that encodes a gRNA or sgRNA;
a promoter segment comprising a nucleotide sequence that encodes a first promoter comprising one or more tetracycline operator sequences, wherein the gRNA segment is operably linked to the promoter segment; and
a shRNA segment comprising a nucleotide sequence that encodes a shRNA that comprises sequence that is complementary to a transcript from the nuclease segment.
   109. The CRISPR/Cas system of clause 108, wherein the first promoter is selected from a group consisting of: a H1 promoter, U6 promoter, 7SK promoter, and portions of any thereof.
   110. The CRISPR/Cas system of clauses 108 or 109, wherein the one or more tetracycline operator sequence comprises a nucleic acid sequence having at least 85% sequence identity to SEQ ID NO: 4.
   111. The CRISPR/Cas system of any one of clauses 108-109, wherein the one or more tetracycline operator sequence comprises SEQ ID NO: 4.
   112. The CRISPR/Cas system of any one of clauses 108-111, further comprising:
a repressor segment comprising a nucleotide sequence that encodes a tetracycline repressor protein.
   113. The CRISPR/Cas system of clause 112, wherein the tetracycline repressor comprises a nucleic acid sequence having at least 85% sequence identity to SEQ ID NO: 62.
   114. The CRISPR/Cas system of clause 112, wherein the tetracycline repressor comprises a nucleic acid sequence comprising SEQ ID NO: 62.
   115. The CRISPR/Cas system of any one of clauses 108-114, wherein the one or more tetracycline operator sequences are capable of being bound by the tetracycline repressor protein.
   116. The CRISPR/Cas system of any one of clauses 108-115, wherein the shRNA comprises sequence having at least 85% sequence identity to any one of SEQ ID NOs: 9-11 or 55-59.
   117. The CRISPR/Cas system any one of clauses 108-116, wherein the shRNA comprises SEQ ID NOs: 9-11 or 55-59.
   118. The CRISPR/Cas system of any one of clauses 108-117, further comprising:
one or more self-inactivating segments comprising a SIN site;
wherein the gRNA or sgRNA is substantially complementary to the SIN site;
wherein the gRNA or sgRNA is substantially complementary to a genomic target sequence within a cell of a patient.
   119. The CRISPR/Cas system of clause 118, wherein the one or more self-inactivating segments are located in at least one of:
(i) at the 5′ end of the codon optimized nucleotide sequence that encodes a Cas nuclease or variant thereof;
(ii) at the 3′ end of the codon optimized nucleotide sequence that encodes a Cas nuclease or variant thereof; and
(iii) in an intron within the codon optimized nucleotide sequence that encodes a Cas nuclease or variant thereof.
   120. The CRISPR/Cas system of clause 118, wherein the one or more self-inactivating segments are located in:
(i) at the 5′ end of the codon optimized nucleotide sequence that encodes a Cas nuclease or variant thereof; and
(ii) in an intron within the codon optimized nucleotide sequence that encodes a Cas nuclease or variant thereof.
   121. The CRISPR/Cas system of any one of clauses 118-120, wherein one of the one or more self-inactivating segments are located upstream of the codon optimized nucleotide sequence that encodes a Cas nuclease or variant thereof and downstream of a NLS.
   122. The CRISPR/Cas system of any one of clauses 118-120, wherein the SIN site comprises a PAM sequence.
   123. The CRISPR/Cas system of clause 122, wherein the PAM sequence in the SIN site is selected from a group consisting of: NNGRRT, NRG, NAAAAN, NAAAAC, NNNNGHTT, YTN, NNNNACAC, NNVRYAC, NNNNVRYM, NNAAAAW, NNAGAAW, and NNGG.
   124. The CRISPR/Cas system of any of clauses 108-122, wherein the gRNA or sgRNA is fully complementary to the nucleotide sequence of the SIN site except for at one base pair.
   125. The CRISPR/Cas system of any of clauses 108-122, wherein the gRNA or sgRNA is fully complementary to the nucleotide sequence of the SIN site except for at two base pairs.
   126. The CRISPR/Cas system of any one of clauses 108-125, wherein the Cas nuclease is a Class 2 Cas nuclease.
   127. The CRISPR/Cas system of any one of clauses 108-126, wherein the Cas nuclease is selected from a group consisting of: S. pyogenes Cas, S. aureus Cas, S. thermolphilus Cas, C. jejuni Cas, T. denticola Cas, N. meningitides Cas, S. lugdunensis Cas, S. hyicus Cas, S. microti Cas, and S. pasteuri Cas.
   128. The CRISPR/Cas system of any one of clauses 108-125, wherein the Cas nuclease is a SRGN.
   129. The CRISPR/Cas system of clause 128, wherein the Cas nuclease comprises an amino acid sequence having at least 85% sequence identity to SEQ ID NO: 60.
   130. The CRISPR/Cas system of any one of clauses 108-129, wherein a nucleic acid sequence encoding a second promoter is operably linked to the codon optimized nucleotide sequence that encodes a Cas nuclease or variant thereof.
   131. The CRISPR/Cas system of clause 130, wherein the second promoter is a spatially-restricted promoter, bidirectional promoter, or an inducible promoter.
   132. The CRISPR/Cas system of clause 131, wherein the spatially-restricted promoter is selected from a group consisting of: any tissue or cell type specific promoter, a hepatocyte-specific promoter, a neuron-specific promoter, an adipocyte-specific promoter, a cardiomyocyte-specific promoter, a skeletal muscle-specific promoter, lung progenitor cell specific promoter, a photoreceptor-specific promoter, and a RPE selective promoter.
   133. The CRISPR/Cas system of any one of clauses 108-132, wherein the gRNA is a sgRNA.
   134. The CRISPR/Cas system of any one of clauses 108-133, wherein the gRNA or sgRNA comprising a spacer sequence comprising 17 to 24 nucleotides.
   135. The CRISPR/Cas system of any one of clauses 108-134, wherein the nuclease segment, the gRNA segment, and the promoter segment are provided together in a first vector and the repressor segment and/or the shRNA segment is provided in a second vector.
   136. The CRISPR/Cas system of any one of clauses 118-134, wherein the nuclease segment, the gRNA segment, the promoter segment, and the one or more self-inactivating segments are provided together in a first vector and the repressor segment and/or the shRNA segment is provided in a second vector.
   137. The CRISPR/Cas system of any one of clauses 118-134, wherein the nuclease segment, the gRNA segment, the promoter segment, the one or more self-inactivating segments, the repressor segment, and the shRNA segment are provided in a vector.
   138. The CRISPR/Cas system of any one of clauses 135-136, wherein the first vector and the second vector are AAV vectors or plasmids.
   139. The CRISPR/Cas system of clause 138, wherein the AAV vectors are AAV2 serotype vectors, AAV5 serotype vectors, or AAV6 serotype vectors.
   140. A pharmaceutical composition comprising the CRISPR/Cas system of any one of clauses 108-139.
   141. A packaging cell comprising the CRISPR/Cas system of any one of clauses 108-139.
   142. A method of controlling transcription of gRNAs and post-transcriptional expression of Cas nuclease during AAV packaging, the method comprising:
contacting a packaging cell with the CRISPR/Cas system of any one of clauses 108-139.
   143. A method of reducing mutagenesis at one or more SIN site in a recombinant AAV vector, the method comprising:
contacting a cell with the CRISPR/Cas system of any one of clauses 108-139.
   144. The method of any one of clauses 142 or 143, wherein the packaging cell is a human cell.
   145. A method of controlling transcription of gRNAs and post-transcriptional expression of Cas nuclease during AAV packaging, the method comprising:
contacting a packaging cell with a nucleic acid comprising sequence encoding the recombinant AAV vector of any one of clauses 34-60; and
contacting the packaging cell with a nucleic acid sequence encoding a tetracycline repressor segment and a shRNA segment.
   146. A method of producing a recombinant AAV vector, the method comprising:
introducing into a packaging cell:
   (i) a first vector comprising a repressor segment, wherein the repressor segment comprises a nucleotide sequence that encodes a tetracycline repressor protein;
   (ii) a second vector comprising a shRNA segment comprising a nucleotide sequence that encodes a shRNA that comprises sequence that is complementary to a transcript from the nuclease segment;
   (iii) a nucleic acid comprising sequence encoding the recombinant AAV vector of any one of clauses 34-60; and
   (iv) one or more viral components for producing the recombinant AAV vector;
culturing the packaging cell; and
isolating the recombinant AAV vector comprising the nucleic acid of (iii) from the packaging cell.
   147. A method of producing a recombinant AAV vector, the method comprising:
introducing into packaging cell a nucleic acid comprising sequence encoding the recombinant AAV vector of any one of clauses 34-60;
introducing into the packaging cell one or more viral components for producing the AAV;
culturing the packaging cell; and
isolating the recombinant AAV vector comprising the nucleic acid from the packaging cell;
wherein the packaging cell expressing a tetracycline repressor protein and a shRNA.
   148. The method of any one of clauses 146-147, wherein the nucleic acid further comprises one or more viral components.
   149. The method of any one of clauses 146-147, wherein the one or more viral components are introduced via separate vector other than the nucleic acid.
   150. The method of any one of clauses 146-147, wherein the one or more viral components are encoded in a cellular genome.
   151. A recombinant AAV vector produced by any one of the methods of clauses 67-71, 103-107, or 146-150.

### Definitions

In addition to the definitions previously set forth herein, the following definitions are relevant to the present disclosure:

The singular forms “a,” “an,” and “the” include plural references, unless the context clearly dictates otherwise.

Any numerical range recited in this specification describes all sub-ranges of the same numerical precision (i.e., having the same number of specified digits) subsumed within the recited range. For example, a recited range of “1.0 to 10.0” describes all sub-ranges between (and including) the recited minimum value of 1.0 and the recited maximum value of 10.0, such as, for example, “2.4 to 7.6,” even if the range of “2.4 to 7.6” is not expressly recited in the text of the specification. Accordingly, the Applicant reserves the right to amend this specification, including the claims, to expressly recite any sub-range of the same numerical precision subsumed within the ranges expressly recited in this specification. All such ranges are inherently described in this specification such that amending to expressly recite any such sub-ranges will comply with written description, sufficiency of description, and added matter requirements, including the requirements under 35 U.S.C. § 112 (a) and Article 123 (2) EPC. Also, unless expressly specified or otherwise required by context, all numerical parameters described in this specification (such as those expressing values, ranges, amounts, percentages, and the like) may be read as if prefaced by the word “about,” even if the word “about” does not expressly appear before a number. Additionally, numerical parameters described in this specification should be construed in light of the number of reported significant digits, numerical precision, and by applying ordinary rounding techniques. It is also understood that numerical parameters described in this specification will necessarily possess the inherent variability characteristic of the underlying measurement techniques used to determine the numerical value of the parameter.

Any patent, publication, or other disclosure material identified herein is incorporated by reference into this specification in its entirety unless otherwise indicated, but only to the extent that the incorporated material does not conflict with existing descriptions, definitions, statements, or other disclosure material expressly set forth in this specification. As such, and to the extent necessary, the express disclosure as set forth in this specification supersedes any conflicting material incorporated by reference. Any material, or portion thereof, that is said to be incorporated by reference into this specification, but which conflicts with existing definitions, statements, or other disclosure material set forth herein, is only incorporated to the extent that no conflict arises between that incorporated material and the existing disclosure material. Applicants reserve the right to amend this specification to expressly recite any subject matter, or portion thereof, incorporated by reference herein.

The details of one or more aspects of the present disclosure are set forth in the accompanying examples below. Although any materials and methods similar or equivalent to those described herein can be used in the practice or testing of the present disclosure, specific examples of the materials and methods contemplated are now described. Other features, objects and advantages of the present disclosure will be apparent from the description. In the description examples, the singular forms also include the plural unless the context clearly dictates otherwise. Unless defined otherwise, all technical and scientific terms used herein have the same meaning as commonly understood by one of ordinary skill in the art to which this present disclosure belongs. In the case of conflict, the present description will control.

### EXAMPLES

The present disclosure will be more fully understood by reference to the following examples, which provide illustrative, non-limiting aspects of the invention.

The examples describe the use of materials and methods for controlling gene editing. Gene editing can be controlled by controlling transcriptional expression of gRNAs and/or controlling post-transcriptional expression of a Cas nuclease, which can thereby reduce mutagenesis at one or more SIN site in a recombinant AAV vector during packaging. The defined control mechanisms represent a novel strategy for inhibiting mutagenesis at one or more SIN site in a recombinant AAV vector during packaging, as described and illustrated herein.

### Example 1—Controlling Transcriptional Expression of gRNAs Using a Tetracycline Operator/Repressor System

An experiment was conducted to determine whether on-target editing of a P23H target site is inhibited by controlling the transcription of gRNAs using a tetracycline operator/tetracycline repressor system.

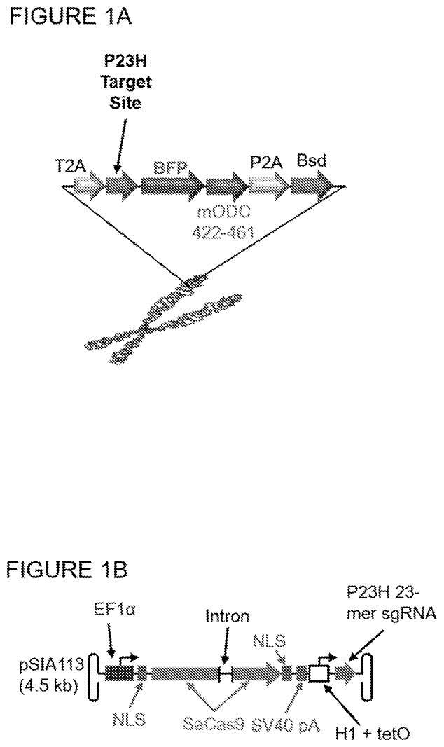

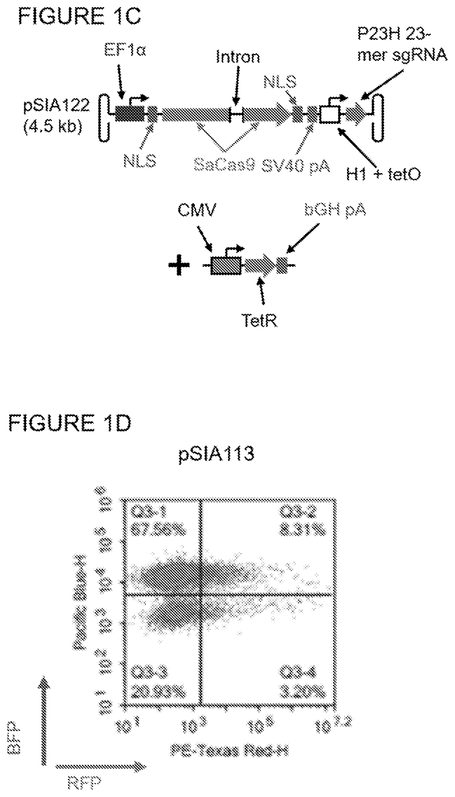

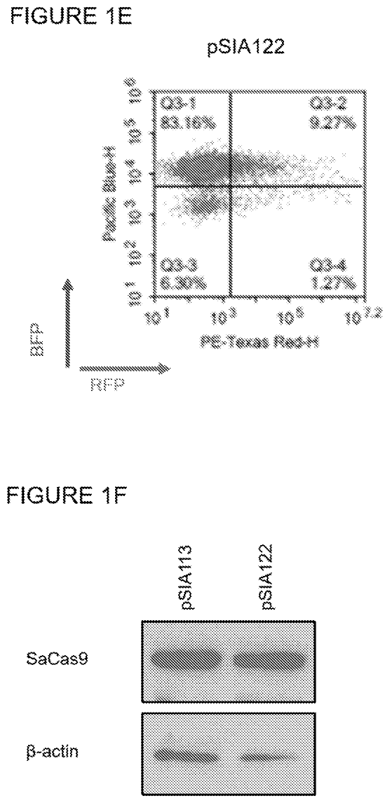

A reporter cell line was generated that contains a reporter construct comprising a P23H target site fused to a BFP coding sequence at the beta-tubulin gene locus of HEK 293 FT cells (FIG. 1A).

pSIA113 (SEQ ID NO: 5), depicted in FIG. 1B, comprises a SaCas9 gene (SEQ ID NO: 64), a sequence that encodes a P23H 23-mer sgRNA (SEQ ID NO: 1), and an H1 promoter comprising two TetO sites (SEQ ID NO: 3) between two AAV ITRs. The P23H 23-mer sgRNA is a sgRNA comprising a spacer sequence (SEQ ID NO: 30) that targets the P23H target site on the reporter construct.

pSIA122 (SEQ ID NO: 6), depicted in FIG. 1C, comprises a SaCas9 gene (SEQ ID NO: 64), a sequence that encodes a P23H 23-mer sgRNA (SEQ ID NO: 1), an H1 promoter comprising two TetO sites (SEQ ID NO: 3) between two AAV ITRs, and a TetR (e.g., SEQ ID NO: 62). The tetracycline repressor is able to bind to the two TetO sites to repress transcription of the P23H 23-mer sgRNA gene.

The HEK 293 FT reporter cell line was transfected with 1.25 μg pSIA113 and 1.25 μg red fluorescence (RFP)-expressing plasmid using Lipofectamine® 3000, available from Thermo Fisher Scientific, Massachusetts, US.

The HEK 293 FT reporter cell line was separately transfected with 2.0 μg pSIA122 and 0.5 μg RFP-expressing plasmid using Lipofectamine® 3000.

At 2 and 5 days post-transfection, HEK 293 FT reporter cells were dissociated from plates by incubation with trypsin-EDTA and analyzed for BFP and RFP by flow cytometry. A frame-shift induced by genome editing at the P23H target site of the HEK 293 FT cell results in loss of BFP. This was analyzed at 5 days post-transfection.

BFP negative means that gene editing occurred at the P23H target site of these transfected HEK 293 FT cells. RFP positive means that these transfected HEK 293 FT cells contain a plasmid that encodes RFP and serves as a control for transfection efficiency. This was analyzed at 2 days post-transfection.

FIG. 1D shows that of the HEK 293 FT reporter cells transfected with pSIA113, 20.93% of these cells had frame-shift mutations introduced at the P23H target site when P23H 23-mer sgRNA was used as the sgRNA.

FIG. 1E shows that of the HEK 293 FT reporter cells transfected with pSIA122, 6.30% of these cells had frame-shift mutations introduced at the P23H target site when P23H 23-mer sgRNA was used as the sgRNA.

The results in FIGS. 1D-1E demonstrate that overexpression of TetR can control gene editing through binding of TetR to the tetracycline operators, which inhibits transcriptional expression of gRNAs.

At 2 days post-transfection, HEK 293 FT reporter cells were dissociated from plates by incubation with trypsin-EDTA. Cells were lysed and protein isolated for western blot analysis. Levels of SaCas9 and β-actin were compared. SaCas9 levels in HEK 293 FT reporter cells transfected with pSIA113 and SaCas9 levels in HEK 293 FT reporter cells transfected with pSIA122 were similar (FIG. 1F) suggesting that expression of TetR did not affect the expression of SaCas9.

These data provide evidence that expression of TetR can control gene editing through binding of TetR to the tetracycline operators, which inhibits transcriptional expression of gRNAs. These data also provide evidence that the overexpression of TetR did not affect the expression of SaCas9. Overall, the data provide evidence that transcriptional control via repression of gRNA transcription is a possible method for inhibiting editing by CRISPR/Cas systems during recombinant AAV vector packaging.

### Example 2—Gene Editing Comparision Between sgRNA Driven by a Human U6 Promoter and a H1 Promoter Comprising Two TetO Sites

To determine how the TetO sites in the H1 promoter affect transcription of the gRNA gene, a second experiment was performed. A comparison was made to another exemplary PolIII promoter, U6, comprising no TetO sites.

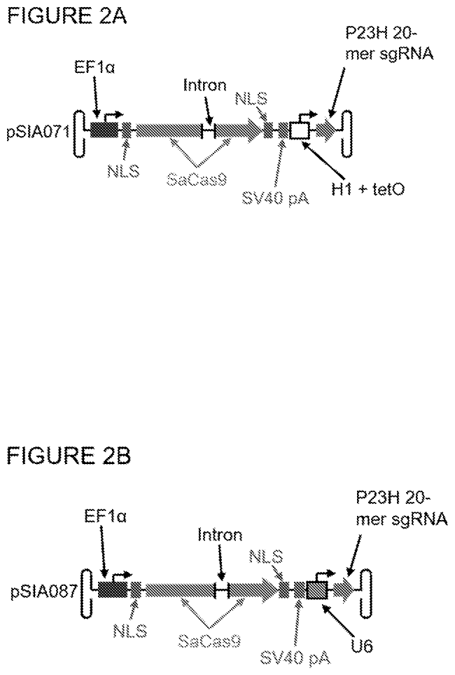

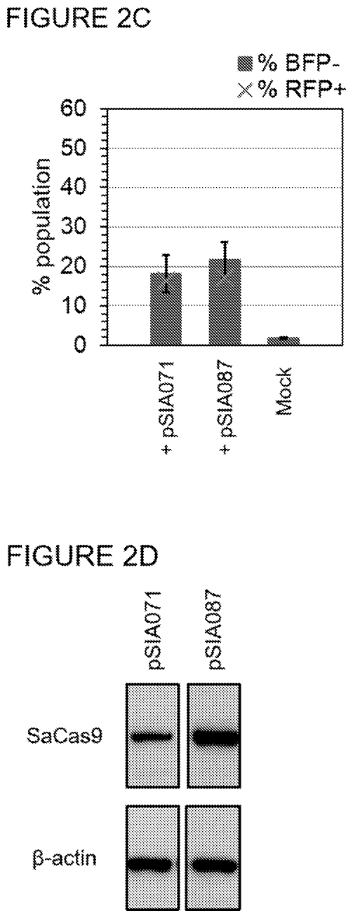

Using the reporter cell line of Example 1, cells were transfected with either pSIA071 or pSIA087. pSIA071 (SEQ ID NO: 7), depicted in FIG. 2A, comprises a SaCas9 gene (SEQ ID NO: 64), a sequence that encodes a P23H 20-mer sgRNA (SEQ ID NO: 2), and an H1 promoter comprising two TetO sites (SEQ ID NO: 3) between two AAV ITRs. The P23H 20-mer sgRNA is a sgRNA comprising a spacer sequence (SEQ ID NO: 33) that targets the P23H target site on the reporter construct.

pSIA087 (SEQ ID NO: 8), depicted in FIG. 2B, comprises a SaCas9 gene (SEQ ID NO: 64) and a sequence that encodes a P23H 20-mer sgRNA (SEQ ID NO: 2) driven by a U6 promoter between two AAV ITRs. No TetO sites are included in pSIA087 and no TetR gene or protein was included in either transfection. Thus, comparison of the relative gRNA promoter strength of each of the two constructs was possible.

The HEK 293 FT reporter cell line was transfected with 2.0 μg of the pSIA071 plasmid, using Lipofectamine® 3000. The HEK 293 FT reporter cell line was separately transfected with 2.0 μg of the pSIA087 plasmid using Lipofectamine® 3000. In both cases, the cells were also transfected with 0.5 μg of an RFP-expressing plasmid.

At 2 and 5 days post-transfection, HEK 293 FT reporter cells were dissociated from plates by incubation with trypsin-EDTA and analyzed for BFP expression and RFP expression by flow cytometry. A frame-shift induced by genome editing at the P23H target site of the HEK 293 FT cell results in loss of BFP. This was analyzed at 5 days post-transfection.

In BFP-cells, gene editing-mediated frame-shift occurred at the P23H target site of these transfected reporter cells. RFP+ cells contain a plasmid that encodes RFP. Transfection with the RFP plasmid allows for determination of whether or not cell populations were transfected at similar rates. This was analyzed at 2 days post-transfection.

FIG. 2C shows that of the HEK 293 FT reporter cells transfected with pSIA071, ˜19% of these cells had frame-shift mutations introduced by the SaCas nuclease-sgRNA complex at the P23H target site. Of the HEK 293 FT reporter cells transfected with pSIA087, ˜21% of these cells had frame-shift mutations introduced by the SaCas nuclease-sgRNA complex at the P23H target site.

FIG. 2C also shows that of the HEK 293 FT reporter cells transfected with pSIA071, ˜16% of these cells expressed RFP. Of the HEK 293 FT reporter cells transfected with pSIA087, ˜18% of these cells expressed RFP.

FIG. 2D shows an image of a western blot which was conducted using lysate from transfected cells harvested 2 days post-transfection. The blot was probed for SaCas9 and, as a loading control, β-actin. The left lane shows lysate from cells transfected with pSIA071, and the right lane shows lysate from cells transfected with pSIA087. SaCas9 is expressed at similar levels in both samples, indicating that the differences in the sgRNA promoters did not alter the expression of the SaCas9 gene.

Because the differences in editing efficiency were minimal (compare % BFP—in FIG. 2C), it is suggested that the strength of the two promoters (H1 variant containing 2 copies of TetO sites and U6) is similar and that, when un-occupied, the TetO sites of the H1 promoter do not negatively impact transcription. Furthermore, it is possible that other PolIII promoters, in addition to H1, such as U6, could be engineered to comprise one or more TetO sites, thereby achieving similar levels of transcriptional control of sgRNA genes during recombinant AAV vector production.

### Example 3—Controlling Post-Transcriptional Expression of SaCas Nuclease Using a shRNAs

To evaluate various shRNAs for their ability to inhibit translation of Cas nuclease gene transcripts, an experiment was performed. Comparisons were made between cell populations of the BFP reporter cell line from Example 1, which was transfected with a vector comprising SaCas9 and a sgRNA gene and another vector carrying either a shRNA gene or no shRNA gene.

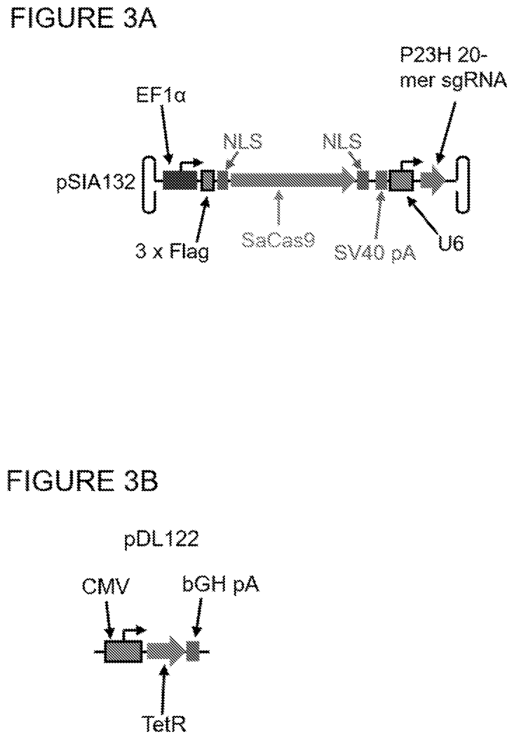

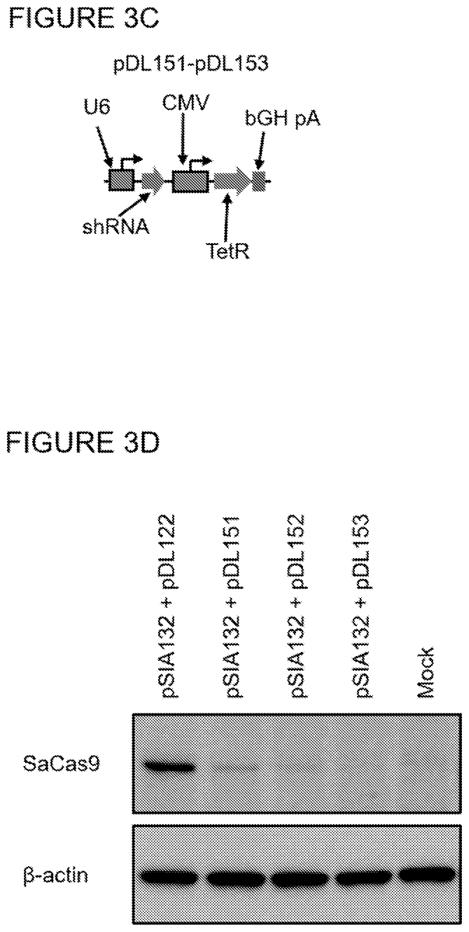

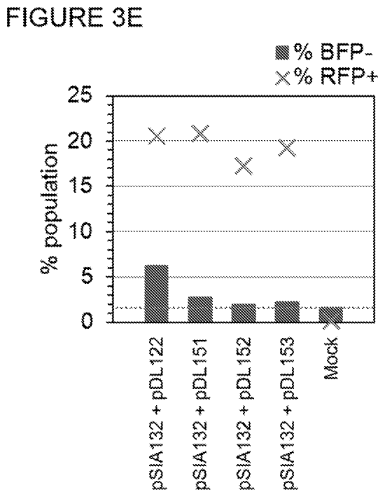

Cells were transfected with pSIA132 (SEQ ID NO: 12), depicted in FIG. 3A. pSIA132 is a plasmid comprising a SaCas9 gene (SEQ ID NO: 64) and a sequence that encodes a P23H 20-mer sgRNA (SEQ ID NO: 2), driven by a U6 promoter between two AAV ITRs. Cells were also transfected with one of 4 other plasmids: pDL122 (a negative control comprising no shRNA), pDL151, pDL152, or pDL153. pDL122 is depicted in FIG. 3B, and comprises a TetR gene. The TetR gene is not expected to affect the results of the experiment since no TetO sites are present on any of the constructs. A generalized view of pDL151, pDL152, and pDL153 is provided in FIG. 3C. Each of the three plasmids encodes a unique shRNA that is complementary to a portion of the SaCas9 transcript. pDL151 comprises a sequence encoding a shRNA comprising the sequence of SEQ ID NO: 9. pDL152 comprises a sequence encoding a shRNA comprising the sequence of SEQ ID NO: 10. pDL153 comprises a sequence encoding a shRNA comprising the sequence of SEQ ID NO: 11.

The HEK 293 FT reporter cell line was transfected with 1.0 μg of the pSIA132 plasmid, using Lipofectamine® 3000. Additionally, the HEK 293 FT reporter cell line was transfected with 1.0 μg of one of either pDL122, pDL151, pDL152, or pDL153 using Lipofectamine® 3000. In all cases, the cells were also transfected with 0.5 μg of an RFP-expressing plasmid.

FIG. 3D shows an image of a western blot conducted using lysate from cells harvested 2 days post-transfection. Mock transfected cells (right-most lane) showed no SaCas9 signal above background. Cells transfected with pSIA132 and pDL122 showed strong expression of SaCas9, consistent with the absence of any shRNA reducing SaCas9 gene expression. Comparisons can be made between the cells transfected with pDL122 and those transfected with the shRNA-expressing plasmids. Cells transfected with pSIA132 and pDL151 showed decreased expression of SaCas9, consistent with the presence of a shRNA reducing SaCas9 gene expression. Cells transfected with pSIA132 and pDL152 showed decreased expression of SaCas9, consistent with the presence of a shRNA reducing SaCas9 gene expression. Cells transfected with pSIA132 and pDL153 showed decreased expression of SaCas9, consistent with the presence of a shRNA reducing SaCas9 gene expression.

While FIG. 3D provides data on Cas nuclease expression, FIG. 3E provides data related to Cas nuclease editing activity, as directed by the P23H 20-mer sgRNA. 2 and 5 days post-transfection, cells were prepared for flow cytometry as described in Example 1. Mock transfected cells showed a baseline percentage of BFP-cells of ˜1.5% at 5 days post-transfection. In contrast, cells transfected with pSIA132 and pDL122 showed an increase in the percentage of BFP-cells, with ˜6% of cells being negative for BFP expression. This increase is consistent with editing of the reporter construct in the absence of post-transcriptional control of SaCas9 expression by shRNAs. For cells transfected with pSIA132 and pDL151, ˜3% of cells were negative for BFP expression. For cells transfected with pSIA132 and pDL152, ˜2% of cells were negative for BFP expression. For cells transfected with pSIA132 and pDL153, ˜2% of cells were negative for BFP expression. Between ˜17% and ˜21% of all transfected cells were positive for RFP at 2 days post-transfection, indicating that transfection efficiency was similar for all cells.

Taken together, FIGS. 3D and 3E provide evidence that expression of Cas nucleases can be controlled by shRNA expression and that this control can reduce editing by a Cas nuclease and a gRNA paired with the nuclease.

### Example 4—Analysis of AAV Vectors Packaged in AAV2 or AAV5

To evaluate the ability to successfully package and prepare various recombinant AAV vectors, an experiment was performed. Specifically, accumulation of deletions in vector genomes was analyzed. Recombinant AAV vectors were prepared in packaging cells. Comparisons were made between recombinant AAV vectors comprising either SIN sites or no SIN sites and between vectors which were or were not packaged in packaging cells expressing TetR repressor protein.

One group of packaging cells was transfected with plasmid pSIA113 (SEQ ID NO: 5), depicted in FIG. 1B.

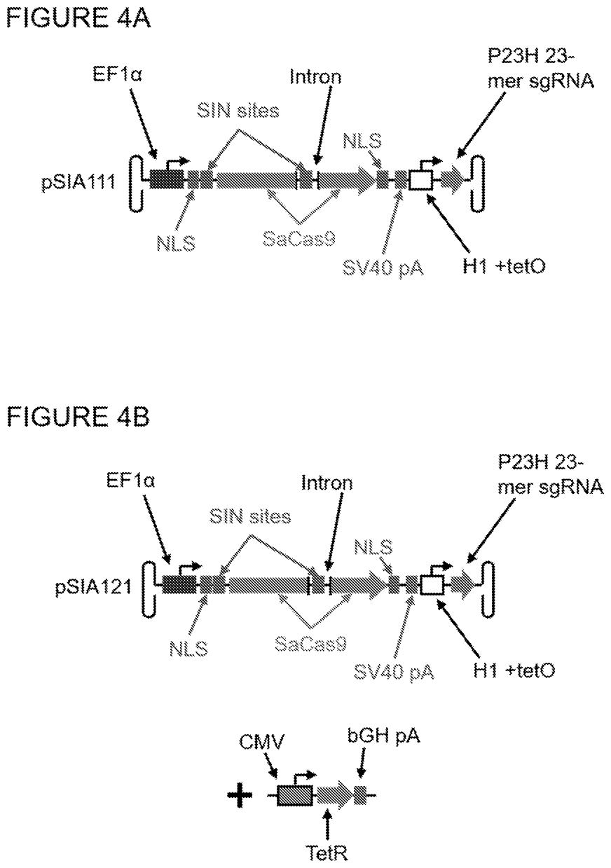

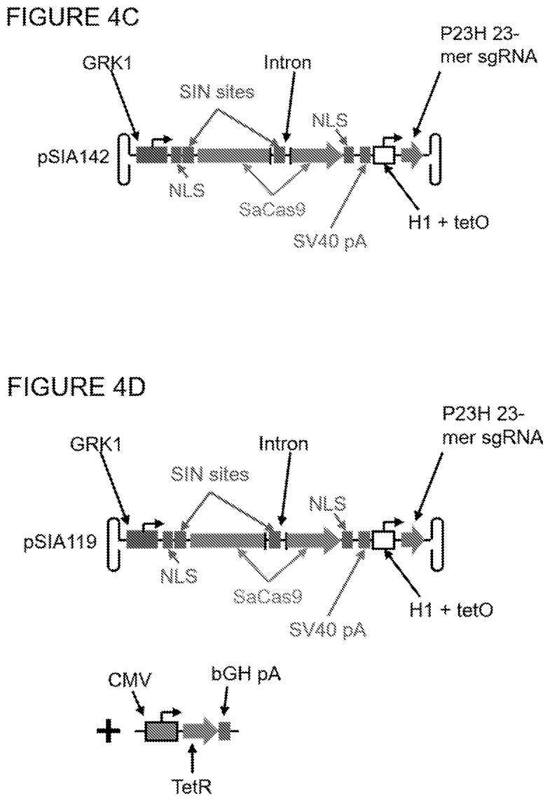

A second group of packaging cells was transfected with plasmid pSIA111 (SEQ ID NO: 17), depicted in FIG. 4A. pSIA111 comprises a SaCas9 gene (SEQ ID NO: 64), driven by an EF1α promoter and a sequence that encodes a P23H 23-mer sgRNA (SEQ ID NO: 1), driven by an H1 promoter comprising two TetO sites (SEQ ID NO: 3) between two AAV ITRs. The sequence between AAV ITRs of pSIA111 further comprises two SIN sites, with a first SIN site located between the NLS and the immediate 5′ end of SaCas9 and a second SIN site located within the intron of SaCas9.

A third group of packaging cells was transfected with plasmid pSIA121 (SEQ ID NO: 18), depicted in FIG. 4B. pSIA121 is a plasmid comprising a SaCas9 gene (SEQ ID NO: 64), driven by an EF1α promoter and a sequence that encodes a P23H 23-mer sgRNA (SEQ ID NO: 1), driven by an H1 promoter comprising two TetO sites (SEQ ID NO: 3) between two AAV ITRs. The sequence between AAV ITRs of pSIA121 further comprises two SIN sites, with a first SIN site located between the NLS and the immediate 5′ end of SaCas9 and a second SIN site located within the intron of SaCas9. pSIA121 further comprises a sequence encoding a TetR protein (e.g., SEQ ID NO: 62). The TetR sequence is located on a separate locus of the plasmid, compared to the sequence between the two AAV ITRs. The TetR gene, while expressed during packaging, is not included in recombinant AAV vectors prepared during packaging because the TetR gene is not located between the AAV inverted terminal repeats.

A fourth group of packaging cells was transfected with plasmid pSIA142 (SEQ ID NO: 19), depicted in FIG. 4C. pSIA142 is a plasmid comprising a SaCas9 gene (SEQ ID NO: 64), driven by a GRK1 promoter and a sequence that encodes a P23H 23-mer sgRNA (SEQ ID NO: 1), driven by an H1 promoter comprising two TetO sites (SEQ ID NO: 3) between two AAV ITRs. The sequence between AAV ITRs of pSIA142 further comprises two SIN sites, with a first SIN site located between the NLS and the immediate 5′ end of SaCas9 and a second SIN site located within the intron of SaCas9.

A fifth group of packaging cells was transfected with plasmid pSIA119 (SEQ ID NO: 20), depicted in FIG. 4D. pSIA119 is a plasmid comprising a SaCas9 gene (SEQ ID NO: 64), driven by a GRK1 promoter and a sequence that encodes a P23H 23-mer sgRNA (SEQ ID NO: 1), driven by an H1 promoter comprising two TetO sites (SEQ ID NO: 3) between two AAV ITRs. The sequence between AAV ITRs of pSIA119 further comprises two SIN sites, with a first SIN site located between the NLS and the immediate 5′ end of SaCas9 and a second SIN site located within the intron of SaCas9. pSIA119 further comprises a sequence encoding a TetR protein (e.g., SEQ ID NO: 62). The TetR sequence is located on a separate locus of the plasmid, compared to the sequence between AAV ITRs. The TetR gene, while expressed during packaging, is not included in recombinant AAV vectors prepared during packaging because the TetR gene is not located between the AAV inverted terminal repeats.

Packaging was performed by a standard ‘triple’ transfection method. Packaging cells were transfected with one of the 5 plasmids (pSIA113, pSIA111, pSIA121, pSIA142, or pSIA119), and each of the 5 groups of packaging cells was also transfected with two other plasmids. One plasmid encodes AAV rep genes and AAV2 or AAV5 cap genes. The other plasmid encodes helper virus genes.

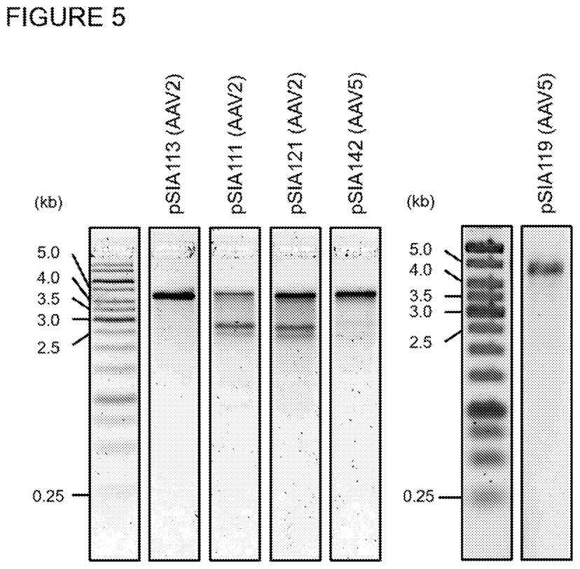

After packaging and recombinant AAV vector particle purification, genomic DNA was isolated from each sample and used for alkaline agarose gel electrophoresis (FIG. 5).

AAV serotype 2 vectors packaged with pSIA113 used as template showed a single band of the expected genome size. This was expected since no SIN sites are present on this vector, and thus it should not be targeted during packaging by the SaCas nuclease-sgRNA complex, even though the complex is expected to be expressed during packaging.

AAV serotype 2 vectors packaged with pSIA111 used as template showed multiple bands. This indicates that a portion of the genomes packaged in these cells sustained a deletion due to cutting by the SaCas nuclease-sgRNA complex at the two SIN sites in the viral vector genome. This cutting was expected since no TetR protein was supplied during packaging.

AAV serotype 2 vectors packaged with pSIA121 used as template showed multiple bands. This indicates that a portion of the genomes packaged in these cells sustained a deletion due to cutting by the SaCas nuclease-sgRNA complex at the two SIN sites in the viral vector genome. However, the full length band comprising intact genomes showed a stronger signal in the pSIA121 lane than in the pSIA111 lane, indicating that the TetR protein supplied during packaging was effective in reducing sgRNA expression and resultant cutting by the SaCas nuclease-sgRNA complex.

AAV serotype 5 vectors packaged with pSIA142 used as template showed multiple bands. This indicates that a portion of the genomes packaged in these cells sustained a deletion due to cutting by the SaCas nuclease-sgRNA complex at the two SIN sites in the viral vector genome. This cutting was expected since no TetR protein was supplied during packaging. It is noted that this viral vector carries a different promoter to drive expression of the SaCas9 gene, GRK1 compared to EF1α. The GRK1 promoter is active in photoreceptor cells but less active than the EF1α promoter in other cells such as the packaging cells used in this Example. Thus, fewer vector genomes with a deletion accumulated in vectors packaged with pSIA142, compared to vector packaged with pSIA111.

Finally, AAV serotype 5 vectors packaged with pSIA119 used as template showed only one visible band. This indicates that the combination of the photoreceptor cell-specific promoter driving SaCas9 expression and the TetR repressor protein decreasing transcription of the sgRNA worked synergistically to reduce vector genome deletion to a level low enough to escape detection by alkaline gel electrophoresis.

FIG. 5 provides evidence that editing activity leading to recombinant AAV vector genome deletion can be reduced during packaging of all-in-one vectors. This can be achieved by at least one of transcriptional control via a repressor of the sgRNA gene and transcriptional control of the Cas nuclease via a cell-type specific promoter.

### Example 5—Analysis of AAV Vectors Packaged in AAV2 or AAV5

To further evaluate the accumulation of deletions in packaged AAV vectors, recombinant AAV vectors were prepared in packaging cells as described in Example 4. PCR was conducted to detect both full length vectors and vectors with deletion between two SIN sites. Comparisons were made between recombinant AAV vectors comprising SIN sites and those without SIN sites. Additionally vectors comprising a cell type-specific promoter driving SaCas9 expression were investigated. The viral vectors were packaged either with or without the presence of TetR protein.

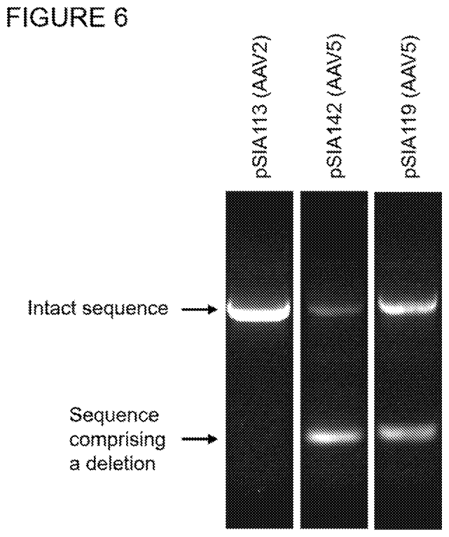

After packaging and recombinant AAV vector particle purification, as described in Example 4, genomic DNA was isolated from each sample and used as a template for a PCR (FIG. 6). Two oligonucleotide primers (SEQ ID NOs: 21 and 22) were used to amplify a sequence comprising the DNA between the two SIN sites. Thus, a longer product would be made by genomic templates with no deletion and a shorter product would be made by genomic templates with a deletion due to cutting at both SIN sites by the SaCas nuclease-sgRNA complex.

FIG. 6 shows that recombinant AVV vector genomes produced from pSIA113 did not comprise any such deletion. This is expected since this plasmid's AAV sequence does not comprise SIN sites. A portion of recombinant AVV vector genomes produced from either from pSIA142 or from pSIA119 did comprise a deletion. However, more intact genomes were produced when pSIA119 was used.

These results provide further evidence that both transcriptional control via a repressor of the sgRNA gene and transcriptional control of the Cas nuclease via a cell-type specific promoter can be successful strategies to control gene editing during packaging of viral vectors. Particularly, genomic deletion generation can be reduced by one or both of the strategies. A combination of these two strategies can be preferable.

### Example 6—Analysis of AAV Vectors Packaged in AAV2 or AAV5

Examples 4 and 5 above relate to analysis of genomic deletions in recombinant AAV vector constructs during packaging and the use of materials and methods of the present disclosure to inhibit such deletions. In addition to deletions, unwanted expression of CRISPR/Cas-related genes during packaging can also cause indels at sites (e.g., SIN sites) on the vector genome. The present disclosure accordingly provides materials and methods to inhibit indel formation on recombinant AAV vectors during packaging, as well as to inhibit generation of genomic deletions.

To analyze the ability to inhibit indel formation on recombinant AAV vectors during packaging, recombinant AAV vectors were generated using either pSIA111, pSIA113, pSIA121, or pSIA142. Generation and packaging of the recombinant AAV vectors was performed similarly to the corresponding procedure described in Examples 4 and 5 above.

Genomic DNA was prepared from the recombinant AAV vectors and was used as a template in two separate PCRs.

In a first PCR, an oligonucleotide primer with the sequence of SEQ ID NO: 23 was used as a forward primer for genomes prepared from pSIA111, pSIA113 and pSIA121, and an oligonucleotide primer with the sequence of SEQ ID NO: 24 was used as a forward primer for genomes prepared from pSIA142. An oligonucleotide primer with the sequence of SEQ ID NO: 25 was used as a reverse primer for genomes prepared from all four plasmids. This PCR reaction amplified a region of each vector comprising the 5′ SIN site of recombinant AAV vectors prepared from pSIA111, pSIA121, and pSIA142. Recombinant AAV vectors prepared from pSIA113 do not comprise a SIN site, but did produce an analogous PCR product.

In a second PCR, an oligonucleotide primer with the sequence of SEQ ID NO: 26 was used as a forward primer for genomes prepared from all four plasmids. An oligonucleotide primer with the sequence of SEQ ID NO: 27 was used as a reverse primer for genomes prepared from all four plasmids. This reaction amplified a region of each genome comprising the 3′ SIN site of recombinant AAV vectors prepared from pSIA111, pSIA121, and pSIA142. Recombinant AAV vectors prepared from pSIA113 do not comprise a SIN site, but did produce an analogous PCR product.

Because indels, unlike the larger deletions examined in Examples 4 and 5, cannot be detected by only gel electrophoresis, both the first and second PCR products were then examined in a T7 Endonuclease I (T7EI) assay. In this assay the PCR products for each sample were denatured and allowed to re-anneal. After the re-annealing, and if indels were generated in the vector genomes used as PCR templates, a population of the re-annealed products would have contained mismatched and/or bulged bases, due to strands with indels pairing with either unaltered strands or strands containing different indels. Such mismatches and/or bulges are a substrate for the T7EI, which would cut the product at the indel site, generating two smaller bands on an agarose gel, relative to the intact re-annealed product.

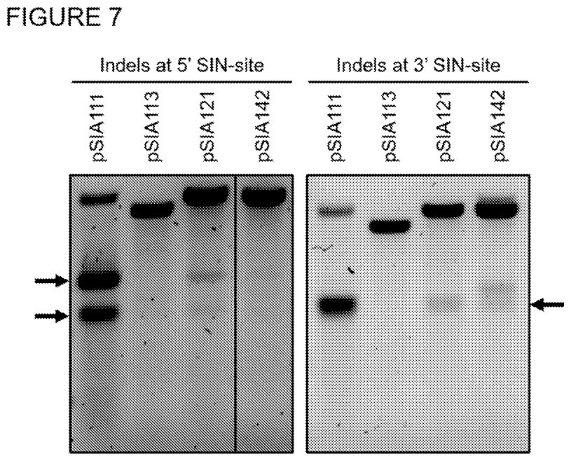

Results from the T7EI assay are shown in FIG. 7. Referring to the assay using the 5′ SIN site PCR product (left panel), PCR product from the vector prepared with the pSIA111 plasmid showed two smaller bands (marked by arrows), indicating that indels were present in this vector, consistent with the SIN sites in the vector and the absence of means for controlling transcription or translation of the SaCas9 gene or the sgRNA.

PCR product from the vector prepared with the pSIA113 plasmid did not show smaller bands, indicating that no indels were present in this vector, consistent with the absence of SIN sites in the vector.

PCR product from the vector prepared with the pSIA121 plasmid showed two smaller bands (marked by arrows), indicating that indels were present in this vector, consistent with the SIN sites in the vector. However, a smaller fraction of the vector contained indels, compared to that prepared from pSIA111. This difference further indicated that repression of transcription of the sgRNA via the TetR protein reduces indel formation via reduction of editing by the SaCas nuclease-sgRNA complex.

PCR product from the vector prepared with the pSIA142 plasmid showed two smaller bands (marked by arrows), indicating that indels were present in this vector, consistent with the SIN sites in the vector. However, a smaller fraction of the vector contained indels, compared to that prepared from pSIA111. This difference further indicated that limitation of transcription via the use of a cell-specific promoter driving expression of the SaCas nuclease gene reduces indel formation via reduction of editing by the Cas nuclease-sgRNA complex.

Referring to the assay using the 3′ SIN site PCR product (right panel), PCR product from the vector prepared with the pSIA111 plasmid showed two smaller bands (marked by arrows), indicating that indels were present in these genomes, consistent with the SIN sites in the genome and the absence of means for controlling transcription or translation of the SaCas9 gene or the sgRNA.

PCR product from the vector prepared with the pSIA113 plasmid did not show smaller bands, indicating that no indels were present in this vector, consistent with the absence of SIN sites in the vector.

PCR product from the vector prepared with the pSIA121 plasmid showed two smaller bands (marked by arrows), indicating that indels were present in this vector, consistent with the SIN sites in the vector. However, a smaller fraction of the vector contained indels, compared to that prepared from pSIA111. This difference further indicated that repression of transcription of the sgRNA via the TetR protein reduces indel formation via reduction of editing by the SaCas nuclease-sgRNA complex.

PCR product from the vector prepared with the pSIA142 plasmid showed two smaller bands (marked by arrows), indicating that indels were present in this vector, consistent with the SIN sites in the vector. However, a smaller fraction of the genomes contained indels, compared to genomes prepared from pSIA111. This difference further indicated that limitation of transcription via the use of a cell-specific promoter driving expression of the SaCas nuclease gene reduces indel formation via reduction of editing by the SaCas nuclease-sgRNA complex.

These results provide evidence that at least one of transcriptional control via a repressor of the sgRNA gene and transcriptional control of the Cas nuclease via a cell-type specific promoter can be successful strategies to control gene editing during packaging of viral vectors. Particularly, indel generation can be reduced by one or both of the strategies. It is possible that a combination of these two strategies could be preferred.

### Example 7-Controlling Both Transcriptional Expression of gRNAs and Post-Transcriptional Expression of SaCas9 Nuclease Using a Tetracycline Operator/Repressor System and shRNAs

To analyze inhibition of genomic deletion in recombinant AAV vector genomes during packaging, recombinant AAV vectors were generated using either pSIA185, pSIA186, pSIA187, or pSIA036. In this experiment, both shRNA encoded on pDL153 (SEQ ID NO: 11) and the TetR protein were used to control expression of the Cas nuclease and the sgRNA, respectively. Generation and packaging of the AAV vectors was performed similarly to the corresponding procedure described in Example 4 above.

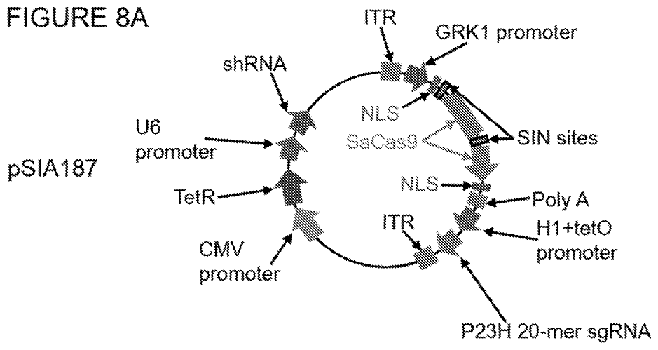

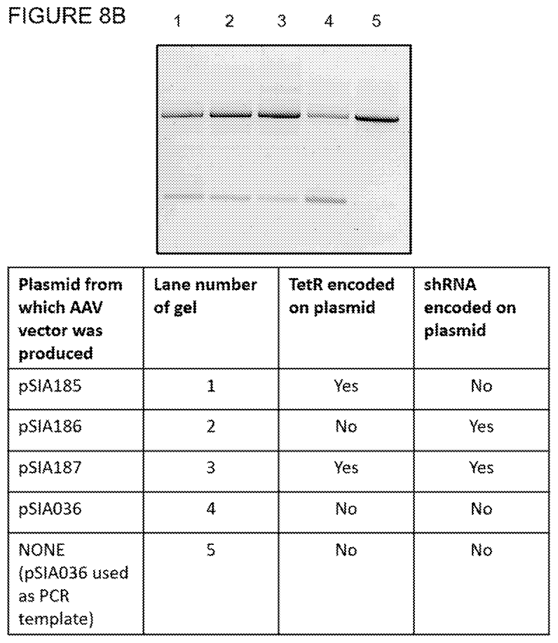

pSIA187 (SEQ ID NO: 38), depicted in FIG. 8A, is a plasmid comprising a SaCas9 gene (SEQ ID NO: 64) and a sequence that encodes a P23H 20-mer sgRNA (SEQ ID NO: 2), driven by an H1 promoter comprising two TetO sites (SEQ ID NO: 3) between two AAV ITRs. The sequence between AAV ITRs of pSIA187 further comprises two SIN sites, with a first SIN site located between the 5′ NLS of SaCas9 and the immediate 5′ end of SaCas9 and a second SIN site located within the intron of SaCas9. pSIA187 further comprises a sequence encoding a TetR protein (e.g., SEQ ID NO: 62) and a sequence encoding a shRNA (SEQ ID NO: 11) coded on pDL153. The TetR sequence and the shRNA sequence are located on a separate locus of the plasmid, compared to the sequence between AAV ITRs. The TetR gene and the shRNA gene, while expressed during packaging, are not included in recombinant AAV vectors prepared during packaging because the two genes are not located between the AAV inverted terminal repeats.

pSIA186 (SEQ ID NO: 37) is a plasmid similar to pSIA187, except that pSIA186 lacks the TetR gene cassette. pSIA185 (SEQ ID NO: 36) is a plasmid similar to pSIA187, except that pSIA185 lacks the shRNA gene cassette. pSIA036 (SEQ ID NO: 39) is a plasmid similar to pSIA187, except that pSIA036 lacks both the shRNA gene cassette and the TetR gene cassette.

After packaging and recombinant AAV vector particle purification, as described in Example 4, genomic DNA was isolated from each sample and used as a template for a PCR (FIG. 8B). Two oligonucleotide primers (SEQ ID NOs: 21 and 22) were used to amplify a sequence comprising the DNA between the two SIN sites. Thus, a longer product would be made by vector templates with no deletion and a shorter product would be made by vector templates with a deletion due to cutting at both SIN sites by the SaCas nuclease-sgRNA complex.

FIG. 8B, lane 5, shows that PCR conducted using a plasmid, pSIA036, as PCR template yielded a single, full length product. This lane serves as a control confirming that a template with no deletion will only yield the single product shown. A portion of recombinant AAV vectors produced from any one of pSIA036, pSIA185, pSIA186, and pSIA187 did comprise a deletion. However, more intact vectors were produced when any one of pSIA185, pSIA186, and pSIA187 were used, compared to pSIA036. Additionally, when pSIA187 was used, an amount of the intact vector approaching that of the control (lane 5) were produced.

These results demonstrate that using at least one of a shRNA targeting the SaCas9 transcript and a TetR protein to control sgRNA transcription can reduce accumulation recombinant AAV vector genomic deletions during packaging. A combination of these two strategies can be preferred.

### Example 8—Controlling Both Transcriptional Expression of gRNAs and Post-Transcriptional Expression of SaCas9 Nuclease Using a Tetracycline Operator/Repressor System and shRNAs

To further analyze inhibition of genomic deletion in recombinant AAV vector genomes during packaging, recombinant AAV vector genomes were generated using either pSIA190, pSIA189, pSIA188, pSIA071, or pSIA069. In this experiment, both shRNAs and the TetR protein were used to control expression of the Cas nuclease and the sgRNA, respectively. Generation and packaging of the genomes was performed similarly to the corresponding procedure described in Example 4 above.

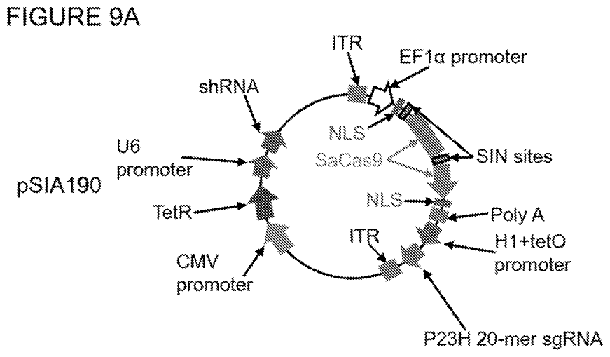

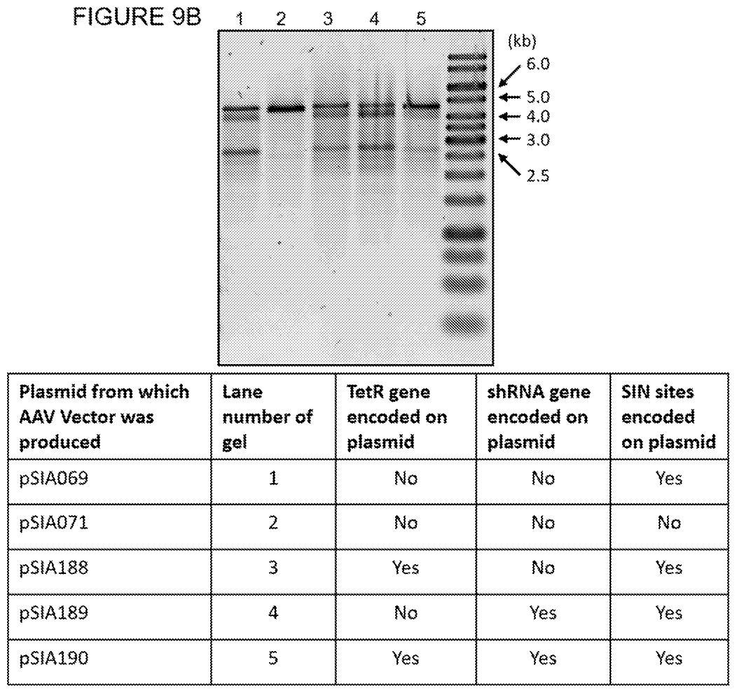

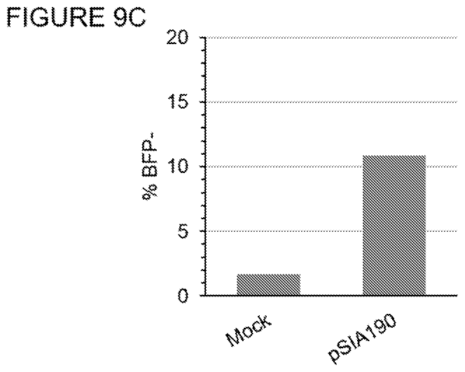

pSIA190 (SEQ ID NO: 43), depicted in FIG. 9A, is a plasmid comprising a SaCas9 gene (SEQ ID NO: 64) and a sequence that encodes a P23H 20-mer sgRNA (SEQ ID NO: 2), driven by an H1 promoter comprising two TetO sites (SEQ ID NO: 3) between two AAV ITRs. The AAV sequence of pSIA190 further comprises two SIN sites, with a first SIN site located between the NLS and the immediate 5′ end of SaCas9 and a second SIN site located within the intron of SaCas9. pSIA190 further comprises a sequence encoding a TetR protein (e.g., SEQ ID NO: 62) and a sequence encoding a shRNA (SEQ ID NO: 11) coded on pDL153. The TetR sequence and the shRNA sequence are located on a separate locus of the plasmid, compared to the sequence between AAV ITRs. The TetR gene and the shRNA gene, while expressed during packaging, are not included in recombinant AAV vectors prepared during packaging because the two genes are not located between the AAV inverted terminal repeats.

pSIA189 (SEQ ID NO: 42) is a plasmid similar to pSIA190, except that pSIA189 lacks the TetR gene cassette. pSIA188 (SEQ ID NO: 41) is a plasmid similar to pSIA190, except that pSIA188 lacks the shRNA gene cassette. pSIA069 (SEQ ID NO: 40) is a plasmid similar to pSIA190, except that pSIA069 lacks both the shRNA gene cassette and the TetR gene cassette. pSIA071 (SEQ ID NO: 7) is a plasmid similar to pSIA190, except that pSIA071 lacks both the shRNA gene cassette and the TetR gene cassette and also lacks the two SIN sites.

After packaging and recombinant AAV vector particle purification, as described in Example 4, vector DNA was isolated from each sample and analyzed by alkaline agarose gel electrophoresis. A band of approximately 4.5 kb corresponds to AAV vectors with no deletion and shorter bands correspond to AAV vectors with a deletion due to cutting at both SIN sites by the SaCas nuclease-sgRNA complex.

FIG. 9B, lane 2, shows that a single sequence of the expected size was packaged in the recombinant AAV vector from pSIA071. This lane serves as a control confirming that a template with no SIN sites will only yield a full length vector. A portion of recombinant AAV vectors produced from any one of pSIA069, pSIA188, pSIA189, and pSIA190 did comprise a deletion, as indicated by the accumulation of shorter (lower) bands in the gel. However, more intact vectors were produced when any one of pSIA188, pSIA189, and pSIA190 were used, compared to pSIA069. Additionally, when pSIA190 was used, an amount of intact genomes approaching that of the control (lane 2) were produced.

FIG. 9C shows results from a BFP reporter cell assay similar to the one conducted in Example 1. However, a recombinant AAV vector (serotype 6) produced from pSIA190 was used to transduce the reporter cell line at 1,000,000 viral vectors per cell, rather than transfection with a plasmid. Mock transduced cells showed a background level loss of BFP fluorescence ˜1.5%. In contrast, about 11% of cells transduced with the recombinant AAV vector produced from pSIA190 showed loss of BFP fluorescence, indicating that many of the all-in-one SIN viral vectors were able to introduce frame-shift mutations in the P23H target site that was positioned upstream of the BFP gene.

These results demonstrate that using at least one of a shRNA targeting the SaCas9 transcript and a TetR protein to control sgRNA transcription can reduce accumulation of deletion on a recombinant AAV vector during packaging. A combination of these two strategies can be preferred. These results further suggest that functional all-in-one SIN viral vectors can be produced using the materials and methods disclosed herein.

### Example 9—Controlling Post-Transcriptional Expression of sRGN Cas Nuclease (Gib11Spa1) Using a shRNAs

To investigate inhibition of expression of sRGN expression during recombinant AAV vector packaging, an experiment was conducted. In this experiment, a BFP reporter cell assay was used to investigate sRGN expression from a plasmid when controlled by shRNAs encoded on a second plasmid.

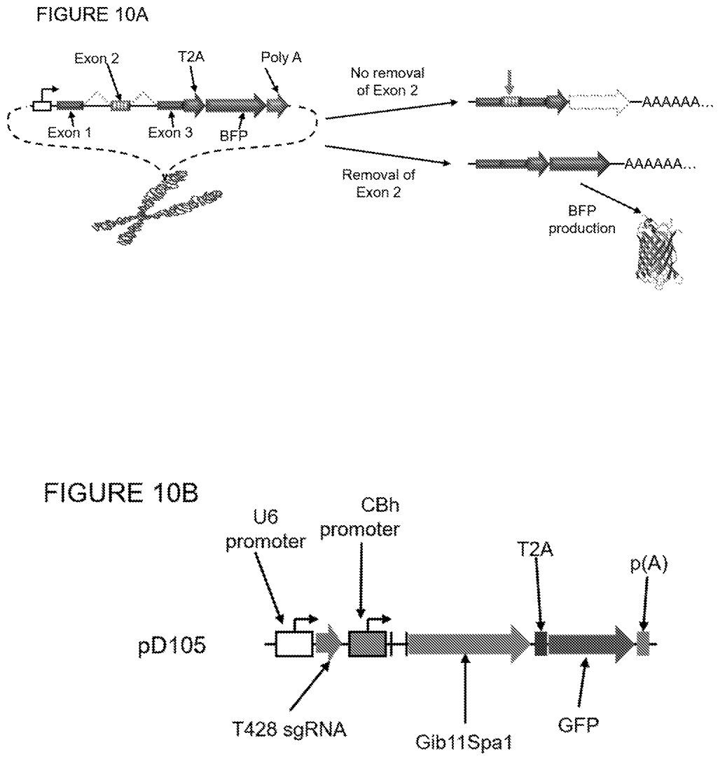

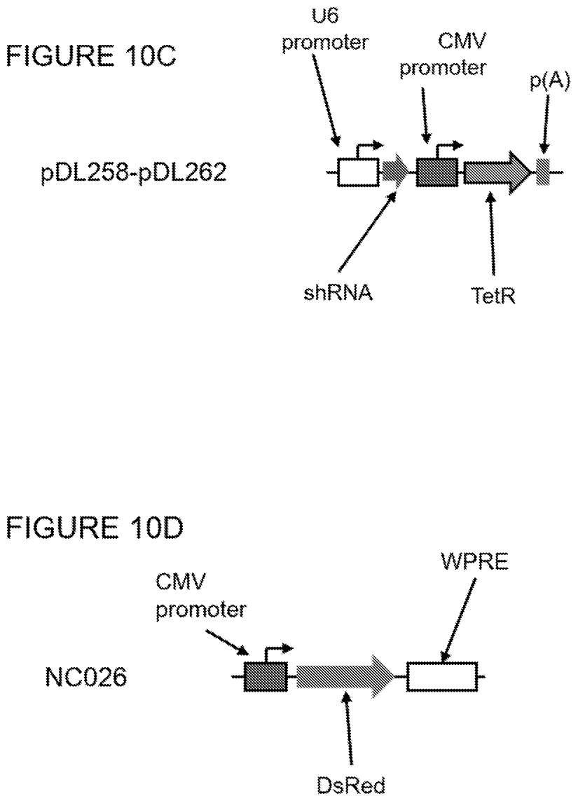

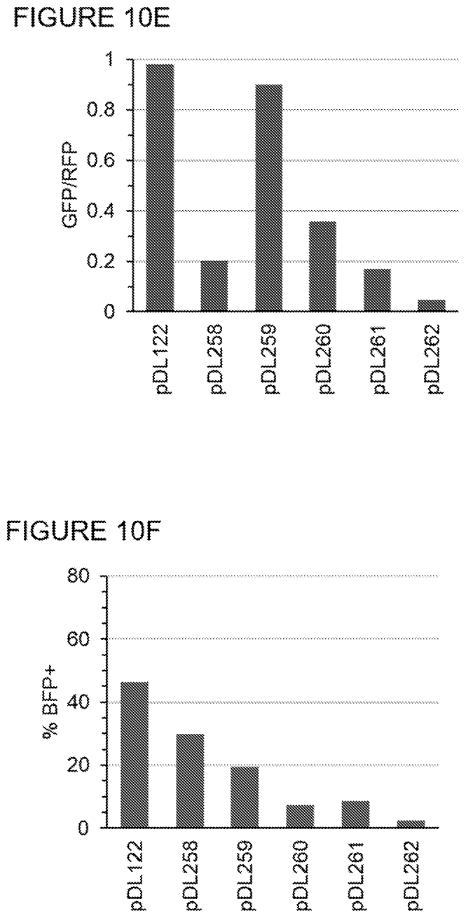

FIG. 10A shows a diagram of the reporter construct (SEQ ID NO: 44) used in this experiment. The reporter cell line was generated from Jump-In™ GripTite™ HEK293 cells, available from Thermo Fisher Scientific, Massachusetts, US. The reporter construct will produce a nonfunctional transcript from the BFP locus if Exon 2 is unedited by the sRGN. If editing at the splice donor site of Exon 2 does occur, Exon 2 will be spliced out of the transcript and a functional protein product will be produced, resulting in T2A cleavage and production of free BFP.

FIG. 10B shows a diagram of pD105, which is a plasmid that comprises a Gib11Spa1 sRGN gene sequence that encodes Gib11Spa1 sRGN protein sequence (SEQ ID NO: 60) and a T428 sgRNA gene (SEQ ID NO: 46). GFP is also produced from the sRGN gene. The GFP protein is separated from the sRGN protein by a T2A self-cleaving peptide.

FIG. 10C shows a generalized diagram of plasmids used to express various shRNAs that target Gib11Spa1 transcripts. Each shRNA is driven by a U6 promoter. Additionally, a CMV promoter drives expression of a TetR protein, but none of the promoters in this example comprise a TetO site, so this aspect of the plasmids is not relevant in the Example.

pDL258 (SEQ ID NO: 50) comprises a shRNA comprising the sequence of SEQ ID NO: 55. pDL259 (SEQ ID NO: 51) comprises a shRNA comprising the sequence of SEQ ID NO: 56. pDL260 (SEQ ID NO: 52) comprises a shRNA comprising the sequence of SEQ ID NO: 57. pDL261 (SEQ ID NO: 53) comprises a shRNA comprising the sequence of SEQ ID NO: 58. pDL262 (SEQ ID NO: 54) comprises a shRNA comprising the sequence of SEQ ID NO: 59.

FIG. 10D shows a diagram of a plasmid (NC026) expressing an RFP gene, which was used as a transfection control. The RFP gene is regulated by the CMV promoter and the woodchuck hepatitis virus posttranscriptional regulatory element (WPRE), which increases translation of the RFP transcript.

FIG. 10E shows results of a BFP reporter cell assay. Flow cytometry was conducted similarly to Example 1. All cells were transfected with NC026 and pD105. Thus, all samples show some GFP and RFP signal. Cells additionally transfected with pDL122, which is a negative control comprising no shRNA gene (SEQ ID NO: 13), showed nearly equal numbers of GFP-expressing and RFP-expressing cells, indicating that the sRGN and GFP were expressed relatively strongly at the protein level.

Cells additionally transfected with pDL258, which expresses a first shRNA gene, showed that GFP-expressing cells were only ˜20% of the RFP-expressing cells, indicating that the sRGN and GFP were expressed relatively weakly at the protein level.

Cells additionally transfected with pDL259, which expresses a second shRNA gene, showed that GFP-expressing cells were ˜90% of RFP-expressing cells, indicating that the sRGN and GFP were expressed only slightly less at the protein level.

Cells additionally transfected with pDL260, which expresses a third shRNA gene, showed that GFP-expressing cells were only ˜35% of RFP-expressing cells, indicating that the sRGN and GFP were expressed relatively weakly at the protein level.

Cells additionally transfected with pDL261, which expresses a fourth shRNA gene, showed that GFP-expressing cells were only ˜15% of the RFP-expressing cells, indicating that the sRGN and GFP were expressed relatively weakly at the protein level.

Cells additionally transfected with pDL262, which expresses a fifth shRNA gene, showed the GFP-expressing cells were only ˜5% of RFP-expressing cells, indicating that the sRGN and GFP were expressed relatively weakly at the protein level.

FIG. 10F shows additional results of the BFP reporter cell assay. ˜ 45% of cells additionally transfected with pDL122 were positive for BFP, indicating a baseline for BFP expression levels resulting from relatively uncontrolled editing by the sRGN-sgRNA complex at the BFP locus.

˜30% of cells additionally transfected with pDL258 were positive for BFP, indicating a reduction from the baseline for BFP expression levels resulting from relatively reduced editing by the sRGN-sgRNA complex at the BFP locus. Thus, the shRNA encoded on pDL258 appears to have reduced sRGN expression.

˜20% of cells additionally transfected with pDL259 were positive for BFP, indicating a reduction from the baseline for BFP expression levels resulting from relatively reduced editing by the sRGN-sgRNA complex at the BFP locus. Thus, the shRNA encoded on pDL259 appears to have reduced sRGN expression.

˜10% of cells additionally transfected with pDL260 were positive for BFP, indicating a reduction from the baseline for BFP expression levels resulting from relatively reduced editing by the sRGN-sgRNA complex at the BFP locus. Thus, the shRNA encoded on pDL260 appears to have reduced sRGN expression.

˜10% of cells additionally transfected with pDL261 were positive for BFP, indicating a reduction from the baseline for BFP expression levels resulting from relatively reduced editing by the sRGN-sgRNA complex at the BFP locus. Thus, the shRNA encoded on pDL261 appears to have reduced sRGN expression.

˜5% of cells additionally transfected with pDL262 were positive for BFP, indicating a reduction from the baseline for BFP expression levels resulting from relatively reduced editing by the sRGN-sgRNA complex at the BFP locus. Thus, the shRNA encoded on pDL262 appears to have reduced sRGN expression.

These results provide evidence that sRGN gene expression can be regulated at the post-transcriptional level by shRNA expression, thereby reducing editing by the sRGN gene.

### Note Regarding Illustrative Examples

While the present disclosure provides descriptions of various specific aspects for the purpose of illustrating various examples of the present disclosure and/or its potential applications, it is understood that variations and modifications will occur to those skilled in the art. Accordingly, the invention or inventions described herein should be understood to be at least as broad as they are claimed, and not as more narrowly defined by particular illustrative examples provided herein.

What is claimed is:

1. A CRISPR/Cas system comprising:
a nuclease segment comprising a codon optimized nucleotide sequence that encodes a Cas nuclease or variant thereof;
a guide RNA (gRNA) segment comprising a nucleotide sequence that encodes a gRNA or sgRNA; and
a promoter segment comprising a nucleotide sequence that encodes a first promoter comprising one or more tetracycline operator sequence, wherein the gRNA segment is operably linked to the promoter segment.

2. The CRISPR/Cas system of claim 1, wherein the first promoter is selected from a group consisting of: H1 promoter, U6 promoter, 7SK promoter, and portions of any thereof.

3. The CRISPR/Cas system of claim 1 or 2, wherein the one or more tetracycline operator sequence comprises a nucleic acid sequence having at least 85% sequence identity to SEQ ID NO: 4.

4. The CRISPR/Cas system of any one of claims 1-3, wherein the one or more tetracycline operator sequence comprises SEQ ID NO: 4.

5. The CRISPR/Cas system of any one of claims 1-4, further comprising:
a repressor segment comprising a nucleotide sequence that encodes a tetracycline repressor protein.

6. The CRISPR/Cas system of claim 5, wherein the tetracycline repressor comprises a nucleic acid sequence having at least 85% sequence identity to SEQ ID NO: 62.

7. The CRISPR/Cas system of claim 5, wherein the tetracycline repressor comprises a nucleic acid sequence comprising SEQ ID NO: 62.

8. The CRISPR/Cas system of any one of claims 1-7, wherein the one or more tetracycline operator sequence is capable of being bound by the tetracycline repressor protein.

9. The CRISPR/Cas system of any one of claims 1-8, further comprising:
one or more self-inactivating segments comprising a SIN site;
wherein the gRNA or sgRNA is substantially complementary to the SIN site;
wherein the gRNA or sgRNA is substantially complementary to a genomic target sequence within a cell of a patient.

10. The CRISPR/Cas system of claim 9, wherein the one or more self-inactivating segments are located in at least one of:
(i) at the 5′ end of the codon optimized nucleotide sequence that encodes a Cas nuclease or variant thereof;
(ii) at the 3′ end of the codon optimized nucleotide sequence that encodes a Cas nuclease or variant thereof; and
(iii) in an intron within the codon optimized nucleotide sequence that encodes a Cas nuclease or variant thereof.

11. The CRISPR/Cas system of claim 9, wherein the one or more self-inactivating segments are located in:
(i) at the 5′ end of the codon optimized nucleotide sequence that encodes a Cas nuclease or variant thereof; and
(ii) in an intron within the codon optimized nucleotide sequence that encodes a Cas nuclease or variant thereof.

12. The CRISPR/Cas system of any one of claims 9-11, wherein one of the one or more self-inactivating segments are located upstream of the codon optimized nucleotide sequence that encodes a Cas nuclease or variant thereof and downstream of a nuclear localization signal (NLS).

13. The CRISPR/Cas system of any one of claims 9-11, wherein the SIN site comprises a protospacer adjacent motif (PAM) sequence.

14. The CRISPR/Cas system of claim 13, wherein the PAM sequence in the SIN site is selected from a group consisting of: NNGRRT, NRG, NAAAAN, NAAAAC, NNNNGHTT, YTN, NNNNACAC, NNVRYAC, NNNNVRYM, NNAAAAW, NNAGAAW, and NNGG.

15. The CRISPR/Cas system of any of claims 1-14, wherein the gRNA or sgRNA is fully complementary to the nucleotide sequence of the SIN site except for at one base pair.

16. The CRISPR/Cas system of any of claims 1-14, wherein the gRNA or sgRNA is fully complementary to the nucleotide sequence of the SIN site except for at two base pairs.

17. The CRISPR/Cas system of any one of claims 1-16, wherein the Cas nuclease is a Class 2 Cas nuclease.

18. The CRISPR/Cas system of any one of claims 1-17, wherein the Cas nuclease is selected from a group consisting of: S. pyogenes Cas, S. aureus Cas, S. thermolphilus Cas, C. jejuni Cas, T. denticola Cas, N. meningitides Cas, S. lugdunensis Cas, S. hyicus Cas, S. microti Cas, and S. pasteuri Cas.

19. The CRISPR/Cas system of any one of claims 1-16, wherein the Cas nuclease is a synthetic, RNA-Guided Nuclease (sRGN).

20. The CRISPR/Cas system of claim 19, wherein the Cas nuclease comprises an amino acid sequence having at least 85% sequence identity to SEQ ID NO: 60.

21. The CRISPR/Cas system of any one of claims 1-20, wherein a nucleic acid sequence encoding a second promoter is operably linked to the codon optimized nucleotide sequence that encodes a Cas nuclease or variant thereof.

22. The CRISPR/Cas system of claim 21, wherein the second promoter is a spatially-restricted promoter, bidirectional promoter, or an inducible promoter.

23. The CRISPR/Cas system of claim 22, wherein the spatially-restricted promoter is selected from a group consisting of: any tissue or cell type specific promoter, a hepatocyte-specific promoter, a neuron-specific promoter, an adipocyte-specific promoter, a cardiomyocyte-specific promoter, a skeletal muscle-specific promoter, lung progenitor cell specific promoter, a photoreceptor-specific promoter, and a retinal pigment epithelial (RPE) selective promoter.

24. The CRISPR/Cas system of any one of claims 1-23, wherein the gRNA or sgRNA comprises a spacer sequence comprising 17 to 24 nucleotides.

25. The CRISPR/Cas system of any one of claims 1-24, wherein the nuclease segment is provided in a first vector, and the gRNA segment and the promoter segment are provided together in a second vector.

26. The CRISPR/Cas system of any of claims 5-24, wherein the nuclease segment, the gRNA segment, and the promoter segment are provided together in a first vector and the repressor segment is provided in a second vector.

27. The CRISPR/Cas system of any one of claims 9-24, wherein the nuclease segment, the gRNA segment, the promoter segment, and the one or more self-inactivating segments are provided together in a first vector and the repressor segment is provided in a second vector.

28. The CRISPR/Cas system of any one of claims 9-24, wherein the nuclease segment, the gRNA segment, the promoter segment, the one or more self-inactivating segments, and the repressor segment are provided in a vector.

29. The CRISPR/Cas system of any one of claims 25-28, wherein the first vector and the second vector are AAV vectors or plasmids.

30. The CRISPR/Cas system of claim 29, wherein the AAV vectors are AAV2 serotype vectors, AAV5 serotype vectors, or AAV6 serotype vectors.

31. A pharmaceutical composition comprising the CRISPR/Cas system of any one of claims 1-30.

32. A packaging cell comprising the CRISPR/Cas system of any one of claims 1-30.

33. A method of controlling transcription of gRNAs during AAV packaging, the method comprising:
contacting a packaging cell with the CRISPR/Cas system of any one of claims 1-30.

34. A method of reducing mutagenesis at one or more SIN site in a recombinant AAV vector, the method comprising:
contacting a packaging cell with the CRISPR/Cas system of any one of claims 1-30.

35. A recombinant AAV vector comprising:
a nuclease segment comprising a codon optimized nucleotide sequence that encodes a Cas nuclease or variant thereof;
a gRNA segment comprising a nucleotide sequence that encodes a gRNA or sgRNA; and
a promoter segment comprising a nucleotide sequence that encodes a first promoter comprising one or more tetracycline operator sequence, wherein the gRNA segment is operably linked to the promoter segment.

36. The recombinant AAV vector of claim 35, wherein the first promoter is selected from a group consisting of: H1 promoter, U6 promoter, 7SK promoter, and portions of any thereof.

37. The recombinant AAV vector of any one of claim 35 or 36, wherein the one or more tetracycline operator sequence comprises a nucleic acid sequence having at least 85% sequence identity to SEQ ID NO: 4.

38. The recombinant AAV vector of any one of claims 35-37, wherein the one or more tetracycline operator sequence comprises SEQ ID NO: 4.

39. The recombinant AAV vector of any one of claims 35-38, wherein the one or more tetracycline operator sequences is capable of being bound by a tetracycline repressor.

40. The recombinant AAV vector of any one of claims 35-39, further comprising:
one or more self-inactivating segments comprising a SIN site;
wherein the gRNA or sgRNA is substantially complementary to the SIN site;
wherein the gRNA or sgRNA is substantially complementary to a genomic target sequence within a cell of a patient.

41. The recombinant AAV vector of claim 40, wherein the one or more self-inactivating segments are located in at least one of:
(i) at the 5′ end of the codon optimized nucleotide sequence that encodes a Cas nuclease or variant thereof;
(ii) at the 3′ end of the codon optimized nucleotide sequence that encodes a Cas nuclease or variant thereof; and
(iii) in an intron within the codon optimized nucleotide sequence that encodes a Cas nuclease or variant thereof.

42. The recombinant AAV vector of claim 40, wherein the one or more self-inactivating segments are located in:
(i) at the 5′ end of the codon optimized nucleotide sequence that encodes a Cas nuclease or variant thereof; and
(ii) in an intron within the codon optimized nucleotide sequence that encodes a Cas nuclease or variant thereof.

43. The recombinant AAV vector of any one of claims 40-42, wherein one of the one or more self-inactivating segments are located upstream of the codon optimized nucleotide sequence that encodes a Cas nuclease or variant thereof and downstream of a NLS.

44. The recombinant AAV vector of any one of claims 40-42, wherein the SIN site comprises a PAM sequence.

45. The recombinant AAV vector of claim 44, wherein the PAM sequence in the SIN site is selected from a group consisting of: NNGRRT, NRG, NAAAAN, NAAAAC, NNNNGHTT, YTN, NNNNACAC, NNVRYAC, NNNNVRYM, NNAAAAW, NNAGAAW, and NNGG.

46. The recombinant AAV vector of any one of claims 35-45, wherein the gRNA or sgRNA is fully complementary to the nucleotide sequence of the SIN site except for at one base pair.

47. The recombinant AAV vector of any one of claims 35-45, wherein the gRNA or sgRNA is fully complementary to the nucleotide sequence of the SIN site except for at two base pairs.

48. The recombinant AAV vector of any one of claims 35-47, wherein the Cas nuclease is a Class 2 Cas nuclease.

49. The recombinant AAV vector of any one of claims 35-48, wherein the Cas nuclease is selected from a group consisting of: S. pyogenes Cas, S. aureus Cas, S. thermolphilus Cas, C. jejuni Cas, T. denticola Cas, N. meningitides Cas, S. lugdunensis Cas, S. hyicus Cas, S. microti Cas, and S. pasteuri Cas.

50. The recombinant AAV vector of any one of claims 35-47, wherein the Cas nuclease is a sRGN.

51. The recombinant AAV vector of claim 50, wherein the Cas nuclease comprises an amino acid sequence having at least 85% sequence identity to SEQ ID NO: 60.

52. The recombinant AAV vector of any one of claims 35-51, wherein a nucleic acid sequence encoding a second promoter is operably linked to the codon optimized nucleotide sequence that encodes a Cas nuclease or variant thereof.

53. The recombinant AAV vector of claim 52, wherein the second promoter is a spatially-restricted promoter, bidirectional promoter, or an inducible promoter.

54. The recombinant AAV vector of claim 53, wherein the spatially-restricted promoter is selected from a group consisting of: any tissue or cell type specific promoter, a hepatocyte-specific promoter, a neuron-specific promoter, an adipocyte-specific promoter, a cardiomyocyte-specific promoter, a skeletal muscle-specific promoter, lung progenitor cell specific promoter, a photoreceptor-specific promoter, and a RPE selective promoter.

55. The recombinant AAV vector of any one of claims 35-54, wherein the gRNA is a sgRNA.

56. The recombinant AAV vector of any one of claims 35-55, wherein the gRNA or sgRNA comprises a spacer sequence comprising 17 to 24 nucleotides.

57. The recombinant AAV vector of any one of claims 35-56, wherein the recombinant AAV vector is a AAV2 serotype vector, AAV5 serotype vector, or AAV6 serotype vector.

58. The recombinant AAV vector of any one of claims 35-57, wherein the recombinant AAV vector comprises a nucleic acid sequence having at least 85% sequence identity to any one of SEQ ID NOs: 66-69.

59. The recombinant AAV vector of claim 58, wherein the recombinant AAV vector comprises a nucleic acid sequence having at least 90% or at least 95% sequence identity to any one of SEQ ID NOs: 66-69.

60. The recombinant AAV vector of any one of claims 35-58, wherein the recombinant AAV vector comprises any one of SEQ ID NOs: 66-69.

61. A pharmaceutical composition comprising the recombinant AAV vector of any of claims 35-60.

62. A genetically modified cell comprising the recombinant AAV vector of any of claims 35-60.

63. The genetically modified cell of claim 62, wherein the genetically modified cell is selected from a group consisting of: a eukaryotic cell, a somatic cell, a germ cell, a stem cell, an animal cell, a mammalian cell, a mouse cell, a non-human primate cell, and a human cell.

64. A method of controlling transcription of gRNAs during AAV packaging, the method comprising:
contacting a packaging cell with a nucleic acid encoding the recombinant AAV vector of any one of claims 35-60; and
contacting the packaging cell with at least one vector comprising nucleic acid sequence encoding a tetracycline repressor.

65. A method of reducing mutagenesis at one or more SIN site in a recombinant AAV vector, the method comprising:
contacting a packaging cell with nucleic acid encoding the recombinant AAV vector of any one of claims 35-60; and
contacting the packaging cell with at least one vector comprising nucleic acid sequence encoding a tetracycline repressor.

66. The method of claim 65, wherein the packaging cell is a human cell.

67. A method of producing a recombinant AAV vector, the method comprising:
introducing into a packaging cell:
(i) a first vector comprising a repressor segment, wherein the repressor segment comprises a nucleotide sequence that encodes a tetracycline repressor protein;
(ii) a nucleic acid comprising a sequence encoding the recombinant AAV vector of any one of claims 35-60; and
(iii) one or more viral components for producing the recombinant AAV vector;
culturing the packaging cell; and
isolating the recombinant AAV vector comprising the nucleic acid from the packaging cell.

68. A method of producing a recombinant AAV vector, the method comprising:
introducing into a packaging cell a nucleic acid comprising a sequence encoding the recombinant AAV vector of any one of claims 35-60;
introducing into the packaging cell one or more viral components for producing the AAV;
culturing the packaging cell; and
isolating the recombinant AAV vector comprising the nucleic acid from the packaging cell wherein the packaging cell expresses a tetracycline repressor protein.

69. The method of any one of claims 67-68, wherein the one or more viral components are encoded by the nucleic acid.

70. The method of any one of claims 67-68, wherein the one or more viral components are introduced via separate vector other than the nucleic acid.

71. The method of any one of claims 67-68, wherein the one or more viral components are encoded in a cellular genome.

72. A CRISPR/Cas system comprising:
a nuclease segment comprising a codon optimized nucleotide sequence that encodes a Cas nuclease or variant thereof;
a gRNA segment comprising a nucleotide sequence that encodes a gRNA or sgRNA; and
a short-hairpin RNA (shRNA) segment comprising a nucleotide sequence that encodes a shRNA that comprises sequence that is complementary to a transcript from the nuclease segment.

73. The CRISPR/Cas system of claim 72, wherein the shRNA comprises a sequence having at least 85% sequence identity to any one of SEQ ID NOs: 9-11 or 55-59.

74. The CRISPR/Cas system of claim 72, wherein the shRNA comprises any one of SEQ ID NOs: 9-11 or 55-59.

75. The CRISPR/Cas system of any one of claims 72-74, further comprising:
one or more self-inactivating segments comprising a SIN site;
wherein the gRNA or sgRNA is substantially complementary to the SIN site;
wherein the gRNA or sgRNA is substantially complementary to a genomic target sequence within a cell of a patient.

76. The CRISPR/Cas system of claim 75, wherein the one or more self-inactivating segments are located in at least one of:
(i) at the 5′ end of the codon optimized nucleotide sequence that encodes a Cas nuclease or variant thereof;
(ii) at the 3′ end of the codon optimized nucleotide sequence that encodes a Cas nuclease or variant thereof; and
(iii) in an intron within the codon optimized nucleotide sequence that encodes a Cas nuclease or variant thereof.

77. The CRISPR/Cas system of claim 75, wherein the one or more self-inactivating segments are located in:
(i) at the 5′ end of the codon optimized nucleotide sequence that encodes a Cas nuclease or variant thereof; and
(ii) in an intron within the codon optimized nucleotide sequence that encodes a Cas nuclease or variant thereof.

78. The CRISPR/Cas system of any one of claims 75-77, wherein one of the one or more self-inactivating segments are located upstream of the codon optimized nucleotide sequence that encodes a Cas nuclease or variant thereof and downstream of a NLS.

79. The CRISPR/Cas system of any one of claims 75-77, wherein the SIN site comprises a PAM sequence.

80. The CRISPR/Cas system of claim 79, wherein the PAM sequence in the SIN site is selected from a group consisting of: NNGRRT, NRG, NAAAAN, NAAAAC, NNNNGHTT, YTN, NNNNACAC, NNVRYAC, NNNNVRYM, NNAAAAW, NNAGAAW, and NNGG.

81. The CRISPR/Cas system of any of claims 72-80, wherein the gRNA or sgRNA is fully complementary to the nucleotide sequence of the SIN site except for at one base pair.

82. The CRISPR/Cas system of any of claims 72-80, wherein the gRNA or sgRNA is fully complementary to the nucleotide sequence of the SIN site except for at two base pairs.

83. The CRISPR/Cas system of any one of claims 72-82, wherein the Cas nuclease is a Class 2 Cas nuclease.

84. The CRISPR/Cas system of any one of claims 72-83, wherein the Cas nuclease is selected from a group consisting of: S. pyogenes Cas, S. aureus Cas, S. thermolphilus Cas, C. jejuni Cas, T. denticola Cas, N. meningitides Cas, S. lugdunensis Cas, S. hyicus Cas, S. microti Cas, and S. pasteuri Cas.

85. The CRISPR/Cas system of any one of claims 72-82, wherein the Cas nuclease is a sRGN.

86. The CRISPR/Cas system of claim 85, wherein the Cas nuclease comprises an amino acid sequence having at least 85% sequence identity to SEQ ID NO: 60.

87. The CRISPR/Cas system of any one of claims 72-86, wherein a nucleic acid sequence encoding a promoter is operably linked to the codon optimized nucleotide sequence that encodes a Cas nuclease or variant thereof.

88. The CRISPR/Cas system of claim 87, wherein the promoter is a spatially-restricted promoter, bidirectional promoter, or an inducible promoter.

89. The CRISPR/Cas system of claim 88, wherein the spatially-restricted promoter is selected from a group consisting of: any tissue or cell type specific promoter, a hepatocyte-specific promoter, a neuron-specific promoter, an adipocyte-specific promoter, a cardiomyocyte-specific promoter, a skeletal muscle-specific promoter, lung progenitor cell specific promoter, a photoreceptor-specific promoter, and a RPE selective promoter.

90. The CRISPR/Cas system of any one of claims 72-89, wherein the gRNA is a sgRNA.

91. The CRISPR/Cas system of any one of claims 72-90, wherein the gRNA or sgRNA comprises a spacer sequence comprising 17 to 24 nucleotides.

92. The CRISPR/Cas system of any of claims 72-91, wherein the nuclease segment and the gRNA segment are provided together in a first vector and the shRNA segment is provided in a second vector.

93. The CRISPR/Cas system of any of claims 75-91, wherein the nuclease segment, the gRNA segment, and the one or more self-inactivating segments are provided together in a first vector and the shRNA segment is provided in a second vector.

94. The CRISPR/Cas system of any of claims 75-91, wherein the nuclease segment, the gRNA segment, the one or more self-inactivating segments, and the shRNA segment are provided in a vector.

95. The CRISPR/Cas system of any of claims 92-93, wherein the first vector and the second vector are AAV vectors or plasmids.

96. The CRISPR/Cas system of claim 95, wherein the AAV vectors are AAV2 serotype vectors, AAV5 serotype vectors, or AAV6 serotype vectors.

97. A pharmaceutical composition comprising the CRISPR/Cas system of any of claims 72-96.

98. A packaging cell comprising the CRISPR/Cas system of any of claims 72-96.

99. A method of controlling post-transcriptional expression of Cas nuclease during AAV packaging, the method comprising:
contacting a packaging cell with the CRISPR/Cas system of any one of claims 72-96.

100. A method of reducing mutagenesis at one or more SIN site in a recombinant AAV vector, the method comprising:
contacting a cell with the CRISPR/Cas system of any one of claims 72-96.

101. The method of claim 99 or 100, wherein the packaging cell is a human cell.

102. A method of controlling post-transcriptional expression of Cas nuclease during AAV packaging, the method comprising:
contacting a packaging cell with a nucleic acid comprising a sequence encoding the recombinant AAV vector of any one of claims 35-60; and
contacting the packaging cell with at least one vector comprising nucleic acid sequence encoding a shRNA segment.

103. A method of producing a recombinant AAV vector, the method comprising:
introducing into a packaging cell:
(i) a first vector comprising a shRNA segment comprising a nucleotide sequence that encodes a shRNA that comprises sequence that is complementary to a transcript from the nuclease segment;
(ii) a nucleic acid comprising sequence encoding the recombinant AAV vector of any one of claims 35-60; and
(iii) one or more viral components for producing the recombinant AAV vector;
culturing the packaging cell; and
isolating the recombinant AAV vector comprising the nucleic acid from the packaging cell.

104. A method of producing a recombinant AAV vector, the method comprising:
introducing into a packaging cell a nucleic acid comprising sequence encoding the recombinant AAV vector of any one of claims 35-60;
introducing into the packaging cell one or more viral components for producing the AAV;
culturing the packaging cell; and
isolating the recombinant AAV vector comprising the nucleic acid from the packaging cell;
wherein the packaging cell expresses a shRNA.

105. The method of any one of claims 103-104, wherein the one or more viral components are encoded by the nucleic acid.

106. The method of any one of claims 103-104, wherein the one or more viral components are introduced via separate vector other than the nucleic acid.

107. The method of any one of claims 103-104, wherein the one or more viral components are encoded in a cellular genome.

108. A CRISPR/Cas system comprising:
a nuclease segment comprising a codon optimized nucleotide sequence that encodes a Cas nuclease or variant thereof;
a gRNA segment comprising a nucleotide sequence that encodes a gRNA or sgRNA;
a promoter segment comprising a nucleotide sequence that encodes a first promoter comprising one or more tetracycline operator sequences, wherein the gRNA segment is operably linked to the promoter segment; and
a short-hairpin RNA (shRNA) segment comprising a nucleotide sequence that encodes a shRNA that comprises sequence that is complementary to a transcript from the nuclease segment.

109. The CRISPR/Cas system of claim 108, wherein the first promoter is selected from a group consisting of: a H1 promoter, U6 promoter, 7SK promoter, and portions of any thereof.

110. The CRISPR/Cas system of claim 108 or 109, wherein the one or more tetracycline operator sequence comprises a nucleic acid sequence having at least 85% sequence identity to SEQ ID NO: 4.

111. The CRISPR/Cas system of any one of claims 108-109, wherein the one or more tetracycline operator sequence comprises SEQ ID NO: 4.

112. The CRISPR/Cas system of any one of claims 108-111, further comprising:
a repressor segment comprising a nucleotide sequence that encodes a tetracycline repressor protein.

113. The CRISPR/Cas system of claim 112, wherein the tetracycline repressor comprises a nucleic acid sequence having at least 85% sequence identity to SEQ ID NO: 62.

114. The CRISPR/Cas system of claim 112, wherein the tetracycline repressor comprises a nucleic acid sequence comprising SEQ ID NO: 62.

115. The CRISPR/Cas system of any one of claims 108-114, wherein the one or more tetracycline operator sequences are capable of being bound by the tetracycline repressor protein.

116. The CRISPR/Cas system of any one of claims 108-115, wherein the shRNA comprises sequence having at least 85% sequence identity to any one of SEQ ID NOs: 9-11 or 55-59.

117. The CRISPR/Cas system any one of claims 108-116, wherein the shRNA comprises SEQ ID NOs: 9-11 or 55-59.

118. The CRISPR/Cas system of any one of claims 108-117, further comprising:
one or more self-inactivating segments comprising a SIN site;
wherein the gRNA or sgRNA is substantially complementary to the SIN site;
wherein the gRNA or sgRNA is substantially complementary to a genomic target sequence within a cell of a patient.

119. The CRISPR/Cas system of claim 118, wherein the one or more self-inactivating segments are located in at least one of:
(i) at the 5′ end of the codon optimized nucleotide sequence that encodes a Cas nuclease or variant thereof;
(ii) at the 3′ end of the codon optimized nucleotide sequence that encodes a Cas nuclease or variant thereof; and
(iii) in an intron within the codon optimized nucleotide sequence that encodes a Cas nuclease or variant thereof.

120. The CRISPR/Cas system of claim 118, wherein the one or more self-inactivating segments are located in:
(i) at the 5′ end of the codon optimized nucleotide sequence that encodes a Cas nuclease or variant thereof; and
(ii) in an intron within the codon optimized nucleotide sequence that encodes a Cas nuclease or variant thereof.

121. The CRISPR/Cas system of any one of claims 118-120, wherein one of the one or more self-inactivating segments are located upstream of the codon optimized nucleotide sequence that encodes a Cas nuclease or variant thereof and downstream of a NLS.

122. The CRISPR/Cas system of any one of claims 118-120, wherein the SIN site comprises a PAM sequence.

123. The CRISPR/Cas system of claim 122, wherein the PAM sequence in the SIN site is selected from a group consisting of: NNGRRT, NRG, NAAAAN, NAAAAC, NNNNGHTT, YTN, NNNNACAC, NNVRYAC, NNNNVRYM, NNAAAAW, NNAGAAW, and NNGG.

124. The CRISPR/Cas system of any of claims 108-122, wherein the gRNA or sgRNA is fully complementary to the nucleotide sequence of the SIN site except for at one base pair.

125. The CRISPR/Cas system of any of claims 108-122, wherein the gRNA or sgRNA is fully complementary to the nucleotide sequence of the SIN site except for at two base pairs.

126. The CRISPR/Cas system of any one of claims 108-125, wherein the Cas nuclease is a Class 2 Cas nuclease.

127. The CRISPR/Cas system of any one of claims 108-126, wherein the Cas nuclease is selected from a group consisting of: S. pyogenes Cas, S. aureus Cas, S. thermolphilus Cas, C. jejuni Cas, T. denticola Cas, N. meningitides Cas, S. lugdunensis Cas, S. hyicus Cas, S. microti Cas, and S. pasteuri Cas.

128. The CRISPR/Cas system of any one of claims 108-125, wherein the Cas nuclease is a sRGN.

129. The CRISPR/Cas system of claim 128, wherein the Cas nuclease comprises an amino acid sequence having at least 85% sequence identity to SEQ ID NO: 60.

130. The CRISPR/Cas system of any one of claims 108-129, wherein a nucleic acid sequence encoding a second promoter is operably linked to the codon optimized nucleotide sequence that encodes a Cas nuclease or variant thereof.

131. The CRISPR/Cas system of claim 130, wherein the second promoter is a spatially-restricted promoter, bidirectional promoter, or an inducible promoter.

132. The CRISPR/Cas system of claim 131, wherein the spatially-restricted promoter is selected from a group consisting of: any tissue or cell type specific promoter, a hepatocyte-specific promoter, a neuron-specific promoter, an adipocyte-specific promoter, a cardiomyocyte-specific promoter, a skeletal muscle-specific promoter, lung progenitor cell specific promoter, a photoreceptor-specific promoter, and a RPE selective promoter.

133. The CRISPR/Cas system of any one of claims 108-132, wherein the gRNA is a sgRNA.

134. The CRISPR/Cas system of any one of claims 108-133, wherein the gRNA or sgRNA comprises a spacer sequence comprising 17 to 24 nucleotides.

135. The CRISPR/Cas system of any one of claims 108-133, wherein the nuclease segment is provided in a first vector, the gRNA segment and the promoter segment are provided together in a second vector, and the repressor segment and/or the shRNA segment is provided in a third vector.

136. The CRISPR/Cas system of any one of claims 112-134, wherein the nuclease segment, the gRNA segment, and the promoter segment are provided together in a first vector and the repressor segment and/or the shRNA segment is provided in a second vector.

137. The CRISPR/Cas system of any one of claims 118-134, wherein the nuclease segment, the gRNA segment, the promoter segment, and the one or more self-inactivating segments are provided together in a first vector and the repressor segment and/or the shRNA segment is provided in a second vector.

138. The CRISPR/Cas system of any one of claims 118-134, wherein the nuclease segment, the gRNA segment, the promoter segment, the one or more self-inactivating segments, the repressor segment, and the shRNA segment are provided in a vector.

139. The CRISPR/Cas system of any one of claims 135-137, wherein the first vector and the second vector are AAV vectors or plasmids.

140. The CRISPR/Cas system of claim 138, wherein the vector is an AAV vector or plasmid.

141. The CRISPR/Cas system of claim 139 or 140, wherein the AAV vectors are AAV2 serotype vectors, AAV5 serotype vectors, or AAV6 serotype vectors.

142. A pharmaceutical composition comprising the CRISPR/Cas system of any one of claims 108-141.

143. A packaging cell comprising the CRISPR/Cas system of any of claims 108-141.

144. A method of controlling transcription of gRNAs and post-transcriptional expression of Cas nuclease during AAV packaging, the method comprising:
contacting a packaging cell with the CRISPR/Cas system of any one of claims 108-141.

145. A method of reducing mutagenesis at one or more SIN site in a recombinant AAV vector, the method comprising:
contacting a cell with the CRISPR/Cas system of any one of claims 108-141.

146. The method of any one of claim 144 or 145, wherein the packaging cell is a human cell.

147. A method of controlling transcription of gRNAs and post-transcriptional expression of Cas nuclease during AAV packaging, the method comprising:
contacting a packaging cell with a nucleic acid comprising sequence encoding the recombinant AAV vector of any one of claims 35-60; and
contacting the packaging cell with a nucleic acid sequence encoding a tetracycline repressor segment and a shRNA segment.

148. A method of producing a recombinant AAV vector, the method comprising:
introducing into a packaging cell:
(i) a first vector comprising a repressor segment, wherein the repressor segment comprises a nucleotide sequence that encodes a tetracycline repressor protein;
(ii) a second vector comprising a shRNA segment comprising a nucleotide sequence that encodes a shRNA that comprises sequence that is complementary to a transcript from the nuclease segment;
(iii) a nucleic acid comprising sequence encoding the recombinant AAV vector of any one of claims 35-60; and
(iv) one or more viral components for producing the recombinant AAV vector;
culturing the packaging cell; and
isolating the recombinant AAV vector comprising the nucleic acid of (iii) from the packaging cell.

149. A method of producing a recombinant AAV vector, the method comprising:
introducing into packaging cell a nucleic acid comprising sequence encoding the recombinant AAV vector of any one of claims 35-60;
introducing into the packaging cell one or more viral components for producing the AAV;
culturing the packaging cell; and
isolating the recombinant AAV vector comprising the nucleic acid from the packaging cell;
wherein the packaging cell expressing a tetracycline repressor protein and a shRNA.

150. The method of any one of claims 148-149, wherein the nucleic acid further comprises one or more viral components.

151. The method of any one of claims 148-149, wherein the one or more viral components are introduced via separate vector other than the nucleic acid.

152. The method of any one of claims 148-149, wherein the one or more viral components are encoded in a cellular genome.

153. A recombinant AAV vector produced by any one of the methods of claim 67-71, 103-107, or 148-152.

154. A CRISPR/Cas system comprising:
(a) a nuclease segment comprising a codon optimized nucleotide sequence that encodes a SaCas9 or variant thereof;
(b) a gRNA segment comprising a nucleotide sequence that encodes a gRNA or sgRNA;
(c) a promoter segment comprising a nucleotide sequence that encodes a first promoter comprising one or more tetracycline operator sequence;
(d) a short-hairpin RNA (shRNA) segment comprising a nucleotide sequence that encodes a shRNA that comprises a sequence that is complementary to a transcript from the nuclease segment; and
(e) a repressor segment comprising a nucleotide sequence that encodes a tetracycline repressor protein,
wherein (a)-(c) are provided together in a first AAV vector or plasmid, and (d)-(e) are provided together in a second AAV vector or plasmid.

155. The CRISPR/Cas system of claim 154, wherein the first vector comprises a first self-inactivating segment comprising a SIN site at the 5′ end of the codon optimized nucleotide sequence that encodes the SaCas9 or variant thereof, and a second self-inactivating segment comprising a SIN site in an intron within the codon optimized nucleotide sequence that encodes the SaCas9 or variant thereof, wherein the gRNA or sgRNA is substantially complementary to the SIN sites.


SEQUENCE LISTING (69 sequences)

SEQ ID NO: 1 - RNA, 115 nt, synthetic construct
  source 1..115: mol_type = other RNA
gacgggtgtggtacgcagccactgtttaagtactctgtgctggaaacagcacagaatctacttaaacaaggcaaaatgccgtgtttatctcgtcaacttgttggcgagatttttt

SEQ ID NO: 2 - RNA, 112 nt, synthetic construct
  source 1..112: mol_type = other RNA
gggtgtggtacgcagccactgtttaagtactctgtgctggaaacagcacagaatctacttaaacaaggcaaaatgccgtgtttatctcgtcaacttgttggcgagatttttt

SEQ ID NO: 3 - DNA, 223 bp, synthetic construct
  source 1..223: mol_type = other DNA
gaacgctgacgtcatcaacccgctccaaggaatcgcgggcccagtgtcactaggcgggaacacccagcgcgcgtgcgccctggcaggaagatggctgtgagggacaggggagtggcgccctgcaatatttgcatgtcgctatgtgttctgggaaatcaccataaacgtgaaatccctatcagtgatagagacttataagttccctatcagtgatagagacacc

SEQ ID NO: 4 - DNA, 19 bp, synthetic construct
  source 1..19: mol_type = other DNA
tccctatcagtgatagaga

SEQ ID NO: 5 - DNA, 6805 bp, synthetic construct
  source 1..6805: mol_type = other DNA
cctgcaggcagctgcgcgctcgctcgctcactgaggccgcccgggcgtcgggcgacctttggtcgcccggcctcagtgagcgagcgagcgcgcagagagggagtggccaactccatcactaggggttcctgcggccgcacgcgtgctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgctagctaactaccggtgccaccatggccccaaagaagaagcggaaggtcggatccaagcggaactatatcctgggactggacatcggaattacctccgtgggatacggcatcatcgattacgagactagggacgtgattgacgccggcgtgagactctttaaggaggccaacgtggaaaacaacgaaggtcgcagatccaagcggggtgcaagacgcctgaagcgccggaggagacatcggatacagcgcgtgaagaagctccttttcgactacaacctcctcactgaccactcggaattgtccggtatcaacccctacgaagcccgcgtgaaaggcctgagccagaagctgtccgaagaggagtttagcgcagccctgctgcacctggctaagcgaaggggggtgcacaacgtgaacgaggtggaggaggacactggcaacgaactgtccaccaaggagcagatttcacggaactcgaaggcgctggaagagaaatatgtggccgagctgcagctggagaggctcaagaaggatggcgaagtccgggggagcatcaatcgcttcaagacctcggactacgtgaaggaagccaaacagctgttgaaggtgcagaaggcctaccaccaactggaccaatcattcattgacacttacatcgatctgcttgaaaccaggcgcacctactacgagggtcctggagaaggcagccctttcggatggaaggacatcaaggagtggtatgagatgctgatgggtcattgcacctactttccggaagaactgcgctcagtgaagtacgcgtacaacgctgacctctacaacgctctcaacgatctgaacaacctcgtgatcacccgggacgagaacgaaaagctggagtactacgaaaagttccagattatcgaaaacgtgttcaagcagaagaagaagcccaccctgaagcagattgcaaaggagatccttgtgaacgaggaggatattaagggctaccgggtcacctccaccgggaaaccagagttcactaatctcaaggtgtaccatgacattaaggacattactgcccgcaaggagatcattgaaaacgcggaactgctggaccaaatcgcgaagatcctgaccatctatcagagctccgaggatatccaggaggaacttactaacctcaattccgagctgacgcaggaagaaatcgagcaaattagcaacctgaagggttacactggaacccacaacctcagcttgaaagcgattaaccttattttggatgaactttggcacactaatgacaatcagatcgccattttcaaccggctgaaactggtgccgaagaaggtggacctgagccaacagaaggaaatcccgaccacccttgtggacgatttcatcctgtcacctgtggtgaagaggagcttcatccagtcgatcaaggtcatcaacgccatcataaagaagtacggccttcccaacgacatcatcatcgaactggcccgcgagaagaactccaaagatgcccagaagatgatcaacgagatgcagaagcgaaaccggcagacgaacgaacggatcgaggagatcatccggaccaccgggaaggaaaacgcgaagtacctgatcgagaaaatcaagctgcatgatatgcaggaagggaagtgtctctactccctggaggccattccgctggaggatttgctgaacaaccctttcaactacgaagtcgatcatatcattcctcgctccgtgtccttcgataactccttcaacaataaggtcctcgtgaagcaggaggagaagtaagtatcaaggttacaagacaggtttaaggagaccaatagaaactgggctcgagcttgtcgagacagagaagactcttgcgtttctgataggcacctattggtcttactgacatccactttgcctttctctccacagctcgaagaagggcaacagaaccccgttccagtacctctcgtcgtccgactccaagatcagctacgaaactttcaagaagcacattctgaacctggccaagggcaaagggagaattagcaagaccaagaaggaatacctcctggaagagagagacatcaaccgcttctcggtgcaaaaggatttcatcaaccgcaacctggtcgataccagatacgccaccaggggactgatgaacctcctgcggtcctacttccgggtcaacaatctggacgtgaaggtcaaatccatcaacgggggctttacttctttcctgcgccggaagtggaagttcaagaaggaacggaacaagggatacaagcaccacgctgaagatgccctgattattgccaacgccgacttcatctttaaggaatggaaaaagctggacaaggctaagaaggtcatggagaaccagatgttcgaagaaaagcaggccgagtccatgcccgaaatcgaaaccgagcaggaatacaaggagatcttcatcacaccgcaccaaatcaagcacatcaaggacttcaaggattacaagtacagccaccgggtggacaagaagcctaacagagagcttatcaacgacaccctgtactccacgcgcaaggacgacaagggaaacacattgatcgtgaacaacctgaacggactgtatgacaaggacaatgacaaactgaagaagctgatcaacaaatcgccggaaaagctcctgatgtaccatcacgaccctcaaacctaccagaaactgaagctcatcatggagcagtacggcgacgaaaagaatcccctgtacaaatactacgaggagactggaaattacctgactaagtactccaagaaggataacggccccgtgatcaagaagattaagtactacggaaacaaactgaacgcacatctcgacatcaccgatgattatccaaactcccgcaacaaagtcgtgaagctctccctcaaaccgtaccgcttcgacgtgtacctggataatggggtgtacaagttcgtgaccgtgaagaacctggacgtcattaagaaggaaaactactacgaagtgaactcaaagtgctacgaggaagccaagaagctcaagaagatcagcaaccaggccgagttcatcgcatcgttttacaacaatgacctcattaagattaatggagaactgtacagagtgatcggcgtgaacaacgacctcctgaaccggattgaagtgaacatgatcgatattacctaccgggagtatctggagaacatgaacgacaagcgcccaccgagaatcatcaaaactattgcctccaagacccaatccattaagaaatactccaccgacatcctgggcaacctgtacgaggtcaagtcgaagaagcacccccagattatcaagaagggaaaaaggccggcggccacgaaaaaggccggccaggcaaaaaagaaaaaggcttaagaattcctagagctcgctgatcagcctcgaaacttgtttattgcagcttataatggttacaaataaagcaatagcatcacaaatttcacaaataaagcatttttttcactgcattctagttgtggtttgtccaaactcatcaatgtatcttatcatgtctgtataccagcaggctttcctaggttcgaacgctgacgtcatcaacccgctccaaggaatcgcgggcccagtgtcactaggcgggaacacccagcgcgcgtgcgccctggcaggaagatggctgtgagggacaggggagtggcgccctgcaatatttgcatgtcgctatgtgttctgggaaatcaccataaacgtgaaatccctatcagtgatagagacttataagttccctatcagtgatagagacaccgacgggtgtggtacgcagccactgtttaagtactctgtgctggaaacagcacagaatctacttaaacaaggcaaaatgccgtgtttatctcgtcaacttgttggcgagattttttcacgtgcggaccgaggctgcagcgtcgtcctccctaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggcctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgaacaataaaactgtctgcttacataaacagtaatacaaggggtgttatgagccatattcaacgggaaacgtcgaggccgcgattaaattccaacatggatgctgatttatatgggtataaatgggctcgcgataatgtcgggcaatcaggtgcgacaatctatcgcttgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccggaaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataaacttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgt

SEQ ID NO: 6 - DNA, 8353 bp, synthetic construct
  source 1..8353: mol_type = other DNA
cctgcaggcagctgcgcgctcgctcgctcactgaggccgcccgggcgtcgggcgacctttggtcgcccggcctcagtgagcgagcgagcgcgcagagagggagtggccaactccatcactaggggttcctgcggccgcacgcgtgctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgctagctaactaccggtgccaccatggccccaaagaagaagcggaaggtcggatccaagcggaactatatcctgggactggacatcggaattacctccgtgggatacggcatcatcgattacgagactagggacgtgattgacgccggcgtgagactctttaaggaggccaacgtggaaaacaacgaaggtcgcagatccaagcggggtgcaagacgcctgaagcgccggaggagacatcggatacagcgcgtgaagaagctccttttcgactacaacctcctcactgaccactcggaattgtccggtatcaacccctacgaagcccgcgtgaaaggcctgagccagaagctgtccgaagaggagtttagcgcagccctgctgcacctggctaagcgaaggggggtgcacaacgtgaacgaggtggaggaggacactggcaacgaactgtccaccaaggagcagatttcacggaactcgaaggcgctggaagagaaatatgtggccgagctgcagctggagaggctcaagaaggatggcgaagtccgggggagcatcaatcgcttcaagacctcggactacgtgaaggaagccaaacagctgttgaaggtgcagaaggcctaccaccaactggaccaatcattcattgacacttacatcgatctgcttgaaaccaggcgcacctactacgagggtcctggagaaggcagccctttcggatggaaggacatcaaggagtggtatgagatgctgatgggtcattgcacctactttccggaagaactgcgctcagtgaagtacgcgtacaacgctgacctctacaacgctctcaacgatctgaacaacctcgtgatcacccgggacgagaacgaaaagctggagtactacgaaaagttccagattatcgaaaacgtgttcaagcagaagaagaagcccaccctgaagcagattgcaaaggagatccttgtgaacgaggaggatattaagggctaccgggtcacctccaccgggaaaccagagttcactaatctcaaggtgtaccatgacattaaggacattactgcccgcaaggagatcattgaaaacgcggaactgctggaccaaatcgcgaagatcctgaccatctatcagagctccgaggatatccaggaggaacttactaacctcaattccgagctgacgcaggaagaaatcgagcaaattagcaacctgaagggttacactggaacccacaacctcagcttgaaagcgattaaccttattttggatgaactttggcacactaatgacaatcagatcgccattttcaaccggctgaaactggtgccgaagaaggtggacctgagccaacagaaggaaatcccgaccacccttgtggacgatttcatcctgtcacctgtggtgaagaggagcttcatccagtcgatcaaggtcatcaacgccatcataaagaagtacggccttcccaacgacatcatcatcgaactggcccgcgagaagaactccaaagatgcccagaagatgatcaacgagatgcagaagcgaaaccggcagacgaacgaacggatcgaggagatcatccggaccaccgggaaggaaaacgcgaagtacctgatcgagaaaatcaagctgcatgatatgcaggaagggaagtgtctctactccctggaggccattccgctggaggatttgctgaacaaccctttcaactacgaagtcgatcatatcattcctcgctccgtgtccttcgataactccttcaacaataaggtcctcgtgaagcaggaggagaagtaagtatcaaggttacaagacaggtttaaggagaccaatagaaactgggctcgagcttgtcgagacagagaagactcttgcgtttctgataggcacctattggtcttactgacatccactttgcctttctctccacagctcgaagaagggcaacagaaccccgttccagtacctctcgtcgtccgactccaagatcagctacgaaactttcaagaagcacattctgaacctggccaagggcaaagggagaattagcaagaccaagaaggaatacctcctggaagagagagacatcaaccgcttctcggtgcaaaaggatttcatcaaccgcaacctggtcgataccagatacgccaccaggggactgatgaacctcctgcggtcctacttccgggtcaacaatctggacgtgaaggtcaaatccatcaacgggggctttacttctttcctgcgccggaagtggaagttcaagaaggaacggaacaagggatacaagcaccacgctgaagatgccctgattattgccaacgccgacttcatctttaaggaatggaaaaagctggacaaggctaagaaggtcatggagaaccagatgttcgaagaaaagcaggccgagtccatgcccgaaatcgaaaccgagcaggaatacaaggagatcttcatcacaccgcaccaaatcaagcacatcaaggacttcaaggattacaagtacagccaccgggtggacaagaagcctaacagagagcttatcaacgacaccctgtactccacgcgcaaggacgacaagggaaacacattgatcgtgaacaacctgaacggactgtatgacaaggacaatgacaaactgaagaagctgatcaacaaatcgccggaaaagctcctgatgtaccatcacgaccctcaaacctaccagaaactgaagctcatcatggagcagtacggcgacgaaaagaatcccctgtacaaatactacgaggagactggaaattacctgactaagtactccaagaaggataacggccccgtgatcaagaagattaagtactacggaaacaaactgaacgcacatctcgacatcaccgatgattatccaaactcccgcaacaaagtcgtgaagctctccctcaaaccgtaccgcttcgacgtgtacctggataatggggtgtacaagttcgtgaccgtgaagaacctggacgtcattaagaaggaaaactactacgaagtgaactcaaagtgctacgaggaagccaagaagctcaagaagatcagcaaccaggccgagttcatcgcatcgttttacaacaatgacctcattaagattaatggagaactgtacagagtgatcggcgtgaacaacgacctcctgaaccggattgaagtgaacatgatcgatattacctaccgggagtatctggagaacatgaacgacaagcgcccaccgagaatcatcaaaactattgcctccaagacccaatccattaagaaatactccaccgacatcctgggcaacctgtacgaggtcaagtcgaagaagcacccccagattatcaagaagggaaaaaggccggcggccacgaaaaaggccggccaggcaaaaaagaaaaaggcttaagaattcctagagctcgctgatcagcctcgaaacttgtttattgcagcttataatggttacaaataaagcaatagcatcacaaatttcacaaataaagcatttttttcactgcattctagttgtggtttgtccaaactcatcaatgtatcttatcatgtctgtataccagcaggctttcctaggttcgaacgctgacgtcatcaacccgctccaaggaatcgcgggcccagtgtcactaggcgggaacacccagcgcgcgtgcgccctggcaggaagatggctgtgagggacaggggagtggcgccctgcaatatttgcatgtcgctatgtgttctgggaaatcaccataaacgtgaaatccctatcagtgatagagacttataagttccctatcagtgatagagacaccgacgggtgtggtacgcagccactgtttaagtactctgtgctggaaacagcacagaatctacttaaacaaggcaaaatgccgtgtttatctcgtcaacttgttggcgagattttttcacgtgcggaccgaggctgcagcgtcgtcctccctaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggcctattggttaaaaaatgagctgatttaacaaaaatttaacgcgaattttaacaaaatattaacgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgttaattaaccggtgccaccatgatgtcccgccttgataaatcaaaagtaataaacagtgctcttgagcttctcaatgaagttggtatagaagggttgacgactcggaaattggcgcaaaaactcggtgttgagcagccaaccttgtattggcatgttaaaaacaaacgagcactcctcgacgctttggcgatagagatgctggacaggcaccacacgcatttctgtcccctcgaaggagagtcatggcaggatttccttagaaataacgcaaagtccttcagatgtgcgctgcttagtcaccgcgacggcgcaaaagttcatctcggcactaggccaaccgagaaacagtacgagactctggagaaccaactggcgtttttgtgtcaacagggttttagtctcgaaaatgcgctctatgctctctctgcggttggccatttcaccctcggatgcgtactggaagatcaggagcaccaagtggccaaagaagaacgggaaacgccgactacggacagcatgcctccgttgctccggcaagctatagagctcttcgatcaccaaggcgctgagccagctttcttgttcggattggaacttattatatgcgggctcgaaaagcagcttaaatgcgagtcaggttaagcggccgctcgagtctagagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgaacaataaaactgtctgcttacataaacagtaatacaaggggtgttatgagccatattcaacgggaaacgtcgaggccgcgattaaattccaacatggatgctgatttatatgggtataaatgggctcgcgataatgtcgggcaatcaggtgcgacaatctatcgcttgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccggaaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataaacttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgt

SEQ ID NO: 7 - DNA, 6802 bp, synthetic construct
  source 1..6802: mol_type = other DNA
cctgcaggcagctgcgcgctcgctcgctcactgaggccgcccgggcgtcgggcgacctttggtcgcccggcctcagtgagcgagcgagcgcgcagagagggagtggccaactccatcactaggggttcctgcggccgcacgcgtgctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgctagctaactaccggtgccaccatggccccaaagaagaagcggaaggtcggatccaagcggaactatatcctgggactggacatcggaattacctccgtgggatacggcatcatcgattacgagactagggacgtgattgacgccggcgtgagactctttaaggaggccaacgtggaaaacaacgaaggtcgcagatccaagcggggtgcaagacgcctgaagcgccggaggagacatcggatacagcgcgtgaagaagctccttttcgactacaacctcctcactgaccactcggaattgtccggtatcaacccctacgaagcccgcgtgaaaggcctgagccagaagctgtccgaagaggagtttagcgcagccctgctgcacctggctaagcgaaggggggtgcacaacgtgaacgaggtggaggaggacactggcaacgaactgtccaccaaggagcagatttcacggaactcgaaggcgctggaagagaaatatgtggccgagctgcagctggagaggctcaagaaggatggcgaagtccgggggagcatcaatcgcttcaagacctcggactacgtgaaggaagccaaacagctgttgaaggtgcagaaggcctaccaccaactggaccaatcattcattgacacttacatcgatctgcttgaaaccaggcgcacctactacgagggtcctggagaaggcagccctttcggatggaaggacatcaaggagtggtatgagatgctgatgggtcattgcacctactttccggaagaactgcgctcagtgaagtacgcgtacaacgctgacctctacaacgctctcaacgatctgaacaacctcgtgatcacccgggacgagaacgaaaagctggagtactacgaaaagttccagattatcgaaaacgtgttcaagcagaagaagaagcccaccctgaagcagattgcaaaggagatccttgtgaacgaggaggatattaagggctaccgggtcacctccaccgggaaaccagagttcactaatctcaaggtgtaccatgacattaaggacattactgcccgcaaggagatcattgaaaacgcggaactgctggaccaaatcgcgaagatcctgaccatctatcagagctccgaggatatccaggaggaacttactaacctcaattccgagctgacgcaggaagaaatcgagcaaattagcaacctgaagggttacactggaacccacaacctcagcttgaaagcgattaaccttattttggatgaactttggcacactaatgacaatcagatcgccattttcaaccggctgaaactggtgccgaagaaggtggacctgagccaacagaaggaaatcccgaccacccttgtggacgatttcatcctgtcacctgtggtgaagaggagcttcatccagtcgatcaaggtcatcaacgccatcataaagaagtacggccttcccaacgacatcatcatcgaactggcccgcgagaagaactccaaagatgcccagaagatgatcaacgagatgcagaagcgaaaccggcagacgaacgaacggatcgaggagatcatccggaccaccgggaaggaaaacgcgaagtacctgatcgagaaaatcaagctgcatgatatgcaggaagggaagtgtctctactccctggaggccattccgctggaggatttgctgaacaaccctttcaactacgaagtcgatcatatcattcctcgctccgtgtccttcgataactccttcaacaataaggtcctcgtgaagcaggaggagaagtaagtatcaaggttacaagacaggtttaaggagaccaatagaaactgggctcgagcttgtcgagacagagaagactcttgcgtttctgataggcacctattggtcttactgacatccactttgcctttctctccacagctcgaagaagggcaacagaaccccgttccagtacctctcgtcgtccgactccaagatcagctacgaaactttcaagaagcacattctgaacctggccaagggcaaagggagaattagcaagaccaagaaggaatacctcctggaagagagagacatcaaccgcttctcggtgcaaaaggatttcatcaaccgcaacctggtcgataccagatacgccaccaggggactgatgaacctcctgcggtcctacttccgggtcaacaatctggacgtgaaggtcaaatccatcaacgggggctttacttctttcctgcgccggaagtggaagttcaagaaggaacggaacaagggatacaagcaccacgctgaagatgccctgattattgccaacgccgacttcatctttaaggaatggaaaaagctggacaaggctaagaaggtcatggagaaccagatgttcgaagaaaagcaggccgagtccatgcccgaaatcgaaaccgagcaggaatacaaggagatcttcatcacaccgcaccaaatcaagcacatcaaggacttcaaggattacaagtacagccaccgggtggacaagaagcctaacagagagcttatcaacgacaccctgtactccacgcgcaaggacgacaagggaaacacattgatcgtgaacaacctgaacggactgtatgacaaggacaatgacaaactgaagaagctgatcaacaaatcgccggaaaagctcctgatgtaccatcacgaccctcaaacctaccagaaactgaagctcatcatggagcagtacggcgacgaaaagaatcccctgtacaaatactacgaggagactggaaattacctgactaagtactccaagaaggataacggccccgtgatcaagaagattaagtactacggaaacaaactgaacgcacatctcgacatcaccgatgattatccaaactcccgcaacaaagtcgtgaagctctccctcaaaccgtaccgcttcgacgtgtacctggataatggggtgtacaagttcgtgaccgtgaagaacctggacgtcattaagaaggaaaactactacgaagtgaactcaaagtgctacgaggaagccaagaagctcaagaagatcagcaaccaggccgagttcatcgcatcgttttacaacaatgacctcattaagattaatggagaactgtacagagtgatcggcgtgaacaacgacctcctgaaccggattgaagtgaacatgatcgatattacctaccgggagtatctggagaacatgaacgacaagcgcccaccgagaatcatcaaaactattgcctccaagacccaatccattaagaaatactccaccgacatcctgggcaacctgtacgaggtcaagtcgaagaagcacccccagattatcaagaagggaaaaaggccggcggccacgaaaaaggccggccaggcaaaaaagaaaaaggcttaagaattcctagagctcgctgatcagcctcgaaacttgtttattgcagcttataatggttacaaataaagcaatagcatcacaaatttcacaaataaagcatttttttcactgcattctagttgtggtttgtccaaactcatcaatgtatcttatcatgtctgtataccagcaggctttcctaggttcgaacgctgacgtcatcaacccgctccaaggaatcgcgggcccagtgtcactaggcgggaacacccagcgcgcgtgcgccctggcaggaagatggctgtgagggacaggggagtggcgccctgcaatatttgcatgtcgctatgtgttctgggaaatcaccataaacgtgaaatccctatcagtgatagagacttataagttccctatcagtgatagagacaccgggtgtggtacgcagccactgtttaagtactctgtgctggaaacagcacagaatctacttaaacaaggcaaaatgccgtgtttatctcgtcaacttgttggcgagattttttcacgtgcggaccgaggctgcagcgtcgtcctccctaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggcctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgaacaataaaactgtctgcttacataaacagtaatacaaggggtgttatgagccatattcaacgggaaacgtcgaggccgcgattaaattccaacatggatgctgatttatatgggtataaatgggctcgcgataatgtcgggcaatcaggtgcgacaatctatcgcttgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccggaaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataaacttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgt

SEQ ID NO: 8 - DNA, 6828 bp, synthetic construct
  source 1..6828: mol_type = other DNA
cctgcaggcagctgcgcgctcgctcgctcactgaggccgcccgggcgtcgggcgacctttggtcgcccggcctcagtgagcgagcgagcgcgcagagagggagtggccaactccatcactaggggttcctgcggccgcacgcgtgctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgctagctaactaccggtgccaccatggccccaaagaagaagcggaaggtcggatccaagcggaactatatcctgggactggacatcggaattacctccgtgggatacggcatcatcgattacgagactagggacgtgattgacgccggcgtgagactctttaaggaggccaacgtggaaaacaacgaaggtcgcagatccaagcggggtgcaagacgcctgaagcgccggaggagacatcggatacagcgcgtgaagaagctccttttcgactacaacctcctcactgaccactcggaattgtccggtatcaacccctacgaagcccgcgtgaaaggcctgagccagaagctgtccgaagaggagtttagcgcagccctgctgcacctggctaagcgaaggggggtgcacaacgtgaacgaggtggaggaggacactggcaacgaactgtccaccaaggagcagatttcacggaactcgaaggcgctggaagagaaatatgtggccgagctgcagctggagaggctcaagaaggatggcgaagtccgggggagcatcaatcgcttcaagacctcggactacgtgaaggaagccaaacagctgttgaaggtgcagaaggcctaccaccaactggaccaatcattcattgacacttacatcgatctgcttgaaaccaggcgcacctactacgagggtcctggagaaggcagccctttcggatggaaggacatcaaggagtggtatgagatgctgatgggtcattgcacctactttccggaagaactgcgctcagtgaagtacgcgtacaacgctgacctctacaacgctctcaacgatctgaacaacctcgtgatcacccgggacgagaacgaaaagctggagtactacgaaaagttccagattatcgaaaacgtgttcaagcagaagaagaagcccaccctgaagcagattgcaaaggagatccttgtgaacgaggaggatattaagggctaccgggtcacctccaccgggaaaccagagttcactaatctcaaggtgtaccatgacattaaggacattactgcccgcaaggagatcattgaaaacgcggaactgctggaccaaatcgcgaagatcctgaccatctatcagagctccgaggatatccaggaggaacttactaacctcaattccgagctgacgcaggaagaaatcgagcaaattagcaacctgaagggttacactggaacccacaacctcagcttgaaagcgattaaccttattttggatgaactttggcacactaatgacaatcagatcgccattttcaaccggctgaaactggtgccgaagaaggtggacctgagccaacagaaggaaatcccgaccacccttgtggacgatttcatcctgtcacctgtggtgaagaggagcttcatccagtcgatcaaggtcatcaacgccatcataaagaagtacggccttcccaacgacatcatcatcgaactggcccgcgagaagaactccaaagatgcccagaagatgatcaacgagatgcagaagcgaaaccggcagacgaacgaacggatcgaggagatcatccggaccaccgggaaggaaaacgcgaagtacctgatcgagaaaatcaagctgcatgatatgcaggaagggaagtgtctctactccctggaggccattccgctggaggatttgctgaacaaccctttcaactacgaagtcgatcatatcattcctcgctccgtgtccttcgataactccttcaacaataaggtcctcgtgaagcaggaggagaagtaagtatcaaggttacaagacaggtttaaggagaccaatagaaactgggctcgagcttgtcgagacagagaagactcttgcgtttctgataggcacctattggtcttactgacatccactttgcctttctctccacagctcgaagaagggcaacagaaccccgttccagtacctctcgtcgtccgactccaagatcagctacgaaactttcaagaagcacattctgaacctggccaagggcaaagggagaattagcaagaccaagaaggaatacctcctggaagagagagacatcaaccgcttctcggtgcaaaaggatttcatcaaccgcaacctggtcgataccagatacgccaccaggggactgatgaacctcctgcggtcctacttccgggtcaacaatctggacgtgaaggtcaaatccatcaacgggggctttacttctttcctgcgccggaagtggaagttcaagaaggaacggaacaagggatacaagcaccacgctgaagatgccctgattattgccaacgccgacttcatctttaaggaatggaaaaagctggacaaggctaagaaggtcatggagaaccagatgttcgaagaaaagcaggccgagtccatgcccgaaatcgaaaccgagcaggaatacaaggagatcttcatcacaccgcaccaaatcaagcacatcaaggacttcaaggattacaagtacagccaccgggtggacaagaagcctaacagagagcttatcaacgacaccctgtactccacgcgcaaggacgacaagggaaacacattgatcgtgaacaacctgaacggactgtatgacaaggacaatgacaaactgaagaagctgatcaacaaatcgccggaaaagctcctgatgtaccatcacgaccctcaaacctaccagaaactgaagctcatcatggagcagtacggcgacgaaaagaatcccctgtacaaatactacgaggagactggaaattacctgactaagtactccaagaaggataacggccccgtgatcaagaagattaagtactacggaaacaaactgaacgcacatctcgacatcaccgatgattatccaaactcccgcaacaaagtcgtgaagctctccctcaaaccgtaccgcttcgacgtgtacctggataatggggtgtacaagttcgtgaccgtgaagaacctggacgtcattaagaaggaaaactactacgaagtgaactcaaagtgctacgaggaagccaagaagctcaagaagatcagcaaccaggccgagttcatcgcatcgttttacaacaatgacctcattaagattaatggagaactgtacagagtgatcggcgtgaacaacgacctcctgaaccggattgaagtgaacatgatcgatattacctaccgggagtatctggagaacatgaacgacaagcgcccaccgagaatcatcaaaactattgcctccaagacccaatccattaagaaatactccaccgacatcctgggcaacctgtacgaggtcaagtcgaagaagcacccccagattatcaagaagggaaaaaggccggcggccacgaaaaaggccggccaggcaaaaaagaaaaaggcttaagaattcctagagctcgctgatcagcctcgaaacttgtttattgcagcttataatggttacaaataaagcaatagcatcacaaatttcacaaataaagcatttttttcactgcattctagttgtggtttgtccaaactcatcaatgtatcttatcatgtctgtataccagcaggctttcctaggttcgagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccgggtgtggtacgcagccactgtttaagtactctgtgctggaaacagcacagaatctacttaaacaaggcaaaatgccgtgtttatctcgtcaacttgttggcgagattttttcacgtgcggaccgaggctgcagcgtcgtcctccctaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggcctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgaacaataaaactgtctgcttacataaacagtaatacaaggggtgttatgagccatattcaacgggaaacgtcgaggccgcgattaaattccaacatggatgctgatttatatgggtataaatgggctcgcgataatgtcgggcaatcaggtgcgacaatctatcgcttgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccggaaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataaacttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgt

SEQ ID NO: 9 - RNA, 52 nt, synthetic construct
  source 1..52: mol_type = other RNA
ggaattacctccgtgggataccgaagtatcccacggaggtaattcctttttt

SEQ ID NO: 10 - RNA, 52 nt, synthetic construct
  source 1..52: mol_type = other RNA
ggatatccaggaggaacttaccgaagtaagttcctcctggatatcctttttt

SEQ ID NO: 11 - RNA, 52 nt, synthetic construct
  source 1..52: mol_type = other RNA
ggagcttcatccagtcgatcacgaatgatcgactggatgaagctcctttttt

SEQ ID NO: 12 - DNA, 6785 bp, synthetic construct
  source 1..6785: mol_type = other DNA
cctgcaggcagctgcgcgctcgctcgctcactgaggccgcccgggcgtcgggcgacctttggtcgcccggcctcagtgagcgagcgagcgcgcagagagggagtggccaactccatcactaggggttcctgcggccgcacgcgtgctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgctagctaactaccggtgccaccatggattacaaagaccacgacggagactacaaggatcacgacattgattacaaggatgatgatgataaggccccaaagaagaagcggaaggtcggtatccacggagtcccagcagccggatccgccatgaagcggaactatatcctgggactggacatcggaattacctccgtgggatacggcatcatcgattacgagactagggacgtgattgacgccggcgtgagactctttaaggaggccaacgtggaaaacaacgaaggtcgcagatccaagcggggtgcaagacgcctgaagcgccggaggagacatcggatacagcgcgtgaagaagctccttttcgactacaacctcctcactgaccactcggaattgtccggtatcaacccctacgaagcccgcgtgaaaggcctgagccagaagctgtccgaagaggagtttagcgcagccctgctgcacctggctaagcgaaggggggtgcacaacgtgaacgaggtggaggaggacactggcaacgaactgtccaccaaggagcagatttcacggaactcgaaggcgctggaagagaaatatgtggccgagctgcagctggagaggctcaagaaggatggcgaagtccgggggagcatcaatcgcttcaagacctcggactacgtgaaggaagccaaacagctgttgaaggtgcagaaggcctaccaccaactggaccaatcattcattgacacttacatcgatctgcttgaaaccaggcgcacctactacgagggtcctggagaaggcagccctttcggatggaaggacatcaaggagtggtatgagatgctgatgggtcattgcacctactttccggaagaactgcgctcagtgaagtacgcgtacaacgctgacctctacaacgctctcaacgatctgaacaacctcgtgatcacccgggacgagaacgaaaagctggagtactacgaaaagttccagattatcgaaaacgtgttcaagcagaagaagaagcccaccctgaagcagattgcaaaggagatccttgtgaacgaggaggatattaagggctaccgggtcacctccaccgggaaaccagagttcactaatctcaaggtgtaccatgacattaaggacattactgcccgcaaggagatcattgaaaacgcggaactgctggaccaaatcgcgaagatcctgaccatctatcagagctccgaggatatccaggaggaacttactaacctcaattccgagctgacgcaggaagaaatcgagcaaattagcaacctgaagggttacactggaacccacaacctcagcttgaaagcgattaaccttattttggatgaactttggcacactaatgacaatcagatcgccattttcaaccggctgaaactggtgccgaagaaggtggacctgagccaacagaaggaaatcccgaccacccttgtggacgatttcatcctgtcacctgtggtgaagaggagcttcatccagtcgatcaaggtcatcaacgccatcataaagaagtacggccttcccaacgacatcatcatcgaactggcccgcgagaagaactccaaagatgcccagaagatgatcaacgagatgcagaagcgaaaccggcagacgaacgaacggatcgaggagatcatccggaccaccgggaaggaaaacgcgaagtacctgatcgagaaaatcaagctgcatgatatgcaggaagggaagtgtctctactccctggaggccattccgctggaggatttgctgaacaaccctttcaactacgaagtcgatcatatcattcctcgctccgtgtccttcgataactccttcaacaataaggtcctcgtgaagcaggaggagaactcgaagaagggcaacagaaccccgttccagtacctctcgtcgtccgactccaagatcagctacgaaactttcaagaagcacattctgaacctggccaagggcaaagggagaattagcaagaccaagaaggaatacctcctggaagagagagacatcaaccgcttctcggtgcaaaaggatttcatcaaccgcaacctggtcgataccagatacgccaccaggggactgatgaacctcctgcggtcctacttccgggtcaacaatctggacgtgaaggtcaaatccatcaacgggggctttacttctttcctgcgccggaagtggaagttcaagaaggaacggaacaagggatacaagcaccacgctgaagatgccctgattattgccaacgccgacttcatctttaaggaatggaaaaagctggacaaggctaagaaggtcatggagaaccagatgttcgaagaaaagcaggccgagtccatgcccgaaatcgaaaccgagcaggaatacaaggagatcttcatcacaccgcaccaaatcaagcacatcaaggacttcaaggattacaagtacagccaccgggtggacaagaagcctaacagagagcttatcaacgacaccctgtactccacgcgcaaggacgacaagggaaacacattgatcgtgaacaacctgaacggactgtatgacaaggacaatgacaaactgaagaagctgatcaacaaatcgccggaaaagctcctgatgtaccatcacgaccctcaaacctaccagaaactgaagctcatcatggagcagtacggcgacgaaaagaatcccctgtacaaatactacgaggagactggaaattacctgactaagtactccaagaaggataacggccccgtgatcaagaagattaagtactacggaaacaaactgaacgcacatctcgacatcaccgatgattatccaaactcccgcaacaaagtcgtgaagctctccctcaaaccgtaccgcttcgacgtgtacctggataatggggtgtacaagttcgtgaccgtgaagaacctggacgtcattaagaaggaaaactactacgaagtgaactcaaagtgctacgaggaagccaagaagctcaagaagatcagcaaccaggccgagttcatcgcatcgttttacaacaatgacctcattaagattaatggagaactgtacagagtgatcggcgtgaacaacgacctcctgaaccggattgaagtgaacatgatcgatattacctaccgggagtatctggagaacatgaacgacaagcgcccaccgagaatcatcaaaactattgcctccaagacccaatccattaagaaatactccaccgacatcctgggcaacctgtacgaggtcaagtcgaagaagcacccccagattatcaagaagggaaaaaggccggcggccacgaaaaaggccggccaggcaaaaaagaaaaaggcttaagaattcctagagctcgctgatcagcctcgaaacttgtttattgcagcttataatggttacaaataaagcaatagcatcacaaatttcacaaataaagcatttttttcactgcattctagttgtggtttgtccaaactcatcaatgtatcttatcatgtctgtataccagcaggctttcctaggttcgagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccgggtgtggtacgcagccactgtttaagtactctgtgctggaaacagcacagaatctacttaaacaaggcaaaatgccgtgtttatctcgtcaacttgttggcgagattttttcacgtgcggaccgaggctgcagcgtcgtcctccctaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggcctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgaacaataaaactgtctgcttacataaacagtaatacaaggggtgttatgagccatattcaacgggaaacgtcgaggccgcgattaaattccaacatggatgctgatttatatgggtataaatgggctcgcgataatgtcgggcaatcaggtgcgacaatctatcgcttgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccggaaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataaacttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgt

SEQ ID NO: 13 - DNA, 6228 bp, synthetic construct
  source 1..6228: mol_type = other DNA
gacggatcgggagatctgagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccggagacgacggcacgtctcattttttacccgatcccctatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgttaattaaccggtgccaccatgatgtcccgccttgataaatcaaaagtaataaacagtgctcttgagcttctcaatgaagttggtatagaagggttgacgactcggaaattggcgcaaaaactcggtgttgagcagccaaccttgtattggcatgttaaaaacaaacgagcactcctcgacgctttggcgatagagatgctggacaggcaccacacgcatttctgtcccctcgaaggagagtcatggcaggatttccttagaaataacgcaaagtccttcagatgtgcgctgcttagtcaccgcgacggcgcaaaagttcatctcggcactaggccaaccgagaaacagtacgagactctggagaaccaactggcgtttttgtgtcaacagggttttagtctcgaaaatgcgctctatgctctctctgcggttggccatttcaccctcggatgcgtactggaagatcaggagcaccaagtggccaaagaagaacgggaaacgccgactacggacagcatgcctccgttgctccggcaagctatagagctcttcgatcaccaaggcgctgagccagctttcttgttcggattggaacttattatatgcgggctcgaaaagcagcttaaatgcgagtcaggttaagcggccgctcgagtctagagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattaattctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtc

SEQ ID NO: 14 - DNA, 6254 bp, synthetic construct
  source 1..6254: mol_type = other DNA
gacggatcgggagatctgagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccggaattacctccgtgggataccgaagtatcccacggaggtaattccttttttacccgatcccctatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgttaattaaccggtgccaccatgatgtcccgccttgataaatcaaaagtaataaacagtgctcttgagcttctcaatgaagttggtatagaagggttgacgactcggaaattggcgcaaaaactcggtgttgagcagccaaccttgtattggcatgttaaaaacaaacgagcactcctcgacgctttggcgatagagatgctggacaggcaccacacgcatttctgtcccctcgaaggagagtcatggcaggatttccttagaaataacgcaaagtccttcagatgtgcgctgcttagtcaccgcgacggcgcaaaagttcatctcggcactaggccaaccgagaaacagtacgagactctggagaaccaactggcgtttttgtgtcaacagggttttagtctcgaaaatgcgctctatgctctctctgcggttggccatttcaccctcggatgcgtactggaagatcaggagcaccaagtggccaaagaagaacgggaaacgccgactacggacagcatgcctccgttgctccggcaagctatagagctcttcgatcaccaaggcgctgagccagctttcttgttcggattggaacttattatatgcgggctcgaaaagcagcttaaatgcgagtcaggttaagcggccgctcgagtctagagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattaattctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtc

SEQ ID NO: 15 - DNA, 6254 bp, synthetic construct
  source 1..6254: mol_type = other DNA
gacggatcgggagatctgagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccggatatccaggaggaacttaccgaagtaagttcctcctggatatccttttttacccgatcccctatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgttaattaaccggtgccaccatgatgtcccgccttgataaatcaaaagtaataaacagtgctcttgagcttctcaatgaagttggtatagaagggttgacgactcggaaattggcgcaaaaactcggtgttgagcagccaaccttgtattggcatgttaaaaacaaacgagcactcctcgacgctttggcgatagagatgctggacaggcaccacacgcatttctgtcccctcgaaggagagtcatggcaggatttccttagaaataacgcaaagtccttcagatgtgcgctgcttagtcaccgcgacggcgcaaaagttcatctcggcactaggccaaccgagaaacagtacgagactctggagaaccaactggcgtttttgtgtcaacagggttttagtctcgaaaatgcgctctatgctctctctgcggttggccatttcaccctcggatgcgtactggaagatcaggagcaccaagtggccaaagaagaacgggaaacgccgactacggacagcatgcctccgttgctccggcaagctatagagctcttcgatcaccaaggcgctgagccagctttcttgttcggattggaacttattatatgcgggctcgaaaagcagcttaaatgcgagtcaggttaagcggccgctcgagtctagagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattaattctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtc

SEQ ID NO: 16 - DNA, 6254 bp, synthetic construct
  source 1..6254: mol_type = other DNA
gacggatcgggagatctgagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccggagcttcatccagtcgatcacgaatgatcgactggatgaagctccttttttacccgatcccctatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgttaattaaccggtgccaccatgatgtcccgccttgataaatcaaaagtaataaacagtgctcttgagcttctcaatgaagttggtatagaagggttgacgactcggaaattggcgcaaaaactcggtgttgagcagccaaccttgtattggcatgttaaaaacaaacgagcactcctcgacgctttggcgatagagatgctggacaggcaccacacgcatttctgtcccctcgaaggagagtcatggcaggatttccttagaaataacgcaaagtccttcagatgtgcgctgcttagtcaccgcgacggcgcaaaagttcatctcggcactaggccaaccgagaaacagtacgagactctggagaaccaactggcgtttttgtgtcaacagggttttagtctcgaaaatgcgctctatgctctctctgcggttggccatttcaccctcggatgcgtactggaagatcaggagcaccaagtggccaaagaagaacgggaaacgccgactacggacagcatgcctccgttgctccggcaagctatagagctcttcgatcaccaaggcgctgagccagctttcttgttcggattggaacttattatatgcgggctcgaaaagcagcttaaatgcgagtcaggttaagcggccgctcgagtctagagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattaattctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtc

SEQ ID NO: 17 - DNA, 6904 bp, synthetic construct
  source 1..6904: mol_type = other DNA
cctgcaggcagctgcgcgctcgctcgctcactgaggccgcccgggcgtcgggcgacctttggtcgcccggcctcagtgagcgagcgagcgcgcagagagggagtggccaactccatcactaggggttcctgcggccgcacgcgtgctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgctagctaactaccggtgccaccatggccccaaagaagaagcggaaggtcggtagtactgtgggtactcgaagtggctgcgtaccacacccgtcgcatggatccaagcggaactatatcctgggactggacatcggaattacctccgtgggatacggcatcatcgattacgagactagggacgtgattgacgccggcgtgagactctttaaggaggccaacgtggaaaacaacgaaggtcgcagatccaagcggggtgcaagacgcctgaagcgccggaggagacatcggatacagcgcgtgaagaagctccttttcgactacaacctcctcactgaccactcggaattgtccggtatcaacccctacgaagcccgcgtgaaaggcctgagccagaagctgtccgaagaggagtttagcgcagccctgctgcacctggctaagcgaaggggggtgcacaacgtgaacgaggtggaggaggacactggcaacgaactgtccaccaaggagcagatttcacggaactcgaaggcgctggaagagaaatatgtggccgagctgcagctggagaggctcaagaaggatggcgaagtccgggggagcatcaatcgcttcaagacctcggactacgtgaaggaagccaaacagctgttgaaggtgcagaaggcctaccaccaactggaccaatcattcattgacacttacatcgatctgcttgaaaccaggcgcacctactacgagggtcctggagaaggcagccctttcggatggaaggacatcaaggagtggtatgagatgctgatgggtcattgcacctactttccggaagaactgcgctcagtgaagtacgcgtacaacgctgacctctacaacgctctcaacgatctgaacaacctcgtgatcacccgggacgagaacgaaaagctggagtactacgaaaagttccagattatcgaaaacgtgttcaagcagaagaagaagcccaccctgaagcagattgcaaaggagatccttgtgaacgaggaggatattaagggctaccgggtcacctccaccgggaaaccagagttcactaatctcaaggtgtaccatgacattaaggacattactgcccgcaaggagatcattgaaaacgcggaactgctggaccaaatcgcgaagatcctgaccatctatcagagctccgaggatatccaggaggaacttactaacctcaattccgagctgacgcaggaagaaatcgagcaaattagcaacctgaagggttacactggaacccacaacctcagcttgaaagcgattaaccttattttggatgaactttggcacactaatgacaatcagatcgccattttcaaccggctgaaactggtgccgaagaaggtggacctgagccaacagaaggaaatcccgaccacccttgtggacgatttcatcctgtcacctgtggtgaagaggagcttcatccagtcgatcaaggtcatcaacgccatcataaagaagtacggccttcccaacgacatcatcatcgaactggcccgcgagaagaactccaaagatgcccagaagatgatcaacgagatgcagaagcgaaaccggcagacgaacgaacggatcgaggagatcatccggaccaccgggaaggaaaacgcgaagtacctgatcgagaaaatcaagctgcatgatatgcaggaagggaagtgtctctactccctggaggccattccgctggaggatttgctgaacaaccctttcaactacgaagtcgatcatatcattcctcgctccgtgtccttcgataactccttcaacaataaggtcctcgtgaagcaggaggagaagtaagtatcaaggttacaagacaggtttaaggagaccaatagaaactgggctcgagaatgcgacgggtgtggtacgcagccacttcgagtacccacagtactacctgcttgtcgagacagagaagactcttgcgtttctgataggcacctattggtcttactgacatccactttgcctttctctccacagctcgaagaagggcaacagaaccccgttccagtacctctcgtcgtccgactccaagatcagctacgaaactttcaagaagcacattctgaacctggccaagggcaaagggagaattagcaagaccaagaaggaatacctcctggaagagagagacatcaaccgcttctcggtgcaaaaggatttcatcaaccgcaacctggtcgataccagatacgccaccaggggactgatgaacctcctgcggtcctacttccgggtcaacaatctggacgtgaaggtcaaatccatcaacgggggctttacttctttcctgcgccggaagtggaagttcaagaaggaacggaacaagggatacaagcaccacgctgaagatgccctgattattgccaacgccgacttcatctttaaggaatggaaaaagctggacaaggctaagaaggtcatggagaaccagatgttcgaagaaaagcaggccgagtccatgcccgaaatcgaaaccgagcaggaatacaaggagatcttcatcacaccgcaccaaatcaagcacatcaaggacttcaaggattacaagtacagccaccgggtggacaagaagcctaacagagagcttatcaacgacaccctgtactccacgcgcaaggacgacaagggaaacacattgatcgtgaacaacctgaacggactgtatgacaaggacaatgacaaactgaagaagctgatcaacaaatcgccggaaaagctcctgatgtaccatcacgaccctcaaacctaccagaaactgaagctcatcatggagcagtacggcgacgaaaagaatcccctgtacaaatactacgaggagactggaaattacctgactaagtactccaagaaggataacggccccgtgatcaagaagattaagtactacggaaacaaactgaacgcacatctcgacatcaccgatgattatccaaactcccgcaacaaagtcgtgaagctctccctcaaaccgtaccgcttcgacgtgtacctggataatggggtgtacaagttcgtgaccgtgaagaacctggacgtcattaagaaggaaaactactacgaagtgaactcaaagtgctacgaggaagccaagaagctcaagaagatcagcaaccaggccgagttcatcgcatcgttttacaacaatgacctcattaagattaatggagaactgtacagagtgatcggcgtgaacaacgacctcctgaaccggattgaagtgaacatgatcgatattacctaccgggagtatctggagaacatgaacgacaagcgcccaccgagaatcatcaaaactattgcctccaagacccaatccattaagaaatactccaccgacatcctgggcaacctgtacgaggtcaagtcgaagaagcacccccagattatcaagaagggaaaaaggccggcggccacgaaaaaggccggccaggcaaaaaagaaaaaggcttaagaattcctagagctcgctgatcagcctcgaaacttgtttattgcagcttataatggttacaaataaagcaatagcatcacaaatttcacaaataaagcatttttttcactgcattctagttgtggtttgtccaaactcatcaatgtatcttatcatgtctgtataccagcaggctttcctaggttcgaacgctgacgtcatcaacccgctccaaggaatcgcgggcccagtgtcactaggcgggaacacccagcgcgcgtgcgccctggcaggaagatggctgtgagggacaggggagtggcgccctgcaatatttgcatgtcgctatgtgttctgggaaatcaccataaacgtgaaatccctatcagtgatagagacttataagttccctatcagtgatagagacaccgacgggtgtggtacgcagccactgtttaagtactctgtgctggaaacagcacagaatctacttaaacaaggcaaaatgccgtgtttatctcgtcaacttgttggcgagattttttcacgtgcggaccgaggctgcagcgtcgtcctccctaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggcctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgaacaataaaactgtctgcttacataaacagtaatacaaggggtgttatgagccatattcaacgggaaacgtcgaggccgcgattaaattccaacatggatgctgatttatatgggtataaatgggctcgcgataatgtcgggcaatcaggtgcgacaatctatcgcttgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccggaaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataaacttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgt

SEQ ID NO: 18 - DNA, 8452 bp, synthetic construct
  source 1..8452: mol_type = other DNA
ctagctaactaccggtgccaccatggccccaaagaagaagcggaaggtcggtagtactgtgggtactcgaagtggctgcgtaccacacccgtcgcatggatccaagcggaactatatcctgggactggacatcggaattacctccgtgggatacggcatcatcgattacgagactagggacgtgattgacgccggcgtgagactctttaaggaggccaacgtggaaaacaacgaaggtcgcagatccaagcggggtgcaagacgcctgaagcgccggaggagacatcggatacagcgcgtgaagaagctccttttcgactacaacctcctcactgaccactcggaattgtccggtatcaacccctacgaagcccgcgtgaaaggcctgagccagaagctgtccgaagaggagtttagcgcagccctgctgcacctggctaagcgaaggggggtgcacaacgtgaacgaggtggaggaggacactggcaacgaactgtccaccaaggagcagatttcacggaactcgaaggcgctggaagagaaatatgtggccgagctgcagctggagaggctcaagaaggatggcgaagtccgggggagcatcaatcgcttcaagacctcggactacgtgaaggaagccaaacagctgttgaaggtgcagaaggcctaccaccaactggaccaatcattcattgacacttacatcgatctgcttgaaaccaggcgcacctactacgagggtcctggagaaggcagccctttcggatggaaggacatcaaggagtggtatgagatgctgatgggtcattgcacctactttccggaagaactgcgctcagtgaagtacgcgtacaacgctgacctctacaacgctctcaacgatctgaacaacctcgtgatcacccgggacgagaacgaaaagctggagtactacgaaaagttccagattatcgaaaacgtgttcaagcagaagaagaagcccaccctgaagcagattgcaaaggagatccttgtgaacgaggaggatattaagggctaccgggtcacctccaccgggaaaccagagttcactaatctcaaggtgtaccatgacattaaggacattactgcccgcaaggagatcattgaaaacgcggaactgctggaccaaatcgcgaagatcctgaccatctatcagagctccgaggatatccaggaggaacttactaacctcaattccgagctgacgcaggaagaaatcgagcaaattagcaacctgaagggttacactggaacccacaacctcagcttgaaagcgattaaccttattttggatgaactttggcacactaatgacaatcagatcgccattttcaaccggctgaaactggtgccgaagaaggtggacctgagccaacagaaggaaatcccgaccacccttgtggacgatttcatcctgtcacctgtggtgaagaggagcttcatccagtcgatcaaggtcatcaacgccatcataaagaagtacggccttcccaacgacatcatcatcgaactggcccgcgagaagaactccaaagatgcccagaagatgatcaacgagatgcagaagcgaaaccggcagacgaacgaacggatcgaggagatcatccggaccaccgggaaggaaaacgcgaagtacctgatcgagaaaatcaagctgcatgatatgcaggaagggaagtgtctctactccctggaggccattccgctggaggatttgctgaacaaccctttcaactacgaagtcgatcatatcattcctcgctccgtgtccttcgataactccttcaacaataaggtcctcgtgaagcaggaggagaagtaagtatcaaggttacaagacaggtttaaggagaccaatagaaactgggctcgagaatgcgacgggtgtggtacgcagccacttcgagtacccacagtactacctgcttgtcgagacagagaagactcttgcgtttctgataggcacctattggtcttactgacatccactttgcctttctctccacagctcgaagaagggcaacagaaccccgttccagtacctctcgtcgtccgactccaagatcagctacgaaactttcaagaagcacattctgaacctggccaagggcaaagggagaattagcaagaccaagaaggaatacctcctggaagagagagacatcaaccgcttctcggtgcaaaaggatttcatcaaccgcaacctggtcgataccagatacgccaccaggggactgatgaacctcctgcggtcctacttccgggtcaacaatctggacgtgaaggtcaaatccatcaacgggggctttacttctttcctgcgccggaagtggaagttcaagaaggaacggaacaagggatacaagcaccacgctgaagatgccctgattattgccaacgccgacttcatctttaaggaatggaaaaagctggacaaggctaagaaggtcatggagaaccagatgttcgaagaaaagcaggccgagtccatgcccgaaatcgaaaccgagcaggaatacaaggagatcttcatcacaccgcaccaaatcaagcacatcaaggacttcaaggattacaagtacagccaccgggtggacaagaagcctaacagagagcttatcaacgacaccctgtactccacgcgcaaggacgacaagggaaacacattgatcgtgaacaacctgaacggactgtatgacaaggacaatgacaaactgaagaagctgatcaacaaatcgccggaaaagctcctgatgtaccatcacgaccctcaaacctaccagaaactgaagctcatcatggagcagtacggcgacgaaaagaatcccctgtacaaatactacgaggagactggaaattacctgactaagtactccaagaaggataacggccccgtgatcaagaagattaagtactacggaaacaaactgaacgcacatctcgacatcaccgatgattatccaaactcccgcaacaaagtcgtgaagctctccctcaaaccgtaccgcttcgacgtgtacctggataatggggtgtacaagttcgtgaccgtgaagaacctggacgtcattaagaaggaaaactactacgaagtgaactcaaagtgctacgaggaagccaagaagctcaagaagatcagcaaccaggccgagttcatcgcatcgttttacaacaatgacctcattaagattaatggagaactgtacagagtgatcggcgtgaacaacgacctcctgaaccggattgaagtgaacatgatcgatattacctaccgggagtatctggagaacatgaacgacaagcgcccaccgagaatcatcaaaactattgcctccaagacccaatccattaagaaatactccaccgacatcctgggcaacctgtacgaggtcaagtcgaagaagcacccccagattatcaagaagggaaaaaggccggcggccacgaaaaaggccggccaggcaaaaaagaaaaaggcttaagaattcctagagctcgctgatcagcctcgaaacttgtttattgcagcttataatggttacaaataaagcaatagcatcacaaatttcacaaataaagcatttttttcactgcattctagttgtggtttgtccaaactcatcaatgtatcttatcatgtctgtataccagcaggctttcctaggttcgaacgctgacgtcatcaacccgctccaaggaatcgcgggcccagtgtcactaggcgggaacacccagcgcgcgtgcgccctggcaggaagatggctgtgagggacaggggagtggcgccctgcaatatttgcatgtcgctatgtgttctgggaaatcaccataaacgtgaaatccctatcagtgatagagacttataagttccctatcagtgatagagacaccgacgggtgtggtacgcagccactgtttaagtactctgtgctggaaacagcacagaatctacttaaacaaggcaaaatgccgtgtttatctcgtcaacttgttggcgagattttttcacgtgcggaccgaggctgcagcgtcgtcctccctaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggcctattggttaaaaaatgagctgatttaacaaaaatttaacgcgaattttaacaaaatattaacgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgttaattaaccggtgccaccatgatgtcccgccttgataaatcaaaagtaataaacagtgctcttgagcttctcaatgaagttggtatagaagggttgacgactcggaaattggcgcaaaaactcggtgttgagcagccaaccttgtattggcatgttaaaaacaaacgagcactcctcgacgctttggcgatagagatgctggacaggcaccacacgcatttctgtcccctcgaaggagagtcatggcaggatttccttagaaataacgcaaagtccttcagatgtgcgctgcttagtcaccgcgacggcgcaaaagttcatctcggcactaggccaaccgagaaacagtacgagactctggagaaccaactggcgtttttgtgtcaacagggttttagtctcgaaaatgcgctctatgctctctctgcggttggccatttcaccctcggatgcgtactggaagatcaggagcaccaagtggccaaagaagaacgggaaacgccgactacggacagcatgcctccgttgctccggcaagctatagagctcttcgatcaccaaggcgctgagccagctttcttgttcggattggaacttattatatgcgggctcgaaaagcagcttaaatgcgagtcaggttaagcggccgctcgagtctagagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgaacaataaaactgtctgcttacataaacagtaatacaaggggtgttatgagccatattcaacgggaaacgtcgaggccgcgattaaattccaacatggatgctgatttatatgggtataaatgggctcgcgataatgtcgggcaatcaggtgcgacaatctatcgcttgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccggaaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataaacttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgtcctgcaggcagctgcgcgctcgctcgctcactgaggccgcccgggcgtcgggcgacctttggtcgcccggcctcagtgagcgagcgagcgcgcagagagggagtggccaactccatcactaggggttcctgcggccgcacgcgtgctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacg

SEQ ID NO: 19 - DNA, 6987 bp, synthetic construct
  source 1..6987: mol_type = other DNA
cctgcaggcagctgcgcgctcgctcgctcactgaggccgcccgggcgtcgggcgacctttggtcgcccggcctcagtgagcgagcgagcgcgcagagagggagtggccaactccatcactaggggttcctgcggccgcacgcgtgggccccagaagcctggtggttgtttgtccttctcaggggaaaagtgaggcggccccttggaggaaggggccgggcagaatgatctaatcggattccaagcagctcaggggattgtctttttctagcaccttcttgccactcctaagcgtcctccgtgaccccggctgggatttagcctggtgctgtgtcagccccggtctcccaggggcttcccagtggtccccaggaaccctcgacagggcccggtctctctcgtccagcaagggcagggacgggccacaggccaagggcgctagctaactaccggtgccaccatggccccaaagaagaagcggaaggtcggtagtactgtgggtactcgaagtggctgcgtaccacacccgtcgcatggatccaagcggaactatatcctgggactggacatcggaattacctccgtgggatacggcatcatcgattacgagactagggacgtgattgacgccggcgtgagactctttaaggaggccaacgtggaaaacaacgaaggtcgcagatccaagcggggtgcaagacgcctgaagcgccggaggagacatcggatacagcgcgtgaagaagctccttttcgactacaacctcctcactgaccactcggaattgtccggtatcaacccctacgaagcccgcgtgaaaggcctgagccagaagctgtccgaagaggagtttagcgcagccctgctgcacctggctaagcgaaggggggtgcacaacgtgaacgaggtggaggaggacactggcaacgaactgtccaccaaggagcagatttcacggaactcgaaggcgctggaagagaaatatgtggccgagctgcagctggagaggctcaagaaggatggcgaagtccgggggagcatcaatcgcttcaagacctcggactacgtgaaggaagccaaacagctgttgaaggtgcagaaggcctaccaccaactggaccaatcattcattgacacttacatcgatctgcttgaaaccaggcgcacctactacgagggtcctggagaaggcagccctttcggatggaaggacatcaaggagtggtatgagatgctgatgggtcattgcacctactttccggaagaactgcgctcagtgaagtacgcgtacaacgctgacctctacaacgctctcaacgatctgaacaacctcgtgatcacccgggacgagaacgaaaagctggagtactacgaaaagttccagattatcgaaaacgtgttcaagcagaagaagaagcccaccctgaagcagattgcaaaggagatccttgtgaacgaggaggatattaagggctaccgggtcacctccaccgggaaaccagagttcactaatctcaaggtgtaccatgacattaaggacattactgcccgcaaggagatcattgaaaacgcggaactgctggaccaaatcgcgaagatcctgaccatctatcagagctccgaggatatccaggaggaacttactaacctcaattccgagctgacgcaggaagaaatcgagcaaattagcaacctgaagggttacactggaacccacaacctcagcttgaaagcgattaaccttattttggatgaactttggcacactaatgacaatcagatcgccattttcaaccggctgaaactggtgccgaagaaggtggacctgagccaacagaaggaaatcccgaccacccttgtggacgatttcatcctgtcacctgtggtgaagaggagcttcatccagtcgatcaaggtcatcaacgccatcataaagaagtacggccttcccaacgacatcatcatcgaactggcccgcgagaagaactccaaagatgcccagaagatgatcaacgagatgcagaagcgaaaccggcagacgaacgaacggatcgaggagatcatccggaccaccgggaaggaaaacgcgaagtacctgatcgagaaaatcaagctgcatgatatgcaggaagggaagtgtctctactccctggaggccattccgctggaggatttgctgaacaaccctttcaactacgaagtcgatcatatcattcctcgctccgtgtccttcgataactccttcaacaataaggtcctcgtgaagcaggaggagaagtaagtatcaaggttacaagacaggtttaaggagaccaatagaaactgggctcgagaatgcgacgggtgtggtacgcagccacttcgagtacccacagtactacctgcttgtcgagacagagaagactcttgcgtttctgataggcacctattggtcttactgacatccactttgcctttctctccacagctcgaagaagggcaacagaaccccgttccagtacctctcgtcgtccgactccaagatcagctacgaaactttcaagaagcacattctgaacctggccaagggcaaagggagaattagcaagaccaagaaggaatacctcctggaagagagagacatcaaccgcttctcggtgcaaaaggatttcatcaaccgcaacctggtcgataccagatacgccaccaggggactgatgaacctcctgcggtcctacttccgggtcaacaatctggacgtgaaggtcaaatccatcaacgggggctttacttctttcctgcgccggaagtggaagttcaagaaggaacggaacaagggatacaagcaccacgctgaagatgccctgattattgccaacgccgacttcatctttaaggaatggaaaaagctggacaaggctaagaaggtcatggagaaccagatgttcgaagaaaagcaggccgagtccatgcccgaaatcgaaaccgagcaggaatacaaggagatcttcatcacaccgcaccaaatcaagcacatcaaggacttcaaggattacaagtacagccaccgggtggacaagaagcctaacagagagcttatcaacgacaccctgtactccacgcgcaaggacgacaagggaaacacattgatcgtgaacaacctgaacggactgtatgacaaggacaatgacaaactgaagaagctgatcaacaaatcgccggaaaagctcctgatgtaccatcacgaccctcaaacctaccagaaactgaagctcatcatggagcagtacggcgacgaaaagaatcccctgtacaaatactacgaggagactggaaattacctgactaagtactccaagaaggataacggccccgtgatcaagaagattaagtactacggaaacaaactgaacgcacatctcgacatcaccgatgattatccaaactcccgcaacaaagtcgtgaagctctccctcaaaccgtaccgcttcgacgtgtacctggataatggggtgtacaagttcgtgaccgtgaagaacctggacgtcattaagaaggaaaactactacgaagtgaactcaaagtgctacgaggaagccaagaagctcaagaagatcagcaaccaggccgagttcatcgcatcgttttacaacaatgacctcattaagattaatggagaactgtacagagtgatcggcgtgaacaacgacctcctgaaccggattgaagtgaacatgatcgatattacctaccgggagtatctggagaacatgaacgacaagcgcccaccgagaatcatcaaaactattgcctccaagacccaatccattaagaaatactccaccgacatcctgggcaacctgtacgaggtcaagtcgaagaagcacccccagattatcaagaagggaaaaaggccggcggccacgaaaaaggccggccaggcaaaaaagaaaaaggcttaagaattcctagagctcgctgatcagcctcgaaacttgtttattgcagcttataatggttacaaataaagcaatagcatcacaaatttcacaaataaagcatttttttcactgcattctagttgtggtttgtccaaactcatcaatgtatcttatcatgtctgtataccagcaggctttcctaggttcgaacgctgacgtcatcaacccgctccaaggaatcgcgggcccagtgtcactaggcgggaacacccagcgcgcgtgcgccctggcaggaagatggctgtgagggacaggggagtggcgccctgcaatatttgcatgtcgctatgtgttctgggaaatcaccataaacgtgaaatccctatcagtgatagagacttataagttccctatcagtgatagagacaccgacgggtgtggtacgcagccactgtttaagtactctgtgctggaaacagcacagaatctacttaaacaaggcaaaatgccgtgtttatctcgtcaacttgttggcgagattttttcacgtgcggaccgaggctgcagcgtcgtcctccctaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggcctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgaacaataaaactgtctgcttacataaacagtaatacaaggggtgttatgagccatattcaacgggaaacgtcgaggccgcgattaaattccaacatggatgctgatttatatgggtataaatgggctcgcgataatgtcgggcaatcaggtgcgacaatctatcgcttgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccggaaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataaacttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgt

SEQ ID NO: 20 - DNA, 8535 bp, synthetic construct
  source 1..8535: mol_type = other DNA
cctgcaggcagctgcgcgctcgctcgctcactgaggccgcccgggcgtcgggcgacctttggtcgcccggcctcagtgagcgagcgagcgcgcagagagggagtggccaactccatcactaggggttcctgcggccgcacgcgtgggccccagaagcctggtggttgtttgtccttctcaggggaaaagtgaggcggccccttggaggaaggggccgggcagaatgatctaatcggattccaagcagctcaggggattgtctttttctagcaccttcttgccactcctaagcgtcctccgtgaccccggctgggatttagcctggtgctgtgtcagccccggtctcccaggggcttcccagtggtccccaggaaccctcgacagggcccggtctctctcgtccagcaagggcagggacgggccacaggccaagggcgctagctaactaccggtgccaccatggccccaaagaagaagcggaaggtcggtagtactgtgggtactcgaagtggctgcgtaccacacccgtcgcatggatccaagcggaactatatcctgggactggacatcggaattacctccgtgggatacggcatcatcgattacgagactagggacgtgattgacgccggcgtgagactctttaaggaggccaacgtggaaaacaacgaaggtcgcagatccaagcggggtgcaagacgcctgaagcgccggaggagacatcggatacagcgcgtgaagaagctccttttcgactacaacctcctcactgaccactcggaattgtccggtatcaacccctacgaagcccgcgtgaaaggcctgagccagaagctgtccgaagaggagtttagcgcagccctgctgcacctggctaagcgaaggggggtgcacaacgtgaacgaggtggaggaggacactggcaacgaactgtccaccaaggagcagatttcacggaactcgaaggcgctggaagagaaatatgtggccgagctgcagctggagaggctcaagaaggatggcgaagtccgggggagcatcaatcgcttcaagacctcggactacgtgaaggaagccaaacagctgttgaaggtgcagaaggcctaccaccaactggaccaatcattcattgacacttacatcgatctgcttgaaaccaggcgcacctactacgagggtcctggagaaggcagccctttcggatggaaggacatcaaggagtggtatgagatgctgatgggtcattgcacctactttccggaagaactgcgctcagtgaagtacgcgtacaacgctgacctctacaacgctctcaacgatctgaacaacctcgtgatcacccgggacgagaacgaaaagctggagtactacgaaaagttccagattatcgaaaacgtgttcaagcagaagaagaagcccaccctgaagcagattgcaaaggagatccttgtgaacgaggaggatattaagggctaccgggtcacctccaccgggaaaccagagttcactaatctcaaggtgtaccatgacattaaggacattactgcccgcaaggagatcattgaaaacgcggaactgctggaccaaatcgcgaagatcctgaccatctatcagagctccgaggatatccaggaggaacttactaacctcaattccgagctgacgcaggaagaaatcgagcaaattagcaacctgaagggttacactggaacccacaacctcagcttgaaagcgattaaccttattttggatgaactttggcacactaatgacaatcagatcgccattttcaaccggctgaaactggtgccgaagaaggtggacctgagccaacagaaggaaatcccgaccacccttgtggacgatttcatcctgtcacctgtggtgaagaggagcttcatccagtcgatcaaggtcatcaacgccatcataaagaagtacggccttcccaacgacatcatcatcgaactggcccgcgagaagaactccaaagatgcccagaagatgatcaacgagatgcagaagcgaaaccggcagacgaacgaacggatcgaggagatcatccggaccaccgggaaggaaaacgcgaagtacctgatcgagaaaatcaagctgcatgatatgcaggaagggaagtgtctctactccctggaggccattccgctggaggatttgctgaacaaccctttcaactacgaagtcgatcatatcattcctcgctccgtgtccttcgataactccttcaacaataaggtcctcgtgaagcaggaggagaagtaagtatcaaggttacaagacaggtttaaggagaccaatagaaactgggctcgagaatgcgacgggtgtggtacgcagccacttcgagtacccacagtactacctgcttgtcgagacagagaagactcttgcgtttctgataggcacctattggtcttactgacatccactttgcctttctctccacagctcgaagaagggcaacagaaccccgttccagtacctctcgtcgtccgactccaagatcagctacgaaactttcaagaagcacattctgaacctggccaagggcaaagggagaattagcaagaccaagaaggaatacctcctggaagagagagacatcaaccgcttctcggtgcaaaaggatttcatcaaccgcaacctggtcgataccagatacgccaccaggggactgatgaacctcctgcggtcctacttccgggtcaacaatctggacgtgaaggtcaaatccatcaacgggggctttacttctttcctgcgccggaagtggaagttcaagaaggaacggaacaagggatacaagcaccacgctgaagatgccctgattattgccaacgccgacttcatctttaaggaatggaaaaagctggacaaggctaagaaggtcatggagaaccagatgttcgaagaaaagcaggccgagtccatgcccgaaatcgaaaccgagcaggaatacaaggagatcttcatcacaccgcaccaaatcaagcacatcaaggacttcaaggattacaagtacagccaccgggtggacaagaagcctaacagagagcttatcaacgacaccctgtactccacgcgcaaggacgacaagggaaacacattgatcgtgaacaacctgaacggactgtatgacaaggacaatgacaaactgaagaagctgatcaacaaatcgccggaaaagctcctgatgtaccatcacgaccctcaaacctaccagaaactgaagctcatcatggagcagtacggcgacgaaaagaatcccctgtacaaatactacgaggagactggaaattacctgactaagtactccaagaaggataacggccccgtgatcaagaagattaagtactacggaaacaaactgaacgcacatctcgacatcaccgatgattatccaaactcccgcaacaaagtcgtgaagctctccctcaaaccgtaccgcttcgacgtgtacctggataatggggtgtacaagttcgtgaccgtgaagaacctggacgtcattaagaaggaaaactactacgaagtgaactcaaagtgctacgaggaagccaagaagctcaagaagatcagcaaccaggccgagttcatcgcatcgttttacaacaatgacctcattaagattaatggagaactgtacagagtgatcggcgtgaacaacgacctcctgaaccggattgaagtgaacatgatcgatattacctaccgggagtatctggagaacatgaacgacaagcgcccaccgagaatcatcaaaactattgcctccaagacccaatccattaagaaatactccaccgacatcctgggcaacctgtacgaggtcaagtcgaagaagcacccccagattatcaagaagggaaaaaggccggcggccacgaaaaaggccggccaggcaaaaaagaaaaaggcttaagaattcctagagctcgctgatcagcctcgaaacttgtttattgcagcttataatggttacaaataaagcaatagcatcacaaatttcacaaataaagcatttttttcactgcattctagttgtggtttgtccaaactcatcaatgtatcttatcatgtctgtataccagcaggctttcctaggttcgaacgctgacgtcatcaacccgctccaaggaatcgcgggcccagtgtcactaggcgggaacacccagcgcgcgtgcgccctggcaggaagatggctgtgagggacaggggagtggcgccctgcaatatttgcatgtcgctatgtgttctgggaaatcaccataaacgtgaaatccctatcagtgatagagacttataagttccctatcagtgatagagacaccgacgggtgtggtacgcagccactgtttaagtactctgtgctggaaacagcacagaatctacttaaacaaggcaaaatgccgtgtttatctcgtcaacttgttggcgagattttttcacgtgcggaccgaggctgcagcgtcgtcctccctaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggcctattggttaaaaaatgagctgatttaacaaaaatttaacgcgaattttaacaaaatattaacgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgttaattaaccggtgccaccatgatgtcccgccttgataaatcaaaagtaataaacagtgctcttgagcttctcaatgaagttggtatagaagggttgacgactcggaaattggcgcaaaaactcggtgttgagcagccaaccttgtattggcatgttaaaaacaaacgagcactcctcgacgctttggcgatagagatgctggacaggcaccacacgcatttctgtcccctcgaaggagagtcatggcaggatttccttagaaataacgcaaagtccttcagatgtgcgctgcttagtcaccgcgacggcgcaaaagttcatctcggcactaggccaaccgagaaacagtacgagactctggagaaccaactggcgtttttgtgtcaacagggttttagtctcgaaaatgcgctctatgctctctctgcggttggccatttcaccctcggatgcgtactggaagatcaggagcaccaagtggccaaagaagaacgggaaacgccgactacggacagcatgcctccgttgctccggcaagctatagagctcttcgatcaccaaggcgctgagccagctttcttgttcggattggaacttattatatgcgggctcgaaaagcagcttaaatgcgagtcaggttaagcggccgctcgagtctagagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgaacaataaaactgtctgcttacataaacagtaatacaaggggtgttatgagccatattcaacgggaaacgtcgaggccgcgattaaattccaacatggatgctgatttatatgggtataaatgggctcgcgataatgtcgggcaatcaggtgcgacaatctatcgcttgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccggaaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataaacttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgt

SEQ ID NO: 21 - DNA, 23 bp, synthetic construct
  source 1..23: mol_type = other DNA
ctagctaactaccggtgccacca

SEQ ID NO: 22 - DNA, 23 bp, synthetic construct
  source 1..23: mol_type = other DNA
gaggtattccttcttggtcttgc

SEQ ID NO: 23 - DNA, 21 bp, synthetic construct
  source 1..21: mol_type = other DNA
cccacagtccccgagaagttg

SEQ ID NO: 24 - DNA, 21 bp, synthetic construct
  source 1..21: mol_type = other DNA
accttcttgccactcctaagc

SEQ ID NO: 25 - DNA, 21 bp, synthetic construct
  source 1..21: mol_type = other DNA
cttctggctcaggcctttcac

SEQ ID NO: 26 - DNA, 23 bp, synthetic construct
  source 1..23: mol_type = other DNA
cctgatcgagaaaatcaagctgc

SEQ ID NO: 27 - DNA, 23 bp, synthetic construct
  source 1..23: mol_type = other DNA
gcaagaccaagaaggaatacctc

SEQ ID NO: 28 - DNA, 1654 bp, synthetic construct
  source 1..1654: mol_type = other DNA
ggtagtggtgaagggcgaggctccttgctgacctgtggagatgttgaagagaatcccggcccaggtaccatgcgacgggtgtggtacgcagccacttcgagtacccacagtactgctagcgtgtctaagggcgaagagctgattaaggagaacatgcacatgaagctgtacatggagggcaccgtggacaaccatcacttcaagtgcacatccgagggcgaaggcaagccctacgagggcacccagaccatgagaatcaaggtggtcgagggcggccctctccccttcgccttcgacatcctggctactagcttcctctacggcagcaagaccttcatcaaccacacccagggcatccccgacttcttcaagcagtccttccctgagggcttcacatgggagagagtcaccacatacgaagacgggggcgtgctgaccgctacccaggacaccagcctccaggacggctgcctcatctacaacgtcaagatcagaggggtgaacttcacatccaacggccctgtgatgcagaagaaaacactcggctgggaggccttcaccgagacgctgtaccccgctgacggcggcctggaaggcagaaacgacatggccctgaagctcgtgggcgggagccatctgatcgcaaacgccaagaccacatatagatccaagaaacccgctaagaacctcaagatgcctggcgtctactatgtggactacagactggaaagaatcaaggaggccaacaacgagacctacgtcgagcagcacgaggtggcagtggccagatactgcgacctccctagcaaactggggcacaaacttaataagcttagccatggcttcccgccggaggtggaggagcaggatgatggcacgctgcccatgtcttgtgcccaggagagcgggatggaccgtcaccctgcagcctgtgcttctgctaggatcaatgtgcacccgggaagcggggctaccaacttcagtctcctcaaacaagctggcgatgtggaggaaaatccaggtccggttcctttgtctcaagaagaatccaccctcattgaaagagcaacggctacaatcaacagcatccccatctctgaagactacagcgtcgccagcgcagctctctctagcgacggccgcatcttcactggtgtcaatgtatatcattttactgggggaccttgtgcagaactcgtggtgctgggcactgctgctgctgcggcagctggcaacctgacttgtatcgtcgcgatcggaaatgagaacaggggcatcttgagcccctgcggacggtgccgacaggtgcttctcgatctgcatcctgggatcaaagccatagtgaaggacagtgatggacagccgacggcagttgggattcgtgaattgctgccctctggttatgtgtgggagggctagagatctcgagctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgg

SEQ ID NO: 29 - DNA, 1542 bp, synthetic construct
  source 1..1542: mol_type = other DNA
gacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgttaattaaccggtgccaccatgatgtcccgccttgataaatcaaaagtaataaacagtgctcttgagcttctcaatgaagttggtatagaagggttgacgactcggaaattggcgcaaaaactcggtgttgagcagccaaccttgtattggcatgttaaaaacaaacgagcactcctcgacgctttggcgatagagatgctggacaggcaccacacgcatttctgtcccctcgaaggagagtcatggcaggatttccttagaaataacgcaaagtccttcagatgtgcgctgcttagtcaccgcgacggcgcaaaagttcatctcggcactaggccaaccgagaaacagtacgagactctggagaaccaactggcgtttttgtgtcaacagggttttagtctcgaaaatgcgctctatgctctctctgcggttggccatttcaccctcggatgcgtactggaagatcaggagcaccaagtggccaaagaagaacgggaaacgccgactacggacagcatgcctccgttgctccggcaagctatagagctcttcgatcaccaaggcgctgagccagctttcttgttcggattggaacttattatatgcgggctcgaaaagcagcttaaatgcgagtcaggttaagcggccgctcgagtctagagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgg

SEQ ID NO: 30 - RNA, 23 nt, synthetic construct
  source 1..23: mol_type = other RNA
gacgggtgtggtacgcagccact

SEQ ID NO: 31 - DNA, 23 bp, synthetic construct
  source 1..23: mol_type = other DNA
gacgggtgtggtacgcagccact

SEQ ID NO: 32 - DNA, 23 bp, synthetic construct
  source 1..23: mol_type = other DNA
agtggctgcgtaccacacccgtc

SEQ ID NO: 33 - RNA, 20 nt, synthetic construct
  source 1..20: mol_type = other RNA
gggtgtggtacgcagccact

SEQ ID NO: 34 - DNA, 20 bp, synthetic construct
  source 1..20: mol_type = other DNA
gggtgtggtacgcagccact

SEQ ID NO: 35 - DNA, 20 bp, synthetic construct
  source 1..20: mol_type = other DNA
agtggctgcgtaccacaccc

SEQ ID NO: 36 - DNA, 8457 bp, synthetic construct
  source 1..8457: mol_type = other DNA
cctgcaggcagctgcgcgctcgctcgctcactgaggccgcccgggcgtcgggcgacctttggtcgcccggcctcagtgagcgagcgagcgcgcagagagggagtggccaactccatcactaggggttcctgcggccgcacgcgtgggccccagaagcctggtggttgtttgtccttctcaggggaaaagtgaggcggccccttggaggaaggggccgggcagaatgatctaatcggattccaagcagctcaggggattgtctttttctagcaccttcttgccactcctaagcgtcctccgtgaccccggctgggatttagcctggtgctgtgtcagccccggctagctaactaccggtgccaccatggccccaaagaagaagcggaaggtcggtagtactgtgggtactcgaagtggctgcgtaccacacccgtcgcatggatccaagcggaactatatcctgggactggacatcggaattacctccgtgggatacggcatcatcgattacgagactagggacgtgattgacgccggcgtgagactctttaaggaggccaacgtggaaaacaacgaaggtcgcagatccaagcggggtgcaagacgcctgaagcgccggaggagacatcggatacagcgcgtgaagaagctccttttcgactacaacctcctcactgaccactcggaattgtccggtatcaacccctacgaagcccgcgtgaaaggcctgagccagaagctgtccgaagaggagtttagcgcagccctgctgcacctggctaagcgaaggggggtgcacaacgtgaacgaggtggaggaggacactggcaacgaactgtccaccaaggagcagatttcacggaactcgaaggcgctggaagagaaatatgtggccgagctgcagctggagaggctcaagaaggatggcgaagtccgggggagcatcaatcgcttcaagacctcggactacgtgaaggaagccaaacagctgttgaaggtgcagaaggcctaccaccaactggaccaatcattcattgacacttacatcgatctgcttgaaaccaggcgcacctactacgagggtcctggagaaggcagccctttcggatggaaggacatcaaggagtggtatgagatgctgatgggtcattgcacctactttccggaagaactgcgctcagtgaagtacgcgtacaacgctgacctctacaacgctctcaacgatctgaacaacctcgtgatcacccgggacgagaacgaaaagctggagtactacgaaaagttccagattatcgaaaacgtgttcaagcagaagaagaagcccaccctgaagcagattgcaaaggagatccttgtgaacgaggaggatattaagggctaccgggtcacctccaccgggaaaccagagttcactaatctcaaggtgtaccatgacattaaggacattactgcccgcaaggagatcattgaaaacgcggaactgctggaccaaatcgcgaagatcctgaccatctatcagagctccgaggatatccaggaggaacttactaacctcaattccgagctgacgcaggaagaaatcgagcaaattagcaacctgaagggttacactggaacccacaacctcagcttgaaagcgattaaccttattttggatgaactttggcacactaatgacaatcagatcgccattttcaaccggctgaaactggtgccgaagaaggtggacctgagccaacagaaggaaatcccgaccacccttgtggacgatttcatcctgtcacctgtggtgaagaggagcttcatccagtcgatcaaggtcatcaacgccatcataaagaagtacggccttcccaacgacatcatcatcgaactggcccgcgagaagaactccaaagatgcccagaagatgatcaacgagatgcagaagcgaaaccggcagacgaacgaacggatcgaggagatcatccggaccaccgggaaggaaaacgcgaagtacctgatcgagaaaatcaagctgcatgatatgcaggaagggaagtgtctctactccctggaggccattccgctggaggatttgctgaacaaccctttcaactacgaagtcgatcatatcattcctcgctccgtgtccttcgataactccttcaacaataaggtcctcgtgaagcaggaggagaagtaagtatcaaggttacaagacaggtttaaggagaccaatagaaactgggctcgagaatgcgacgggtgtggtacgcagccacttcgagtacccacagtactacctgcttgtcgagacagagaagactcttgcgtttctgataggcacctattggtcttactgacatccactttgcctttctctccacagctcgaagaagggcaacagaaccccgttccagtacctctcgtcgtccgactccaagatcagctacgaaactttcaagaagcacattctgaacctggccaagggcaaagggagaattagcaagaccaagaaggaatacctcctggaagagagagacatcaaccgcttctcggtgcaaaaggatttcatcaaccgcaacctggtcgataccagatacgccaccaggggactgatgaacctcctgcggtcctacttccgggtcaacaatctggacgtgaaggtcaaatccatcaacgggggctttacttctttcctgcgccggaagtggaagttcaagaaggaacggaacaagggatacaagcaccacgctgaagatgccctgattattgccaacgccgacttcatctttaaggaatggaaaaagctggacaaggctaagaaggtcatggagaaccagatgttcgaagaaaagcaggccgagtccatgcccgaaatcgaaaccgagcaggaatacaaggagatcttcatcacaccgcaccaaatcaagcacatcaaggacttcaaggattacaagtacagccaccgggtggacaagaagcctaacagagagcttatcaacgacaccctgtactccacgcgcaaggacgacaagggaaacacattgatcgtgaacaacctgaacggactgtatgacaaggacaatgacaaactgaagaagctgatcaacaaatcgccggaaaagctcctgatgtaccatcacgaccctcaaacctaccagaaactgaagctcatcatggagcagtacggcgacgaaaagaatcccctgtacaaatactacgaggagactggaaattacctgactaagtactccaagaaggataacggccccgtgatcaagaagattaagtactacggaaacaaactgaacgcacatctcgacatcaccgatgattatccaaactcccgcaacaaagtcgtgaagctctccctcaaaccgtaccgcttcgacgtgtacctggataatggggtgtacaagttcgtgaccgtgaagaacctggacgtcattaagaaggaaaactactacgaagtgaactcaaagtgctacgaggaagccaagaagctcaagaagatcagcaaccaggccgagttcatcgcatcgttttacaacaatgacctcattaagattaatggagaactgtacagagtgatcggcgtgaacaacgacctcctgaaccggattgaagtgaacatgatcgatattacctaccgggagtatctggagaacatgaacgacaagcgcccaccgagaatcatcaaaactattgcctccaagacccaatccattaagaaatactccaccgacatcctgggcaacctgtacgaggtcaagtcgaagaagcacccccagattatcaagaagggaaaaaggccggcggccacgaaaaaggccggccaggcaaaaaagaaaaaggcttaagaattcctagagctcgctgatcagcctcgaaacttgtttattgcagcttataatggttacaaataaagcaatagcatcacaaatttcacaaataaagcatttttttcactgcattctagttgtggtttgtccaaactcatcaatgtatcttatcatgtctgtataccagcaggctttcctaggttcgaacgctgacgtcatcaacccgctccaaggaatcgcgggcccagtgtcactaggcgggaacacccagcgcgcgtgcgccctggcaggaagatggctgtgagggacaggggagtggcgccctgcaatatttgcatgtcgctatgtgttctgggaaatcaccataaacgtgaaatccctatcagtgatagagacttataagttccctatcagtgatagagacaccgggtgtggtacgcagccactgtttaagtactctgtgctggaaacagcacagaatctacttaaacaaggcaaaatgccgtgtttatctcgtcaacttgttggcgagattttttcacgtgcggaccgaggctgcagcgtcgtcctccctaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccttagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaactctatctcgggctattcttttgatttataagggattttgccgatttcggtctattggttaaaaaatgagctgatttaacaaaaatttaacgcgaattttaacaaaatattaacgttagcgctgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgttaattaaccggtgccaccatgatgtcccgccttgataaatcaaaagtaataaacagtgctcttgagcttctcaatgaagttggtatagaagggttgacgactcggaaattggcgcaaaaactcggtgttgagcagccaaccttgtattggcatgttaaaaacaaacgagcactcctcgacgctttggcgatagagatgctggacaggcaccacacgcatttctgtcccctcgaaggagagtcatggcaggatttccttagaaataacgcaaagtccttcagatgtgcgctgcttagtcaccgcgacggcgcaaaagttcatctcggcactaggccaaccgagaaacagtacgagactctggagaaccaactggcgtttttgtgtcaacagggttttagtctcgaaaatgcgctctatgctctctctgcggttggccatttcaccctcggatgcgtactggaagatcaggagcaccaagtggccaaagaagaacgggaaacgccgactacggacagcatgcctccgttgctccggcaagctatagagctcttcgatcaccaaggcgctgagccagctttcttgttcggattggaacttattatatgcgggctcgaaaagcagcttaaatgcgagtcaggttaagtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgagttagtccattgtcgacttccgaggaccgcggaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgaacaataaaactgtctgcttacataaacagtaatacaaggggtgttatgagccatattcaacgggaaacgtcgaggccgcgattaaattccaacatggatgctgatttatatgggtataaatgggctcgcgataatgtcgggcaatcaggtgcgacaatctatcgcttgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccggaaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataaacttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgt

SEQ ID NO: 37 - DNA, 7230 bp, synthetic construct
  source 1..7230: mol_type = other DNA
cctgcaggcagctgcgcgctcgctcgctcactgaggccgcccgggcgtcgggcgacctttggtcgcccggcctcagtgagcgagcgagcgcgcagagagggagtggccaactccatcactaggggttcctgcggccgcacgcgtgggccccagaagcctggtggttgtttgtccttctcaggggaaaagtgaggcggccccttggaggaaggggccgggcagaatgatctaatcggattccaagcagctcaggggattgtctttttctagcaccttcttgccactcctaagcgtcctccgtgaccccggctgggatttagcctggtgctgtgtcagccccggctagctaactaccggtgccaccatggccccaaagaagaagcggaaggtcggtagtactgtgggtactcgaagtggctgcgtaccacacccgtcgcatggatccaagcggaactatatcctgggactggacatcggaattacctccgtgggatacggcatcatcgattacgagactagggacgtgattgacgccggcgtgagactctttaaggaggccaacgtggaaaacaacgaaggtcgcagatccaagcggggtgcaagacgcctgaagcgccggaggagacatcggatacagcgcgtgaagaagctccttttcgactacaacctcctcactgaccactcggaattgtccggtatcaacccctacgaagcccgcgtgaaaggcctgagccagaagctgtccgaagaggagtttagcgcagccctgctgcacctggctaagcgaaggggggtgcacaacgtgaacgaggtggaggaggacactggcaacgaactgtccaccaaggagcagatttcacggaactcgaaggcgctggaagagaaatatgtggccgagctgcagctggagaggctcaagaaggatggcgaagtccgggggagcatcaatcgcttcaagacctcggactacgtgaaggaagccaaacagctgttgaaggtgcagaaggcctaccaccaactggaccaatcattcattgacacttacatcgatctgcttgaaaccaggcgcacctactacgagggtcctggagaaggcagccctttcggatggaaggacatcaaggagtggtatgagatgctgatgggtcattgcacctactttccggaagaactgcgctcagtgaagtacgcgtacaacgctgacctctacaacgctctcaacgatctgaacaacctcgtgatcacccgggacgagaacgaaaagctggagtactacgaaaagttccagattatcgaaaacgtgttcaagcagaagaagaagcccaccctgaagcagattgcaaaggagatccttgtgaacgaggaggatattaagggctaccgggtcacctccaccgggaaaccagagttcactaatctcaaggtgtaccatgacattaaggacattactgcccgcaaggagatcattgaaaacgcggaactgctggaccaaatcgcgaagatcctgaccatctatcagagctccgaggatatccaggaggaacttactaacctcaattccgagctgacgcaggaagaaatcgagcaaattagcaacctgaagggttacactggaacccacaacctcagcttgaaagcgattaaccttattttggatgaactttggcacactaatgacaatcagatcgccattttcaaccggctgaaactggtgccgaagaaggtggacctgagccaacagaaggaaatcccgaccacccttgtggacgatttcatcctgtcacctgtggtgaagaggagcttcatccagtcgatcaaggtcatcaacgccatcataaagaagtacggccttcccaacgacatcatcatcgaactggcccgcgagaagaactccaaagatgcccagaagatgatcaacgagatgcagaagcgaaaccggcagacgaacgaacggatcgaggagatcatccggaccaccgggaaggaaaacgcgaagtacctgatcgagaaaatcaagctgcatgatatgcaggaagggaagtgtctctactccctggaggccattccgctggaggatttgctgaacaaccctttcaactacgaagtcgatcatatcattcctcgctccgtgtccttcgataactccttcaacaataaggtcctcgtgaagcaggaggagaagtaagtatcaaggttacaagacaggtttaaggagaccaatagaaactgggctcgagaatgcgacgggtgtggtacgcagccacttcgagtacccacagtactacctgcttgtcgagacagagaagactcttgcgtttctgataggcacctattggtcttactgacatccactttgcctttctctccacagctcgaagaagggcaacagaaccccgttccagtacctctcgtcgtccgactccaagatcagctacgaaactttcaagaagcacattctgaacctggccaagggcaaagggagaattagcaagaccaagaaggaatacctcctggaagagagagacatcaaccgcttctcggtgcaaaaggatttcatcaaccgcaacctggtcgataccagatacgccaccaggggactgatgaacctcctgcggtcctacttccgggtcaacaatctggacgtgaaggtcaaatccatcaacgggggctttacttctttcctgcgccggaagtggaagttcaagaaggaacggaacaagggatacaagcaccacgctgaagatgccctgattattgccaacgccgacttcatctttaaggaatggaaaaagctggacaaggctaagaaggtcatggagaaccagatgttcgaagaaaagcaggccgagtccatgcccgaaatcgaaaccgagcaggaatacaaggagatcttcatcacaccgcaccaaatcaagcacatcaaggacttcaaggattacaagtacagccaccgggtggacaagaagcctaacagagagcttatcaacgacaccctgtactccacgcgcaaggacgacaagggaaacacattgatcgtgaacaacctgaacggactgtatgacaaggacaatgacaaactgaagaagctgatcaacaaatcgccggaaaagctcctgatgtaccatcacgaccctcaaacctaccagaaactgaagctcatcatggagcagtacggcgacgaaaagaatcccctgtacaaatactacgaggagactggaaattacctgactaagtactccaagaaggataacggccccgtgatcaagaagattaagtactacggaaacaaactgaacgcacatctcgacatcaccgatgattatccaaactcccgcaacaaagtcgtgaagctctccctcaaaccgtaccgcttcgacgtgtacctggataatggggtgtacaagttcgtgaccgtgaagaacctggacgtcattaagaaggaaaactactacgaagtgaactcaaagtgctacgaggaagccaagaagctcaagaagatcagcaaccaggccgagttcatcgcatcgttttacaacaatgacctcattaagattaatggagaactgtacagagtgatcggcgtgaacaacgacctcctgaaccggattgaagtgaacatgatcgatattacctaccgggagtatctggagaacatgaacgacaagcgcccaccgagaatcatcaaaactattgcctccaagacccaatccattaagaaatactccaccgacatcctgggcaacctgtacgaggtcaagtcgaagaagcacccccagattatcaagaagggaaaaaggccggcggccacgaaaaaggccggccaggcaaaaaagaaaaaggcttaagaattcctagagctcgctgatcagcctcgaaacttgtttattgcagcttataatggttacaaataaagcaatagcatcacaaatttcacaaataaagcatttttttcactgcattctagttgtggtttgtccaaactcatcaatgtatcttatcatgtctgtataccagcaggctttcctaggttcgaacgctgacgtcatcaacccgctccaaggaatcgcgggcccagtgtcactaggcgggaacacccagcgcgcgtgcgccctggcaggaagatggctgtgagggacaggggagtggcgccctgcaatatttgcatgtcgctatgtgttctgggaaatcaccataaacgtgaaatccctatcagtgatagagacttataagttccctatcagtgatagagacaccgggtgtggtacgcagccactgtttaagtactctgtgctggaaacagcacagaatctacttaaacaaggcaaaatgccgtgtttatctcgtcaacttgttggcgagattttttcacgtgcggaccgaggctgcagcgtcgtcctccctaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccttagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaactctatctcgggctattcttttgatttataagggattttgccgatttcggtctattggttaaaaaatgagctgatttaacaaaaatttaacgcgaattttaacaaaatattaacgttagcgcttccattgtcgacttccgaggacgagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccggagcttcatccagtcgatcacgaatgatcgactggatgaagctccttttttccgcggaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgaacaataaaactgtctgcttacataaacagtaatacaaggggtgttatgagccatattcaacgggaaacgtcgaggccgcgattaaattccaacatggatgctgatttatatgggtataaatgggctcgcgataatgtcgggcaatcaggtgcgacaatctatcgcttgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccggaaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataaacttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgt

SEQ ID NO: 38 - DNA, 8759 bp, synthetic construct
  source 1..8759: mol_type = other DNA
cctgcaggcagctgcgcgctcgctcgctcactgaggccgcccgggcgtcgggcgacctttggtcgcccggcctcagtgagcgagcgagcgcgcagagagggagtggccaactccatcactaggggttcctgcggccgcacgcgtgggccccagaagcctggtggttgtttgtccttctcaggggaaaagtgaggcggccccttggaggaaggggccgggcagaatgatctaatcggattccaagcagctcaggggattgtctttttctagcaccttcttgccactcctaagcgtcctccgtgaccccggctgggatttagcctggtgctgtgtcagccccggctagctaactaccggtgccaccatggccccaaagaagaagcggaaggtcggtagtactgtgggtactcgaagtggctgcgtaccacacccgtcgcatggatccaagcggaactatatcctgggactggacatcggaattacctccgtgggatacggcatcatcgattacgagactagggacgtgattgacgccggcgtgagactctttaaggaggccaacgtggaaaacaacgaaggtcgcagatccaagcggggtgcaagacgcctgaagcgccggaggagacatcggatacagcgcgtgaagaagctccttttcgactacaacctcctcactgaccactcggaattgtccggtatcaacccctacgaagcccgcgtgaaaggcctgagccagaagctgtccgaagaggagtttagcgcagccctgctgcacctggctaagcgaaggggggtgcacaacgtgaacgaggtggaggaggacactggcaacgaactgtccaccaaggagcagatttcacggaactcgaaggcgctggaagagaaatatgtggccgagctgcagctggagaggctcaagaaggatggcgaagtccgggggagcatcaatcgcttcaagacctcggactacgtgaaggaagccaaacagctgttgaaggtgcagaaggcctaccaccaactggaccaatcattcattgacacttacatcgatctgcttgaaaccaggcgcacctactacgagggtcctggagaaggcagccctttcggatggaaggacatcaaggagtggtatgagatgctgatgggtcattgcacctactttccggaagaactgcgctcagtgaagtacgcgtacaacgctgacctctacaacgctctcaacgatctgaacaacctcgtgatcacccgggacgagaacgaaaagctggagtactacgaaaagttccagattatcgaaaacgtgttcaagcagaagaagaagcccaccctgaagcagattgcaaaggagatccttgtgaacgaggaggatattaagggctaccgggtcacctccaccgggaaaccagagttcactaatctcaaggtgtaccatgacattaaggacattactgcccgcaaggagatcattgaaaacgcggaactgctggaccaaatcgcgaagatcctgaccatctatcagagctccgaggatatccaggaggaacttactaacctcaattccgagctgacgcaggaagaaatcgagcaaattagcaacctgaagggttacactggaacccacaacctcagcttgaaagcgattaaccttattttggatgaactttggcacactaatgacaatcagatcgccattttcaaccggctgaaactggtgccgaagaaggtggacctgagccaacagaaggaaatcccgaccacccttgtggacgatttcatcctgtcacctgtggtgaagaggagcttcatccagtcgatcaaggtcatcaacgccatcataaagaagtacggccttcccaacgacatcatcatcgaactggcccgcgagaagaactccaaagatgcccagaagatgatcaacgagatgcagaagcgaaaccggcagacgaacgaacggatcgaggagatcatccggaccaccgggaaggaaaacgcgaagtacctgatcgagaaaatcaagctgcatgatatgcaggaagggaagtgtctctactccctggaggccattccgctggaggatttgctgaacaaccctttcaactacgaagtcgatcatatcattcctcgctccgtgtccttcgataactccttcaacaataaggtcctcgtgaagcaggaggagaagtaagtatcaaggttacaagacaggtttaaggagaccaatagaaactgggctcgagaatgcgacgggtgtggtacgcagccacttcgagtacccacagtactacctgcttgtcgagacagagaagactcttgcgtttctgataggcacctattggtcttactgacatccactttgcctttctctccacagctcgaagaagggcaacagaaccccgttccagtacctctcgtcgtccgactccaagatcagctacgaaactttcaagaagcacattctgaacctggccaagggcaaagggagaattagcaagaccaagaaggaatacctcctggaagagagagacatcaaccgcttctcggtgcaaaaggatttcatcaaccgcaacctggtcgataccagatacgccaccaggggactgatgaacctcctgcggtcctacttccgggtcaacaatctggacgtgaaggtcaaatccatcaacgggggctttacttctttcctgcgccggaagtggaagttcaagaaggaacggaacaagggatacaagcaccacgctgaagatgccctgattattgccaacgccgacttcatctttaaggaatggaaaaagctggacaaggctaagaaggtcatggagaaccagatgttcgaagaaaagcaggccgagtccatgcccgaaatcgaaaccgagcaggaatacaaggagatcttcatcacaccgcaccaaatcaagcacatcaaggacttcaaggattacaagtacagccaccgggtggacaagaagcctaacagagagcttatcaacgacaccctgtactccacgcgcaaggacgacaagggaaacacattgatcgtgaacaacctgaacggactgtatgacaaggacaatgacaaactgaagaagctgatcaacaaatcgccggaaaagctcctgatgtaccatcacgaccctcaaacctaccagaaactgaagctcatcatggagcagtacggcgacgaaaagaatcccctgtacaaatactacgaggagactggaaattacctgactaagtactccaagaaggataacggccccgtgatcaagaagattaagtactacggaaacaaactgaacgcacatctcgacatcaccgatgattatccaaactcccgcaacaaagtcgtgaagctctccctcaaaccgtaccgcttcgacgtgtacctggataatggggtgtacaagttcgtgaccgtgaagaacctggacgtcattaagaaggaaaactactacgaagtgaactcaaagtgctacgaggaagccaagaagctcaagaagatcagcaaccaggccgagttcatcgcatcgttttacaacaatgacctcattaagattaatggagaactgtacagagtgatcggcgtgaacaacgacctcctgaaccggattgaagtgaacatgatcgatattacctaccgggagtatctggagaacatgaacgacaagcgcccaccgagaatcatcaaaactattgcctccaagacccaatccattaagaaatactccaccgacatcctgggcaacctgtacgaggtcaagtcgaagaagcacccccagattatcaagaagggaaaaaggccggcggccacgaaaaaggccggccaggcaaaaaagaaaaaggcttaagaattcctagagctcgctgatcagcctcgaaacttgtttattgcagcttataatggttacaaataaagcaatagcatcacaaatttcacaaataaagcatttttttcactgcattctagttgtggtttgtccaaactcatcaatgtatcttatcatgtctgtataccagcaggctttcctaggttcgaacgctgacgtcatcaacccgctccaaggaatcgcgggcccagtgtcactaggcgggaacacccagcgcgcgtgcgccctggcaggaagatggctgtgagggacaggggagtggcgccctgcaatatttgcatgtcgctatgtgttctgggaaatcaccataaacgtgaaatccctatcagtgatagagacttataagttccctatcagtgatagagacaccgggtgtggtacgcagccactgtttaagtactctgtgctggaaacagcacagaatctacttaaacaaggcaaaatgccgtgtttatctcgtcaacttgttggcgagattttttcacgtgcggaccgaggctgcagcgtcgtcctccctaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccttagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaactctatctcgggctattcttttgatttataagggattttgccgatttcggtctattggttaaaaaatgagctgatttaacaaaaatttaacgcgaattttaacaaaatattaacgttagcgctgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgttaattaaccggtgccaccatgatgtcccgccttgataaatcaaaagtaataaacagtgctcttgagcttctcaatgaagttggtatagaagggttgacgactcggaaattggcgcaaaaactcggtgttgagcagccaaccttgtattggcatgttaaaaacaaacgagcactcctcgacgctttggcgatagagatgctggacaggcaccacacgcatttctgtcccctcgaaggagagtcatggcaggatttccttagaaataacgcaaagtccttcagatgtgcgctgcttagtcaccgcgacggcgcaaaagttcatctcggcactaggccaaccgagaaacagtacgagactctggagaaccaactggcgtttttgtgtcaacagggttttagtctcgaaaatgcgctctatgctctctctgcggttggccatttcaccctcggatgcgtactggaagatcaggagcaccaagtggccaaagaagaacgggaaacgccgactacggacagcatgcctccgttgctccggcaagctatagagctcttcgatcaccaaggcgctgagccagctttcttgttcggattggaacttattatatgcgggctcgaaaagcagcttaaatgcgagtcaggttaagtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgagttagtccattgtcgacttccgaggacgagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccggagcttcatccagtcgatcacgaatgatcgactggatgaagctccttttttccgcggaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgaacaataaaactgtctgcttacataaacagtaatacaaggggtgttatgagccatattcaacgggaaacgtcgaggccgcgattaaattccaacatggatgctgatttatatgggtataaatgggctcgcgataatgtcgggcaatcaggtgcgacaatctatcgcttgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccggaaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataaacttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgt

SEQ ID NO: 39 - DNA, 6889 bp, synthetic construct
  source 1..6889: mol_type = other DNA
cctgcaggcagctgcgcgctcgctcgctcactgaggccgcccgggcgtcgggcgacctttggtcgcccggcctcagtgagcgagcgagcgcgcagagagggagtggccaactccatcactaggggttcctgcggccgcacgcgtgggccccagaagcctggtggttgtttgtccttctcaggggaaaagtgaggcggccccttggaggaaggggccgggcagaatgatctaatcggattccaagcagctcaggggattgtctttttctagcaccttcttgccactcctaagcgtcctccgtgaccccggctgggatttagcctggtgctgtgtcagccccggctagctaactaccggtgccaccatggccccaaagaagaagcggaaggtcggtagtactgtgggtactcgaagtggctgcgtaccacacccgtcgcatggatccaagcggaactatatcctgggactggacatcggaattacctccgtgggatacggcatcatcgattacgagactagggacgtgattgacgccggcgtgagactctttaaggaggccaacgtggaaaacaacgaaggtcgcagatccaagcggggtgcaagacgcctgaagcgccggaggagacatcggatacagcgcgtgaagaagctccttttcgactacaacctcctcactgaccactcggaattgtccggtatcaacccctacgaagcccgcgtgaaaggcctgagccagaagctgtccgaagaggagtttagcgcagccctgctgcacctggctaagcgaaggggggtgcacaacgtgaacgaggtggaggaggacactggcaacgaactgtccaccaaggagcagatttcacggaactcgaaggcgctggaagagaaatatgtggccgagctgcagctggagaggctcaagaaggatggcgaagtccgggggagcatcaatcgcttcaagacctcggactacgtgaaggaagccaaacagctgttgaaggtgcagaaggcctaccaccaactggaccaatcattcattgacacttacatcgatctgcttgaaaccaggcgcacctactacgagggtcctggagaaggcagccctttcggatggaaggacatcaaggagtggtatgagatgctgatgggtcattgcacctactttccggaagaactgcgctcagtgaagtacgcgtacaacgctgacctctacaacgctctcaacgatctgaacaacctcgtgatcacccgggacgagaacgaaaagctggagtactacgaaaagttccagattatcgaaaacgtgttcaagcagaagaagaagcccaccctgaagcagattgcaaaggagatccttgtgaacgaggaggatattaagggctaccgggtcacctccaccgggaaaccagagttcactaatctcaaggtgtaccatgacattaaggacattactgcccgcaaggagatcattgaaaacgcggaactgctggaccaaatcgcgaagatcctgaccatctatcagagctccgaggatatccaggaggaacttactaacctcaattccgagctgacgcaggaagaaatcgagcaaattagcaacctgaagggttacactggaacccacaacctcagcttgaaagcgattaaccttattttggatgaactttggcacactaatgacaatcagatcgccattttcaaccggctgaaactggtgccgaagaaggtggacctgagccaacagaaggaaatcccgaccacccttgtggacgatttcatcctgtcacctgtggtgaagaggagcttcatccagtcgatcaaggtcatcaacgccatcataaagaagtacggccttcccaacgacatcatcatcgaactggcccgcgagaagaactccaaagatgcccagaagatgatcaacgagatgcagaagcgaaaccggcagacgaacgaacggatcgaggagatcatccggaccaccgggaaggaaaacgcgaagtacctgatcgagaaaatcaagctgcatgatatgcaggaagggaagtgtctctactccctggaggccattccgctggaggatttgctgaacaaccctttcaactacgaagtcgatcatatcattcctcgctccgtgtccttcgataactccttcaacaataaggtcctcgtgaagcaggaggagaagtaagtatcaaggttacaagacaggtttaaggagaccaatagaaactgggctcgagaatgcgacgggtgtggtacgcagccacttcgagtacccacagtactacctgcttgtcgagacagagaagactcttgcgtttctgataggcacctattggtcttactgacatccactttgcctttctctccacagctcgaagaagggcaacagaaccccgttccagtacctctcgtcgtccgactccaagatcagctacgaaactttcaagaagcacattctgaacctggccaagggcaaagggagaattagcaagaccaagaaggaatacctcctggaagagagagacatcaaccgcttctcggtgcaaaaggatttcatcaaccgcaacctggtcgataccagatacgccaccaggggactgatgaacctcctgcggtcctacttccgggtcaacaatctggacgtgaaggtcaaatccatcaacgggggctttacttctttcctgcgccggaagtggaagttcaagaaggaacggaacaagggatacaagcaccacgctgaagatgccctgattattgccaacgccgacttcatctttaaggaatggaaaaagctggacaaggctaagaaggtcatggagaaccagatgttcgaagaaaagcaggccgagtccatgcccgaaatcgaaaccgagcaggaatacaaggagatcttcatcacaccgcaccaaatcaagcacatcaaggacttcaaggattacaagtacagccaccgggtggacaagaagcctaacagagagcttatcaacgacaccctgtactccacgcgcaaggacgacaagggaaacacattgatcgtgaacaacctgaacggactgtatgacaaggacaatgacaaactgaagaagctgatcaacaaatcgccggaaaagctcctgatgtaccatcacgaccctcaaacctaccagaaactgaagctcatcatggagcagtacggcgacgaaaagaatcccctgtacaaatactacgaggagactggaaattacctgactaagtactccaagaaggataacggccccgtgatcaagaagattaagtactacggaaacaaactgaacgcacatctcgacatcaccgatgattatccaaactcccgcaacaaagtcgtgaagctctccctcaaaccgtaccgcttcgacgtgtacctggataatggggtgtacaagttcgtgaccgtgaagaacctggacgtcattaagaaggaaaactactacgaagtgaactcaaagtgctacgaggaagccaagaagctcaagaagatcagcaaccaggccgagttcatcgcatcgttttacaacaatgacctcattaagattaatggagaactgtacagagtgatcggcgtgaacaacgacctcctgaaccggattgaagtgaacatgatcgatattacctaccgggagtatctggagaacatgaacgacaagcgcccaccgagaatcatcaaaactattgcctccaagacccaatccattaagaaatactccaccgacatcctgggcaacctgtacgaggtcaagtcgaagaagcacccccagattatcaagaagggaaaaaggccggcggccacgaaaaaggccggccaggcaaaaaagaaaaaggcttaagaattcctagagctcgctgatcagcctcgaaacttgtttattgcagcttataatggttacaaataaagcaatagcatcacaaatttcacaaataaagcatttttttcactgcattctagttgtggtttgtccaaactcatcaatgtatcttatcatgtctgtataccagcaggctttcctaggttcgaacgctgacgtcatcaacccgctccaaggaatcgcgggcccagtgtcactaggcgggaacacccagcgcgcgtgcgccctggcaggaagatggctgtgagggacaggggagtggcgccctgcaatatttgcatgtcgctatgtgttctgggaaatcaccataaacgtgaaatccctatcagtgatagagacttataagttccctatcagtgatagagacaccgggtgtggtacgcagccactgtttaagtactctgtgctggaaacagcacagaatctacttaaacaaggcaaaatgccgtgtttatctcgtcaacttgttggcgagattttttcacgtgcggaccgaggctgcagcgtcgtcctccctaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggcctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgaacaataaaactgtctgcttacataaacagtaatacaaggggtgttatgagccatattcaacgggaaacgtcgaggccgcgattaaattccaacatggatgctgatttatatgggtataaatgggctcgcgataatgtcgggcaatcaggtgcgacaatctatcgcttgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccggaaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataaacttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgt

SEQ ID NO: 40 - DNA, 6901 bp, synthetic construct
  source 1..6901: mol_type = other DNA
cctgcaggcagctgcgcgctcgctcgctcactgaggccgcccgggcgtcgggcgacctttggtcgcccggcctcagtgagcgagcgagcgcgcagagagggagtggccaactccatcactaggggttcctgcggccgcacgcgtgctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgctagctaactaccggtgccaccatggccccaaagaagaagcggaaggtcggtagtactgtgggtactcgaagtggctgcgtaccacacccgtcgcatggatccaagcggaactatatcctgggactggacatcggaattacctccgtgggatacggcatcatcgattacgagactagggacgtgattgacgccggcgtgagactctttaaggaggccaacgtggaaaacaacgaaggtcgcagatccaagcggggtgcaagacgcctgaagcgccggaggagacatcggatacagcgcgtgaagaagctccttttcgactacaacctcctcactgaccactcggaattgtccggtatcaacccctacgaagcccgcgtgaaaggcctgagccagaagctgtccgaagaggagtttagcgcagccctgctgcacctggctaagcgaaggggggtgcacaacgtgaacgaggtggaggaggacactggcaacgaactgtccaccaaggagcagatttcacggaactcgaaggcgctggaagagaaatatgtggccgagctgcagctggagaggctcaagaaggatggcgaagtccgggggagcatcaatcgcttcaagacctcggactacgtgaaggaagccaaacagctgttgaaggtgcagaaggcctaccaccaactggaccaatcattcattgacacttacatcgatctgcttgaaaccaggcgcacctactacgagggtcctggagaaggcagccctttcggatggaaggacatcaaggagtggtatgagatgctgatgggtcattgcacctactttccggaagaactgcgctcagtgaagtacgcgtacaacgctgacctctacaacgctctcaacgatctgaacaacctcgtgatcacccgggacgagaacgaaaagctggagtactacgaaaagttccagattatcgaaaacgtgttcaagcagaagaagaagcccaccctgaagcagattgcaaaggagatccttgtgaacgaggaggatattaagggctaccgggtcacctccaccgggaaaccagagttcactaatctcaaggtgtaccatgacattaaggacattactgcccgcaaggagatcattgaaaacgcggaactgctggaccaaatcgcgaagatcctgaccatctatcagagctccgaggatatccaggaggaacttactaacctcaattccgagctgacgcaggaagaaatcgagcaaattagcaacctgaagggttacactggaacccacaacctcagcttgaaagcgattaaccttattttggatgaactttggcacactaatgacaatcagatcgccattttcaaccggctgaaactggtgccgaagaaggtggacctgagccaacagaaggaaatcccgaccacccttgtggacgatttcatcctgtcacctgtggtgaagaggagcttcatccagtcgatcaaggtcatcaacgccatcataaagaagtacggccttcccaacgacatcatcatcgaactggcccgcgagaagaactccaaagatgcccagaagatgatcaacgagatgcagaagcgaaaccggcagacgaacgaacggatcgaggagatcatccggaccaccgggaaggaaaacgcgaagtacctgatcgagaaaatcaagctgcatgatatgcaggaagggaagtgtctctactccctggaggccattccgctggaggatttgctgaacaaccctttcaactacgaagtcgatcatatcattcctcgctccgtgtccttcgataactccttcaacaataaggtcctcgtgaagcaggaggagaagtaagtatcaaggttacaagacaggtttaaggagaccaatagaaactgggctcgagaatgcgacgggtgtggtacgcagccacttcgagtacccacagtactacctgcttgtcgagacagagaagactcttgcgtttctgataggcacctattggtcttactgacatccactttgcctttctctccacagctcgaagaagggcaacagaaccccgttccagtacctctcgtcgtccgactccaagatcagctacgaaactttcaagaagcacattctgaacctggccaagggcaaagggagaattagcaagaccaagaaggaatacctcctggaagagagagacatcaaccgcttctcggtgcaaaaggatttcatcaaccgcaacctggtcgataccagatacgccaccaggggactgatgaacctcctgcggtcctacttccgggtcaacaatctggacgtgaaggtcaaatccatcaacgggggctttacttctttcctgcgccggaagtggaagttcaagaaggaacggaacaagggatacaagcaccacgctgaagatgccctgattattgccaacgccgacttcatctttaaggaatggaaaaagctggacaaggctaagaaggtcatggagaaccagatgttcgaagaaaagcaggccgagtccatgcccgaaatcgaaaccgagcaggaatacaaggagatcttcatcacaccgcaccaaatcaagcacatcaaggacttcaaggattacaagtacagccaccgggtggacaagaagcctaacagagagcttatcaacgacaccctgtactccacgcgcaaggacgacaagggaaacacattgatcgtgaacaacctgaacggactgtatgacaaggacaatgacaaactgaagaagctgatcaacaaatcgccggaaaagctcctgatgtaccatcacgaccctcaaacctaccagaaactgaagctcatcatggagcagtacggcgacgaaaagaatcccctgtacaaatactacgaggagactggaaattacctgactaagtactccaagaaggataacggccccgtgatcaagaagattaagtactacggaaacaaactgaacgcacatctcgacatcaccgatgattatccaaactcccgcaacaaagtcgtgaagctctccctcaaaccgtaccgcttcgacgtgtacctggataatggggtgtacaagttcgtgaccgtgaagaacctggacgtcattaagaaggaaaactactacgaagtgaactcaaagtgctacgaggaagccaagaagctcaagaagatcagcaaccaggccgagttcatcgcatcgttttacaacaatgacctcattaagattaatggagaactgtacagagtgatcggcgtgaacaacgacctcctgaaccggattgaagtgaacatgatcgatattacctaccgggagtatctggagaacatgaacgacaagcgcccaccgagaatcatcaaaactattgcctccaagacccaatccattaagaaatactccaccgacatcctgggcaacctgtacgaggtcaagtcgaagaagcacccccagattatcaagaagggaaaaaggccggcggccacgaaaaaggccggccaggcaaaaaagaaaaaggcttaagaattcctagagctcgctgatcagcctcgaaacttgtttattgcagcttataatggttacaaataaagcaatagcatcacaaatttcacaaataaagcatttttttcactgcattctagttgtggtttgtccaaactcatcaatgtatcttatcatgtctgtataccagcaggctttcctaggttcgaacgctgacgtcatcaacccgctccaaggaatcgcgggcccagtgtcactaggcgggaacacccagcgcgcgtgcgccctggcaggaagatggctgtgagggacaggggagtggcgccctgcaatatttgcatgtcgctatgtgttctgggaaatcaccataaacgtgaaatccctatcagtgatagagacttataagttccctatcagtgatagagacaccgggtgtggtacgcagccactgtttaagtactctgtgctggaaacagcacagaatctacttaaacaaggcaaaatgccgtgtttatctcgtcaacttgttggcgagattttttcacgtgcggaccgaggctgcagcgtcgtcctccctaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggcctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgaacaataaaactgtctgcttacataaacagtaatacaaggggtgttatgagccatattcaacgggaaacgtcgaggccgcgattaaattccaacatggatgctgatttatatgggtataaatgggctcgcgataatgtcgggcaatcaggtgcgacaatctatcgcttgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccggaaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataaacttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgt

SEQ ID NO: 41 - DNA, 8469 bp, synthetic construct
  source 1..8469: mol_type = other DNA
cctgcaggcagctgcgcgctcgctcgctcactgaggccgcccgggcgtcgggcgacctttggtcgcccggcctcagtgagcgagcgagcgcgcagagagggagtggccaactccatcactaggggttcctgcggccgcacgcgtgctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgctagctaactaccggtgccaccatggccccaaagaagaagcggaaggtcggtagtactgtgggtactcgaagtggctgcgtaccacacccgtcgcatggatccaagcggaactatatcctgggactggacatcggaattacctccgtgggatacggcatcatcgattacgagactagggacgtgattgacgccggcgtgagactctttaaggaggccaacgtggaaaacaacgaaggtcgcagatccaagcggggtgcaagacgcctgaagcgccggaggagacatcggatacagcgcgtgaagaagctccttttcgactacaacctcctcactgaccactcggaattgtccggtatcaacccctacgaagcccgcgtgaaaggcctgagccagaagctgtccgaagaggagtttagcgcagccctgctgcacctggctaagcgaaggggggtgcacaacgtgaacgaggtggaggaggacactggcaacgaactgtccaccaaggagcagatttcacggaactcgaaggcgctggaagagaaatatgtggccgagctgcagctggagaggctcaagaaggatggcgaagtccgggggagcatcaatcgcttcaagacctcggactacgtgaaggaagccaaacagctgttgaaggtgcagaaggcctaccaccaactggaccaatcattcattgacacttacatcgatctgcttgaaaccaggcgcacctactacgagggtcctggagaaggcagccctttcggatggaaggacatcaaggagtggtatgagatgctgatgggtcattgcacctactttccggaagaactgcgctcagtgaagtacgcgtacaacgctgacctctacaacgctctcaacgatctgaacaacctcgtgatcacccgggacgagaacgaaaagctggagtactacgaaaagttccagattatcgaaaacgtgttcaagcagaagaagaagcccaccctgaagcagattgcaaaggagatccttgtgaacgaggaggatattaagggctaccgggtcacctccaccgggaaaccagagttcactaatctcaaggtgtaccatgacattaaggacattactgcccgcaaggagatcattgaaaacgcggaactgctggaccaaatcgcgaagatcctgaccatctatcagagctccgaggatatccaggaggaacttactaacctcaattccgagctgacgcaggaagaaatcgagcaaattagcaacctgaagggttacactggaacccacaacctcagcttgaaagcgattaaccttattttggatgaactttggcacactaatgacaatcagatcgccattttcaaccggctgaaactggtgccgaagaaggtggacctgagccaacagaaggaaatcccgaccacccttgtggacgatttcatcctgtcacctgtggtgaagaggagcttcatccagtcgatcaaggtcatcaacgccatcataaagaagtacggccttcccaacgacatcatcatcgaactggcccgcgagaagaactccaaagatgcccagaagatgatcaacgagatgcagaagcgaaaccggcagacgaacgaacggatcgaggagatcatccggaccaccgggaaggaaaacgcgaagtacctgatcgagaaaatcaagctgcatgatatgcaggaagggaagtgtctctactccctggaggccattccgctggaggatttgctgaacaaccctttcaactacgaagtcgatcatatcattcctcgctccgtgtccttcgataactccttcaacaataaggtcctcgtgaagcaggaggagaagtaagtatcaaggttacaagacaggtttaaggagaccaatagaaactgggctcgagaatgcgacgggtgtggtacgcagccacttcgagtacccacagtactacctgcttgtcgagacagagaagactcttgcgtttctgataggcacctattggtcttactgacatccactttgcctttctctccacagctcgaagaagggcaacagaaccccgttccagtacctctcgtcgtccgactccaagatcagctacgaaactttcaagaagcacattctgaacctggccaagggcaaagggagaattagcaagaccaagaaggaatacctcctggaagagagagacatcaaccgcttctcggtgcaaaaggatttcatcaaccgcaacctggtcgataccagatacgccaccaggggactgatgaacctcctgcggtcctacttccgggtcaacaatctggacgtgaaggtcaaatccatcaacgggggctttacttctttcctgcgccggaagtggaagttcaagaaggaacggaacaagggatacaagcaccacgctgaagatgccctgattattgccaacgccgacttcatctttaaggaatggaaaaagctggacaaggctaagaaggtcatggagaaccagatgttcgaagaaaagcaggccgagtccatgcccgaaatcgaaaccgagcaggaatacaaggagatcttcatcacaccgcaccaaatcaagcacatcaaggacttcaaggattacaagtacagccaccgggtggacaagaagcctaacagagagcttatcaacgacaccctgtactccacgcgcaaggacgacaagggaaacacattgatcgtgaacaacctgaacggactgtatgacaaggacaatgacaaactgaagaagctgatcaacaaatcgccggaaaagctcctgatgtaccatcacgaccctcaaacctaccagaaactgaagctcatcatggagcagtacggcgacgaaaagaatcccctgtacaaatactacgaggagactggaaattacctgactaagtactccaagaaggataacggccccgtgatcaagaagattaagtactacggaaacaaactgaacgcacatctcgacatcaccgatgattatccaaactcccgcaacaaagtcgtgaagctctccctcaaaccgtaccgcttcgacgtgtacctggataatggggtgtacaagttcgtgaccgtgaagaacctggacgtcattaagaaggaaaactactacgaagtgaactcaaagtgctacgaggaagccaagaagctcaagaagatcagcaaccaggccgagttcatcgcatcgttttacaacaatgacctcattaagattaatggagaactgtacagagtgatcggcgtgaacaacgacctcctgaaccggattgaagtgaacatgatcgatattacctaccgggagtatctggagaacatgaacgacaagcgcccaccgagaatcatcaaaactattgcctccaagacccaatccattaagaaatactccaccgacatcctgggcaacctgtacgaggtcaagtcgaagaagcacccccagattatcaagaagggaaaaaggccggcggccacgaaaaaggccggccaggcaaaaaagaaaaaggcttaagaattcctagagctcgctgatcagcctcgaaacttgtttattgcagcttataatggttacaaataaagcaatagcatcacaaatttcacaaataaagcatttttttcactgcattctagttgtggtttgtccaaactcatcaatgtatcttatcatgtctgtataccagcaggctttcctaggttcgaacgctgacgtcatcaacccgctccaaggaatcgcgggcccagtgtcactaggcgggaacacccagcgcgcgtgcgccctggcaggaagatggctgtgagggacaggggagtggcgccctgcaatatttgcatgtcgctatgtgttctgggaaatcaccataaacgtgaaatccctatcagtgatagagacttataagttccctatcagtgatagagacaccgggtgtggtacgcagccactgtttaagtactctgtgctggaaacagcacagaatctacttaaacaaggcaaaatgccgtgtttatctcgtcaacttgttggcgagattttttcacgtgcggaccgaggctgcagcgtcgtcctccctaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccttagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaactctatctcgggctattcttttgatttataagggattttgccgatttcggtctattggttaaaaaatgagctgatttaacaaaaatttaacgcgaattttaacaaaatattaacgttagcgctgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgttaattaaccggtgccaccatgatgtcccgccttgataaatcaaaagtaataaacagtgctcttgagcttctcaatgaagttggtatagaagggttgacgactcggaaattggcgcaaaaactcggtgttgagcagccaaccttgtattggcatgttaaaaacaaacgagcactcctcgacgctttggcgatagagatgctggacaggcaccacacgcatttctgtcccctcgaaggagagtcatggcaggatttccttagaaataacgcaaagtccttcagatgtgcgctgcttagtcaccgcgacggcgcaaaagttcatctcggcactaggccaaccgagaaacagtacgagactctggagaaccaactggcgtttttgtgtcaacagggttttagtctcgaaaatgcgctctatgctctctctgcggttggccatttcaccctcggatgcgtactggaagatcaggagcaccaagtggccaaagaagaacgggaaacgccgactacggacagcatgcctccgttgctccggcaagctatagagctcttcgatcaccaaggcgctgagccagctttcttgttcggattggaacttattatatgcgggctcgaaaagcagcttaaatgcgagtcaggttaagtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgagttagtccattgtcgacttccgaggaccgcggaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgaacaataaaactgtctgcttacataaacagtaatacaaggggtgttatgagccatattcaacgggaaacgtcgaggccgcgattaaattccaacatggatgctgatttatatgggtataaatgggctcgcgataatgtcgggcaatcaggtgcgacaatctatcgcttgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccggaaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataaacttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgt

SEQ ID NO: 42 - DNA, 7242 bp, synthetic construct
  source 1..7242: mol_type = other DNA
cctgcaggcagctgcgcgctcgctcgctcactgaggccgcccgggcgtcgggcgacctttggtcgcccggcctcagtgagcgagcgagcgcgcagagagggagtggccaactccatcactaggggttcctgcggccgcacgcgtgctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgctagctaactaccggtgccaccatggccccaaagaagaagcggaaggtcggtagtactgtgggtactcgaagtggctgcgtaccacacccgtcgcatggatccaagcggaactatatcctgggactggacatcggaattacctccgtgggatacggcatcatcgattacgagactagggacgtgattgacgccggcgtgagactctttaaggaggccaacgtggaaaacaacgaaggtcgcagatccaagcggggtgcaagacgcctgaagcgccggaggagacatcggatacagcgcgtgaagaagctccttttcgactacaacctcctcactgaccactcggaattgtccggtatcaacccctacgaagcccgcgtgaaaggcctgagccagaagctgtccgaagaggagtttagcgcagccctgctgcacctggctaagcgaaggggggtgcacaacgtgaacgaggtggaggaggacactggcaacgaactgtccaccaaggagcagatttcacggaactcgaaggcgctggaagagaaatatgtggccgagctgcagctggagaggctcaagaaggatggcgaagtccgggggagcatcaatcgcttcaagacctcggactacgtgaaggaagccaaacagctgttgaaggtgcagaaggcctaccaccaactggaccaatcattcattgacacttacatcgatctgcttgaaaccaggcgcacctactacgagggtcctggagaaggcagccctttcggatggaaggacatcaaggagtggtatgagatgctgatgggtcattgcacctactttccggaagaactgcgctcagtgaagtacgcgtacaacgctgacctctacaacgctctcaacgatctgaacaacctcgtgatcacccgggacgagaacgaaaagctggagtactacgaaaagttccagattatcgaaaacgtgttcaagcagaagaagaagcccaccctgaagcagattgcaaaggagatccttgtgaacgaggaggatattaagggctaccgggtcacctccaccgggaaaccagagttcactaatctcaaggtgtaccatgacattaaggacattactgcccgcaaggagatcattgaaaacgcggaactgctggaccaaatcgcgaagatcctgaccatctatcagagctccgaggatatccaggaggaacttactaacctcaattccgagctgacgcaggaagaaatcgagcaaattagcaacctgaagggttacactggaacccacaacctcagcttgaaagcgattaaccttattttggatgaactttggcacactaatgacaatcagatcgccattttcaaccggctgaaactggtgccgaagaaggtggacctgagccaacagaaggaaatcccgaccacccttgtggacgatttcatcctgtcacctgtggtgaagaggagcttcatccagtcgatcaaggtcatcaacgccatcataaagaagtacggccttcccaacgacatcatcatcgaactggcccgcgagaagaactccaaagatgcccagaagatgatcaacgagatgcagaagcgaaaccggcagacgaacgaacggatcgaggagatcatccggaccaccgggaaggaaaacgcgaagtacctgatcgagaaaatcaagctgcatgatatgcaggaagggaagtgtctctactccctggaggccattccgctggaggatttgctgaacaaccctttcaactacgaagtcgatcatatcattcctcgctccgtgtccttcgataactccttcaacaataaggtcctcgtgaagcaggaggagaagtaagtatcaaggttacaagacaggtttaaggagaccaatagaaactgggctcgagaatgcgacgggtgtggtacgcagccacttcgagtacccacagtactacctgcttgtcgagacagagaagactcttgcgtttctgataggcacctattggtcttactgacatccactttgcctttctctccacagctcgaagaagggcaacagaaccccgttccagtacctctcgtcgtccgactccaagatcagctacgaaactttcaagaagcacattctgaacctggccaagggcaaagggagaattagcaagaccaagaaggaatacctcctggaagagagagacatcaaccgcttctcggtgcaaaaggatttcatcaaccgcaacctggtcgataccagatacgccaccaggggactgatgaacctcctgcggtcctacttccgggtcaacaatctggacgtgaaggtcaaatccatcaacgggggctttacttctttcctgcgccggaagtggaagttcaagaaggaacggaacaagggatacaagcaccacgctgaagatgccctgattattgccaacgccgacttcatctttaaggaatggaaaaagctggacaaggctaagaaggtcatggagaaccagatgttcgaagaaaagcaggccgagtccatgcccgaaatcgaaaccgagcaggaatacaaggagatcttcatcacaccgcaccaaatcaagcacatcaaggacttcaaggattacaagtacagccaccgggtggacaagaagcctaacagagagcttatcaacgacaccctgtactccacgcgcaaggacgacaagggaaacacattgatcgtgaacaacctgaacggactgtatgacaaggacaatgacaaactgaagaagctgatcaacaaatcgccggaaaagctcctgatgtaccatcacgaccctcaaacctaccagaaactgaagctcatcatggagcagtacggcgacgaaaagaatcccctgtacaaatactacgaggagactggaaattacctgactaagtactccaagaaggataacggccccgtgatcaagaagattaagtactacggaaacaaactgaacgcacatctcgacatcaccgatgattatccaaactcccgcaacaaagtcgtgaagctctccctcaaaccgtaccgcttcgacgtgtacctggataatggggtgtacaagttcgtgaccgtgaagaacctggacgtcattaagaaggaaaactactacgaagtgaactcaaagtgctacgaggaagccaagaagctcaagaagatcagcaaccaggccgagttcatcgcatcgttttacaacaatgacctcattaagattaatggagaactgtacagagtgatcggcgtgaacaacgacctcctgaaccggattgaagtgaacatgatcgatattacctaccgggagtatctggagaacatgaacgacaagcgcccaccgagaatcatcaaaactattgcctccaagacccaatccattaagaaatactccaccgacatcctgggcaacctgtacgaggtcaagtcgaagaagcacccccagattatcaagaagggaaaaaggccggcggccacgaaaaaggccggccaggcaaaaaagaaaaaggcttaagaattcctagagctcgctgatcagcctcgaaacttgtttattgcagcttataatggttacaaataaagcaatagcatcacaaatttcacaaataaagcatttttttcactgcattctagttgtggtttgtccaaactcatcaatgtatcttatcatgtctgtataccagcaggctttcctaggttcgaacgctgacgtcatcaacccgctccaaggaatcgcgggcccagtgtcactaggcgggaacacccagcgcgcgtgcgccctggcaggaagatggctgtgagggacaggggagtggcgccctgcaatatttgcatgtcgctatgtgttctgggaaatcaccataaacgtgaaatccctatcagtgatagagacttataagttccctatcagtgatagagacaccgggtgtggtacgcagccactgtttaagtactctgtgctggaaacagcacagaatctacttaaacaaggcaaaatgccgtgtttatctcgtcaacttgttggcgagattttttcacgtgcggaccgaggctgcagcgtcgtcctccctaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccttagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaactctatctcgggctattcttttgatttataagggattttgccgatttcggtctattggttaaaaaatgagctgatttaacaaaaatttaacgcgaattttaacaaaatattaacgttagcgcttccattgtcgacttccgaggacgagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccggagcttcatccagtcgatcacgaatgatcgactggatgaagctccttttttccgcggaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgaacaataaaactgtctgcttacataaacagtaatacaaggggtgttatgagccatattcaacgggaaacgtcgaggccgcgattaaattccaacatggatgctgatttatatgggtataaatgggctcgcgataatgtcgggcaatcaggtgcgacaatctatcgcttgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccggaaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataaacttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgt

SEQ ID NO: 43 - DNA, 8771 bp, synthetic construct
  source 1..8771: mol_type = other DNA
cctgcaggcagctgcgcgctcgctcgctcactgaggccgcccgggcgtcgggcgacctttggtcgcccggcctcagtgagcgagcgagcgcgcagagagggagtggccaactccatcactaggggttcctgcggccgcacgcgtgctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgctagctaactaccggtgccaccatggccccaaagaagaagcggaaggtcggtagtactgtgggtactcgaagtggctgcgtaccacacccgtcgcatggatccaagcggaactatatcctgggactggacatcggaattacctccgtgggatacggcatcatcgattacgagactagggacgtgattgacgccggcgtgagactctttaaggaggccaacgtggaaaacaacgaaggtcgcagatccaagcggggtgcaagacgcctgaagcgccggaggagacatcggatacagcgcgtgaagaagctccttttcgactacaacctcctcactgaccactcggaattgtccggtatcaacccctacgaagcccgcgtgaaaggcctgagccagaagctgtccgaagaggagtttagcgcagccctgctgcacctggctaagcgaaggggggtgcacaacgtgaacgaggtggaggaggacactggcaacgaactgtccaccaaggagcagatttcacggaactcgaaggcgctggaagagaaatatgtggccgagctgcagctggagaggctcaagaaggatggcgaagtccgggggagcatcaatcgcttcaagacctcggactacgtgaaggaagccaaacagctgttgaaggtgcagaaggcctaccaccaactggaccaatcattcattgacacttacatcgatctgcttgaaaccaggcgcacctactacgagggtcctggagaaggcagccctttcggatggaaggacatcaaggagtggtatgagatgctgatgggtcattgcacctactttccggaagaactgcgctcagtgaagtacgcgtacaacgctgacctctacaacgctctcaacgatctgaacaacctcgtgatcacccgggacgagaacgaaaagctggagtactacgaaaagttccagattatcgaaaacgtgttcaagcagaagaagaagcccaccctgaagcagattgcaaaggagatccttgtgaacgaggaggatattaagggctaccgggtcacctccaccgggaaaccagagttcactaatctcaaggtgtaccatgacattaaggacattactgcccgcaaggagatcattgaaaacgcggaactgctggaccaaatcgcgaagatcctgaccatctatcagagctccgaggatatccaggaggaacttactaacctcaattccgagctgacgcaggaagaaatcgagcaaattagcaacctgaagggttacactggaacccacaacctcagcttgaaagcgattaaccttattttggatgaactttggcacactaatgacaatcagatcgccattttcaaccggctgaaactggtgccgaagaaggtggacctgagccaacagaaggaaatcccgaccacccttgtggacgatttcatcctgtcacctgtggtgaagaggagcttcatccagtcgatcaaggtcatcaacgccatcataaagaagtacggccttcccaacgacatcatcatcgaactggcccgcgagaagaactccaaagatgcccagaagatgatcaacgagatgcagaagcgaaaccggcagacgaacgaacggatcgaggagatcatccggaccaccgggaaggaaaacgcgaagtacctgatcgagaaaatcaagctgcatgatatgcaggaagggaagtgtctctactccctggaggccattccgctggaggatttgctgaacaaccctttcaactacgaagtcgatcatatcattcctcgctccgtgtccttcgataactccttcaacaataaggtcctcgtgaagcaggaggagaagtaagtatcaaggttacaagacaggtttaaggagaccaatagaaactgggctcgagaatgcgacgggtgtggtacgcagccacttcgagtacccacagtactacctgcttgtcgagacagagaagactcttgcgtttctgataggcacctattggtcttactgacatccactttgcctttctctccacagctcgaagaagggcaacagaaccccgttccagtacctctcgtcgtccgactccaagatcagctacgaaactttcaagaagcacattctgaacctggccaagggcaaagggagaattagcaagaccaagaaggaatacctcctggaagagagagacatcaaccgcttctcggtgcaaaaggatttcatcaaccgcaacctggtcgataccagatacgccaccaggggactgatgaacctcctgcggtcctacttccgggtcaacaatctggacgtgaaggtcaaatccatcaacgggggctttacttctttcctgcgccggaagtggaagttcaagaaggaacggaacaagggatacaagcaccacgctgaagatgccctgattattgccaacgccgacttcatctttaaggaatggaaaaagctggacaaggctaagaaggtcatggagaaccagatgttcgaagaaaagcaggccgagtccatgcccgaaatcgaaaccgagcaggaatacaaggagatcttcatcacaccgcaccaaatcaagcacatcaaggacttcaaggattacaagtacagccaccgggtggacaagaagcctaacagagagcttatcaacgacaccctgtactccacgcgcaaggacgacaagggaaacacattgatcgtgaacaacctgaacggactgtatgacaaggacaatgacaaactgaagaagctgatcaacaaatcgccggaaaagctcctgatgtaccatcacgaccctcaaacctaccagaaactgaagctcatcatggagcagtacggcgacgaaaagaatcccctgtacaaatactacgaggagactggaaattacctgactaagtactccaagaaggataacggccccgtgatcaagaagattaagtactacggaaacaaactgaacgcacatctcgacatcaccgatgattatccaaactcccgcaacaaagtcgtgaagctctccctcaaaccgtaccgcttcgacgtgtacctggataatggggtgtacaagttcgtgaccgtgaagaacctggacgtcattaagaaggaaaactactacgaagtgaactcaaagtgctacgaggaagccaagaagctcaagaagatcagcaaccaggccgagttcatcgcatcgttttacaacaatgacctcattaagattaatggagaactgtacagagtgatcggcgtgaacaacgacctcctgaaccggattgaagtgaacatgatcgatattacctaccgggagtatctggagaacatgaacgacaagcgcccaccgagaatcatcaaaactattgcctccaagacccaatccattaagaaatactccaccgacatcctgggcaacctgtacgaggtcaagtcgaagaagcacccccagattatcaagaagggaaaaaggccggcggccacgaaaaaggccggccaggcaaaaaagaaaaaggcttaagaattcctagagctcgctgatcagcctcgaaacttgtttattgcagcttataatggttacaaataaagcaatagcatcacaaatttcacaaataaagcatttttttcactgcattctagttgtggtttgtccaaactcatcaatgtatcttatcatgtctgtataccagcaggctttcctaggttcgaacgctgacgtcatcaacccgctccaaggaatcgcgggcccagtgtcactaggcgggaacacccagcgcgcgtgcgccctggcaggaagatggctgtgagggacaggggagtggcgccctgcaatatttgcatgtcgctatgtgttctgggaaatcaccataaacgtgaaatccctatcagtgatagagacttataagttccctatcagtgatagagacaccgggtgtggtacgcagccactgtttaagtactctgtgctggaaacagcacagaatctacttaaacaaggcaaaatgccgtgtttatctcgtcaacttgttggcgagattttttcacgtgcggaccgaggctgcagcgtcgtcctccctaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccttagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaactctatctcgggctattcttttgatttataagggattttgccgatttcggtctattggttaaaaaatgagctgatttaacaaaaatttaacgcgaattttaacaaaatattaacgttagcgctgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgttaattaaccggtgccaccatgatgtcccgccttgataaatcaaaagtaataaacagtgctcttgagcttctcaatgaagttggtatagaagggttgacgactcggaaattggcgcaaaaactcggtgttgagcagccaaccttgtattggcatgttaaaaacaaacgagcactcctcgacgctttggcgatagagatgctggacaggcaccacacgcatttctgtcccctcgaaggagagtcatggcaggatttccttagaaataacgcaaagtccttcagatgtgcgctgcttagtcaccgcgacggcgcaaaagttcatctcggcactaggccaaccgagaaacagtacgagactctggagaaccaactggcgtttttgtgtcaacagggttttagtctcgaaaatgcgctctatgctctctctgcggttggccatttcaccctcggatgcgtactggaagatcaggagcaccaagtggccaaagaagaacgggaaacgccgactacggacagcatgcctccgttgctccggcaagctatagagctcttcgatcaccaaggcgctgagccagctttcttgttcggattggaacttattatatgcgggctcgaaaagcagcttaaatgcgagtcaggttaagtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgagttagtccattgtcgacttccgaggacgagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccggagcttcatccagtcgatcacgaatgatcgactggatgaagctccttttttccgcggaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgaacaataaaactgtctgcttacataaacagtaatacaaggggtgttatgagccatattcaacgggaaacgtcgaggccgcgattaaattccaacatggatgctgatttatatgggtataaatgggctcgcgataatgtcgggcaatcaggtgcgacaatctatcgcttgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccggaaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataaacttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgt

SEQ ID NO: 44 - DNA, 5823 bp, synthetic construct
  source 1..5823: mol_type = other DNA
ggggttggggttgcgccttttccaaggcagccctgggtttgcgcagggacgcggctgctctgggcgtggttccgggaaacgcagcggcgccgaccctgggtctcgcacattcttcacgtccgttcgcagcgtcacccggatcttcgccgctacccttgtgggccccccggcgacgcttcctgctccgcccctaagtcgggaaggttccttgcggttcgcggcgtgccggacgtgacaaacggaagccgcacgtctcactagtaccctcgcagacggacagcgccagggagcaatggcagcgcgccgaccgcgatgggctgtggccaatagcggctgctcagcagggcgcgccgagagcagcggccgggaaggggcggtgcgggaggcggggtgtggggcggtagtgtgggccctgttcctgcccgcgcggtgttccgcattctgcaagcctccggagcgcacgtcggcagtcggctccctcgttgaccgaatcaccgacctctctccccaggctagccaccatgggcctgaggctacagctctccctgggcatcatcccagttgaggaggagaacccggacttctggaaccgcgaggcagccgaggccctgggtgccgccaagaagctgcagcctgcacagacagccgccaagaacctcatcatcttcctgggcgatgggatgggggtgtctacggtgacagctgccaggatcgatctgcttcctggccctgctgggcctgctcatcctctgggagccctggcctgcccaggcttttgtcaacagcacctttgtggttctcacttggtggaagctctctacctggtgtgtggggagcgtggattcttctacacacccatgtcccgccgcgaagtggaggacccacaaggtaagctctgctcctgaattaattctatcccaagtgctaactaccctgtttgtctttcacccttgagaccttgtaaattgtgccctaggtgtggagggtctcaggctaaccagtggggggcacatttctgtgggcagctagacatatgtaaacatggtagctgccaggaaggagtgagaatccttccttaagtctcctaggtggtgacgggtggctaggccccaggataggtaccgggccccccctcgagcattaaataatgttacttatgttatacagcccatttccttttttatcagagtcaggctgaaagttcatttttctgcaaaactgcagtgcccattcttaatttttgttttgagaaataactgttgtaccctctttattatcacaagattaataatatttctttattttattgctcctcttacatttggatcttttattatgagctgtcaaaaattcttttgggaagtagttggggtataaataataaatcgataaaagaattcatgattaaagacaagatagaatccattattctgttaacatatcagtagtgaataacaaaaggctacattgacaattagcatgcaatatattgctttatataattatacatccatatatttgccactttgatttttaattcactggtaaatttaggggatactcatttatggagttaatattaccaaaaagagtttttaaactgagcgaggctttaattagaatggtaaaatattgcttcagggcaacaatgagaatgtaacaagaaaactgactgatcagtcatgggtaactcgaatagcaaacaagtagagaagactcacaaaaccactgaaagttatatacacactcaagttgtgagtcattcaaactgggtattcatacacatgtatacaaatacaaacaagtgtgtgcgcacacaccctttccaatatacacacctgaatataaaactaaataagcagattcctctgttaacaattttattactctattttaggctggggctgaacttttgaagctgatgagcaaaatagtttcccataaaagataatttcatttgccaaaatgaaactactttccgttattaatgttgcaatgaaatggtttgaaatatgagtctgtgatctggacagaaaggatgctcctgttcccagctccaagagggagttctctaaacaattacacccacggagagcgctgtaaggttgtattttcatcaccggccagctgagctgtgtgcctgtcatactcccagtctcccctccagtgacagccgcattgacaacatgttatactccacattgattttacagttaaggagctgattgtgatatatagagattttaagaattcagctgagaggctgacttttgccttttatcattccattaaaattttataaccatctttttcatgtcatttcatccatgaattcttactttagaaagtgatagctactgctgtgagcataacttttctcatgttcagagctttttattgtattagcaaactaccccaattatagttattactcatgttttacataattgtggtggccctttcaaccatgcagttgcaggccagttgatttgtatatagaattagatgattcggcttatcattttaaagcactaaattgaaagagtgccaggagtcaggttttaacacttccctagccaaaggagctaattaagctgctttcagcttcctctccagaatcacacaagttaaaggacccttctgcaacaagagcagcgaatctactcagccagagcaggaagctaataaaatgtatgctggcttttaagggggaaacaaatcatgaaattgaaattgaacacctctcctttcccaaggtaagagatcatctttaagaaaaggctgtgtattgtgggggtttgaagtgcaagttcatctcattatcatggatgtttcacccataatactatcatcatatgcaggagaaataaaagccttatcccccaatcacagagaacaaactgcatcattgttgtttcttttccctgaagatgctcacagtttgatagttcacatatgaggaataattatgcaagacccagatgaaaaccctaataaaaaatattctgatttttaagtgtatattactctctttggggaagagagggtgagataaggttagacattaaagacttgaatctccaccattgattttacgaaacccaatctcaggggatttgggggtttttttagatttcaaaacaacattgatatcttacaagactcgtacttgcttttctctgggaaatagccaaatctagatgacaagcataaataagaaaggagctgtatccccattttgatataataattgctactgctactgtataccagagagcaaaagtaagaaatggcatagtggttttaagaaagctttaaaatgaaagctaacaagagactgaaacggaaagtgtgatttataccatgactgctggttttagggttttttttagggatttctcttcaaataggatacattaccaaattgtcagaaatagcttgatttcagagttagtgcactataacatcttttgcttcgaaaagtgattatgattctaccatcagtccagttggtgacaaatacgtggatcctgttgtgtgaaagtgtactcatataaccaactattattgcagtcactggccctgcaaagcagtggttgcatttaaaatttcaaccttctttgtaacactgaatcgcatacacacactcacaagatcaacgctgttttaaacaattactaaatccaagtatatcttttttaaagattttcaataaaatctaatgttcacagaaaaatctcataaatgtcaaaattgctgtgggcagaaaaggggggatacagtattttcttgatctgttttagcaatttatagatttcaggccgtggtttataagaatgctaaatgttctggaattacagctgttactgatcagtgattgagccagatttgtgttattgcaccatgttcttgcttgtacctagagagcagtaaagcctcaatataataaaaagcacctgcattttaaatgaaatttcaattgaaaaacttaacagcccttcaaattgcagaatttaacgcagcccagtaaagcttaaataacactttgcccccgcaggctgaggggtttgcattcaaggtgacttgattgtgtcgcatggtgaaattatttacaattaaggaatttctctggagggctgcagaatatttgctgttccacaacacctatgcaggcatatggtgggcctttttcagtattcatggagcctcctgaacctttatgataattgaatcagttagagtgctgagtggagcaagcataaaacctgttaacctaaaggtcagatctgagctcggtacctatttggggaccccatagagcactgcactgactgagggatggtaacaggatgtgtaggttttggaggcccatatgtccattcatgaccagtgacttgtctcacagccatgcaacccttgcctcctgtgctgacttagcaggggataaagtgagagaaagcctgggctaatcagggggtcgctcagctcctcctaactggattgtcctatgtgtctttgcttctgtgctgctgatgctctgccctgtgctgacatgacctccctggcagtggcacaactggagctgggtggaggcccgggaagcggggctaccaacttcagtctcctcaaacaagctggcgatgtggaggaaaatccaggtccggtgtctaagggcgaagagctgattaaggagaacatgcacatgaagctgtacatggagggcaccgtggacaaccatcacttcaagtgcacatccgagggcgaaggcaagccctacgagggcacccagaccatgagaatcaaggtggtcgagggcggccctctccccttcgccttcgacatcctggctactagcttcctctacggcagcaagaccttcatcaaccacacccagggcatccccgacttcttcaagcagtccttccctgagggcttcacatgggagagagtcaccacatacgaagacgggggcgtgctgaccgctacccaggacaccagcctccaggacggctgcctcatctacaacgtcaagatcagaggggtgaacttcacatccaacggccctgtgatgcagaagaaaacactcggctgggaggccttcaccgagacgctgtaccccgctgacggcggcctggaaggcagaaacgacatggccctgaagctcgtgggcgggagccatctgatcgcaaacgccaagaccacatatagatccaagaaacccgctaagaacctcaagatgcctggcgtctactatgtggactacagactggaaagaatcaaggaggccaacaacgagacctacgtcgagcagcacgaggtggcagtggccagatactgcgacctccctagcaaactggggcacaaacttaattgaccttcagaccttggcactggaggtggcccggcagaagcgcggcatcgtggatcagtgctgcaccagcatctgctctctctaccaactggagaactactgcaactaggcccaccactaccctgtccacccctctgcaatgaataaaacctttgaaagagcactacaagttgtgtgtacatgcgtgcatgtgcatatgtggtgcggggggaacatgagtggggctggctggagtggcgatgataagctgtcaaacatgagaattcttgaagacgaaagggcctcgtgatacgcctatttttataggttaatgtcatgataataatggtttcttagacgtttaaacgggggaggctaactgaaacacggaaggagacaataccggaaggaacccgcgctatgacggcaataaaaagacagaataaaacgcacgggtgttgggtcgtttgttc

SEQ ID NO: 45 - DNA, 8229 bp, synthetic construct
  source 1..8229: mol_type = other DNA
ctgggcggttctgataacgagtaatcgttaatccgcaaataacgtaaaaacccgcttcggcgggtttttttatggggggagtttagggaaagagcatttgtcagaatatttaagggcgcctgtcactttgcttgatatatgagaattatttaaccttataaatgagaaaaaagcaacgcactttaaataagatacgttgctttttcgattgatgaacacctataattaaactattcatctattatttatgattttttgtatatacaatatttctagtttgttaaagagaattaagaaaataaatctcgaaaataataaagggaaaatcagtttttgatatcaaaattatacatgtcaacgataatacaaaatataatacaaactataagatgttatcagtatttattatgcatttagaataaattttgtgtcgcccttattcgactcactatagaagttcctattctctagaaagtataggaacttcacttcattttgctagcggtgtgagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccgttaaagatgatctcttacctgtttcagtactctggaaacagaatctactgaaacaagacaatatgtcgtgtttatcccatcaatttattggtgggatttttttcaacaccggtaccgcgttacataacttacggtaaatggcccgcctggctgaccgcccaacgacccccgcccattgacgtcaatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattgtgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggcgcgcgccaggtggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagctgagcaagaggtaagggtttaagggatggttggttggtggggtattaatgtttaattacctggagcacctgcctgaaatcactttttttcaggttggcaaccgcggacacccaagcggccgccaccatggattacaaagaccacgacggagactacaaggatcacgacattgattacaaggatgatgatgataaggccccaaagaagaagcggaaggtcggtatccacggagtcccagcagccggatccgccatgaaccagaagttcattctcggtctggacattggcattactagcgtgggatacggcttgattgactacgagactaagaacatcatcgatgccggcgtccgcctgttcccggaagccaacgtggagaacaatgagggccggaggtcgaagagaggctcccgccgcctgaagcggcggcgaatccaccggctggagagagtgaagctgctgctcaccgaatacgacctgatcaacaaagaacagatcccgacctccaacaacccgtaccagatcagagtgaagggtctgtcagaaatcctgtccaaggacgaactggcaatcgccctgctgcacctggccaagcggcgcggaatccacaacgtggatgtggctgccgacaaggaagaaaccgcttccgactccctgagcactaaggaccagatcaacaagaacgccaagttcttggagtcccgctacgtgtgcgagctgcagaaggaacggctggaaaacgaaggtcacgtgcgcggagtggaaaaccggttcctgacaaaggacattgtgcgcgaagcgaagaagatcattgatacccaaatgcagtactaccctgaaatcgacgagactttcaaggaaaagtacatttccctggtggaaacccggcgggaatacttcgaaggccccggacagggatcgccgttcggatggaacggggacctcaagaagtggtacgagatgctgatggggcactgtacctactttccgcaagaactgcgctccgtgaagtacgcgtactccgcggatctcttcaacgcgttgaatgacctgaacaacctgatcattcagagagacaattccgaaaagctcgagtaccacgagaagtatcacatcatcgagaatgtgttcaagcagaagaagaaaccgaccctcaagcaaatcgccaaggagattggcgtcaacccagaggacatcaagggatatcggattaccaagagcggcactcccgagtttacctctttcaagctgtttcatgatctgaagaaagtcgtgaaggaccatgccattctcgacgacattgatctcctgaatcagatcgcagagatcctgactatctaccaagacaaggactcgattgtggcagagctgggtcagctcgaatacctgatgtccgaggccgacaagcagtccatctccgaactgacagggtacacggggactcatagcctgtcgctgaagtgcatgaacatgatcattgatgaactgtggcacagctccatgaaccaaatggaagtgtttacctacctcaacatgcgccctaagaagtacgaactgaaaggctaccagcgcatccccaccgacatgatcgacgacgcgatcttgtcccctgtggtcaagaggaccttcattcaatccatcaacgtgatcaacaaggtcatcgaaaagtacggaataccagaagatatcatcattgagctcgctcgggagaacaactcggatgaccggaagaagttcatcaacaatcttcagaagaagaacgaagcgactcggaaacggatcaacgagatcatcggacagaccggaaaccagaacgccaaacggattgtcgaaaagattagactgcacgaccagcaggaagggaagtgcctgtactcactcgagtcaataccgctcgaggacctgttgaacaaccctaaccactatgaagtggaccacatcatccctcggtccgtgagcttcgacaactcgtaccacaacaaagtgctcgtgaagcagtccgaaaactccaagaaatccaacctgaccccgtaccaatacttcaattcgggaaagtcgaagctgtcgtacaaccagttcaaacaacacatactcaaccttagcaaaagccaggatcgcatttccaagaagaagaaggaatacctcctcgaggaaagggacatcaacaagttcgaagtgcagaaagagttcatcaatcgcaacttggtggataccagatatgccacccgggaactcaccaactatctcaaggcctacttttccgccaacaacatgaacgtgaaggtcaagaccatcaacgggtccttcactgactacctgagaaaggtctggaagttcaagaaggaacgcaaccacggatacaagcaccacgctgaggacgctctgatcatcgccaatgccgacttcctgttcaaggaaaacaagaagctgaaagctgtcaactcagtgctggaaaagcctgaaatcgagactaagcagctggatatccaagtggactctgaggacaactacagcgagatgttcatcatccctaaacaagtgcaggatatcaaggactttcgcaacttcaagtactcacaccgggtggacaagaaaccgaatagacagctgatcaacgacacgttgtattccacccggaagaaggataactcaacctacattgtgcagactatcaaggatatctacgccaaagataacactactctgaagaaacaattcgacaagtccccagagaagttcctgatgtaccagcacgacccccgaacctttgagaagcttgaagtgatcatgaagcagtacgccaacgagaagaacccgctggccaagtaccatgaagaaaccggagaatacctgaccaagtacagcaagaagaacaacggtcccattgtcaagagcctgaagtacatcggcaacaagctgggatcccacctcgacgtgacacatcagttcaagtcgtcgactaagaagcttgtgaagctgtcaatcaagaactatagattcgacgtgtacttgaccgaaaagggatacaagttcgtgaccatagcctatctgaacgtgttcaagaaagataactactactacatccccaaggacaagtaccaggagctcaaagaaaagaagaagatcaaagacaccgaccagttcattgcctccttctacaagaacgacctgatcaaactgaacggcgacctctacaagatcattggagtgaacagcgatgacaggaacatcattgagctggactactacgacatcaagtacaaggactactgcgagatcaacaacatcaagggcgaaccccggatcaagaaaactattggaaagaaaaccgagtccattgagaagttcaccactgacgtgctgggaaacctttacctccactccaccgagaaggcaccacaactgatcttcaagcgcggcctgggtggaaagcttaaaaggccggcggccacgaaaaaggccggccaggcaaaaaagaaaaagggtactagtgagggcaggggaagtctgctaacatgcggggacgtggaggaaaatcccggccccatgactgccctgaccgaaggtgctaagctgtttgagaaggagattccgtacatcaccgagctggaaggggacgtcgaaggaatgaagttcatcatcaagggagaaggaaccggggacgctacgactggaaccattaaggccaagtatatctgtaccactggagatctgccagtgccttgggccacccttgtgtcaaccctctcgtatggagtgcagtgttttgctaagtaccctagccacattaaggacttcttcaaatccgccatgccggaaggttatacccaagagcgcaccatttcttttgagggagatggagtgtacaagacccgcgcgatggtcacctatgagaggggatctatctacaaccgggtgactctgactggagaaaactttaagaaggacgggcatattcttcggaagaatgtcgccttccagtgccctcccagcatcctttacattctccccgacactgtgaacaacggaatccgcgtggagttcaatcaagcctacgacatcgagggggtgacggagaagctggtgaccaagtgtagccagatgaatcggccactggccggttcagcggctgtccacattccgcgctaccatcatatcacttatcacactaagctctccaaagaccgcgatgagaggagagatcacatgtgcctggtggaagtggtcaaggccgtcgatctcgatacctatcagtaagtcgactgaataagtgaactcgagatcagcctcgactgtgccttctagttgccagccatctgttgtttgcccctcccccgtgccttccttgaccctggaaggtgccactcccactgtcctttcctaataaaatgaggaaattgcatccccacttcagaagttcctatactttctagagaataggaacttcactatagagtcgaataagggcgacaccccctaattagcccgggcgaaaggcccagtctttcgactgagcctttcgttttatttgatgcctggcagttccctactctcgcatggggagtccccacactaccatcggcgctacggcgtttcacttctgagttcggcatggggtcaggtgggaccaccgcgctactgccgccaggcaaacaaggggtgttatgagccatattcaggtataaatgggctcgcgataatgttcagaattggttaattggttgtaacactgacccctatttgtttatttttctaaatacattcaaatatgtatccgctcatgagacaataaccctgataaatgcttcaataatattgaaaaaggaagaatatgagccatattcaacgggaaacgtcgaggccgcgattaaattccaacatggatgctgatttatatgggtataaatgggctcgcgataatgtcgggcaatcaggtgcgacaatctatcgcttgtatgggaagcccgatgcgccagagttgtttctgaaacatggcaaaggtagcgttgccaatgatgttacagatgagatggtcagactaaactggctgacggaatttatgccacttccgaccatcaagcattttatccgtactcctgatgatgcatggttactcaccactgcgatccccggaaaaacagcgttccaggtattagaagaatatcctgattcaggtgaaaatattgttgatgcgctggcagtgttcctgcgccggttgcactcgattcctgtttgtaattgtccttttaacagcgatcgcgtatttcgcctcgctcaggcgcaatcacgaatgaataacggtttggttgatgcgagtgattttgatgacgagcgtaatggctggcctgttgaacaagtctggaaagaaatgcataaacttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaaaagcagagcattacgctgacttgacgggacggcgcaagctcatgaccaaaatcccttaacgtgagttacgcgcgcgtcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttagcccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaaggcgagagtagggaactgccaggcatcaaactaagcagaaggcccctgacggatggcctttttgcgtttctacaaactctttctgtgttgtaaaacgacggccagtcttaagctcgggcccc

SEQ ID NO: 46 - RNA, 104 nt, synthetic construct
  source 1..104: mol_type = other RNA
gttaaagatgatctcttacctgtttcagtactctggaaacagaatctactgaaacaagacaatatgtcgtgtttatcccatcaatttattggtgggattttttt

SEQ ID NO: 47 - RNA, 21 nt, synthetic construct
  source 1..21: mol_type = other RNA
gttaaagatgatctcttacct

SEQ ID NO: 48 - DNA, 21 bp, synthetic construct
  source 1..21: mol_type = other DNA
gttaaagatgatctcttacct

SEQ ID NO: 49 - DNA, 21 bp, synthetic construct
  source 1..21: mol_type = other DNA
aggtaagagatcatctttaac

SEQ ID NO: 50 - DNA, 6254 bp, synthetic construct
  source 1..6254: mol_type = other DNA
gacggatcgggagatctgagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccgcgaagaagatcattgatacccgaaggtatcaatgatcttcttcgcttttttacccgatcccctatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgttaattaaccggtgccaccatgatgtcccgccttgataaatcaaaagtaataaacagtgctcttgagcttctcaatgaagttggtatagaagggttgacgactcggaaattggcgcaaaaactcggtgttgagcagccaaccttgtattggcatgttaaaaacaaacgagcactcctcgacgctttggcgatagagatgctggacaggcaccacacgcatttctgtcccctcgaaggagagtcatggcaggatttccttagaaataacgcaaagtccttcagatgtgcgctgcttagtcaccgcgacggcgcaaaagttcatctcggcactaggccaaccgagaaacagtacgagactctggagaaccaactggcgtttttgtgtcaacagggttttagtctcgaaaatgcgctctatgctctctctgcggttggccatttcaccctcggatgcgtactggaagatcaggagcaccaagtggccaaagaagaacgggaaacgccgactacggacagcatgcctccgttgctccggcaagctatagagctcttcgatcaccaaggcgctgagccagctttcttgttcggattggaacttattatatgcgggctcgaaaagcagcttaaatgcgagtcaggttaagcggccgctcgagtctagagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattaattctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtc

SEQ ID NO: 51 - DNA, 6254 bp, synthetic construct
  source 1..6254: mol_type = other DNA
gacggatcgggagatctgagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccgcaaatcgccaaggagattggcgaaccaatctccttggcgatttgcttttttacccgatcccctatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgttaattaaccggtgccaccatgatgtcccgccttgataaatcaaaagtaataaacagtgctcttgagcttctcaatgaagttggtatagaagggttgacgactcggaaattggcgcaaaaactcggtgttgagcagccaaccttgtattggcatgttaaaaacaaacgagcactcctcgacgctttggcgatagagatgctggacaggcaccacacgcatttctgtcccctcgaaggagagtcatggcaggatttccttagaaataacgcaaagtccttcagatgtgcgctgcttagtcaccgcgacggcgcaaaagttcatctcggcactaggccaaccgagaaacagtacgagactctggagaaccaactggcgtttttgtgtcaacagggttttagtctcgaaaatgcgctctatgctctctctgcggttggccatttcaccctcggatgcgtactggaagatcaggagcaccaagtggccaaagaagaacgggaaacgccgactacggacagcatgcctccgttgctccggcaagctatagagctcttcgatcaccaaggcgctgagccagctttcttgttcggattggaacttattatatgcgggctcgaaaagcagcttaaatgcgagtcaggttaagcggccgctcgagtctagagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattaattctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtc

SEQ ID NO: 52 - DNA, 6254 bp, synthetic construct
  source 1..6254: mol_type = other DNA
gacggatcgggagatctgagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccgcacagctccatgaaccaaatcgaaatttggttcatggagctgtgcttttttacccgatcccctatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgttaattaaccggtgccaccatgatgtcccgccttgataaatcaaaagtaataaacagtgctcttgagcttctcaatgaagttggtatagaagggttgacgactcggaaattggcgcaaaaactcggtgttgagcagccaaccttgtattggcatgttaaaaacaaacgagcactcctcgacgctttggcgatagagatgctggacaggcaccacacgcatttctgtcccctcgaaggagagtcatggcaggatttccttagaaataacgcaaagtccttcagatgtgcgctgcttagtcaccgcgacggcgcaaaagttcatctcggcactaggccaaccgagaaacagtacgagactctggagaaccaactggcgtttttgtgtcaacagggttttagtctcgaaaatgcgctctatgctctctctgcggttggccatttcaccctcggatgcgtactggaagatcaggagcaccaagtggccaaagaagaacgggaaacgccgactacggacagcatgcctccgttgctccggcaagctatagagctcttcgatcaccaaggcgctgagccagctttcttgttcggattggaacttattatatgcgggctcgaaaagcagcttaaatgcgagtcaggttaagcggccgctcgagtctagagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattaattctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtc

SEQ ID NO: 53 - DNA, 6254 bp, synthetic construct
  source 1..6254: mol_type = other DNA
gacggatcgggagatctgagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccggaccttcattcaatccatcacgaatgatggattgaatgaaggtccttttttacccgatcccctatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgttaattaaccggtgccaccatgatgtcccgccttgataaatcaaaagtaataaacagtgctcttgagcttctcaatgaagttggtatagaagggttgacgactcggaaattggcgcaaaaactcggtgttgagcagccaaccttgtattggcatgttaaaaacaaacgagcactcctcgacgctttggcgatagagatgctggacaggcaccacacgcatttctgtcccctcgaaggagagtcatggcaggatttccttagaaataacgcaaagtccttcagatgtgcgctgcttagtcaccgcgacggcgcaaaagttcatctcggcactaggccaaccgagaaacagtacgagactctggagaaccaactggcgtttttgtgtcaacagggttttagtctcgaaaatgcgctctatgctctctctgcggttggccatttcaccctcggatgcgtactggaagatcaggagcaccaagtggccaaagaagaacgggaaacgccgactacggacagcatgcctccgttgctccggcaagctatagagctcttcgatcaccaaggcgctgagccagctttcttgttcggattggaacttattatatgcgggctcgaaaagcagcttaaatgcgagtcaggttaagcggccgctcgagtctagagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattaattctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtc

SEQ ID NO: 54 - DNA, 6254 bp, synthetic construct
  source 1..6254: mol_type = other DNA
gacggatcgggagatctgagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccggaagaagttcatcaacaatccgaagattgttgatgaacttcttccttttttacccgatcccctatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgttaattaaccggtgccaccatgatgtcccgccttgataaatcaaaagtaataaacagtgctcttgagcttctcaatgaagttggtatagaagggttgacgactcggaaattggcgcaaaaactcggtgttgagcagccaaccttgtattggcatgttaaaaacaaacgagcactcctcgacgctttggcgatagagatgctggacaggcaccacacgcatttctgtcccctcgaaggagagtcatggcaggatttccttagaaataacgcaaagtccttcagatgtgcgctgcttagtcaccgcgacggcgcaaaagttcatctcggcactaggccaaccgagaaacagtacgagactctggagaaccaactggcgtttttgtgtcaacagggttttagtctcgaaaatgcgctctatgctctctctgcggttggccatttcaccctcggatgcgtactggaagatcaggagcaccaagtggccaaagaagaacgggaaacgccgactacggacagcatgcctccgttgctccggcaagctatagagctcttcgatcaccaaggcgctgagccagctttcttgttcggattggaacttattatatgcgggctcgaaaagcagcttaaatgcgagtcaggttaagcggccgctcgagtctagagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattaattctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtc

SEQ ID NO: 55 - RNA, 52 nt, synthetic construct
  source 1..52: mol_type = other RNA
gcgaagaagatcattgatacccgaaggtatcaatgatcttcttcgctttttt

SEQ ID NO: 56 - RNA, 52 nt, synthetic construct
  source 1..52: mol_type = other RNA
gcaaatcgccaaggagattggcgaaccaatctccttggcgatttgctttttt

SEQ ID NO: 57 - RNA, 52 nt, synthetic construct
  source 1..52: mol_type = other RNA
gcacagctccatgaaccaaatcgaaatttggttcatggagctgtgctttttt

SEQ ID NO: 58 - RNA, 52 nt, synthetic construct
  source 1..52: mol_type = other RNA
ggaccttcattcaatccatcacgaatgatggattgaatgaaggtcctttttt

SEQ ID NO: 59 - RNA, 52 nt, synthetic construct
  source 1..52: mol_type = other RNA
ggaagaagttcatcaacaatccgaagattgttgatgaacttcttcctttttt

SEQ ID NO: 60 - protein, 1057 aa, synthetic construct
MNQKFILGLDIGITSVGYGLIDYETKNIIDAGVRLFPEANVENNEGRRSKRGSRRLKRRRIHRLERVKLLLTEYDLINKEQIPTSNNPYQIRVKGLSEILSKDELAIALLHLAKRRGIHNVDVAADKEETASDSLSTKDQINKNAKFLESRYVCELQKERLENEGHVRGVENRFLTKDIVREAKKIIDTQMQYYPEIDETFKEKYISLVETRREYFEGPGQGSPFGWNGDLKKWYEMLMGHCTYFPQELRSVKYAYSADLFNALNDLNNLIIQRDNSEKLEYHEKYHIIENVFKQKKKPTLKQIAKEIGVNPEDIKGYRITKSGTPEFTSFKLFHDLKKVVKDHAILDDIDLLNQIAEILTIYQDKDSIVAELGQLEYLMSEADKQSISELTGYTGTHSLSLKCMNMIIDELWHSSMNQMEVFTYLNMRPKKYELKGYQRIPTDMIDDAILSPVVKRTFIQSINVINKVIEKYGIPEDIIIELARENNSDDRKKFINNLQKKNEATRKRINEIIGQTGNQNAKRIVEKIRLHDQQEGKCLYSLESIPLEDLLNNPNHYEVDHIIPRSVSFDNSYHNKVLVKQSENSKKSNLTPYQYFNSGKSKLSYNQFKQHILNLSKSQDRISKKKKEYLLEERDINKFEVQKEFINRNLVDTRYATRELTNYLKAYFSANNMNVKVKTINGSFTDYLRKVWKFKKERNHGYKHHAEDALIIANADFLFKENKKLKAVNSVLEKPEIETKQLDIQVDSEDNYSEMFIIPKQVQDIKDFRNFKYSHRVDKKPNRQLINDTLYSTRKKDNSTYIVQTIKDIYAKDNTTLKKQFDKSPEKFLMYQHDPRTFEKLEVIMKQYANEKNPLAKYHEETGEYLTKYSKKNNGPIVKSLKYIGNKLGSHLDVTHQFKSSTKKLVKLSIKNYRFDVYLTEKGYKFVTIAYLNVFKKDNYYYIPKDKYQELKEKKKIKDTDQFIASFYKNDLIKLNGDLYKIIGVNSDDRNIIELDYYDIKYKDYCEINNIKGEPRIKKTIGKKTESIEKFTTDVLGNLYLHSTEKAPQLIFKRGL

SEQ ID NO: 61 - DNA, 3171 bp, synthetic construct
  source 1..3171: mol_type = other DNA
atgaaccagaagttcattctcggtctggacattggcattactagcgtgggatacggcttgattgactacgagactaagaacatcatcgatgccggcgtccgcctgttcccggaagccaacgtggagaacaatgagggccggaggtcgaagagaggctcccgccgcctgaagcggcggcgaatccaccggctggagagagtgaagctgctgctcaccgaatacgacctgatcaacaaagaacagatcccgacctccaacaacccgtaccagatcagagtgaagggtctgtcagaaatcctgtccaaggacgaactggcaatcgccctgctgcacctggccaagcggcgcggaatccacaacgtggatgtggctgccgacaaggaagaaaccgcttccgactccctgagcactaaggaccagatcaacaagaacgccaagttcttggagtcccgctacgtgtgcgagctgcagaaggaacggctggaaaacgaaggtcacgtgcgcggagtggaaaaccggttcctgacaaaggacattgtgcgcgaagcgaagaagatcattgatacccaaatgcagtactaccctgaaatcgacgagactttcaaggaaaagtacatttccctggtggaaacccggcgggaatacttcgaaggccccggacagggatcgccgttcggatggaacggggacctcaagaagtggtacgagatgctgatggggcactgtacctactttccgcaagaactgcgctccgtgaagtacgcgtactccgcggatctcttcaacgcgttgaatgacctgaacaacctgatcattcagagagacaattccgaaaagctcgagtaccacgagaagtatcacatcatcgagaatgtgttcaagcagaagaagaaaccgaccctcaagcaaatcgccaaggagattggcgtcaacccagaggacatcaagggatatcggattaccaagagcggcactcccgagtttacctctttcaagctgtttcatgatctgaagaaagtcgtgaaggaccatgccattctcgacgacattgatctcctgaatcagatcgcagagatcctgactatctaccaagacaaggactcgattgtggcagagctgggtcagctcgaatacctgatgtccgaggccgacaagcagtccatctccgaactgacagggtacacggggactcatagcctgtcgctgaagtgcatgaacatgatcattgatgaactgtggcacagctccatgaaccaaatggaagtgtttacctacctcaacatgcgccctaagaagtacgaactgaaaggctaccagcgcatccccaccgacatgatcgacgacgcgatcttgtcccctgtggtcaagaggaccttcattcaatccatcaacgtgatcaacaaggtcatcgaaaagtacggaataccagaagatatcatcattgagctcgctcgggagaacaactcggatgaccggaagaagttcatcaacaatcttcagaagaagaacgaagcgactcggaaacggatcaacgagatcatcggacagaccggaaaccagaacgccaaacggattgtcgaaaagattagactgcacgaccagcaggaagggaagtgcctgtactcactcgagtcaataccgctcgaggacctgttgaacaaccctaaccactatgaagtggaccacatcatccctcggtccgtgagcttcgacaactcgtaccacaacaaagtgctcgtgaagcagtccgaaaactccaagaaatccaacctgaccccgtaccaatacttcaattcgggaaagtcgaagctgtcgtacaaccagttcaaacaacacatactcaaccttagcaaaagccaggatcgcatttccaagaagaagaaggaatacctcctcgaggaaagggacatcaacaagttcgaagtgcagaaagagttcatcaatcgcaacttggtggataccagatatgccacccgggaactcaccaactatctcaaggcctacttttccgccaacaacatgaacgtgaaggtcaagaccatcaacgggtccttcactgactacctgagaaaggtctggaagttcaagaaggaacgcaaccacggatacaagcaccacgctgaggacgctctgatcatcgccaatgccgacttcctgttcaaggaaaacaagaagctgaaagctgtcaactcagtgctggaaaagcctgaaatcgagactaagcagctggatatccaagtggactctgaggacaactacagcgagatgttcatcatccctaaacaagtgcaggatatcaaggactttcgcaacttcaagtactcacaccgggtggacaagaaaccgaatagacagctgatcaacgacacgttgtattccacccggaagaaggataactcaacctacattgtgcagactatcaaggatatctacgccaaagataacactactctgaagaaacaattcgacaagtccccagagaagttcctgatgtaccagcacgacccccgaacctttgagaagcttgaagtgatcatgaagcagtacgccaacgagaagaacccgctggccaagtaccatgaagaaaccggagaatacctgaccaagtacagcaagaagaacaacggtcccattgtcaagagcctgaagtacatcggcaacaagctgggatcccacctcgacgtgacacatcagttcaagtcgtcgactaagaagcttgtgaagctgtcaatcaagaactatagattcgacgtgtacttgaccgaaaagggatacaagttcgtgaccatagcctatctgaacgtgttcaagaaagataactactactacatccccaaggacaagtaccaggagctcaaagaaaagaagaagatcaaagacaccgaccagttcattgcctccttctacaagaacgacctgatcaaactgaacggcgacctctacaagatcattggagtgaacagcgatgacaggaacatcattgagctggactactacgacatcaagtacaaggactactgcgagatcaacaacatcaagggcgaaccccggatcaagaaaactattggaaagaaaaccgagtccattgagaagttcaccactgacgtgctgggaaacctttacctccactccaccgagaaggcaccacaactgatcttcaagcgcggcctg

SEQ ID NO: 62 - DNA, 624 bp, synthetic construct
  source 1..624: mol_type = other DNA
atgatgtcccgccttgataaatcaaaagtaataaacagtgctcttgagcttctcaatgaagttggtatagaagggttgacgactcggaaattggcgcaaaaactcggtgttgagcagccaaccttgtattggcatgttaaaaacaaacgagcactcctcgacgctttggcgatagagatgctggacaggcaccacacgcatttctgtcccctcgaaggagagtcatggcaggatttccttagaaataacgcaaagtccttcagatgtgcgctgcttagtcaccgcgacggcgcaaaagttcatctcggcactaggccaaccgagaaacagtacgagactctggagaaccaactggcgtttttgtgtcaacagggttttagtctcgaaaatgcgctctatgctctctctgcggttggccatttcaccctcggatgcgtactggaagatcaggagcaccaagtggccaaagaagaacgggaaacgccgactacggacagcatgcctccgttgctccggcaagctatagagctcttcgatcaccaaggcgctgagccagctttcttgttcggattggaacttattatatgcgggctcgaaaagcagcttaaatgcgagtcaggttaa

SEQ ID NO: 63 - protein, 207 aa, synthetic construct
MMSRLDKSKVINSALELLNEVGIEGLTTRKLAQKLGVEQPTLYWHVKNKRALLDALAIEMLDRHHTHFCPLEGESWQDFLRNNAKSFRCALLSHRDGAKVHLGTRPTEKQYETLENQLAFLCQQGFSLENALYALSAVGHFTLGCVLEDQEHQVAKEERETPTTDSMPPLLRQAIELFDHQGAEPAFLFGLELIICGLEKQLKCESG

SEQ ID NO: 64 - DNA, 3156 bp, synthetic construct
  source 1..3156: mol_type = other DNA
aagcggaactatatcctgggactggacatcggaattacctccgtgggatacggcatcatcgattacgagactagggacgtgattgacgccggcgtgagactctttaaggaggccaacgtggaaaacaacgaaggtcgcagatccaagcggggtgcaagacgcctgaagcgccggaggagacatcggatacagcgcgtgaagaagctccttttcgactacaacctcctcactgaccactcggaattgtccggtatcaacccctacgaagcccgcgtgaaaggcctgagccagaagctgtccgaagaggagtttagcgcagccctgctgcacctggctaagcgaaggggggtgcacaacgtgaacgaggtggaggaggacactggcaacgaactgtccaccaaggagcagatttcacggaactcgaaggcgctggaagagaaatatgtggccgagctgcagctggagaggctcaagaaggatggcgaagtccgggggagcatcaatcgcttcaagacctcggactacgtgaaggaagccaaacagctgttgaaggtgcagaaggcctaccaccaactggaccaatcattcattgacacttacatcgatctgcttgaaaccaggcgcacctactacgagggtcctggagaaggcagccctttcggatggaaggacatcaaggagtggtatgagatgctgatgggtcattgcacctactttccggaagaactgcgctcagtgaagtacgcgtacaacgctgacctctacaacgctctcaacgatctgaacaacctcgtgatcacccgggacgagaacgaaaagctggagtactacgaaaagttccagattatcgaaaacgtgttcaagcagaagaagaagcccaccctgaagcagattgcaaaggagatccttgtgaacgaggaggatattaagggctaccgggtcacctccaccgggaaaccagagttcactaatctcaaggtgtaccatgacattaaggacattactgcccgcaaggagatcattgaaaacgcggaactgctggaccaaatcgcgaagatcctgaccatctatcagagctccgaggatatccaggaggaacttactaacctcaattccgagctgacgcaggaagaaatcgagcaaattagcaacctgaagggttacactggaacccacaacctcagcttgaaagcgattaaccttattttggatgaactttggcacactaatgacaatcagatcgccattttcaaccggctgaaactggtgccgaagaaggtggacctgagccaacagaaggaaatcccgaccacccttgtggacgatttcatcctgtcacctgtggtgaagaggagcttcatccagtcgatcaaggtcatcaacgccatcataaagaagtacggccttcccaacgacatcatcatcgaactggcccgcgagaagaactccaaagatgcccagaagatgatcaacgagatgcagaagcgaaaccggcagacgaacgaacggatcgaggagatcatccggaccaccgggaaggaaaacgcgaagtacctgatcgagaaaatcaagctgcatgatatgcaggaagggaagtgtctctactccctggaggccattccgctggaggatttgctgaacaaccctttcaactacgaagtcgatcatatcattcctcgctccgtgtccttcgataactccttcaacaataaggtcctcgtgaagcaggaggagaactcgaagaagggcaacagaaccccgttccagtacctctcgtcgtccgactccaagatcagctacgaaactttcaagaagcacattctgaacctggccaagggcaaagggagaattagcaagaccaagaaggaatacctcctggaagagagagacatcaaccgcttctcggtgcaaaaggatttcatcaaccgcaacctggtcgataccagatacgccaccaggggactgatgaacctcctgcggtcctacttccgggtcaacaatctggacgtgaaggtcaaatccatcaacgggggctttacttctttcctgcgccggaagtggaagttcaagaaggaacggaacaagggatacaagcaccacgctgaagatgccctgattattgccaacgccgacttcatctttaaggaatggaaaaagctggacaaggctaagaaggtcatggagaaccagatgttcgaagaaaagcaggccgagtccatgcccgaaatcgaaaccgagcaggaatacaaggagatcttcatcacaccgcaccaaatcaagcacatcaaggacttcaaggattacaagtacagccaccgggtggacaagaagcctaacagagagcttatcaacgacaccctgtactccacgcgcaaggacgacaagggaaacacattgatcgtgaacaacctgaacggactgtatgacaaggacaatgacaaactgaagaagctgatcaacaaatcgccggaaaagctcctgatgtaccatcacgaccctcaaacctaccagaaactgaagctcatcatggagcagtacggcgacgaaaagaatcccctgtacaaatactacgaggagactggaaattacctgactaagtactccaagaaggataacggccccgtgatcaagaagattaagtactacggaaacaaactgaacgcacatctcgacatcaccgatgattatccaaactcccgcaacaaagtcgtgaagctctccctcaaaccgtaccgcttcgacgtgtacctggataatggggtgtacaagttcgtgaccgtgaagaacctggacgtcattaagaaggaaaactactacgaagtgaactcaaagtgctacgaggaagccaagaagctcaagaagatcagcaaccaggccgagttcatcgcatcgttttacaacaatgacctcattaagattaatggagaactgtacagagtgatcggcgtgaacaacgacctcctgaaccggattgaagtgaacatgatcgatattacctaccgggagtatctggagaacatgaacgacaagcgcccaccgagaatcatcaaaactattgcctccaagacccaatccattaagaaatactccaccgacatcctgggcaacctgtacgaggtcaagtcgaagaagcacccccagattatcaagaaggga

SEQ ID NO: 65 - protein, 1052 aa, synthetic construct
KRNYILGLDIGITSVGYGIIDYETRDVIDAGVRLFKEANVENNEGRRSKRGARRLKRRRRHRIQRVKKLLFDYNLLTDHSELSGINPYEARVKGLSQKLSEEEFSAALLHLAKRRGVHNVNEVEEDTGNELSTKEQISRNSKALEEKYVAELQLERLKKDGEVRGSINRFKTSDYVKEAKQLLKVQKAYHQLDQSFIDTYIDLLETRRTYYEGPGEGSPFGWKDIKEWYEMLMGHCTYFPEELRSVKYAYNADLYNALNDLNNLVITRDENEKLEYYEKFQIIENVFKQKKKPTLKQIAKEILVNEEDIKGYRVTSTGKPEFTNLKVYHDIKDITARKEIIENAELLDQIAKILTIYQSSEDIQEELTNLNSELTQEEIEQISNLKGYTGTHNLSLKAINLILDELWHTNDNQIAIFNRLKLVPKKVDLSQQKEIPTTLVDDFILSPVVKRSFIQSIKVINAIIKKYGLPNDIIIELAREKNSKDAQKMINEMQKRNRQTNERIEEIIRTTGKENAKYLIEKIKLHDMQEGKCLYSLEAIPLEDLLNNPFNYEVDHIIPRSVSFDNSFNNKVLVKQEENSKKGNRTPFQYLSSSDSKISYETFKKHILNLAKGKGRISKTKKEYLLEERDINRFSVQKDFINRNLVDTRYATRGLMNLLRSYFRVNNLDVKVKSINGGFTSFLRRKWKFKKERNKGYKHHAEDALIIANADFIFKEWKKLDKAKKVMENQMFEEKQAESMPEIETEQEYKEIFITPHQIKHIKDFKDYKYSHRVDKKPNRELINDTLYSTRKDDKGNTLIVNNLNGLYDKDNDKLKKLINKSPEKLLMYHHDPQTYQKLKLIMEQYGDEKNPLYKYYEETGNYLTKYSKKDNGPVIKKIKYYGNKLNAHLDITDDYPNSRNKVVKLSLKPYRFDVYLDNGVYKFVTVKNLDVIKKENYYEVNSKCYEEAKKLKKISNQAEFIASFYNNDLIKINGELYRVIGVNNDLLNRIEVNMIDITYREYLENMNDKRPPRIIKTIASKTQSIKKYSTDILGNLYEVKSKKHPQIIKKG

SEQ ID NO: 66 - DNA, 4557 bp, synthetic construct
  source 1..4557: mol_type = other DNA
cctgcaggcagctgcgcgctcgctcgctcactgaggccgcccgggcgtcgggcgacctttggtcgcccggcctcagtgagcgagcgagcgcgcagagagggagtggccaactccatcactaggggttcctgcggccgcacgcgtgctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgctagctaactaccggtgccaccatggccccaaagaagaagcggaaggtcggtagtactgtgggtactcgaagtggctgcgtaccacacccgtcgcatggatccaagcggaactatatcctgggactggacatcggaattacctccgtgggatacggcatcatcgattacgagactagggacgtgattgacgccggcgtgagactctttaaggaggccaacgtggaaaacaacgaaggtcgcagatccaagcggggtgcaagacgcctgaagcgccggaggagacatcggatacagcgcgtgaagaagctccttttcgactacaacctcctcactgaccactcggaattgtccggtatcaacccctacgaagcccgcgtgaaaggcctgagccagaagctgtccgaagaggagtttagcgcagccctgctgcacctggctaagcgaaggggggtgcacaacgtgaacgaggtggaggaggacactggcaacgaactgtccaccaaggagcagatttcacggaactcgaaggcgctggaagagaaatatgtggccgagctgcagctggagaggctcaagaaggatggcgaagtccgggggagcatcaatcgcttcaagacctcggactacgtgaaggaagccaaacagctgttgaaggtgcagaaggcctaccaccaactggaccaatcattcattgacacttacatcgatctgcttgaaaccaggcgcacctactacgagggtcctggagaaggcagccctttcggatggaaggacatcaaggagtggtatgagatgctgatgggtcattgcacctactttccggaagaactgcgctcagtgaagtacgcgtacaacgctgacctctacaacgctctcaacgatctgaacaacctcgtgatcacccgggacgagaacgaaaagctggagtactacgaaaagttccagattatcgaaaacgtgttcaagcagaagaagaagcccaccctgaagcagattgcaaaggagatccttgtgaacgaggaggatattaagggctaccgggtcacctccaccgggaaaccagagttcactaatctcaaggtgtaccatgacattaaggacattactgcccgcaaggagatcattgaaaacgcggaactgctggaccaaatcgcgaagatcctgaccatctatcagagctccgaggatatccaggaggaacttactaacctcaattccgagctgacgcaggaagaaatcgagcaaattagcaacctgaagggttacactggaacccacaacctcagcttgaaagcgattaaccttattttggatgaactttggcacactaatgacaatcagatcgccattttcaaccggctgaaactggtgccgaagaaggtggacctgagccaacagaaggaaatcccgaccacccttgtggacgatttcatcctgtcacctgtggtgaagaggagcttcatccagtcgatcaaggtcatcaacgccatcataaagaagtacggccttcccaacgacatcatcatcgaactggcccgcgagaagaactccaaagatgcccagaagatgatcaacgagatgcagaagcgaaaccggcagacgaacgaacggatcgaggagatcatccggaccaccgggaaggaaaacgcgaagtacctgatcgagaaaatcaagctgcatgatatgcaggaagggaagtgtctctactccctggaggccattccgctggaggatttgctgaacaaccctttcaactacgaagtcgatcatatcattcctcgctccgtgtccttcgataactccttcaacaataaggtcctcgtgaagcaggaggagaagtaagtatcaaggttacaagacaggtttaaggagaccaatagaaactgggctcgagaatgcgacgggtgtggtacgcagccacttcgagtacccacagtactacctgcttgtcgagacagagaagactcttgcgtttctgataggcacctattggtcttactgacatccactttgcctttctctccacagctcgaagaagggcaacagaaccccgttccagtacctctcgtcgtccgactccaagatcagctacgaaactttcaagaagcacattctgaacctggccaagggcaaagggagaattagcaagaccaagaaggaatacctcctggaagagagagacatcaaccgcttctcggtgcaaaaggatttcatcaaccgcaacctggtcgataccagatacgccaccaggggactgatgaacctcctgcggtcctacttccgggtcaacaatctggacgtgaaggtcaaatccatcaacgggggctttacttctttcctgcgccggaagtggaagttcaagaaggaacggaacaagggatacaagcaccacgctgaagatgccctgattattgccaacgccgacttcatctttaaggaatggaaaaagctggacaaggctaagaaggtcatggagaaccagatgttcgaagaaaagcaggccgagtccatgcccgaaatcgaaaccgagcaggaatacaaggagatcttcatcacaccgcaccaaatcaagcacatcaaggacttcaaggattacaagtacagccaccgggtggacaagaagcctaacagagagcttatcaacgacaccctgtactccacgcgcaaggacgacaagggaaacacattgatcgtgaacaacctgaacggactgtatgacaaggacaatgacaaactgaagaagctgatcaacaaatcgccggaaaagctcctgatgtaccatcacgaccctcaaacctaccagaaactgaagctcatcatggagcagtacggcgacgaaaagaatcccctgtacaaatactacgaggagactggaaattacctgactaagtactccaagaaggataacggccccgtgatcaagaagattaagtactacggaaacaaactgaacgcacatctcgacatcaccgatgattatccaaactcccgcaacaaagtcgtgaagctctccctcaaaccgtaccgcttcgacgtgtacctggataatggggtgtacaagttcgtgaccgtgaagaacctggacgtcattaagaaggaaaactactacgaagtgaactcaaagtgctacgaggaagccaagaagctcaagaagatcagcaaccaggccgagttcatcgcatcgttttacaacaatgacctcattaagattaatggagaactgtacagagtgatcggcgtgaacaacgacctcctgaaccggattgaagtgaacatgatcgatattacctaccgggagtatctggagaacatgaacgacaagcgcccaccgagaatcatcaaaactattgcctccaagacccaatccattaagaaatactccaccgacatcctgggcaacctgtacgaggtcaagtcgaagaagcacccccagattatcaagaagggaaaaaggccggcggccacgaaaaaggccggccaggcaaaaaagaaaaaggcttaagaattcctagagctcgctgatcagcctcgaaacttgtttattgcagcttataatggttacaaataaagcaatagcatcacaaatttcacaaataaagcatttttttcactgcattctagttgtggtttgtccaaactcatcaatgtatcttatcatgtctgtataccagcaggctttcctaggttcgaacgctgacgtcatcaacccgctccaaggaatcgcgggcccagtgtcactaggcgggaacacccagcgcgcgtgcgccctggcaggaagatggctgtgagggacaggggagtggcgccctgcaatatttgcatgtcgctatgtgttctgggaaatcaccataaacgtgaaatccctatcagtgatagagacttataagttccctatcagtgatagagacaccgacgggtgtggtacgcagccactgtttaagtactctgtgctggaaacagcacagaatctacttaaacaaggcaaaatgccgtgtttatctcgtcaacttgttggcgagattttttcacgtgcggaccgaggctgcagcgtcgtcctccctaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 67 - DNA, 4640 bp, synthetic construct
  source 1..4640: mol_type = other DNA
cctgcaggcagctgcgcgctcgctcgctcactgaggccgcccgggcgtcgggcgacctttggtcgcccggcctcagtgagcgagcgagcgcgcagagagggagtggccaactccatcactaggggttcctgcggccgcacgcgtgggccccagaagcctggtggttgtttgtccttctcaggggaaaagtgaggcggccccttggaggaaggggccgggcagaatgatctaatcggattccaagcagctcaggggattgtctttttctagcaccttcttgccactcctaagcgtcctccgtgaccccggctgggatttagcctggtgctgtgtcagccccggtctcccaggggcttcccagtggtccccaggaaccctcgacagggcccggtctctctcgtccagcaagggcagggacgggccacaggccaagggcgctagctaactaccggtgccaccatggccccaaagaagaagcggaaggtcggtagtactgtgggtactcgaagtggctgcgtaccacacccgtcgcatggatccaagcggaactatatcctgggactggacatcggaattacctccgtgggatacggcatcatcgattacgagactagggacgtgattgacgccggcgtgagactctttaaggaggccaacgtggaaaacaacgaaggtcgcagatccaagcggggtgcaagacgcctgaagcgccggaggagacatcggatacagcgcgtgaagaagctccttttcgactacaacctcctcactgaccactcggaattgtccggtatcaacccctacgaagcccgcgtgaaaggcctgagccagaagctgtccgaagaggagtttagcgcagccctgctgcacctggctaagcgaaggggggtgcacaacgtgaacgaggtggaggaggacactggcaacgaactgtccaccaaggagcagatttcacggaactcgaaggcgctggaagagaaatatgtggccgagctgcagctggagaggctcaagaaggatggcgaagtccgggggagcatcaatcgcttcaagacctcggactacgtgaaggaagccaaacagctgttgaaggtgcagaaggcctaccaccaactggaccaatcattcattgacacttacatcgatctgcttgaaaccaggcgcacctactacgagggtcctggagaaggcagccctttcggatggaaggacatcaaggagtggtatgagatgctgatgggtcattgcacctactttccggaagaactgcgctcagtgaagtacgcgtacaacgctgacctctacaacgctctcaacgatctgaacaacctcgtgatcacccgggacgagaacgaaaagctggagtactacgaaaagttccagattatcgaaaacgtgttcaagcagaagaagaagcccaccctgaagcagattgcaaaggagatccttgtgaacgaggaggatattaagggctaccgggtcacctccaccgggaaaccagagttcactaatctcaaggtgtaccatgacattaaggacattactgcccgcaaggagatcattgaaaacgcggaactgctggaccaaatcgcgaagatcctgaccatctatcagagctccgaggatatccaggaggaacttactaacctcaattccgagctgacgcaggaagaaatcgagcaaattagcaacctgaagggttacactggaacccacaacctcagcttgaaagcgattaaccttattttggatgaactttggcacactaatgacaatcagatcgccattttcaaccggctgaaactggtgccgaagaaggtggacctgagccaacagaaggaaatcccgaccacccttgtggacgatttcatcctgtcacctgtggtgaagaggagcttcatccagtcgatcaaggtcatcaacgccatcataaagaagtacggccttcccaacgacatcatcatcgaactggcccgcgagaagaactccaaagatgcccagaagatgatcaacgagatgcagaagcgaaaccggcagacgaacgaacggatcgaggagatcatccggaccaccgggaaggaaaacgcgaagtacctgatcgagaaaatcaagctgcatgatatgcaggaagggaagtgtctctactccctggaggccattccgctggaggatttgctgaacaaccctttcaactacgaagtcgatcatatcattcctcgctccgtgtccttcgataactccttcaacaataaggtcctcgtgaagcaggaggagaagtaagtatcaaggttacaagacaggtttaaggagaccaatagaaactgggctcgagaatgcgacgggtgtggtacgcagccacttcgagtacccacagtactacctgcttgtcgagacagagaagactcttgcgtttctgataggcacctattggtcttactgacatccactttgcctttctctccacagctcgaagaagggcaacagaaccccgttccagtacctctcgtcgtccgactccaagatcagctacgaaactttcaagaagcacattctgaacctggccaagggcaaagggagaattagcaagaccaagaaggaatacctcctggaagagagagacatcaaccgcttctcggtgcaaaaggatttcatcaaccgcaacctggtcgataccagatacgccaccaggggactgatgaacctcctgcggtcctacttccgggtcaacaatctggacgtgaaggtcaaatccatcaacgggggctttacttctttcctgcgccggaagtggaagttcaagaaggaacggaacaagggatacaagcaccacgctgaagatgccctgattattgccaacgccgacttcatctttaaggaatggaaaaagctggacaaggctaagaaggtcatggagaaccagatgttcgaagaaaagcaggccgagtccatgcccgaaatcgaaaccgagcaggaatacaaggagatcttcatcacaccgcaccaaatcaagcacatcaaggacttcaaggattacaagtacagccaccgggtggacaagaagcctaacagagagcttatcaacgacaccctgtactccacgcgcaaggacgacaagggaaacacattgatcgtgaacaacctgaacggactgtatgacaaggacaatgacaaactgaagaagctgatcaacaaatcgccggaaaagctcctgatgtaccatcacgaccctcaaacctaccagaaactgaagctcatcatggagcagtacggcgacgaaaagaatcccctgtacaaatactacgaggagactggaaattacctgactaagtactccaagaaggataacggccccgtgatcaagaagattaagtactacggaaacaaactgaacgcacatctcgacatcaccgatgattatccaaactcccgcaacaaagtcgtgaagctctccctcaaaccgtaccgcttcgacgtgtacctggataatggggtgtacaagttcgtgaccgtgaagaacctggacgtcattaagaaggaaaactactacgaagtgaactcaaagtgctacgaggaagccaagaagctcaagaagatcagcaaccaggccgagttcatcgcatcgttttacaacaatgacctcattaagattaatggagaactgtacagagtgatcggcgtgaacaacgacctcctgaaccggattgaagtgaacatgatcgatattacctaccgggagtatctggagaacatgaacgacaagcgcccaccgagaatcatcaaaactattgcctccaagacccaatccattaagaaatactccaccgacatcctgggcaacctgtacgaggtcaagtcgaagaagcacccccagattatcaagaagggaaaaaggccggcggccacgaaaaaggccggccaggcaaaaaagaaaaaggcttaagaattcctagagctcgctgatcagcctcgaaacttgtttattgcagcttataatggttacaaataaagcaatagcatcacaaatttcacaaataaagcatttttttcactgcattctagttgtggtttgtccaaactcatcaatgtatcttatcatgtctgtataccagcaggctttcctaggttcgaacgctgacgtcatcaacccgctccaaggaatcgcgggcccagtgtcactaggcgggaacacccagcgcgcgtgcgccctggcaggaagatggctgtgagggacaggggagtggcgccctgcaatatttgcatgtcgctatgtgttctgggaaatcaccataaacgtgaaatccctatcagtgatagagacttataagttccctatcagtgatagagacaccgacgggtgtggtacgcagccactgtttaagtactctgtgctggaaacagcacagaatctacttaaacaaggcaaaatgccgtgtttatctcgtcaacttgttggcgagattttttcacgtgcggaccgaggctgcagcgtcgtcctccctaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 68 - DNA, 4542 bp, synthetic construct
  source 1..4542: mol_type = other DNA
cctgcaggcagctgcgcgctcgctcgctcactgaggccgcccgggcgtcgggcgacctttggtcgcccggcctcagtgagcgagcgagcgcgcagagagggagtggccaactccatcactaggggttcctgcggccgcacgcgtgggccccagaagcctggtggttgtttgtccttctcaggggaaaagtgaggcggccccttggaggaaggggccgggcagaatgatctaatcggattccaagcagctcaggggattgtctttttctagcaccttcttgccactcctaagcgtcctccgtgaccccggctgggatttagcctggtgctgtgtcagccccggctagctaactaccggtgccaccatggccccaaagaagaagcggaaggtcggtagtactgtgggtactcgaagtggctgcgtaccacacccgtcgcatggatccaagcggaactatatcctgggactggacatcggaattacctccgtgggatacggcatcatcgattacgagactagggacgtgattgacgccggcgtgagactctttaaggaggccaacgtggaaaacaacgaaggtcgcagatccaagcggggtgcaagacgcctgaagcgccggaggagacatcggatacagcgcgtgaagaagctccttttcgactacaacctcctcactgaccactcggaattgtccggtatcaacccctacgaagcccgcgtgaaaggcctgagccagaagctgtccgaagaggagtttagcgcagccctgctgcacctggctaagcgaaggggggtgcacaacgtgaacgaggtggaggaggacactggcaacgaactgtccaccaaggagcagatttcacggaactcgaaggcgctggaagagaaatatgtggccgagctgcagctggagaggctcaagaaggatggcgaagtccgggggagcatcaatcgcttcaagacctcggactacgtgaaggaagccaaacagctgttgaaggtgcagaaggcctaccaccaactggaccaatcattcattgacacttacatcgatctgcttgaaaccaggcgcacctactacgagggtcctggagaaggcagccctttcggatggaaggacatcaaggagtggtatgagatgctgatgggtcattgcacctactttccggaagaactgcgctcagtgaagtacgcgtacaacgctgacctctacaacgctctcaacgatctgaacaacctcgtgatcacccgggacgagaacgaaaagctggagtactacgaaaagttccagattatcgaaaacgtgttcaagcagaagaagaagcccaccctgaagcagattgcaaaggagatccttgtgaacgaggaggatattaagggctaccgggtcacctccaccgggaaaccagagttcactaatctcaaggtgtaccatgacattaaggacattactgcccgcaaggagatcattgaaaacgcggaactgctggaccaaatcgcgaagatcctgaccatctatcagagctccgaggatatccaggaggaacttactaacctcaattccgagctgacgcaggaagaaatcgagcaaattagcaacctgaagggttacactggaacccacaacctcagcttgaaagcgattaaccttattttggatgaactttggcacactaatgacaatcagatcgccattttcaaccggctgaaactggtgccgaagaaggtggacctgagccaacagaaggaaatcccgaccacccttgtggacgatttcatcctgtcacctgtggtgaagaggagcttcatccagtcgatcaaggtcatcaacgccatcataaagaagtacggccttcccaacgacatcatcatcgaactggcccgcgagaagaactccaaagatgcccagaagatgatcaacgagatgcagaagcgaaaccggcagacgaacgaacggatcgaggagatcatccggaccaccgggaaggaaaacgcgaagtacctgatcgagaaaatcaagctgcatgatatgcaggaagggaagtgtctctactccctggaggccattccgctggaggatttgctgaacaaccctttcaactacgaagtcgatcatatcattcctcgctccgtgtccttcgataactccttcaacaataaggtcctcgtgaagcaggaggagaagtaagtatcaaggttacaagacaggtttaaggagaccaatagaaactgggctcgagaatgcgacgggtgtggtacgcagccacttcgagtacccacagtactacctgcttgtcgagacagagaagactcttgcgtttctgataggcacctattggtcttactgacatccactttgcctttctctccacagctcgaagaagggcaacagaaccccgttccagtacctctcgtcgtccgactccaagatcagctacgaaactttcaagaagcacattctgaacctggccaagggcaaagggagaattagcaagaccaagaaggaatacctcctggaagagagagacatcaaccgcttctcggtgcaaaaggatttcatcaaccgcaacctggtcgataccagatacgccaccaggggactgatgaacctcctgcggtcctacttccgggtcaacaatctggacgtgaaggtcaaatccatcaacgggggctttacttctttcctgcgccggaagtggaagttcaagaaggaacggaacaagggatacaagcaccacgctgaagatgccctgattattgccaacgccgacttcatctttaaggaatggaaaaagctggacaaggctaagaaggtcatggagaaccagatgttcgaagaaaagcaggccgagtccatgcccgaaatcgaaaccgagcaggaatacaaggagatcttcatcacaccgcaccaaatcaagcacatcaaggacttcaaggattacaagtacagccaccgggtggacaagaagcctaacagagagcttatcaacgacaccctgtactccacgcgcaaggacgacaagggaaacacattgatcgtgaacaacctgaacggactgtatgacaaggacaatgacaaactgaagaagctgatcaacaaatcgccggaaaagctcctgatgtaccatcacgaccctcaaacctaccagaaactgaagctcatcatggagcagtacggcgacgaaaagaatcccctgtacaaatactacgaggagactggaaattacctgactaagtactccaagaaggataacggccccgtgatcaagaagattaagtactacggaaacaaactgaacgcacatctcgacatcaccgatgattatccaaactcccgcaacaaagtcgtgaagctctccctcaaaccgtaccgcttcgacgtgtacctggataatggggtgtacaagttcgtgaccgtgaagaacctggacgtcattaagaaggaaaactactacgaagtgaactcaaagtgctacgaggaagccaagaagctcaagaagatcagcaaccaggccgagttcatcgcatcgttttacaacaatgacctcattaagattaatggagaactgtacagagtgatcggcgtgaacaacgacctcctgaaccggattgaagtgaacatgatcgatattacctaccgggagtatctggagaacatgaacgacaagcgcccaccgagaatcatcaaaactattgcctccaagacccaatccattaagaaatactccaccgacatcctgggcaacctgtacgaggtcaagtcgaagaagcacccccagattatcaagaagggaaaaaggccggcggccacgaaaaaggccggccaggcaaaaaagaaaaaggcttaagaattcctagagctcgctgatcagcctcgaaacttgtttattgcagcttataatggttacaaataaagcaatagcatcacaaatttcacaaataaagcatttttttcactgcattctagttgtggtttgtccaaactcatcaatgtatcttatcatgtctgtataccagcaggctttcctaggttcgaacgctgacgtcatcaacccgctccaaggaatcgcgggcccagtgtcactaggcgggaacacccagcgcgcgtgcgccctggcaggaagatggctgtgagggacaggggagtggcgccctgcaatatttgcatgtcgctatgtgttctgggaaatcaccataaacgtgaaatccctatcagtgatagagacttataagttccctatcagtgatagagacaccgggtgtggtacgcagccactgtttaagtactctgtgctggaaacagcacagaatctacttaaacaaggcaaaatgccgtgtttatctcgtcaacttgttggcgagattttttcacgtgcggaccgaggctgcagcgtcgtcctccctaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 69 - DNA, 4554 bp, synthetic construct
  source 1..4554: mol_type = other DNA
cctgcaggcagctgcgcgctcgctcgctcactgaggccgcccgggcgtcgggcgacctttggtcgcccggcctcagtgagcgagcgagcgcgcagagagggagtggccaactccatcactaggggttcctgcggccgcacgcgtgctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgctagctaactaccggtgccaccatggccccaaagaagaagcggaaggtcggtagtactgtgggtactcgaagtggctgcgtaccacacccgtcgcatggatccaagcggaactatatcctgggactggacatcggaattacctccgtgggatacggcatcatcgattacgagactagggacgtgattgacgccggcgtgagactctttaaggaggccaacgtggaaaacaacgaaggtcgcagatccaagcggggtgcaagacgcctgaagcgccggaggagacatcggatacagcgcgtgaagaagctccttttcgactacaacctcctcactgaccactcggaattgtccggtatcaacccctacgaagcccgcgtgaaaggcctgagccagaagctgtccgaagaggagtttagcgcagccctgctgcacctggctaagcgaaggggggtgcacaacgtgaacgaggtggaggaggacactggcaacgaactgtccaccaaggagcagatttcacggaactcgaaggcgctggaagagaaatatgtggccgagctgcagctggagaggctcaagaaggatggcgaagtccgggggagcatcaatcgcttcaagacctcggactacgtgaaggaagccaaacagctgttgaaggtgcagaaggcctaccaccaactggaccaatcattcattgacacttacatcgatctgcttgaaaccaggcgcacctactacgagggtcctggagaaggcagccctttcggatggaaggacatcaaggagtggtatgagatgctgatgggtcattgcacctactttccggaagaactgcgctcagtgaagtacgcgtacaacgctgacctctacaacgctctcaacgatctgaacaacctcgtgatcacccgggacgagaacgaaaagctggagtactacgaaaagttccagattatcgaaaacgtgttcaagcagaagaagaagcccaccctgaagcagattgcaaaggagatccttgtgaacgaggaggatattaagggctaccgggtcacctccaccgggaaaccagagttcactaatctcaaggtgtaccatgacattaaggacattactgcccgcaaggagatcattgaaaacgcggaactgctggaccaaatcgcgaagatcctgaccatctatcagagctccgaggatatccaggaggaacttactaacctcaattccgagctgacgcaggaagaaatcgagcaaattagcaacctgaagggttacactggaacccacaacctcagcttgaaagcgattaaccttattttggatgaactttggcacactaatgacaatcagatcgccattttcaaccggctgaaactggtgccgaagaaggtggacctgagccaacagaaggaaatcccgaccacccttgtggacgatttcatcctgtcacctgtggtgaagaggagcttcatccagtcgatcaaggtcatcaacgccatcataaagaagtacggccttcccaacgacatcatcatcgaactggcccgcgagaagaactccaaagatgcccagaagatgatcaacgagatgcagaagcgaaaccggcagacgaacgaacggatcgaggagatcatccggaccaccgggaaggaaaacgcgaagtacctgatcgagaaaatcaagctgcatgatatgcaggaagggaagtgtctctactccctggaggccattccgctggaggatttgctgaacaaccctttcaactacgaagtcgatcatatcattcctcgctccgtgtccttcgataactccttcaacaataaggtcctcgtgaagcaggaggagaagtaagtatcaaggttacaagacaggtttaaggagaccaatagaaactgggctcgagaatgcgacgggtgtggtacgcagccacttcgagtacccacagtactacctgcttgtcgagacagagaagactcttgcgtttctgataggcacctattggtcttactgacatccactttgcctttctctccacagctcgaagaagggcaacagaaccccgttccagtacctctcgtcgtccgactccaagatcagctacgaaactttcaagaagcacattctgaacctggccaagggcaaagggagaattagcaagaccaagaaggaatacctcctggaagagagagacatcaaccgcttctcggtgcaaaaggatttcatcaaccgcaacctggtcgataccagatacgccaccaggggactgatgaacctcctgcggtcctacttccgggtcaacaatctggacgtgaaggtcaaatccatcaacgggggctttacttctttcctgcgccggaagtggaagttcaagaaggaacggaacaagggatacaagcaccacgctgaagatgccctgattattgccaacgccgacttcatctttaaggaatggaaaaagctggacaaggctaagaaggtcatggagaaccagatgttcgaagaaaagcaggccgagtccatgcccgaaatcgaaaccgagcaggaatacaaggagatcttcatcacaccgcaccaaatcaagcacatcaaggacttcaaggattacaagtacagccaccgggtggacaagaagcctaacagagagcttatcaacgacaccctgtactccacgcgcaaggacgacaagggaaacacattgatcgtgaacaacctgaacggactgtatgacaaggacaatgacaaactgaagaagctgatcaacaaatcgccggaaaagctcctgatgtaccatcacgaccctcaaacctaccagaaactgaagctcatcatggagcagtacggcgacgaaaagaatcccctgtacaaatactacgaggagactggaaattacctgactaagtactccaagaaggataacggccccgtgatcaagaagattaagtactacggaaacaaactgaacgcacatctcgacatcaccgatgattatccaaactcccgcaacaaagtcgtgaagctctccctcaaaccgtaccgcttcgacgtgtacctggataatggggtgtacaagttcgtgaccgtgaagaacctggacgtcattaagaaggaaaactactacgaagtgaactcaaagtgctacgaggaagccaagaagctcaagaagatcagcaaccaggccgagttcatcgcatcgttttacaacaatgacctcattaagattaatggagaactgtacagagtgatcggcgtgaacaacgacctcctgaaccggattgaagtgaacatgatcgatattacctaccgggagtatctggagaacatgaacgacaagcgcccaccgagaatcatcaaaactattgcctccaagacccaatccattaagaaatactccaccgacatcctgggcaacctgtacgaggtcaagtcgaagaagcacccccagattatcaagaagggaaaaaggccggcggccacgaaaaaggccggccaggcaaaaaagaaaaaggcttaagaattcctagagctcgctgatcagcctcgaaacttgtttattgcagcttataatggttacaaataaagcaatagcatcacaaatttcacaaataaagcatttttttcactgcattctagttgtggtttgtccaaactcatcaatgtatcttatcatgtctgtataccagcaggctttcctaggttcgaacgctgacgtcatcaacccgctccaaggaatcgcgggcccagtgtcactaggcgggaacacccagcgcgcgtgcgccctggcaggaagatggctgtgagggacaggggagtggcgccctgcaatatttgcatgtcgctatgtgttctgggaaatcaccataaacgtgaaatccctatcagtgatagagacttataagttccctatcagtgatagagacaccgggtgtggtacgcagccactgtttaagtactctgtgctggaaacagcacagaatctacttaaacaaggcaaaatgccgtgtttatctcgtcaacttgttggcgagattttttcacgtgcggaccgaggctgcagcgtcgtcctccctaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg